(19) United States
(12) Patent Application Publication
(10) Pub. No.: US 2026/0325347 A1
(43) Pub. Date: Oct. 22, 2026

(54) ELASTOMERIC MATRICES COMPRISING POLYVINYL ALCOHOL

(71) Applicant: ABLETX LTD., Tel-Aviv (IL)

(72) Inventors: Nir BETSER, Tel-Aviv (IL); Itai ADIN, Beer-Sheva (IL); Keren HADAR, Holon (IL)

(73) Assignee: AbleTx Ltd., Tel-Aviv (IL)

(21) Appl. No.: 19/478,957

(22) PCT Filed: Apr. 18, 2024
(86) PCT No.: PCT/IL2024/050391

§ 371 Date: Oct. 27, 2025

Related U.S. Application Data
(60) Provisional application No. 63/462,269, filed on Apr. 27, 2023.
(60) Provisional application No. 63/463,034, filed on Apr. 30, 2023.

(51) Int. Cl. A61F 9/00

(52) U.S. Cl. CPC A61F 9/0017; A61F 2210/0004; A61F 2210/0057; A61F 2210/0061; A61F 2220/0008; A61F 2250/0067

(57) ABSTRACT

The present invention relates to shaped polyvinyl alcohol (PVOH) based elastomeric matrixes for ophthalmic uses and, more particularly, but not exclusively, to such matrixes that are degradable.

### RELATED APPLICATION/S

This application claims the benefit of priority of U.S. Provisional Patent Application Nos. 63/462,269 filed on Apr. 27, 2023 and 63/463,034, filed on Apr. 30, 2023, the contents of which are incorporated herein by reference in their entirety.

### FIELD AND BACKGROUND OF THE INVENTION

The present invention, in some embodiments thereof, relates to shaped polyvinyl alcohol (PVOH) based elastomeric matrixes for ophthalmic uses and, more particularly, but not exclusively, to such matrixes that are biodegradable or bioerodible.

Polyvinyl alcohol (PVOH) is water-soluble synthetic polymer represented by the formula (C₂H₄O)n. It is prepared by hydrolysis of polyvinyl acetates, which replaces the acetate groups with hydroxyl groups. PVOH has numerous applications, including biomedical and pharmaceutical applications such as artificial heart linings, artificial cartilages, catheters, skin and pancreas membranes. PVOH may be chemically or physically (i.e., not by covalent bonds) crosslinked.

During physical crosslinking, hydroxyl groups interact to form intra- and intermolecular hydrogen bonds resulting in formation of crystallites. Physical crosslinking is typically carried out by freeze-thaw with or without addition of solvents, resulting in the well-known cryogels. Alternatively, solutions of polyvinyl alcohol can be dried (Otsuka, E., & Suzuki, A., Journal of Applied Polymer Science, 2009, 114(1), 10-16, doi:10.1002/app.30546]. Various plasticizers can be added to improve flexibility, and the amount added is commonly less than 30%, often less than 10% [Mohsin, M., et al., Journal of Applied Polymer Science, 2011, 122(5), 3102-3109. doi:10.1002/app.34229; Lim, L. Y. et al., Drug Development and Industrial Pharmacy, 1994, 20(6), 1007-1020. doi:10.3109/03639049409038347; and Wu, W. et al., Journal of Polymers and the Environment, 2011, 20(1), 63-69. doi:10.1007/s10924-011-0364-7].

Zhang, B. et al., [Ceramics International, 2020, doi:10.1016/j.ceramint.2020.03.286] teach about the instructive role of mechanical properties of polyvinyl alcohol film in the process of YSZ tape calendaring.

U.S. Pat. No. 4,874,562 discloses a method of molding a polyvinyl alcohol contact lens.

U.S. Pat. No. 4,663,358 discloses a porous and transparent hydrated gel that is prepared from a poly(vinyl alcohol) solution in a mixed solvent consisting of water and a water-miscible organic solvent.

U.S. Pat. No. 10,513,588 discloses water-soluble polyvinyl alcohol film with plasticizer blend.

U.S. Pat. No. 9,724,230 discloses a dry eye treatment apparatus and methods which generally includes a patch or strip affixed to the skin of the upper and/or lower eyelids.

U.S. Pat. No. 5,137,728 discloses an ocular insert for use behind an eyelid, comprising a substantially circular disc having a concave posterior surface and a convex anterior surface.

U.S. Pat. No. 6,217,896 disclosing a conjunctival insert for topical delivery of medication or lubrication into the conjunctival spaces and upon the ocular surface of human eye.

U.S. Pat. No. 8,679,078 disclosing an ocular device for insertion into an eye and includes a body having an anterior surface and a posterior surface for placement on one of superior sclera and inferior sclera of the eye.

U.S. Pat. No. 3,416,530 disclosing a medication dispensing tablet for a human eye ball, the tablet comprising a cross-sectionally concavo-convex body of flexible material for insertion into the cul-de-sac of the conjunctiva.

U.S. Pat. No. 3,828,777 disclosing an ocular device for the controlled and continuous administration of drug to the eye comprising a body of microporous drug release rate controlling material which is insoluble in tear fluid.

U.S. Pat. No. 4,014,335 disclosing an ocular drug dispensing device for administering a drug at a controlled and continuous dosage unit rate to the eye to produce a local or systemic physiological or pharmacological effect.

U.S. Pat. No. 5,773,021 disclosing a bioadhesive ophthalmic insert, which is intended for the prolonged and controlled release of a medicinal substance. The insert comprises a composite polymeric material matrix in which the medicinal substance is incorporated.

U.S. Pat. No. 10,010,502 disclosing a non-degradable topical ocular drug delivery device includes about 0.001% to about 10% w/w of at least one myopia-suppressing agent complexed with an immobile acid within a crosslinked polymer matrix.

International Patent Application No. WO2022/016268 disclosing biocompatible polyvinyl alcohol (PVA) matrices, composed of blends of differently hydrolyzed PVAs.

U.S. Patent Application Publication No. US20220168142A1 disclosing implantable bioerodible inserts for delivering an active pharmaceutical ingredient to the eye.

U.S. Patent Application Publication No. US20170226298A1 disclosing water-soluble films including a polyvinyl alcohol (PVOH) resin blend and optionally one or more additional components such as plasticizers, fillers, surfactants, and other additives.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. An ophthalmic device having an elastomeric matrix, said elastomeric matrix comprising:
a. poly(vinyl alcohol) (PVOH);
b. one or more organic plasticizers; where a combined mass ratio of said one or more organic plasticizers to said PVOH is at least 2:1; and
c. water,
wherein said PVOH comprises PVOH of two or more types that differ from one another in one or both of hydrolysis degree (HD) and chain length.

Example 2. The ophthalmic device according to example 1, wherein each of said two or more types of PVOH constitutes at least 10% of the total amount of PVOH in the matrix.

Example 3. The ophthalmic device according to example 1 or example 2, wherein the two or more types of PVOH include one type that has long chains and is fully hydrolyzed, and one type that has at least one of:
a. short chains and is partially hydrolyzed;
b. long chains and is partially hydrolyzed;
c. short chains and is fully hydrolyzed.

Example 4. The ophthalmic device according to any one of examples 1-3, wherein:
a. short chains are of 200 to 2000 monomeric units;
b. long chains are of 2200 to 5000 monomeric units;
c. fully hydrolyzed PVOH is of hydrolysis degree of 97% or more; and
d. partially hydrolyzed is of hydrolysis degree of 95% or less.

Example 5. The ophthalmic device according to any one of examples 1-4, wherein said PVOH comprises PVOH of two or more types that differ from one another in hydrolysis degree (HD).

Example 6. The ophthalmic device according to any one of examples 1-5, wherein two types of the PVOH differ in chain length by at least 1000, and have similar degrees of hydrolysis, both between 97% and 100%.

Example 7. The ophthalmic device according to any one of examples 1-6, wherein a first type of said two or more types has a hydrolysis degree from 97% to 100%.

Example 8. The ophthalmic device according to any one of examples 1-7, wherein a second type of said two or more types has a hydrolysis degree is lower than 93%.

Example 9. The ophthalmic device according to any one of examples 1-8, wherein a second type of said two or more types has a hydrolysis degree from 80% to 93%.

Example 10. The ophthalmic device according to any one of examples 1-9, wherein a first type of said two or more types has chains with more than 2500 units.

Example 11. The ophthalmic device according to any one of examples 1-10, wherein a second type of said two or more types has chains with less than 1500 units.

Example 12. The ophthalmic device according to any one of examples 1-11, wherein a relation between a first type of PVOH and a second type of PVOH of said two or more types is from about 3:1 to about 1:3.

Example 13. The ophthalmic device according to any one of examples 1-12, wherein a first type of said two or more types has chains with more than 2500 units and hydrolysis degree of 97% to 100%, and a second type of said two or more types has chains with less than 1000 units and hydrolysis degree of 80% to 93%.

Example 14. The ophthalmic device according to any one of examples 1-13, wherein said second type of PVOH makes more than 50% of said PVOH.

Example 15. The ophthalmic device according to any one of examples 1-14, wherein said at least two types of PVOH determine rate of degradation of said ophthalmic device.

Example 16. The ophthalmic device according to any one of examples 1-15, wherein said rate of degradation comprises a rate by which a predetermined change in a predetermined mechanical property takes place in simulated tear fluid (STF).

Example 17. The ophthalmic device according to any one of examples 1-16, wherein a combined mass content of said PVOH and said one or more organic plasticizers is at least 70% wt of the total weight of the matrix excluding said water.

Example 18. The ophthalmic device according to any one of examples 1-17, wherein said elastomeric matrix is characterized by substantially isotropic swelling when being immersed in simulated tear fluid and at room temperature for 5 minutes.

Example 19. The ophthalmic device according to any one of examples 1-18, wherein said elastomeric matrix swells by less than 50% by volume when immersed in simulated tear fluid for 5 minutes at room temperature.

Example 20. The ophthalmic device according to any one of examples 1-19, wherein said water constitute less than 50% wt of the total mass content of the matrix.

Example 21. The ophthalmic device according to any one of examples 1-20, essentially devoid of covalent crosslinking.

Example 22. The ophthalmic device according to any one of examples 1-21, further comprising an active pharmaceutically agent.

Example 23. The ophthalmic device according to any one of examples 1-22, wherein the elastomeric matrix comprises the active pharmaceutically agent.

Example 24. The ophthalmic device according to any one of examples 1-23, wherein said ophthalmic device consists of ophthalmic acceptable ingredients.

Example 25. The ophthalmic device according to any one of examples 1-24, wherein at least 90% of said plasticizers (w/w) are ophthalmic demulcents.

Example 26. The ophthalmic device according to any one of examples 1-25, wherein said device weights between 1 mg and 50 mg or between 1 mg and 30 mg.

Example 27. The ophthalmic device according to any one of examples 1-28, wherein said ophthalmic device is configured to be positioned on a surface of an eye.

Example 28. The ophthalmic device according to any one of examples 1-27, wherein said surface of the eye is outside a cornea of the eye, and at least partially underneath only one of the upper eyelid or the lower eyelid.

Example 29. The ophthalmic device according to any one of examples 1-28, wherein said device is configured for wearing away from the fornix.

Example 30. The ophthalmic device according to any one of examples 1-29, configured to deliver at least one pharmaceutically active agent to a surface an eye for between 5 minutes and 24 hours.

Example 31. The ophthalmic device according to any one of examples 1-30, configured to reside between an eyeball and an eyelid, and to move with the eyelid when the eyelid moves in respect to the eyeball.

Example 32. The ophthalmic device of any one of examples 1-31, configured to reside between an eyeball and an eyelid, and to stay with the eyelid when the eyeball moves in respect to the eyelid.

Example 33. The ophthalmic device according to any one of examples 1-32, having:
a. a posterior surface, which in use is adjacent to an eye surface; and
b. an anterior surface, which defines heights above said posterior surface;
wherein two of said heights, distanced from each other by a distance of at most 1 mm, differ from one another by a height difference of at least 1 mm.

Example 34. The ophthalmic device according to any one of examples 1-32, having:
a. a posterior surface; and
b. an anterior surface that when the ophthalmic device is disposed in a relaxed configuration on a planar surface with the posterior surface adjacent to the planar surface defines heights above the planar surface,
wherein two of said heights, distanced from each other by a distance of at most 1 mm, differ from one another by a height difference of at least 1 mm.

Example 35. An ophthalmic device according to any of examples 1 to 32, having:
a. a posterior surface which is adjacent to an eye surface when said ophthalmic device is worn;
b. an anterior surface defining heights above said posterior surface;
wherein each of two points on said anterior surface, distanced from one another along said anterior surface by at least 1 mm, has a respective slope of at least 450 with respect to said eye surface.

Example 36. An ophthalmic device according to any of examples 1 to 32, having:
a. a posterior surface; and
b. an anterior surface that when the ophthalmic device is disposed in a relaxed configuration on a planar surface with the posterior surface adjacent to the planar surface defines heights above the planar surface,
wherein two points on said anterior surface are distanced from one another along said anterior surface by at least 1 mm, and each has a respective slope of at least 45° with respect to said planar surface.

Example 37. An ophthalmic device, configured to reside between an eye surface and an eyelid, comprising:
a. a body made of an elastomeric matrix, wherein said elastomeric matrix comprises: water, one or more types of PVOH, and one or more organic plasticizers, said body characterized by:
i. a posterior surface which is adjacent to an eye surface when said ophthalmic device is worn;
ii. an anterior surface, which is adjacent to said eyelid covering said eye surface when said ophthalmic device is worn;
iii. a retention portion, defined between a portion of said anterior surface and a portion of said posterior surface;
wherein said retention portion has an edge structured to cause the ophthalmic device to move with the eyelid when the eyelid moves in respect to the eyeball.

Example 38. The ophthalmic device according to example 37, wherein said edge is structured with a slope of said anterior surface extending from said posterior surface, said slope is characterized by an angle larger than 450 between said posterior surface and said anterior surface.

Example 39. The ophthalmic device according to example 37 or example 38, configured to completely reside between the eye surface and the eyelid.

Example 40. The ophthalmic device according to example any one of example 37-39, wherein said slope of more than 45 degrees is for at least of a length of 1 mm.

Example 41. The ophthalmic device according to any one of examples 37-40, wherein said angle of a slope of said anterior surface extending from said posterior surface is from 45 degrees to 90 degrees.

Example 42. The ophthalmic device according to any one of examples 37-41, wherein said retention portion comprises a maximal height of from 0.5 mm to 5 mm.

Example 43. The ophthalmic device according to any one of examples 37-42, wherein said ophthalmic device has a length from about 3 mm to about 10 mm.

Example 44. The ophthalmic device according to any one of examples 37-43, wherein said ophthalmic device has a width from about 3 mm to about 10 mm.

Example 45. The ophthalmic device according to any one of examples 37-44, wherein a relation between a width and a length of said ophthalmic device is from about 1:1 to about 1:3.

Example 46. The ophthalmic device according to any one of examples 37-45, wherein a combined mass ratio of said one or more organic plasticizers to said one or more types of PVOH is at least 2:1.

Example 47. The ophthalmic device according to any one of examples 37-46, wherein said body swells isotropically when exposed to wet conditions.

Example 48. The ophthalmic device according to any one of examples 37-47, wherein said PVOH comprises PVOH of two or more types that differ from one another in one or both of hydrolysis degree (HD) and chain length.

Example 49. The ophthalmic device according to any one of examples 37-48, wherein the two or more types of PVOH include one type that has long chains and is fully hydrolyzed, and one type that has at least one of:
a. short chains and is partially hydrolyzed;
b. long chains and is partially hydrolyzed;
c. short chains and is fully hydrolyzed.

Example 50. The ophthalmic device according to any one of examples 37-49, wherein:
a. short chains are of 200 to 2000 monomeric units;
b. long chains are of 2200 to 5000 monomeric units;
c. fully hydrolyzed PVOH is of hydrolysis degree of 97% or more; and
d. partially hydrolyzed is of hydrolysis degree of 95% or less.

Example 51. The ophthalmic device according to any one of examples 37-50, wherein said PVOH comprises PVOH of two or more types that differ from one another in hydrolysis degree (HD).

Example 52. The ophthalmic device according to any one of examples 37-51, wherein two types of the PVOH differ in chain length by at least 1000, and have similar degrees of hydrolysis, both between 97% and 100%.

Example 53. The ophthalmic device according to any one of examples 37-52, wherein a first type of said two or more types has a hydrolysis degree from 97% to 100%.

Example 54. The ophthalmic device according to any one of examples 37-53, wherein a second type of said two or more types has a hydrolysis degree is lower than 93%.

Example 55. The ophthalmic device according to any one of examples 37-54, wherein a second type of said two or more types has a hydrolysis degree from 80% to 93%.

Example 56. The ophthalmic device according to any one of examples 37-55, wherein a first type of said two or more types has chains with more than 2500 units.

Example 57. The ophthalmic device according to any one of examples 37-56, wherein a second type of said two or more types has chains with less than 1500 units.

Example 58. The ophthalmic device according to any one of examples 37-57, wherein a relation between a first type of PVOH and a second type of PVOH of said two or more types is from about 3:1 to about 1:3.

Example 59. The ophthalmic device according to any one of examples 37-58, wherein a first type of said two or more types has chains with more than 2500 units and hydrolysis degree of 97% to 100%, and a second type of said two or more types has chains with less than 1000 units and hydrolysis degree of 80% to 93%.

Example 60. The ophthalmic device according to any one of examples 37-59, wherein said second type of PVOH makes more than 50% of said PVOH.

Example 61. The ophthalmic device according to any one of examples 37-60, wherein said at least two types of PVOH determine rate of degradation of said ophthalmic device.

Example 62. The ophthalmic device according to any one of examples 37-61, wherein said rate of degradation comprises a rate by which a predetermined change in a predetermined mechanical property takes place in simulated tear fluid (STF).

Example 63. The ophthalmic device according to any one of examples 37-62, wherein a combined mass content of said PVOH and said one or more organic plasticizers is at least 70% wt of the total weight of the matrix excluding said water.

Example 64. The ophthalmic device according to any one of examples 37-63, wherein said elastomeric matrix is characterized by substantially isotropic swelling when being immersed in simulated tear fluid and at room temperature for 5 minutes.

Example 65. The ophthalmic device according to any one of examples 37-64, wherein said elastomeric matrix swells by less than 50% by volume when immersed in simulated tear fluid for 5 minutes at room temperature.

Example 66. The ophthalmic device according to any one of examples 37-65, wherein said water constitute less than 50% wt of the total mass content of the matrix.

Example 67. The ophthalmic device according to any one of examples 37-66, essentially devoid of covalent crosslinking.

Example 68. The ophthalmic device according to any one of examples 37-67, further comprising at least one ophthalmically acceptable pharmaceutically active ingredient.

Example 69. The ophthalmic device according to any one of examples 37-68, wherein said at least one ophthalmic acceptable pharmaceutically active ingredient is carried by said elastomeric matrix.

Example 70. The ophthalmic device according to any one of examples 37-69, wherein said at least one ophthalmic acceptable pharmaceutically active ingredient is dispersed within said elastomeric matrix.

Example 71. The ophthalmic device according to any one of examples 37-70, wherein said at least one ophthalmic acceptable pharmaceutically active ingredient is encapsulated and then carried by said elastomeric matrix.

Example 72. The ophthalmic device according to any one of examples 37-71, wherein said water makes between 5 wt % and 50 wt % of said elastomeric matrix.

Example 73. The ophthalmic device according to any one of examples 37-72, wherein said ophthalmic device is configured to gain no more than 50% weight when immersed in simulated tear fluid for 5 minutes.

Example 74. The ophthalmic device according to any one of examples 37-73, wherein said ophthalmic device is obtainable from a liquid solution comprising water, a pharmaceutically active ingredient, the PVOH, and the organic plasticizers, without freezing.

Example 75. The ophthalmic device according to any one of examples 37-74, wherein said ophthalmic device is obtained from a liquid solution comprising water, a pharmaceutically active ingredient, the PVOH, and the water-soluble organic liquids, without freezing.

Example 76. The ophthalmic device according to any one of examples 37-75, wherein said ophthalmic device is characterized by having a Young modulus of between 0.05 MPa and 10 MPa.

Example 77. The ophthalmic device according to any one of examples 37-76, wherein said ophthalmic device is stable under 25° C.±2° C. and relative humidity of 60%±5% for at least three months.

Example 78. The ophthalmic device according to any one of examples 37-77, comprising two or more types of PVOH that differ from each other in a degree of hydrolysis.

Example 79. The ophthalmic device according to any one of examples 37-78, wherein a ratio between said two or more types of PVOH is such that said ophthalmic device degrades at least 50% of a weight of said ophthalmic device under wet conditions within 24 hours.

Example 80. The ophthalmic device according to any one of examples 37-79, wherein a ratio between said two or more types of PVOH is such that said ophthalmic device remains intact under wet conditions during the first 10 minutes.

Example 81. The ophthalmic device according to any one of examples 37-80, wherein said ophthalmic device is visible to naked eye on a background of bulbar conjunctiva.

Example 82. The ophthalmic device according to any one of examples 37-81, wherein said retention portion is defined by heights between said posterior surface and said anterior surface; and wherein a first height of said heights measured from a first point on said posterior surface to a corresponding point on said anterior surface differs by at least 1 mm, from a second height of said heights measured from a second point on said posterior surface to a corresponding point on said anterior surface, said second point being distanced at most 1 mm from said first point.

Example 83. The ophthalmic device according to any one of examples 37-82, wherein said device is configured for wearing away from the fornix.

Example 84. An ophthalmic device, configured to reside between an eye surface and an eyelid, comprising a body made of an elastomeric matrix, wherein said elastomeric matrix comprises: water, one or more types of PVOH, and one or more organic plasticizers,
said body characterized by:
i. a posterior surface which is adjacent to an eye surface when said ophthalmic device is worn;
ii. an anterior surface, which is adjacent to said eyelid covering said eye surface when said ophthalmic device is worn;
iii. a retention portion, defined between a portion of said anterior surface and a portion of said posterior surface;
wherein a combined mass ratio of said one or more organic plasticizers to said one or more types of PVOH is at least 2:1.

Example 85. An ophthalmic device, configured to reside between an eye surface and an eyelid, comprising a body made of an elastomeric matrix, wherein said elastomeric matrix comprises: water, one or more types of PVOH, and one or more organic plasticizers,
said body characterized by:
i. a posterior surface which is adjacent to an eye surface when said ophthalmic device is worn;
ii. an anterior surface, which is adjacent to said eyelid covering said eye surface when said ophthalmic device is worn;
iii. a retention portion, defined between a portion of said anterior surface and a portion of said posterior surface;
wherein ratios between said one or more organic plasticizers to said one or more types of PVOH is such that upon staying still in simulated tear fluid for a period of 15 minutes at room temperature, the device swells by less than 50% (v/v).

Example 86. The ophthalmic device according to example 84 or example 85, wherein said retention portion is configured to cause the ophthalmic device to move with the eyelid when the eyelid moves in respect to the eyeball.

Example 87. The ophthalmic device according to any one of examples 84-86, wherein an angle of a slope of said anterior surface extending from said posterior surface is larger than 45 degrees.

Example 88. The ophthalmic device according to any one of examples 84-87, wherein said angle of a slope of said anterior surface extending from said posterior surface is from 45 degrees to 90 degrees.

Example 89. The ophthalmic device according to any one of examples 84-88, wherein said retention portion comprises a maximal height of from 0.5 mm to 5 mm.

Example 90. The ophthalmic device according to any one of examples 84-89, wherein said ophthalmic device comprises a length from about 3 mm to about 10 mm.

Example 91. The ophthalmic device according to any one of examples 84-90, wherein said ophthalmic device comprises a width from about 3 mm to about 10 mm.

Example 92. The ophthalmic device according to any one of examples 84-91, wherein a relation between a width and a length of said ophthalmic device is from about 1:1 to about 1:3.

Example 93. The ophthalmic device according to any one of examples 84-92, wherein said body is characterized by having an elliptical form.

Example 94. The ophthalmic device according to any one of examples 84-93, wherein said body is characterized by having a round form.

Example 95. The ophthalmic device according to any one of examples 84-94, wherein said body is characterized by having a dome-like form.

Example 96. The ophthalmic device according to any one of examples 84-95, wherein said body is characterized by having a half-elliptical sphere form.

Example 97. The ophthalmic device according to any one of examples 84-96, wherein said body swells isotropically when exposed to wet conditions.

Example 98. The ophthalmic device according to any one of examples 84-97, wherein said PVOH comprises PVOH of two or more types that differ from one another in one or both of hydrolysis degree (HD) and chain length.

Example 99. The ophthalmic device according to any one of examples 84-98, wherein each of said two or more types of PVOH constitutes at least 10% of the total amount of PVOH in the matrix.

Example 100. The ophthalmic device according to any one of examples 84-99, wherein the two or more types of PVOH include one type that has long chains and is fully hydrolyzed, and one type that has at least one of:
a. short chains and is partially hydrolyzed;
b. long chains and is partially hydrolyzed;
c. short chains and is fully hydrolyzed.

Example 101. The ophthalmic device according to any one of examples 84-100, wherein:
a. short chains are of 200 to 2000 monomeric units;
b. long chains are of 2200 to 5000 monomeric units;
c. fully hydrolyzed PVOH is of hydrolysis degree of 97% or more; and
d. partially hydrolyzed is of hydrolysis degree of 95% or less.

Example 102. The ophthalmic device according to any one of examples 84-101, wherein said PVOH comprises PVOH of two or more types that differ from one another in hydrolysis degree (HD).

Example 103. The ophthalmic device according to any one of examples 84-102, wherein two types of the PVOH differ in chain length by at least 1000, and have similar degrees of hydrolysis, both between 97% and 100%.

Example 104. The ophthalmic device according to any one of examples 84-103, wherein a first type of said two or more types has a hydrolysis degree from 97% to 100%.

Example 105. The ophthalmic device according to any one of examples 84-104, wherein a second type of said two or more types has a hydrolysis degree is lower than 93%.

Example 106. The ophthalmic device according to any one of examples 84-105, wherein a second type of said two or more types has a hydrolysis degree from 80% to 93%.

Example 107. The ophthalmic device according to any one of examples 84-106, wherein a first type of said two or more types has chains with more than 2500 units.

Example 108. The ophthalmic device according to any one of examples 84-107, wherein a second type of said two or more types has chains with less than 1500 units.

Example 109. The ophthalmic device according to any one of examples 84-108, wherein a relation between a first type of PVOH and a second type of PVOH of said two or more types is from about 3:1 to about 1:3.

Example 110. The ophthalmic device according to any one of examples 84-109, wherein a first type of said two or more types has chains with more than 2500 units and hydrolysis degree of 97% to 100%, and a second type of said two or more types has chains with less than 1000 units and hydrolysis degree of 80% to 93%.

Example 111. The ophthalmic device according to any one of examples 84-110, wherein said second type of PVOH makes more than 50% of said PVOH.

Example 112. The ophthalmic device according to any one of examples 84-111, wherein said at least two types of PVOH determine rate of degradation of said ophthalmic device.

Example 113. The ophthalmic device according to any one of examples 84-112, wherein said rate of degradation comprises a rate by which a predetermined change in a predetermined mechanical property takes place in simulated tear fluid (STF).

Example 114. The ophthalmic device according to any one of examples 84-113, wherein a combined mass content of said PVOH and said one or more organic plasticizers is at least 70% wt of the total weight of the matrix excluding said water.

Example 115. The ophthalmic device according to any one of examples 84-114, wherein said elastomeric matrix is characterized by substantially isotropic swelling when being immersed in simulated tear fluid and at room temperature for 5 minutes.

Example 116. The ophthalmic device according to any one of examples 84-115, wherein said elastomeric matrix swells by less than 50% by volume when immersed in simulated tear fluid for 5 minutes at room temperature.

Example 117. The ophthalmic device according to any one of examples 84-116, wherein said water constitute less than 50% wt of the total mass content of the matrix.

Example 118. The ophthalmic device according to any one of examples 84-117, essentially devoid of covalent crosslinking.

Example 119. The ophthalmic device according to any one of examples 84-118, further comprising at least one ophthalmic acceptable pharmaceutically active ingredient.

Example 120. The ophthalmic device according to any one of examples 84-119, wherein said at least one ophthalmic acceptable pharmaceutically active ingredient is carried by said elastomeric matrix.

Example 121. The ophthalmic device according to any one of examples 84-120, wherein said at least one ophthalmically acceptable pharmaceutically active ingredient is dispersed within said elastomeric matrix.

Example 122. The ophthalmic device according to any one of examples 84-121, wherein said at least one ophthalmically acceptable pharmaceutically active ingredient is encapsulated and then carried by said elastomeric matrix.

Example 123. The ophthalmic device according to any one of examples 84-122, wherein said water makes between 5 wt % and 50 wt % of said elastomeric matrix.

Example 124. The ophthalmic device according to any one of examples 84-123, wherein said ophthalmic device is configured to gain no more than 50% weight when exposed to wet conditions.

Example 125. The ophthalmic device according to any one of examples 84-124, wherein said ophthalmic device is obtainable from a liquid solution comprising water, the active ingredient, the PVOH, and the water-soluble organic liquids, without freezing.

Example 126. The ophthalmic device according to any one of examples 84-125, wherein said ophthalmic device is obtained from a liquid solution comprising water, the active ingredient, the PVOH, and the water-soluble organic liquids, without freezing.

Example 127. The ophthalmic device according to any one of examples 84-126, wherein said ophthalmic device is characterized by having a Young modulus of between 0.05 MPa and 10 MPa.

Example 128. The ophthalmic device according to any one of examples 84-127, wherein said ophthalmic device is stable under dry conditions for at least three months.

Example 129. The ophthalmic device according to any one of examples 84-128, comprising two or more types of PVOH that differ from each other in a degree of hydrolysis.

Example 130. The ophthalmic device according to any one of examples 84-129, wherein a ratio between said two or more types of PVOH is such that said ophthalmic device disintegrates under wet conditions within 24 hours.

Example 131. The ophthalmic device according to any one of examples 84-130, wherein a ratio between said two or more types of PVOH is such that said ophthalmic device remains intact under wet conditions during the first 10 minutes.

Example 132. The ophthalmic device according to any one of examples 84-131, wherein said ophthalmic device is visible to naked eye on a background of bulbar conjunctiva.

Example 133. The ophthalmic device according to any one of examples 84-132, wherein said ophthalmic device is elongated and symmetrical, so that all orientations of the device are equivalent as long as an elongated side of the device lies at a given direction.

Example 134. The ophthalmic device according to any one of examples 84-133, wherein said retention portion is defined by heights between said posterior surface and said anterior surface; and wherein a first height of said heights measured from a first point on said posterior surface to a corresponding point on said anterior surface differs by at least 1 mm, from a second height of said heights measured from a second point on said posterior surface to a corresponding point on said anterior surface, said second point being distanced at most 1 mm from said first point.

Example 135. The ophthalmic device according to any one of examples 84-134, wherein said device is configured for wearing away from the fornix.

Example 136. An ophthalmic device, comprising:
a. a body comprising an elastomeric matrix; said elastomeric matrix comprising between 5% wt and 50% wt water, and at least 50% wt ophthalmic lubricants, of which between 5% wt and 33% wt is polyvinyl alcohol (PVOH);
said body characterized by:
i. a posterior surface, configured to conform to a surface of an eye under an eyelid; and
ii. an anterior surface defining a height of said body above said eye surface;
iii. a retention portion, defined between a portion of said anterior surface and a portion of said posterior surface; wherein an angle of a slope of said anterior surface extending from said posterior surface is larger than 45 degrees; and
b. an active pharmaceutical ingredient (API) for treating an eye condition, said API is carried by said elastomeric matrix, and released from the matrix under wet conditions,
wherein said PVOH comprises PVOH of two or more types that differ from one another in one or both of hydrolysis degree (HD) and chain length.

Example 137. The ophthalmic device according to example 136, wherein each of said two or more types of PVOH constitutes at least 10% of the total amount of PVOH in the matrix.

Example 138. The ophthalmic device according to example 136 or example 137, wherein the two or more types of PVOH include one type that has long chains and is fully hydrolyzed, and one type that has at least one of:
a. short chains and is partially hydrolyzed;
b. long chains and is partially hydrolyzed;
c. short chains and is fully hydrolyzed.

Example 139. The ophthalmic device according to any one of examples 136-138, wherein:
a. short chains are of 200 to 2000 monomeric units;
b. long chains are of 2200 to 5000 monomeric units;
c. fully hydrolyzed PVOH is of hydrolysis degree of 97% or more; and
d. partially hydrolyzed is of hydrolysis degree of 95% or less.

Example 140. The ophthalmic device according to any one of examples 136-139, wherein said PVOH comprises PVOH of two or more types that differ from one another in hydrolysis degree (HD).

Example 141. The ophthalmic device according to any one of examples 136-140, wherein two types of the PVOH differ in chain length by at least 1000, and have similar degrees of hydrolysis, both between 97% and 100%.

Example 142. The ophthalmic device according to any one of examples 136-141, wherein a first type of said two or more types has a hydrolysis degree from 97% to 100%.

Example 143. The ophthalmic device according to any one of examples 136-142, wherein a second type of said two or more types has a hydrolysis degree is lower than 93%.

Example 144. The ophthalmic device according to any one of examples 136-143, wherein a second type of said two or more types has a hydrolysis degree from 80% to 93%.

Example 145. The ophthalmic device according to any one of examples 136-144, wherein a first type of said two or more types has chains with more than 2500 units.

Example 146. The ophthalmic device according to any one of examples 136-145, wherein a second type of said two or more types has chains with less than 1500 units.

Example 147. The ophthalmic device according to any one of examples 136-146, wherein a relation between a first type of PVOH and a second type of PVOH of said two or more types is from about 3:1 to about 1:3.

Example 148. The ophthalmic device according to any one of examples 136-147, wherein a first type of said two or more types has chains with more than 2500 units and hydrolysis degree of 97% to 100%, and a second type of said two or more types has chains with less than 1000 units and hydrolysis degree of 80% to 93%.

Example 149. The ophthalmic device according to any one of examples 136-148, wherein said second type of PVOH makes more than 50% of said PVOH.

Example 150. The ophthalmic device according to any one of examples 136-149, wherein said at least two types of PVOH determine rate of degradation of said ophthalmic device.

Example 151. The ophthalmic device according to any one of examples 136-150, wherein said rate of degradation comprises a rate by which a predetermined change in a predetermined mechanical property takes place in simulated tear fluid (STF).

Example 152. The ophthalmic device according to any one of examples 136-151, wherein a combined mass content of said PVOH and said one or more organic plasticizers is at least 70% wt of the total weight of the matrix excluding said water.

Example 153. The ophthalmic device according to any one of examples 136-152, wherein said elastomeric matrix is characterized by substantially isotropic swelling when being immersed in simulated tear fluid and at room temperature for 5 minutes.

Example 154. The ophthalmic device according to any one of examples 136-153, wherein said elastomeric matrix swells by less than 50% by volume when immersed in simulated tear fluid for 5 minutes at room temperature.

Example 155. The ophthalmic device according to any one of examples 136-154, wherein said water constitute less than 50% wt of the total mass content of the matrix.

Example 156. The ophthalmic device according to any one of examples 136-155, essentially devoid of covalent crosslinking.

Example 157. The ophthalmic device according to any one of examples 136-156, wherein each of said plurality of ophthalmic lubricants is selected from the group consisting of polyvinyl alcohol, a polyol, glycerol, polyethylene glycol, propylene glycol, polysorbate, hyaluronic acid or pharmaceutically acceptable salt thereof, cellulose, carboxymethyl cellulose sodium, hydroxyethyl cellulose, methylcellulose, dextran, gelatin, polysorbate, hydroxypropyl methylcellulose, and povidone.

Example 158. The ophthalmic device according to any one of examples 136-157, wherein said ophthalmic lubricants comprise polyethylene glycol (PEG), and the mass content of the PEG in the elastomeric matrix is about equal that of the PVOH.

Example 159. The ophthalmic device according to any one of examples 136-158, wherein at least 35% of said ophthalmic acceptable lubricants consist of glycerol and propylene glycol.

Example 160. The ophthalmic device according to any one of examples 136-159, wherein said elastomeric matrix is impregnated with said API.

Example 161. The ophthalmic device according to any one of examples 136-160, obtainable by mixing the API with water and ophthalmic demulcents to obtain a mother solution; and drying the mother solution at room temperature.

Example 162. The ophthalmic device according to any one of examples 136-161, obtainable without heating the mother solution.

Example 163. The ophthalmic device according to any one of examples 136-162, obtainable without heating the API.

Example 164. The ophthalmic device according to any one of examples 136-163, wherein said ophthalmic device is characterized, when under dry conditions, by at least one of the following mechanical properties:
a. tensile strength of between 0.01 MPa and 10 MPa;
b. Young modulus of between 0.05 MPa and 10 MPa; and
c. elongation at break of between 50% and 1000%.

Example 165. The ophthalmic device according to any one of examples 136-164, wherein said ophthalmic device is configured to degrade in the eye and to lose at least 50% of its weight within 2 hours in an eye.

Example 166. The ophthalmic device according to any one of examples 136-165, wherein said retention portion is configured to keep said ophthalmic device in place when the device is worn, and the eye is rolled.

Example 167. The ophthalmic device according to any one of examples 136-166, wherein a first height perpendicular to a reference surface and measured from a first point on said posterior surface to a corresponding point on said anterior surface differs by at least 1 mm, from a second height perpendicular to the reference surface and measured from a second point on said posterior surface to a corresponding point on said anterior surface, said second point being distanced at most 1 mm from said first point.

Example 168. The ophthalmic device according to any one of examples 136-167, sized and shaped to be fully covered by the eyelid when worn under a lower eyelid.

Example 169. The ophthalmic device according to any one of examples 136-168, wherein said ophthalmic device is configured for self-administration.

Example 170. The ophthalmic device according to any one of examples 136-169, comprising an active ingredient for treating presbyopia.

Example 171. The ophthalmic device according to any one of examples 136-170, for use in the treating of presbyopia.

Example 172. The ophthalmic device according to any one of examples 136-171, wherein said body is configured to minimize disturbance to the eyelid when the posterior surface resides on said surface of said eye under said eyelid

Example 173. The ophthalmic device according to any one of examples 136-172, wherein said device is configured for wearing away from the fornix.

Example 174. An ophthalmic drug product for use in the treatment of presbyopia, comprising:
a. an elastomeric matrix comprising
i. at most 45% wt water, and
ii. at least 50% wt ophthalmic lubricants, of which between 5% wt and 33% wt is polyvinyl alcohol (PVOH); and
b. a pharmacological agent for treating presbyopia.

Example 175. The ophthalmic drug product according to example 174, wherein said ophthalmic drug product is an ophthalmic device according to any one of examples 1 to 173.

Example 176. An ophthalmic drug product consisting essentially of one or more pharmaceutically active ingredients (APIs), ophthalmic demulcents, and water, wherein the water is no more than 50% wt of the drug product.

Example 177. The ophthalmic drug product according to example 176, wherein 95% wt of said ophthalmic drug product consists only of API, ophthalmic demulcents, and water.

Example 178. The ophthalmic drug product according to example 176 or example 177, wherein said ophthalmic drug product is characterized by at least one of the following mechanical properties:
tensile strength between 0.01 MPa and 10 MPa;
elastic module between 0.05 MPa and 10 MPa; and
elongation at break between 50% and 1000%.

Example 179. The ophthalmic drug product according to any one of examples 176-178, wherein no less than 3% wt and no more than 33% wt of said ophthalmic demulcents consist of PVOH.

Example 180. The ophthalmic drug product according to any one of examples 176-179, wherein said PVOH comprises two types of PVOH, which differ from one another in their degree of hydrolysis.

Example 181. The ophthalmic drug product according to any one of examples 176-180, wherein said two types of PVOH, differ from one another in their chain length.

Example 182. The ophthalmic drug product according to any one of examples 176-181, comprising PVOH having a degree of hydrolysis of 97% or larger and PVOH having a degree of hydrolysis of 93% or lower.

Example 183. The ophthalmic drug product according to any one of examples 176-182, wherein a majority of said PVOH has a degree of hydrolysis of 93% or lower.

Example 184. The ophthalmic drug product according to any one of examples 176-183, wherein at least two thirds of said PVOH have a degree of hydrolysis of 93% or lower.

Example 185. The ophthalmic drug product according to any one of examples 176-184, wherein at least one of said one or more APIs is for treating presbyopia.

Example 186. The ophthalmic drug product according to any one of examples 176-185, wherein at least one of said one or more APIs is pilocarpine, phentolamine mesylate, oxymetazoline, carbachol, phospholine Iodine, brimonidine, aceclidine, and lipoic acid choline ester.

Example 187. The ophthalmic drug product according to any one of examples 176-186, wherein said ophthalmic drug product is configured to reside between a bulbar conjunctiva and an eyelid covering an eyeball, the device having a retention portion, sized and shaped so that when between the bulbar conjunctiva and the eyelid, the retention portion is pressed against the eyelid at a force hard enough so that movement of the eyelid in respect to the eyeball forces the ophthalmic device to move with the eyelid across the eyeball.

Example 188. The ophthalmic drug product according to any one of examples 176-186, wherein said ophthalmic drug product comprises:
a posterior surface configured to be adjacent to an eye surface when said ophthalmic drug product is in the eye;
an anterior surface defining heights above said eye surface; and
a retention portion defined between a portion of said posterior surface and a corresponding portion of said anterior surface, wherein two of said heights, defined by said corresponding portion of the anterior surface and distanced from each other by at most 1 mm, differ from one another by a height difference of at least 1 mm.

Example 189. The ophthalmic drug product according to any one of examples 176-188, wherein said ophthalmic drug product comprises:
a posterior surface configured to be adjacent to an eye surface when said ophthalmic drug product is in the eye;
an anterior surface defining heights above said eye surface; and
a retention portion defined between a portion of said posterior surface and a corresponding portion of said anterior surface, wherein two points on said portion of said anterior surface are distanced from one another along the anterior surface by at least 1 mm, and each having a respective slope of at least 450 with respect to said eye surface.

Example 190. The ophthalmic drug product according to any one of examples 176-189, wherein said ophthalmic drug product is an ophthalmic device according to at least one of examples 1, 37, 84, 85 and 136.

Example 191. A method of treating presbyopia, comprising continuously eluting a presbyopia treating drug for at least 5 minutes from a drug product according to any one of examples 174 to 189 to a surface of an eye diagnosed with presbyopia.

Example 192. An ophthalmic device according to any one of examples 1 to 173, configured to elute on an eye surface ophthalmic demulcents, said ophthalmic demulcents having a mass content that is at least half the mass content of the ophthalmic device.

Example 193. An ophthalmic device having a mass content, and being configured to elute on an eye surface ophthalmic demulcents having a mass content that is at least half the mass content of the ophthalmic device.

Example 194. A method of treating dry eye disease, comprising administering to a patient in need of said treatment an ophthalmic device according to example 192 or 193.

Example 195. An ophthalmic device, comprising:
a. a body comprising an elastomeric matrix; said elastomeric matrix comprising between 5% wt and 45% wt water, and at least 50% wt ophthalmic lubricants, of which between 5% wt and 33% wt is polyvinyl alcohol (PVOH);
said body characterized by:
i. a posterior surface, configured to conform to a surface of an eye under an eyelid;
ii. an anterior surface defining a height of said body above said eye surface; and
iii. a retention portion, defined between a portion of said anterior surface and a portion of said posterior surface; wherein an angle of a slope of said anterior surface extending from said posterior surface is larger than 45 degrees;
b. an active pharmaceutical ingredients (API) for treating an eye condition, said API is carried by said elastomeric matrix, and released from the matrix under wet conditions.

Example 196. The ophthalmic device according to example 195, wherein said PVOH comprises PVOH of two or more types that differ from one another in one or both of hydrolysis degree (HD) and chain length.

Example 197. The ophthalmic device according to example 195 or example 196, wherein each of said two or more types of PVOH constitutes at least 10% of the total amount of PVOH in the matrix.

Example 198. The ophthalmic device according to any one of examples 195-197, wherein the two or more types of PVOH include one type that has long chains and is fully hydrolyzed, and one type that has at least one of:
a. short chains and is partially hydrolyzed;
b. long chains and is partially hydrolyzed;
c. short chains and is fully hydrolyzed.

Example 199. The ophthalmic device according to any one of examples 195-198, wherein:
a. short chains are of 200 to 2000 monomeric units;
b. long chains are of 2200 to 5000 monomeric units;
c. fully hydrolyzed PVOH is of hydrolysis degree of 97% or more; and
d. partially hydrolyzed is of hydrolysis degree of 95% or less.

Example 200. The ophthalmic device according to any one of examples 195-199, wherein said PVOH comprises PVOH of two or more types that differ from one another in hydrolysis degree (HD).

Example 201. The ophthalmic device according to any one of examples 195-200, wherein two types of the PVOH differ in chain length by at least 1000, and have similar degrees of hydrolysis, both between 97% and 100%.

Example 202. The ophthalmic device according to any one of examples 195-201, wherein a first type of said two or more types has a hydrolysis degree from 97% to 100%.

Example 203. The ophthalmic device according to any one of examples 195-202, wherein a second type of said two or more types has a hydrolysis degree is lower than 93%.

Example 204. The ophthalmic device according to any one of examples 195-203, wherein a second type of said two or more types has a hydrolysis degree from 80% to 93%.

Example 205. The ophthalmic device according to any one of examples 195-204, wherein a first type of said two or more types has chains with more than 2500 units.

Example 206. The ophthalmic device according to any one of examples 195-205, wherein a second type of said two or more types has chains with less than 1500 units.

Example 207. The ophthalmic device according to any one of examples 195-206, wherein a relation between a first type of PVOH and a second type of PVOH of said two or more types is from about 3:1 to about 1:3.

Example 208. The ophthalmic device according to any one of examples 195-207, wherein a first type of said two or more types has chains with more than 2500 units and hydrolysis degree of 97% to 100%, and a second type of said two or more types has chains with less than 1000 units and hydrolysis degree of 80% to 93%.

Example 209. The ophthalmic device according to any one of examples 195-208, wherein said second type of PVOH makes more than 50% of said PVOH.

Example 210. The ophthalmic device according to any one of examples 195-209, wherein said at least two types of PVOH determine rate of degradation of said ophthalmic device.

Example 211. The ophthalmic device according to any one of examples 195-210, wherein said rate of degradation comprises a rate by which a predetermined change in a predetermined mechanical property takes place in simulated tear fluid (STF).

Example 212. The ophthalmic device according to any one of examples 195-211, wherein a combined mass content of said PVOH and said one or more organic plasticizers is at least 70% wt of the total weight of the matrix excluding said water.

Example 213. The ophthalmic device according to any one of examples 195-212, wherein said elastomeric matrix is characterized by substantially isotropic swelling when being immersed in simulated tear fluid and at room temperature for 5 minutes.

Example 214. The ophthalmic device according to any one of examples 195-213, wherein said elastomeric matrix swells by less than 50% by volume when immersed in simulated tear fluid for 5 minutes at room temperature.

Example 215. The ophthalmic device according to any one of examples 195-214, wherein said water constitute less than 50% wt of the total mass content of the matrix.

Example 216. The ophthalmic device according to any one of examples 195-215, essentially devoid of covalent crosslinking.

Example 217. The ophthalmic device according to any one of examples 195-216, wherein each of said plurality of ophthalmic lubricants is selected from the group consisting of polyvinyl alcohol, a polyol, glycerol, polyethylene glycol, propylene glycol, polysorbate, hyaluronic acid or pharmaceutically acceptable salt thereof, cellulose, carboxymethyl cellulose sodium, hydroxyethyl cellulose, methylcellulose, dextran, gelatin, polysorbate, hydroxypropyl methylcellulose, and povidone.

Example 218. The ophthalmic device according to any one of examples 195-217, wherein said ophthalmic acceptable lubricants or demulcents comprise polyethylene glycol (PEG), and the mass content of the PEG in the elastomeric matrix is about equal that of the PVOH.

Example 219. The ophthalmic device according to any one of examples 195-218, wherein at least 35% of said ophthalmic acceptable lubricants or demulcents consist of glycerol and propylene glycol.

Example 220. The ophthalmic device according to any one of examples 195-219, wherein said elastomeric matrix is impregnated with an ophthalmically acceptable active pharmaceutical ingredient.

Example 221. The ophthalmic device according to any one of examples 195-220, obtainable by mixing the API with water and ophthalmic demulcents to obtain a mother solution; and drying the mother solution at room temperature.

Example 222. The ophthalmic device according to any one of examples 195-221, obtainable without heating the mother solution.

Example 223. The ophthalmic device according to any one of examples 195-222, wherein said ophthalmic device is characterized by at least one of the following mechanical properties:
a. tensile strength of between 0.01 MPa and 10 MPa;
b. Young modulus of between 0.05 MPa and 10 MPa; and
c. elongation at break of between 50% and 1000%.

Example 224. The ophthalmic device according to any one of examples 195-223, wherein said ophthalmic device is configured to degrade in the eye and to lose at least 50% of its weight within 2 hours in the eye.

Example 225. The ophthalmic device according to any one of examples 195-224, wherein said retention portion is configured to keep said ophthalmic device in place when the device is worn, and the eye is rolled.

Example 226. The ophthalmic device according to any one of examples 195-225, wherein a first height perpendicular to a reference surface and measured from a first point on said posterior surface to a corresponding point on said anterior surface differs by at least 1 mm, from a second height perpendicular to the reference surface and measured from a second point on said posterior surface to a corresponding point on said anterior surface, said second point being distanced at most 1 mm from said first point.

Example 227. The ophthalmic device according to any one of examples 195-226, sized and shaped to be fully covered by the eyelid when worn under a lower eyelid.

Example 228. The ophthalmic device according to any one of examples 195-227, wherein said ophthalmic device is configured for self-administration.

Example 229. The ophthalmic device according to any one of examples 195-228, wherein said device is configured for wearing away from the fornix.

Example 230. An ophthalmic drug product for treating presbyopia, comprising:
a. an elastomeric matrix comprising
   i. between 5% wt and 45% wt water, and
   ii. at least 50% wt ophthalmic lubricants, of which between 5% wt and 33% wt is polyvinyl alcohol (PVOH); and
b. an active pharmaceutical ingredient (API) usable in treating presbyopia.

Example 231. The ophthalmic drug product according to example 230, wherein said ophthalmic drug product is an ophthalmic device according to any one of examples 1 to 229.

Example 232. A method of treating presbyopia, comprising continuously eluting an active pharmacological ingredient usable in treating presbyopia for at least 5 minutes from a drug product according to any one of examples 174 to 189 to a conjunctiva of an eye diagnosed with presbyopia.

Example 233. A method of treating an eye inflicted with dry eye syndrome, the method comprising topically administering to the eye an ophthalmic device comprising an elastomeric matrix having a mass content that elutes on a surface of the eye ophthalmic demulcents, said eluted ophthalmic demulcents making 50% or more of the mass content of the matrix.

Example 234. A method of treating an eye inflicted with dry eye syndrome, the method comprising topically administering to the eye an ophthalmic device according to any one of examples 36 to 64, and 160 to 204

Example 235. The method according to example 234, wherein the ophthalmic device comprises no API other than polyvinyl alcohol, glycerol, polyethylene glycol, propylene glycol, polysorbate, hyaluronic acid or pharmaceutically acceptable salt thereof, cellulose, carboxymethyl cellulose sodium, hydroxyethyl cellulose, methylcellulose, dextran, gelatin, polysorbate, hydroxypropyl methylcellulose, and povidone.

Example 236. The method according to example 234 or example 235, wherein the ophthalmic device is according to the ophthalmic device according to anyone of the preceding claims.

Example 237. An ophthalmic drug product for use in the treatment of presbyopia, comprising:
a. an elastomeric matrix comprising
   i. between 5% wt and 45% wt water, and
   ii. at least 50% wt ophthalmic lubricants, of which between 5% wt and 33% wt is polyvinyl alcohol (PVOH); and
b. an active pharmaceutical ingredient (API) usable in treating presbyopia.

Example 238. The ophthalmic drug product according to example 237, wherein said ophthalmic drug product is an ophthalmic device according to any one of the preceding claims.

Example 239. A method of treating dry eye disease, the method comprising administering to a patient in need of such treatment an ophthalmic device according to any one of the preceding claims.

Example 240. The method according to example 238, wherein the ophthalmic device comprises no API other than one or more of polyvinyl alcohol, glycerol, polyethylene glycol, propylene glycol, polysorbate, hyaluronic acid or pharmaceutically acceptable salt thereof, cellulose, carboxymethyl cellulose sodium, hydroxyethyl cellulose, methylcellulose, dextran, gelatin, polysorbate, hydroxypropyl methylcellulose, and povidone.

Example 241. An ophthalmic drug product for use in the treatment of presbyopia, comprising:
a. an elastomeric matrix comprising
   i. between 5% wt and 45% wt water, and
   ii. at least 50% wt ophthalmic lubricants, of which between 5% wt and 33% wt is polyvinyl alcohol (PVOH); and
b. an active pharmaceutical ingredient (API) usable in treating presbyopia.

Example 242. The ophthalmic drug product according to example 241, wherein said ophthalmic drug product is an ophthalmic device according to any one of the preceding claims.

Example 243. A method of treating dry eye disease, the method comprising administering to a patient in need of such treatment an ophthalmic device according to any one of the preceding claims.

Example 244. The method according to example 243, wherein the ophthalmic device comprises no API other than one or more of polyvinyl alcohol, glycerol, polyethylene glycol, propylene glycol, polysorbate, hyaluronic acid or pharmaceutically acceptable salt thereof, cellulose, carboxymethyl cellulose sodium, hydroxyethyl cellulose, methylcellulose, dextran, gelatin, polysorbate, hydroxypropyl methylcellulose, and povidone.

Example 245. An ophthalmic device, configured to reside between an eye surface of an eyeball and an eyelid, comprising:
a body made of an elastomeric matrix, wherein said elastomeric matrix comprises: water, one or more types of PVOH, and one or more organic plasticizers,
said body characterized by:
i. a posterior surface which is adjacent to an eye surface when said ophthalmic device is worn;
ii. an anterior surface, which is adjacent to said eyelid covering said eye surface when said ophthalmic device is worn;
iii. a retention portion, defined between a portion of said anterior surface and a portion of said posterior surface;
wherein a combined mass ratio of said one or more organic plasticizers to said one or more types of PVOH is at least 2:1.

Example 246. The ophthalmic device according to example 245, wherein said retention portion is configured to cause the ophthalmic device to move with the eyelid when the eyelid moves in respect to the eyeball.

Example 247. The ophthalmic device according to example 245 or example 246, wherein an angle of a slope of said anterior surface extending from said posterior surface is larger than 45 degrees.

Example 248. The ophthalmic device according to any one of examples 245-247, wherein a relation between a width and a length of said ophthalmic device is from about 1:1 to about 1:3.

Example 249. The ophthalmic device according to any one of examples 245-248, wherein said body swells isotropically when immersed in simulated tear fluid for 15 minutes or less.

Example 250. The ophthalmic device according to any one of examples 245-249, further comprising at least one ophthalmic acceptable active pharmaceutical ingredients (API).

Example 251. The ophthalmic device according to any one of examples 245-250, wherein said water makes between 5 wt % and 50 wt % of said elastomeric matrix.

Example 252. The ophthalmic device according to any one of examples 245-251, wherein said ophthalmic device is configured to gain no more than 50% weight when immersed in simulated tear fluid for 15 minutes or less.

Example 253. The ophthalmic device according to any one of examples 245-252, wherein said ophthalmic device is obtainable from a liquid solution comprising water, the ophthalmically acceptable active pharmaceutical ingredient (API), the PVOH, and the water-soluble organic liquids, without freezing.

Example 254. The ophthalmic device according to any one of examples 245-253, comprising two or more types of PVOH.

Example 255. The ophthalmic device according to any one of examples 245-254, wherein a ratio between said two or more types of PVOH is such that said ophthalmic device disintegrates under simulated tear fluid (STF) within 24 hours.

Example 256. The ophthalmic device according to any one of examples 245-255, wherein a ratio between said two or more types of PVOH is such that said ophthalmic device remains intact in simulated tear fluid (STF) during the first 10 minutes.

Example 257. The ophthalmic device according to any one of examples 245-256, wherein said ophthalmic device is visible to naked eye on a background of bulbar conjunctiva.

Example 258. The ophthalmic device according to any one of examples 245-257, wherein said ophthalmic device is elongated and symmetrical, so that all orientations of the device are equivalent as long as an elongated side of the device lies at a given direction.

Example 259. The ophthalmic device according to any one of examples 245-258, wherein said retention portion is defined by heights between said posterior surface and said anterior surface; and wherein a first height of said heights measured from a first point on said posterior surface to a corresponding point on said anterior surface differs by at least 1 mm, from a second height of said heights measured from a second point on said posterior surface to a corresponding point on said anterior surface, said second point being distanced at most 1 mm from said first point.

Example 260. The ophthalmic device, according to any one of examples 245-259, wherein said body includes a plurality of ophthalmic lubricants.

Example 261. The ophthalmic device, according to any one of examples 245-260, wherein each of said plurality of ophthalmic lubricants is selected from the group consisting of polyvinyl alcohol, a polyol, glycerol, polyethylene glycol, propylene glycol, polysorbate, hyaluronic acid or pharmaceutically acceptable salt thereof, cellulose, carboxymethyl cellulose sodium, hydroxyethyl cellulose, methylcellulose, dextran, gelatin, polysorbate, hydroxypropyl methylcellulose, and povidone.

Example 262. The ophthalmic device according to any one of examples 245-261, wherein said PVOH comprises PVOH of two or more types that differ from each other by chain length, degree of hydrolysis, or both.

Example 263. The ophthalmic device according to any one of examples 245-262, wherein at least 35% wt of said two or more types of PVOH has a degree of hydrolysis lower than 90%.

Example 264. The ophthalmic device according to any one of examples 245-263, wherein said ophthalmic lubricants comprise polyethylene glycol (PEG), and the mass content of the PEG in the elastomeric matrix is about equal that of the PVOH.

Example 265. The ophthalmic device according to any one of examples 245-264, wherein at least 35% of said ophthalmic lubricants consist of glycerol and propylene glycol.

Example 266. The ophthalmic device according to any one of examples 245-265, wherein said elastomeric matrix is impregnated with an ophthalmically acceptable active pharmaceutical ingredient.

Example 267. The ophthalmic device according to any one of examples 245-266, obtainable by mixing the API with water and ophthalmic demulcents to obtain a mother solution; and drying the mother solution at room temperature.

Example 268. The ophthalmic device according to any one of examples 245-267, obtainable without heating the mother solution.

Example 269. The ophthalmic device according to any one of examples 245-268, obtainable without heating the API.

Example 270. The ophthalmic device according to any one of examples 245-269, wherein said ophthalmic device is characterized by at least one of the following mechanical properties:
a. tensile strength of between 0.05 MPa and 10 MPa;
b. Young modulus of between 0.05 MPa and 10 MPa; and
c. elongation at break of between 50% and 1000%.

Example 271. The ophthalmic device according to any one of examples 245-270, wherein said ophthalmic device is configured to lose at least 50% of its weight within 2 hours in an eye.

Example 272. The ophthalmic device according to any one of examples 245-271, wherein said retention portion is configured to keep said ophthalmic device in place when the device is worn, and the eye is rolled.

Example 273. The ophthalmic device according to any one of examples 245-272, wherein a first height perpendicular to a reference surface and measured from a first point on said posterior surface to a corresponding point on said anterior surface differs by at least 1 mm, from a second height perpendicular to the reference surface and measured from a second point on said posterior surface to a corresponding point on said anterior surface, said second point being distanced at most 1 mm from said first point.

Example 274. The ophthalmic device according to any one of examples 245-273, sized and shaped to be fully covered by the lower eyelid when worn under a lower eyelid.

Example 275. The ophthalmic device according to any one of examples 245-274, wherein said ophthalmic device is configured for self-administration.

Example 276. The ophthalmic device according to any one of examples 245-275, wherein said device is configured for wearing away from a fornix' eye.

Example 277. The ophthalmic device according to any one of examples 245-276, comprising two or more types of PVOH and wherein each of said two or more types of PVOH constitutes at least 10% of the total amount of PVOH in the matrix.

Example 278. The ophthalmic device according to any one of examples 245-277, comprising two or more types of PVOH and wherein the two or more types of PVOH include one type that has long chains and is fully hydrolyzed, and one type that has at least one of:
a. short chains and is partially hydrolyzed;
b. long chains and is partially hydrolyzed;
c. short chains and is fully hydrolyzed.

Example 279. The ophthalmic device according to any one of examples 245-278, wherein:
a. short chains are of 200 to 2000 monomeric units;
b. long chains are of 2200 to 5000 monomeric units;
c. fully hydrolyzed PVOH is of hydrolysis degree of 97% or more; and
d. partially hydrolyzed is of hydrolysis degree of 95% or less.

Example 280. The ophthalmic device according to any one of examples 245-279, comprising two or more types of PVOH and wherein two types of the PVOH differ in chain length by at least 1000, and have similar degrees of hydrolysis, both between 97% and 100%.

Example 281. The ophthalmic device according to any one of examples 245-280, comprising two or more types of PVOH and wherein a first type of said two or more types has a hydrolysis degree from 97% to 100%.

Example 282. The ophthalmic device according to any one of examples 245-281, wherein a second type of said two or more types has a hydrolysis degree is lower than 93%.

Example 283. The ophthalmic device according to any one of examples 245-282, comprising two or more types of PVOH and wherein a second type of said two or more types has a hydrolysis degree from 80% to 93%.

Example 284. The ophthalmic device according to any one of examples 245-283, comprising two or more types of PVOH and wherein a first type of said two or more types has chains with more than 2500 units.

Example 285. The ophthalmic device according to any one of examples 245-284, comprising two or more types of PVOH and wherein a second type of said two or more types has chains with less than 1500 units.

Example 286. The ophthalmic device according to any one of examples 245-285, comprising two or more types of PVOH and wherein a relation between a first type of PVOH and a second type of PVOH of said two or more types is from about 3:1 to about 1:3.

Example 287. The ophthalmic device according to any one of examples 245-286, comprising two or more types of PVOH and wherein a first type of said two or more types has chains with more than 2500 units and hydrolysis degree of 97% to 100%, and a second type of said two or more types has chains with less than 1000 units and hydrolysis degree of 80% to 93%.

Example 288. The ophthalmic device according to any one of examples 245-287, comprising two or more types of PVOH and wherein said second type of PVOH makes more than 50% of said PVOH.

Example 289. The ophthalmic device according to any one of examples 245-288, comprising two or more types of PVOH and wherein said at least two types of PVOH determine rate of degradation of said ophthalmic device.

Example 290. The ophthalmic device according to any one of examples 245-289, wherein said rate of degradation comprises a rate by which a predetermined change in a predetermined mechanical property takes place in simulated tear fluid (STF).

Example 291. The ophthalmic device according to any one of examples 245-290, wherein a combined mass content of said PVOH and said one or more organic plasticizers is at least 70% wt of the total weight of the matrix excluding said water.

Example 292. The ophthalmic device according to any one of examples 245-291, wherein said elastomeric matrix is characterized by substantially isotropic swelling when being immersed in simulated tear fluid and at room temperature for 5 minutes.

Example 293. The ophthalmic device according to any one of examples 245-292, wherein said elastomeric matrix swells by less than 50% by volume when immersed in simulated tear fluid for 5 minutes at room temperature.

Example 294. The ophthalmic device according to any one of examples 245-293, wherein said water constitute less than 50% wt of the total mass content of the matrix.

Example 295. The ophthalmic device according to any one of examples 245-294, essentially devoid of covalent crosslinking.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a method of use of an ophthalmic device, according to some embodiments of the invention;

FIG. 2 is a method of treatment using an ophthalmic device, according to some embodiment of the invention;

FIG. 3A is a simplified schematic cross sectional view of an eye;

FIG. 3B is a simplified schematic cross sectional view of an ophthalmic device in position on an eye surface, when the eye is closed, according to some embodiments of the invention;

FIG. 3C is a simplified schematic cross sectional view of an ophthalmic device in position on an eye surface, when the eye is open, according to some embodiments of the invention;

FIG. 3D is a simplified schematic top view of an ophthalmic device in position on an eye surface, according to some embodiments of the invention;

FIG. 4A is a simplified schematic top view of an ophthalmic device in position on an eye surface, according to some embodiments of the invention;

FIG. 4B is a simplified schematic cross sectional view of an ophthalmic device in position on an eye surface, when the eye is closed, according to some embodiments of the invention;

FIG. 5 is a method of use of an ophthalmic device, according to some embodiments of the invention;

FIG. 6A is a simplified schematic top view of an ophthalmic device 600 in position on an eye surface underneath an eyelid, according to some embodiments of the invention;

FIG. 6B is a simplified schematic cross section of an ophthalmic device, according to some embodiments of the invention;

FIG. 7 is a simplified schematic illustrating cross sections of portions of devices, according to some embodiments of the invention;

FIGS. 8, 9, 10, 11 and 12 are simplified schematic top views of devices, according to some embodiments of the invention;

FIG. 13A is a simplified schematic view of an ophthalmic device, according to some embodiments of the invention;

FIG. 13B is a simplified schematic cross sectional view of an ophthalmic device, according to some embodiments of the invention;

FIG. 13C is a simplified schematic top view of an ophthalmic device, according to some embodiments of the invention;

FIG. 14 is a simplified schematic cross sectional view of an ophthalmic device, according to some embodiments of the invention;

FIG. 15A is a simplified schematic top view of an ophthalmic device, according to some embodiments of the invention;

FIG. 15B is a simplified schematic view of an ophthalmic device, according to some embodiments of the invention;

FIG. 15C is a simplified schematic cross sectional view of an ophthalmic device, according to some embodiments of the invention;

FIG. 15D is a simplified schematic cross sectional view of an ophthalmic device, according to some embodiments of the invention;

FIGS. 16, 17, 18, 19 and 20 are simplified schematic top views of devices, according to some embodiments of the invention;

FIGS. 21, 22, 23 and 24 are simplified schematic cross sections of devices, according to some embodiments of the invention;

FIG. 25A is a simplified schematic cross sectional view of a device positioned on an eye surface, according to some embodiments of the invention;

FIG. 25B is a simplified schematic cross sectional view of a device, according to some embodiments of the invention;

FIG. 26 is a simplified schematic cross sectional view of a device, according to some embodiments of the invention;

FIG. 27A is a simplified schematic top view of an ophthalmic device, according to some embodiments of the invention;

FIG. 27B is a simplified schematic cross sectional view of an ophthalmic device, according to some embodiments of the invention;

FIG. 27C is a simplified schematic cross sectional view of a device 2700, according to some embodiments of the invention;

FIG. 28 is a process of ophthalmic device degradation, according to some embodiments of the invention;

FIG. 29 is a simplified schematic cross sectional view of a device, according to some embodiments of the invention;

FIGS. 30A-B are simplified schematic cross sectional views of a device, according to some embodiments of the invention;

FIG. 31A is a simplified schematic cross section view of an ophthalmic device on an eye surface, according to some embodiments of the invention;

FIG. 31B is a simplified schematic view of an ophthalmic device, according to some embodiments of the invention;

FIG. 31C is a simplified schematic to view of an ophthalmic device, according to some embodiments of the invention;

FIGS. 32, 33, 34, 35, 36 and 37 are simplified schematic cross section views of portions of ophthalmic devices, according to some embodiments of the invention;

FIGS. 38, 39, 40, 41, 42, 43 and 44 are simplified schematic cross sectional views of devices, according to some embodiments of the invention;

FIGS. 45, 46, 47, 48, 49 and 50 are simplified schematic cross sectional views of double layer devices according to some embodiments of the invention;

FIGS. 51, 52, 53, 54, 55, 56, 57 and 58 are simplified schematic cross sectional views of multi-layer devices, according to some embodiments of the invention;

FIG. 59A is a simplified schematic cross section of an ophthalmic device, according to some embodiments of the invention;

FIG. 59B is a simplified schematic cross section of an ophthalmic device, according to some embodiments of the invention;

FIG. 60 is a simplified schematic cross section of an ophthalmic device, according to some embodiments of the invention;

FIG. 61 is a simplified schematic cross section of an ophthalmic device, according to some embodiments of the invention;

FIG. 62A is a simplified schematic cross sectional view of a portion of an eye surface, according to some embodiments of the invention;

FIG. 62B is a simplified schematic cross section view of an ophthalmic device on an eye surface, according to some embodiments of the invention;

FIG. 62C is a simplified schematic cross section view of an ophthalmic device on an eye surface, according to some embodiments of the invention;

FIGS. 63A-C are simplified schematic cross sectional views of a portion of a device on an eye surface, according to some embodiments of the invention;

FIGS. 64, 65 and 66 are simplified schematic cross sectional views of multi-layer devices, according to some embodiments of the invention;

FIGS. 67A-F are simplified schematic cross sections of a portion of a device as residence time of the device progresses, according to some embodiments of the invention;

FIG. 68 is a flowchart of a disintegration progression of a device, according to some embodiments of the invention;

FIGS. 69A-C are simplified schematic cross sections of a device as residence time of the device progresses, according to some embodiments of the invention;

FIGS. 70A-D are simplified schematic cross sections of a device as residence time of the device progresses, according to some embodiments of the invention;

FIG. 71 is a simplified schematic cross section of a device, according to some embodiments of the invention;

FIG. 72 is a simplified schematic cross section of a device, according to some embodiments of the invention;

FIGS. 73A-B are simplified schematic cross sectional views of a device on an eye surface 7304, according to some embodiments of the invention;

FIGS. 74A-D are simplified schematic cross sectional views of a film, according to some embodiments of the invention

FIG. 75 is a method of manufacture, according to some embodiments of the invention;

FIGS. 76A-C are simplified schematic cross sectional views of a device, according to some embodiments of the invention;

FIG. 77 is a graph illustrating exemplary degradation characteristics of three different elastomeric matrix compositions, as they evolve in time under wet conditions, according to some embodiments of the invention;

FIG. 78 is a graph illustrating degradation of some matrices during wetting and drying cycle, according to some embodiments of the invention;

FIG. 79 is a graph plotting clinical results obtained according to embodiments of the present invention in comparison with published data related to eye-drops; and

FIG. 80 is a graph showing the effect of an ophthalmic device according to embodiments of the present invention on near visual acuity (NVA) of two patients.

### DESCRIPTION OF SPECIFIC EMBODIMENTS OF THE INVENTION

The present invention, in some embodiments thereof, relates to shaped polyvinyl alcohol (PVOH) based elastomeric matrixes for ophthalmic uses and, more particularly, but not exclusively, to such matrixes that are biodegradable or bioerodible. The terms “biodegradable” and “bioerodible” (as well as their inflictions, such as biodegradability and bioerodibility) are used herein interchangeably. For example, a matrix may be considered biodegradable if it erodes mechanically in the body.

### Overview

The present invention, in some embodiments thereof, provides an ophthalmic drug product, uses thereof, and methods of its manufacturing. The ophthalmic drug product is preferably an ophthalmic device. All the features of the drug products described herein may also be found in any ophthalmic device described herein, and vice versa. In some embodiments, the ophthalmic device is configured to remain between a bulbar conjunctiva (ocular conjunctiva) and an eyelid, more particularly, between the ocular conjunctiva covering the sclera and the palpebral conjunctiva (tarsal conjunctiva), covering the eyelid. The tarsal and ocular conjunctivae meet at the fornix conjunctiva, and, in some embodiments, the device is configured for wearing away from the fornix, closer to the opening of the eyelid. In some embodiments, the ophthalmic device extends under either the lower eyelid or the upper eyelid. A potential advantage of having a device positioned under either and not both is that it potentially provides a smaller and more comfortable device, which in some embodiments may be inserted, and optionally taken out, by the patient himself. In some embodiments, the ophthalmic device is configured to reside between an eyeball and an eyelid, and to move with the eyelid when the eyelid moves in respect to the eyeball. In some embodiments, the ophthalmic device is configured to reside between an eyeball and an eyelid, and to stay with the eyelid when the eyeball moves in respect to the eyelid. In some embodiments, the ophthalmic device is configured to reside between an eye surface and an eyelid, and it comprises a body made of an elastomeric matrix, wherein said elastomeric matrix comprises: water, one or more types of PVOH, and one or more organic plasticizers. In some embodiments, the body characterized by: a posterior surface which is adjacent to an eye surface when said ophthalmic device is worn; an anterior surface, which is adjacent to said eyelid covering said eye surface when said ophthalmic device is worn; a retention portion, defined between a portion of said anterior surface and a portion of said posterior surface; where the retention portion has an edge structured to cause the ophthalmic device to move with the eyelid when the eyelid moves in respect to the eyeball.

In some embodiments, the ophthalmic device is configured to remain between the ocular and tarsal conjunctivae even when the eyelid or eyeball moves one in respect to the other. In some embodiments, during movement between the eyelid and the eyeball, the device moves with the eyelid across the eyeball, and in other embodiments, the device moves with the eyeball.

The embodiments in which the device moves with the eyeball or with the eyelid differ from one another in the balance between a grabbing force and an attaching force. The grabbing force acts between the edge of the eyelid and the edge of the device adjacent to the eyelid; and the attaching force acts on the posterior surface of the device (facing the eye surface). The attaching force may include, for example, adhesive friction force (traction force), and/or suction force.

In some embodiments, the grabbing force is larger than the attaching force, and the ophthalmic device moves with the eyelid across the eyeball. In some embodiments, the attaching force is larger than the grabbing force, and the ophthalmic device moves (or stays) with the eyeball.

In some embodiments, the ophthalmic device is configured to have enlarged grabbing force by including a retention portion with a steep leading edge. In some embodiments, the leading edge is the edge adjacent to the opening of the eyelid. In some embodiments, the retention portion is a portion of the ophthalmic device, defined between a portion of the posterior surface and a corresponding portion of the anterior surface, which is the surface of the device facing the eyelid. In some embodiments, the anterior surface defines heights above the eye surface. In some embodiments, to have enlarged grabbing force, the retention portion is optionally structured with two of the heights, defined by the corresponding portion of the anterior surface, being both close to each other, and having between them a large height difference, so as to create an angle of 45 degrees or more between the anterior surface and the eye surface. For example, the two heights may be distanced from each other by at most 1 mm, while differ from one another by a height difference of at least 1 mm.

In some embodiments, the ophthalmic device is configured to have enlarged attaching force, for example, by including one or more cavities, configured to receive therein a portion of the ocular conjunctiva, so as to enlarge the friction between the device and the eyeball. Similarly, in some embodiments, the ophthalmic device is configured to have enlarged grabbing force, for example, by including one or more cavities, configured to receive therein a portion of the tarsal conjunctiva, so as to enlarge the friction between the device and the eyelid.

In some embodiments, an ophthalmic device may be configured to move with the eyelid across the eyeball by having a steep retention portion, with no cavities at the posterior surface.

In some embodiments, an ophthalmic device may be configured to move with the eyeball by having a retention portion which is not steep, and having cavities at the posterior surface.

In some embodiments, the ophthalmic device has both a posterior surface with one or more cavities and a steep retention portion, and the device behavior when the eyeball moves in respect of the eyelid is determined by the relative effects of the cavities and steepness of the retention portion.

In some embodiments with steep retention portion, the retention portion is pressed hard against the eyelid, optionally, at a force hard enough to overcome friction between the posterior surface and the eye surface. Such hard press between the eyelid and the retention portion may cause significant discomfort to the wearer of the device. In some embodiments, to prevent such discomfort, the retention portion is made of a material soft enough not to irritate the eye when pressed against the eyelid even at the hard force required to drag the device with the eyelid.

In some embodiments, the required softness may be achieved with an elastomeric matrix formulated with water, PVOH, and a large amount plasticizers. The amount of the plasticizers may be considered large if it is larger than, or, preferably, at least double (in mass) than the mass amount of the PVOH. In some such embodiments, the mass of the PVOH and the plasticizers together is 70% or more of the total mass of the matrix excluding the water. In some embodiments, said PVOH comprises PVOH of two or more types that differ from one another in one or both of hydrolysis degree (HD) and chain length. In some embodiments, each of said two or more types of PVOH constitutes at least 10% of the total amount of PVOH in the matrix. In some embodiments, the two or more types of PVOH include one type that has long chains and is fully hydrolyzed, and one type that has at least one of: short chains and is partially hydrolyzed; long chains and is partially hydrolyzed; short chains and is fully hydrolyzed. In some embodiments, short chains are of 200 to 2000 monomeric units; long chains are of 2200 to 5000 monomeric units; fully hydrolyzed PVOH is of hydrolysis degree of 97% or more; and partially hydrolyzed is of hydrolysis degree of 95% or less.

In some embodiments, the required softness may be achieved with an elastomeric matrix, having at least one of the following mechanical characteristics:
Tensile strength of between 0.01 MPa and 10 MPa, optionally 0.05 MPa to 10 MPa. Optionally, the tensile strength is smaller than 10 MPa, smaller than 4 MPa, smaller than 3 MPa or smaller than 2 MPa;
Elastic modulus of between 0.01 MPa and 2.5 MPa, optionally between 0.05 MPa and 10 MPa. Optionally, the elastic modulus is smaller than 10 MPa, smaller than 4 MPa, smaller than 3 MPa or smaller than 2 MPa;
Elongation at breakage of between 50% and 1000%, or larger. See also Mechanical Properties shown in Example 8, below.

In some embodiments, after the ophthalmic device is in the eye for some time (e.g., 5 or 10 minutes, 30 minutes, 1 hour, or four hours, or intermediate durations), the properties of the elastomeric matrix change. In some embodiments, this process, generally referred to herein as “degradation”, may change the balance of forces between the device and the tarsal conjunctiva on the one hand, and the device and the ocular conjunctiva on the other hand. In some embodiments, the degradation improves the fit to the anatomical locale at which the device seats. For example, the device may loosen its attachment to the eye surface (ocular conjunctiva), e.g., due to change in shape, and strengthen its attachment to the eyelid surface (tarsal conjunctiva), e.g., due to development of mucoadhesive properties of the matrix or better fitting to the conjunctiva. Thus, in some embodiments, after staying for some time in the eye, the degradation of the device may have an effect of releasing the device from the eye surface, while adhering the device to the eyelid and preventing the device from arriving (e.g., by floating on tear fluid) to the cornea, from which it might be quickly expelled by natural expulsion mechanisms.

In some embodiments, the ophthalmic device is configured for residing in the eye, e.g., in contact with an outer surface of an eyeball and/or with an inner surface of an eyelid, for long periods, for example, of up to 24 hours without activating mechanisms that naturally expel foreign bodies from the eye, such as, eyeball movements, blinking, and increased tendency to rub the eye. In some embodiments, while such mechanisms may be activated for a short period immediately after administering the ophthalmic device, they are soon lessened to an extent that the eye is not irritated, and the ophthalmic device remains in the eye without expulsion.

In some embodiments, this fast calming of the eye is achieved, inter alia, by the matrix being soft and supple even before being inserted to the eye, and particularly in its dry form. In some embodiments, the suppleness of the matrix may be expressed in being readily adaptable to new environments, and capable of being bent and/or folded without creases, cracks, or breaks. In some embodiments, the softness, in combination with the pliability (or suppleness) allows the matrix to flex and adapt in shape to the space available in the anatomical space at which it is inserted, e.g., in the lid wiper space between the lower eyelid and bulbar conjunctiva. In some embodiments, flexing and shape adaptation improves the fit between the ophthalmic device and the space in the eye over time, thus potentially lessen discomfort and allowing long residence of the ophthalmic device on the eye.

In some embodiments, the ophthalmic device is configured to be stored for long periods under dry conditions without losing its shape, softness and/or elasticity, and thus enjoys particularly long shelf life.

In some embodiments, the ophthalmic device is configured to shrink and swell, when taken out from the dry conditions and into inserted into water (or wet conditions).

In some embodiments, shrinking and swelling is characterized by being substantially isotropical. As used herein, shrinking or swelling substantially isotropically means going through size change along each dimension by the same amount±about 20%. For example, in ophthalmic devices having rectangular shape, the length, width, and thickness (as may be measured, e.g., by a caliber) change by the same amount±about 20%. In ophthalmic devices having cylindrical shape, the diameter and the height change by the same amount±about 20%. In some embodiments, the total swelling, in addition to being isotropic, is not larger than 50% by volume, that is, each dimension (e.g., length, width, and depth) may be enlarged by not more than about 15% in the first minute or two after being immersed in water. In some embodiments, after the first minute or two, the drug product does not continue to swell, but may get smaller, e.g., due to degradation. In some embodiments, the requirement for isotropic swelling of 50% or less means that the enlargement along each direction (e.g., as measured by a caliber) is of 18% (15t3)% or less. In some embodiments, the ophthalmic device, and/or the elastomeric matrix comprised in it, is characterized by substantially isotropic swelling when being immersed in simulated tear fluid and at room temperature for 5, 10, or 15 minutes. In some embodiments, the ophthalmic device, or the elastomeric matrix, swells by less than 50% by volume when immersed in simulated tear fluid for 5, 10, or 15 minutes at room temperature.

In some embodiments, the isotropic swelling and shrinking cause the ophthalmic device to have substantially the same shape (even if not the same volume) under dry and wet conditions (at least for the first minutes under wet conditions). As used herein, dry conditions are conditions in which the drug product is kept under ambient humidity, but with no direct contact with liquids, including water and aqueous liquids. Wet conditions are conditions in which the drug product is kept in contact with aqueous liquids, such as water, tear fluid, tear simulation fluid, or the like.

In some embodiments, the ability to stay in the eye with only limited and short irritation may also benefit from the shape of the ophthalmic device. For example, in some embodiments, the soft and pliable matrix is shaped to include a retention portion, adapted to hold the drug product in its place, e.g., in a space between the bulbar conjunctiva and the eyelid. In some embodiments, the retention portion is adapted to hold the drug product in a space between the bulbar conjunctiva and the eyelid when the device is worn, and the eye is rolled. In some embodiments, the retention portion may prevent the ophthalmic device from escaping to other portions of the eye, for example, to under the upper eyelid, where the ophthalmic device might cause more discomfort, despite its high level of softness and pliability. In some embodiments, the softness, pliability, and suppleness of drug product allows making it relatively large (e.g., having an area of between 15 mm² and 35 mm² parallel to the eye surface and up to 5 mm thickness) without disturbing the eye.

In some embodiments, mechanical properties associated with the softness and elasticity of the ophthalmic device usually include at least one of the following: (i) a tensile strength of between 0.01 MPa and 2 MPa, optionally between 0.05 MPa and 1 MPa; (ii) elastic modulus (also known as Young modulus under tension) of between 0.01 MPa and 2.5 MPa, optionally between 0.05 MPa and 2 MPa; (iii) elongation at break of from about 50% up to about 1000%, optionally from about 100% to about 900%, optionally between 200% and 600%; and (iv) compression module (also known as Young's modulus under compression) of between 0.05 MPa and 2.5 MPa, optionally between 0.1 MPa and 2.0 MPa.

An aspect of some embodiments of the invention relates to elastomeric matrices (referred hereinafter just as “matrix” or “matrices/matrixes”), which comprise one or more types of PVOH in order to achieve a matrix having determined and/or desired mechanical properties. In some embodiments, the elastomeric matrices are PVOH-based, in the sense that PVOH is the sole or main film forming polymer in the matrix.

In some embodiments, a matrix is designed to dissolve and/or degrade under wet conditions according to predetermined timeline requirements. In some embodiments, the timeline of degradation is controlled by choosing a composition of the one more types of PVOH in the matrix. In some embodiments, the composition is optionally characterized by the various types of PVOH included in the composition and their relative masses, or the mass portion of the total PVOH provided by each of the types.

In some embodiments, when referring to “wet conditions”, it should be understood to relate to conditions wherein a matrix of the invention is in direct contact with liquid (immersed in the liquid or after immersion in the liquid and before the liquid evaporated or wiped away). In some embodiments, the liquid is a naturally occurring bodily fluid, including but not limited to blood, saliva, tears, excreta, body tissue, tissue fluids), an aqueous solution (e.g., of one or more organic plasticizers), a buffer solution, and any combinations thereof. In some embodiments, the liquid is naturally occurring or simulating a naturally occurring fluid, such as artificial tear fluid. In some embodiments, artificial tear fluid is based on an aqueous solution containing about 0.67% sodium chloride, about 0.2% sodium bicarbonate and about 0.008% calcium chloride. In some embodiments, “wet conditions” refer to the conditions when the matrix is positioned on mucous tissue of a human, for example, on buccal tissue or an eye surface.

In some embodiments, the term degradation of a matrix is defined as the change in properties of the matrix over time under wet conditions, and “degradability” as the tendency to degrade or the rate of degradation.

In some embodiments, the change in properties may include, for example, a change or loss in mass of one or more of the matrix components or of the matrix as a whole. For example, while plasticizers that leave the ophthalmic device may be replaced by water, in some embodiments, PVOH itself leaves the ophthalmic device, and thus, the device loses weight. Alternatively, or additionally, the change may include change in mechanical properties, shape, and/or size of the matrix. Alternatively, or additionally, the degradation may be expressed as dissolution, breakage, tear, and/or crumbling of the matrix. In general, the degradation may be due to breakage of bonds between the different constituents of the matrix (e.g., PVOH, plasticizers, and water) while each of the constituents stays intact. In some embodiments, the degradation allows clearing the ophthalmic device, or the elastomeric matrix it comprises, from the eye, for example, by tears, that may wash the matrix constituents, crumbs, and/or remains out of the eye.

In some embodiments, the kind of degradation, e.g., the change from soft solid to slime, dissolution, mass loss, or change in tearability, may depend on the types of PVOH included in the matrix. For example, the inventors found that some matrices that include long chain fully hydrolyzed (LCFH) PVOH and short chain partially hydrolyzed (SCPH) PVOH tend to become “slimy” when immersed in simulated tear fluid (STF). In such embodiments, the increased sliminess may be accompanied by a decrease in compression modulus with time under STF. In some embodiments, some matrices that include LCFH PVOH and long chain partially hydrolyzed (LCPH) PVOH tend to become more easily dissolvable in STF, in the sense that they may dissolve to clear solution in cold water. In some embodiments, some matrices that include LCFH PVOH and short chain fully hydrolyzed (SCFH) PVOH tend to be torn more easily the longer they are immersed in STF. In some embodiments, increased susceptibility to tearing of such matrices may be accompanied by decrease in compression strength with time under STF.

See also Exemplary Mechanical Properties in Example 8 below.

In some embodiments, the degradation may obviate the need to take the matrix out of the body. For example, the products of the degradation may be expelled from the body naturally, e.g., by tear fluid (if the matrix is used in the eye) by urea (if the matrix is used in the bladder), etc. In case of ophthalmic devices, after some time in the eye, an ophthalmic device made of a degradable matrix may leave the eye naturally, with no need to take it out actively from the eye. For example, the device may fully dissolve and leave the eye with tear fluid, or disintegrate to such small parts that may be expelled from the eye as any other foreign object.

In some embodiments, slightly pressing a degradable matrix (e.g., used as ophthalmic device) may bring to immediate change in the form or integrity of the matrix, so an ophthalmic device may be naturally expelled shortly after being pressed.

In some embodiments, the degradation causes the matrix to acquire a shape and/or consistency that are comfortable to the anatomic locale at which it is used, so it may remain there. For example, an ophthalmic device may acquire a shape conforming to the space between the ocular and tarsal conjunctivae, where it may rest without discomforting the patient until it is degraded and expelled from the eye. In some embodiments, on the way out from the eye a degraded device might come across the cornea and cause temporary discomfort.

In some embodiments, the terms “dissolve” and “degrade” are interchangeable and refer to the process by which the elastomeric matrices change, e.g., under wet conditions. The change may be, for example, from a solid state to a completely degraded state. The change may be, for example, loss in mass. In some embodiments, a completely degraded state can be defined, for example, as a degradation state of the matrix when the matrix cannot be seen by the naked eye, for example because the matrix has disintegrated to particles of very small size, and/or, for example, a state of the matrix when the matrix elastically changes shape and/or does not hold a defined form under certain external pressures, for example, in matrices used in or applied to any tissue, for example on the eye, pressure from the eyelid on the matrix. The terms “shape” and “geometry” are interchangeable and refer to the form of the matrix.

In some embodiments, the matrixes comprise a mixture of two or more types of PVOH. In some embodiments, the differences in the types of PVOH are in one or more of hydrolysis degree and length of PVOH chains. In some embodiments, the higher the quantity of highly hydrolyzed PVOH in the matrix, the longer the time that will take for the matrix to fully degrade. In some embodiments, said PVOH comprises PVOH of two or more types that differ from one another in one or both of hydrolysis degree (HD) and chain length. In some embodiments, each of said two or more types of PVOH constitutes at least 10% of the total amount of PVOH in the matrix. In some embodiments, the two or more types of PVOH include one type that has long chains and is fully hydrolyzed, and one type that has at least one of: short chains and is partially hydrolyzed; long chains and is partially hydrolyzed; short chains and is fully hydrolyzed. In some embodiments, short chains are of 200 to 2000 monomeric units; long chains are of 2200 to 5000 monomeric units; fully hydrolyzed PVOH is of hydrolysis degree of 97% or more; and partially hydrolyzed is of hydrolysis degree of 95% or less.

In some embodiments, the number of PVOH types is three or more.

In some embodiments, two of the types of PVOH differ in chain length, e.g., by at least 1000 or 1500 units. In some such embodiments, the two of the types of PVOH have similar degrees of hydrolysis, e.g., between 97% and 100%.

In some embodiments, two of the types of PVOH differ in degree of hydrolysis, e.g., one has D.H of 97% or more, and the other of 93%, 90%, or less. In some such embodiments, the two types of PVOH differ also in chain length, e.g., by at least 1000 or 1500 units. For example, the PVOH type with the higher degree of hydrolysis may have longer chains. In another example, the PVOH type with the higher degree of hydrolysis may have shorter chains. In some embodiments, in which two of the types of PVOH differ in degree of hydrolysis, the two types of PVOH have similar lengths, differing from each other by, e.g., less than 500 units.

In some embodiments, the PVOH with short chains has an average chain length of between about 200 and about 2000, monomeric units; and the PVOH with long chains has average chain length between about 2200 and about 5000 monomeric units. In some embodiments, the fully hydrolyzed PVOH has a hydrolysis degree of 97% or more; and the partially hydrolyzed PVOH has hydrolysis degree of 94% or less. In some embodiments, short chains have a molecular weight lower than about 50 Kg/Mol to about 80 Kg/Mol, while long chains have a molecular weight higher than about 50 Kg/Mol to about 80 Kg/Mol.

In some embodiments, the matrices are specifically designed to keep their geometry/shape under wet conditions, before the initiation of the degradation process. The degradation process may begin, in some embodiments, after 1 minute under wet conditions and in other embodiments after several hours under wet conditions, or within intermediate times. A matrix that keeps its geometry may be shaped outside the body (e.g., the eye), knowing that the same shape will be retained at least for some period after contacting the body.

In some embodiments, the elastomeric matrix comprises PVOH, one or more organic plasticizers, and optionally water. In some embodiments, the elastomeric matrix is characterized by a ratio between the combined mass of the one or more plasticizers to a combined mass of the PVOH of the various types that is at least 2:1.

$$That is , \frac{total mass of plasticizer ( s )}{total mass of PVOH} \geq \frac{2}{1} .$$

This ratio is referred to herein as “combined mass ratio”.

In some embodiments, the elastomeric matrix comprises more than one type of plasticizer.

In some embodiments, the matrix is characterized by keeping a geometry when coming in contact with a wet environment, for example when put in contact with the eye or other body tissue. In some embodiments, in addition to keeping its geometry when coming in contact with a wet environment, optionally, the matrix increases its volume. In some embodiments, the volume increase is by no more than 50%. In some embodiments, the swelling is the same in all directions, so that the volume changes, but the geometry is kept. In some embodiments, the elastomeric matrix is characterized by substantially isotropic swelling when being immersed in simulated tear fluid and at room temperature for 5, 10, or 15 minutes. In some embodiments, the elastomeric matrix swells by less than 50% by volume when immersed in simulated tear fluid for 5, 10, or 15 minutes at room temperature.

In some embodiments, the mechanical properties and/or the degradability of the elastomeric matrix depends on the types of PVOH used to form the matrix, and their respective masses. For example, when the PVOH includes mainly long chain fully hydrolyzed (hereinafter LCFH) PVOH and a minority of short chain partially hydrolyzed (hereinafter SCPH) PVOH the matrix degrades more slowly than when the LCFH PVOH is the minority of the PVOH in the matrix and SCPH PVOH is the majority thereof.

The following table (Table 1) provides examples of some commercially available types of PVOH. The list is taken from the brand Mowiol™, but similar materials are available from other sources. The PVOH type named “30 k” in the table below (Table 1) is not of the Mowiol brand. All PVOH types used in the examples and measurements recited herein were carried out with PVOH sold under the brand of Emprove™ or with the 30 k PVOH (which is Sigma Aldrich 8.21039).

TABLE 1

| | | | MW | Chain | Hydrolysis |
|---|---|---|---|---|---|
| | | Name | [kg/mol] | length | degree [%] |
| Partially | Short chain | 3-83 | 14 | 270 | 83 |
| hydrolyzed | (SCPH) | 4-88 | 31 | 630 | 88 |
| | | 5-88 | 37 | 750 | 88 |
| | | 8-88 | 67 | 1400 | 88 |
| | Long chain | 18-88 | 130 | 2700 | 88 |
| | (LCPH) | 23-88 | 150 | 3100 | 88 |
| | | 26-88 | 160 | 3300 | 88 |
| | | 40-88 | 205 | 4200 | 88 |
| | | 30-92 | 175 | 3700 | 92 |
| Fully | Short chain | 3-98 | 16 | 360 | 98 |
| hydrolyzed | (SCFH) | 4-98 | 27 | 600 | 98 |
| | | 6-98 | 47 | 1000 | 98 |
| | | 10-98 | 61 | 1400 | 98 |
| | | 30k | 30 | 670 | 99 |
| | Long chain | 20-98 | 125 | 2800 | 98 |
| | (LCFH) | 56-98 | 195 | 4300 | 98 |
| | | 28-99 | 145 | 3300 | 99 |

In some embodiments, the elastomeric matrix is used as an ophthalmic device or make part of an ophthalmic device. In some embodiments, the ophthalmic device may be configured to be positioned on the surface of the eye, preferably on the sclera. In some embodiments, options of positioning the ophthalmic device include one or more of: topical (sclera, cornea), subconjunctiva, conjunctiva, suprachoroidal, intraviteral, intra cameral, subretinal, or any other way to provide to the eye or onto or into other body parts. E.g., body cavities, (bladder, stomach) joints, etc. by intramuscular injection. In some embodiments, a potential advantage of positioning on the sclera is that the sclera is potentially less sensitive (to pain, irritation, etc.) than the cornea, so designing the matrix to contact only the sclera potentially results in a matrix that is more convenient for the user. In some embodiments, depending on the composition of the PVOH types included in the elastomeric matrix, complete degradation occurs, for example, between 0.5 hours and 12 hours from contacting the eye. Optionally from between 0.5 hours and 24 hours. Optionally between 0.5 hours and 48 hours. Optionally within longer than 48 hours. In some embodiments, a potential advantage of controlling the degradation time is that it allows a user to use the ophthalmic device during convenient and/or chosen hours (for example: while the user is sleeping, or for example to allow the device to act at a desired time window).

In some embodiments, the elastomeric matrix is characterized under dry conditions by a tensile strength below 10, 4, 3, MPa, preferably below 2 MPa. Additionally or alternatively, the elastomeric matrix is characterized under dry conditions by elasticity (aka Young Module) of 0.01 MPa to 10, 4, or 3 MPa, preferably 0.01 MPa to 2 MPa. Additionally or alternatively, the elastomeric matrix is characterized under dry conditions by an elongation at breakage of at least 50%, for example, 50% to 900% or 50% to 1000%. In some embodiments, a potential advantage to have a matrix characterized the above-mentioned mechanical properties is that it potentially reduces discomfort in the user, thereby potentially increasing compliance.

A broad aspect of some embodiments relates to an ophthalmic device, made with the elastomeric matrices as disclosed herein, for treatment and/or protection of the eye which, when positioned on an eye remains within the eye, e.g., between an eyelid and an eye surface. In some embodiments, the device stays on the eye for at least 15 minutes. In some embodiments, the device remains in the eye. For example, without moving from a region of the eye to which it has been positioned and/or without being expelled from the eye during a treatment time period. Where, in some embodiments, the device is positioned underneath the eyelid and/or on a portion of the eyeball. Whilst, in some embodiments, the device remains comfortable for a user to wear.

Preferably, the device does not cover even a portion of the cornea. Not touching the cornea may have an effect of reducing irritation (in comparison to devices that do touch the cornea), because the cornea is very sensitive to foreign bodies. In some embodiments, the device is configured to be put on the bulbar conjunctiva, which is relatively insensitive to irritation. A device configured to be worn on the bulbar conjunctiva may also be effective as it is relatively easy to wear and take off, in comparison to devices in other parts of the eye.

In some embodiments, the device is in residence on the eye and/or remains in position (e.g. underneath an eyelid) during regular activities of a user/patient, for example, one or more of, blinking, tearing, crying, sweating, washing (e.g. showering), walking, exercising, sleeping. In some embodiments, the device is in residence on the eye and/or remains in position while the eyes are mainly closed e.g. during sleeping and/or for a semi-conscious or unconscious patient. In some embodiments, the ophthalmic device includes one or more active pharmaceutical ingredient (API) and/or other compound/s which are released (e.g. eluted from the device) while the device resides in the eye.

An aspect of some embodiments relates to an ophthalmic device, made with the elastomeric matrices as disclosed herein, which is configured to remain in position, underneath an eyelid of an eye. In some embodiments, optionally, part of the ophthalmic device remains in position underneath an eyelid of an eye while another part of the ophthalmic device extends outside the eyelid, optionally without reaching the cornea. In some embodiments, the ophthalmic device is configured to completely reside and remain between the surface of the eye and the eyelid. In some embodiments, the ophthalmic device does not comprise any part that extends outside the eyelid. In some embodiments, the device is configured to reside on a bulbar conjunctiva and underneath the eyelid, optionally, even during opening and closing of the eye (e.g. blinking).

In some embodiments, the ophthalmic device includes a retention portion which is sized and/or shaped to maintain the device underneath the eyelid. In some embodiments, the retention portion is the whole ophthalmic device. In some embodiments, the device have parts additional to the retention portion. In some embodiments, a device may include more than one retention portion. Where, in some embodiments, the retention portion is sufficiently thick and/or presents a sufficient height and/or a sufficiently steep slope to eye tissue that the device remains in position underneath the eyelid. For example, an anterior surface of the device presents a sufficient height and/or slope to eye tissue e.g. to an edge of the eyelid and/or to the tarsal plate.

In some embodiments, the thickness and/or height extends sufficiently in one or more directions. In some embodiments, the retention portion includes an anterior surface which presents a sufficient slope to eye tissue. In some embodiments, properties of the retention portion are dependent on one or more of a shape of the anterior surface at the retention portion, a thickness of the device (e.g. as defined between the anterior and posterior surfaces), a shape of the posterior surface. In some embodiments, when on an eye surface, the properties of the retention portion additionally depend on material properties (e.g. flexibility) of the device, for example as disclosed in relation to the exemplary elastomeric matrices disclosed herein, and the forces which the device experiences when in position on an eye surface and/or underneath an eyelid. In some embodiments, if the anterior surface of the retention portion is planar, the height of the retention portion is the same as a thickness of the device at the retention portion. In some embodiments, heights and/or slopes of the retention portion, in relation to the anterior surface, when in a dry configuration and/or a hydrated configuration, for example, are defined as for the device in a relaxed configuration and in relation to a plane on which the device rests, and/or on a plane connecting a majority of an edge circumference of the device. In some embodiments, heights and/or slopes of the retention portion, when in a dry configuration and/or a hydrated configuration, for example, are defined as for the device when in a relaxed configuration on a surface with radius of curvature (in one or more directions e.g. a sphere with the radius of curvature) of that expected of the eyeball onto which the device is to be positioned e.g. a radius of curvature of an adult human sclera, e.g. in both directions, e.g. radius of curvature of about 12 mm. In some embodiments, heights and/or slopes of the retention portion, when in a dry configuration and/or a hydrated configuration, for example, are defined as for the device when on an eye surface where, in some embodiments, the device is deformed (e.g. flattened) by suction between the device and the eye surface and/or pressure onto the device (e.g. by the eyelid).

In some embodiments, height of portion/s of the device is affected by curvature of the posterior surface, the higher the curvature the higher the height, for a same thickness device. In some embodiments, the retention portion has a part with a sufficiently steep slope followed by a part with a sufficient thickness having a sufficient length, and/or area to allow retention of the device under the eyelid. Where, for example, the retention portion has heights with distance from the leading edge (or edge of the retention portion) as described herein and may also include a portion having a height higher than a holding height, where the portion has a sufficient extent to allow retention of the device under the eyelid. For example, the portion with the holding height may have an area of at least 1 mm², or 0.5-3 mm², or 0.5-2 mm². Where the holding height is 0.5-5 mm, or 1-3 mm. In some embodiments, a maximum height of the device is less than 3 times, or less than 2 times, or less than 1.5 times the holding height. In some embodiments, the device retention portion has a height of at least 1 mm at 1 mm inwards from an edge (e.g. leading edge) of the device and/or from an edge of the retention portion. In some embodiments, the device retention portion has a height of 50-2500 micrometers or 500-1000 micrometers at 0.5 mm, or at 1 mm, or 2 mm from a leading edge of the device.

In some embodiments, the ophthalmic device comprises a body having a posterior surface, and an anterior surface. In some embodiments, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

A retention portion may be defined in between corresponding portions of the posterior and anterior surfaces. When the device is properly put in an eye, the posterior surface is adjacent the surface of the eye and the anterior surface is adjacent to an eyelid of the eye, defining heights above the eye surface.

Before being put in the eye, the device may be disposed in a relaxed configuration on a planar reference surface, with the posterior surface facing the planar reference surface. In such a position, the anterior surface defines heights above the planar reference surface. In some embodiments, the reference surface is defined as a surface (either real or imaginary) facing the posterior surface of a device in a relaxed configuration.

In some embodiments, two of said heights, measured from the eye surface and distanced from each other by a distance of at most 1 mm, differ from one another by a height difference of at least 1 mm. Alternatively or additionally, two of said heights, measured from the planar reference surface and distanced from each other by a distance of at most 1 mm, differ from one another by a height difference of at least 1 mm. In some embodiments, the reference surface is defined as a surface (either real or imaginary) facing the posterior surface of a device in a relaxed configuration.

In some embodiments, a first height measured from a first point on the posterior surface to a corresponding point on the anterior surface differs by at least 1 mm, from a second height measured from a second point on the posterior surface to a corresponding point on the anterior surface, where the second point is distanced at most 1 mm from the first point.

In some embodiments, the ophthalmic device comprises a body that comprises a posterior surface which is adjacent to an eye surface when the ophthalmic device is worn, an anterior surface which is adjacent to an eyelid when the ophthalmic device is worn. A retention portion may be defined by heights between the posterior surface and the anterior surface. In some embodiments, at least some of the heights between the posterior surface and the anterior surface differ from each other by at least 1 mm when measured from two points on the posterior surface distanced from each other by at most 1 mm.

In some embodiments, the device retention portion is formed, at least partially, once the device is in the eye. For example, in some embodiments, the device crumples and/or squashes and/or folds to provide a retention portion. In some embodiments, under movement of the eyelid. In some embodiments, the retention portion of the ophthalmic device allows the ophthalmic device to remain between an eyelid and an eye surface. In some embodiments, when the ophthalmic device is positioned under the lower eyelid, the retention portion prevents the ophthalmic device from exiting the lower eyelid. In some embodiments, when the ophthalmic device is positioned under the upper eyelid, the retention portion prevents the ophthalmic device from exiting the upper eyelid.

In some embodiments, the retention portion is configured to prevent the ophthalmic device from sliding under an upper eyelid when the ophthalmic device is positioned (advertently or not) on the surface of the eye but not under either the lower eyelid or the upper eyelid (for example positioned in a location on the surface of the eye that is visible). Optionally, such retention portion allows the ophthalmic device to slide under the lower eyelid when a user closes his eyes. Thus, in some embodiments, a device residing on a visible portion of the eye may find its way to the bulbar conjunctiva and stay there, thanks to the retention portion.

In some embodiments, the device is thick, for example having a maximal and/or average thickness between the anterior and posterior surface of 400-2500 microns, or of at least about 1 mm. A potential advantage of such thicknesses (optionally, in combination with an extent of the retention portion and/or of the device) is a corresponding volume of the device which, in some embodiments, enables loading the device with therapeutic material. In some embodiments, the device has a volume of 1-500 microliters, or 1-100 microliters, or 1-50 microliters, or 10-50 microliters, or 20-100 microliters. Additionally, another potential advantage is that such thickness of the device, especially in a device made of a soft elastomeric matrix as described herein, potentially does not disturb the user, which potentially increases the compliance for the use of the device. In some embodiments, the device has a mass of 1-500 mg, or 1-100 mg, or 1-50 mg, or 10-50 mg, or 10-30 mg, or 15-20 mg, or 20-100 mg. In some embodiments, the ophthalmic device includes 1-500 mg, or 20-500 mg or 20-150 mg, or 20-120 mg of active pharmaceutical ingredient/s (API). In some embodiments, the ophthalmic device is formed of 1% to 50%, or 1-20% of active pharmaceutical ingredient/s (API). In some embodiments, an entirety or majority of the ophthalmic device is active pharmaceutical ingredient/s (API), for example, the device being at least 80%, or at least 90%, or at least 95%, or at least 99% active pharmaceutical ingredient (API), by weight and/or volume. In some embodiments, the ophthalmic device includes large amounts of active pharmaceutical ingredient/s (API) (e.g. about 1 mg to about 20 mg or more than 1 mg, or more than 20 mg, or 0.2-1 mg, or 1-4 mg, or 4-10 mg, or 10-20 mg, or 20-50 mg) and/or is able to elute such ingredient/s to a surface of the eye.

A potential advantage of the ophthalmic device is the ability to dispense active pharmaceutical ingredient (API), (e.g. a larger amount than that dispensed by topically applied preparation/s), to the surface of the eye and/or into the eyeball (potentially replacing eyeball injections). Where for example, a topically applied preparation having up to 1% concentration of active pharmaceutical ingredients (API) applied as eye drops, for example, 30 microliter is, 0.3 mg where a significant portion of the active pharmaceutical ingredient (API) is expected to be flushed naturally out of the eye within a few minutes. A potential benefit of an ophthalmic device which elutes large amount of medication while residing on an eye surface is the ability to provide and/or maintain adequate concentration of drugs in intranasal regions and/or to the brain and/or to the head and/or to another target organ and/or the ability to provide systemic drug delivery (for example for dosing e.g. “micro dosing” of cannabinoids and/or opioids).

In some embodiments the device is used for recreational drug use.

In some embodiments, the retention portion of the device is a portion of the device adjacent to and, in some embodiments, including a leading edge of the device. Where, in some embodiments, the leading edge of a device is defined as a portion of a circumferential edge of the device which, when the device is underneath the eyelid, is nearest to an edge and/or opening of the eyelid. Without being bound by theory, in some embodiments, it is theorized that interaction between the tarsal plate of the eyelid with the slope of the device (and/or the retention portion of the device) acts to retain the device within the eyelid, behind the tarsal plate. In some embodiments, interaction between the tarsal plate and the device is associated with deformation of tissue of the eyelid around the device. In some embodiments, alternatively or additionally to force of the tarsal plate, reactive force of the tissue from deformation of the eyelid acts on the device to prevent movement of the device e.g. out of the eyelid and/or deeper into the cul-de-sac of the eye. Alternatively or additionally, in some embodiments, force between the eyeball and a posterior surface of the device acts to prevent movement of the device, where, in some embodiments, the force includes reactive force of pressure of the eyelid onto the device and then to the eyeball and/or suction forces between the device and eyeball. In some embodiments, the retention portion extends around the ophthalmic device, for example, 20-100% of a perimeter of the ophthalmic device. A potential advantage of a device having a retention portion which extends around a large proportion (e.g. more than 50%) of the device is increased retention forces on the device (e.g. of the tarsal plate) and/or in the event of device rotation, increased likelihood that the edge of the eyelid continues to interact with the retention portion.

In some embodiments, the ophthalmic device is symmetrical, for example, having rotational symmetry about one or more axes. For example, the ophthalmic device may be symmetrical about an axis perpendicular to an eye surface on which the device is disposed. Additionally or alternatively, the ophthalmic device may be symmetrical about a central axis e.g. a central axis defined as an axis connecting centers of the anterior and posterior surfaces. In some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.). A potential advantage of rotational symmetry being maintaining of retention portion interaction with the edge of the eyelid and/or tarsal plate if the ophthalmic device rotates while being worn within the eye.

In some embodiments, the ophthalmic device includes more than one retention portion. A potential advantage of multiple retention portion may be increased area of retention portion/eyelid interaction to hold the device in position. A potential advantage of such increased area may be that if a first retention portion fails and the device escapes from underneath the eyelid and/or the first retention portion escapes from underneath the tarsal plate, the ophthalmic device is potentially retained at least partially underneath the eyelid by a second retention portion. A potential advantage of having more than one raised portion of the device (e.g. as provided by retention portions) is that raised surfaces ease pinch-removal. Pinch-removal being, in some embodiments, where two opposing forces are applied on different portions of the device to break suction between the device and the eye surface and/or push portion/s (e.g. between the portions to which the forces are applied) of device away from the eye surface. Where, in some embodiments, the opposing forces are applied manually by a user e.g. by a thumb and other finger e.g. index finger.

In some embodiments, an ophthalmic device including a plurality of retention portions includes one or more cavities on a posterior surface of the device e.g. a cavity for each retention portion.

In some embodiments, for example, where the device is symmetrical, the retention portion is formed by entire edge of the device or a large proportion e.g. at least 50%, or at least 80% or at least 90% of the perimeter of the device. A potential benefit of all or a large proportion of edges of the device being increased likelihood of retention of the device underneath the eyelid in the circumstance of rotation of the device.

In some embodiments, the ophthalmic device is configured to adhere to an eye surface onto which it is positioned. In some embodiments, if the device escapes from underneath the eyelid, partially or fully, adhesion forces of the device to the eye surface (e.g. sclera) are sufficient to maintain the device on the eye, e.g. for at least a short period of time e.g. during blinking (in some embodiments, during a few blinks e.g. 1-10). Where, in some embodiments, upon escape and/or emergence of the device from underneath the eyelid, the device is returned (e.g. manually) to underneath the eyelid. In some embodiments, adherence of the ophthalmic device to an eye surface is sufficient to prevent rotation of the device. For example, for devices which are not rotationally symmetrical and/or for devices which have a localized retention portion and/or a retention portion disposed asymmetrically on the device. In some embodiments, adherence of the ophthalmic device to an eye surface (e.g. sclera) is associated with suction forces between the device and the eye surface. For example, in some embodiments, a posterior surface of the ophthalmic device adjacent to the eye surface is curved, e.g. is concave, in one or more directions. The curve potentially increasing suction forces between the device and the eye surface. In some embodiments, a curvature of the posterior surface is 0.1-1 that of the sclera, in one or more directions. In some embodiments, a radius of curvature of the posterior surface, in one or more directions, is 1-15 mm, or 1-10 mm, and/or has a ratio to that of the sclera being 0.1-2, or 0.5-1.

In some embodiments, a lower curvature of the posterior surface is associated with reduced suction forces between the eye surface and the posterior surface, potentially enabling re-positioning and/or increasing comfort to a user. In some embodiments, a radius of curvature of the posterior surface, in one or more directions, is lower than that of the sclera, for example a ratio of the radius of curvature of the posterior surface to that of the sclera being greater than 0.8, for 3 example between 0.8 and 1. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

In some embodiments, different portions of the posterior surface have different radii of curvature, but, in some embodiments, each of these have a curvature lower than that of the sclera. In some embodiments, dimensions and/or ratios thereof (e.g. curvatures) as described in this document relate to average adult human eye curvatures. Where, for example, in some embodiments a radius of curvature of the sclera, RS is about 12 mm. In some embodiments, dimensions and/or curvatures are selected for different anatomies, for example for a child's eye, for example for an animal eye, for example for a different surface (e.g. mucosal surface). Where, for example, when the device is used in an eye having different dimensions and/or curvatures (e.g. animal eye, child's eye) size/s and/or curvature/s of the device are scaled for use in the eye type and/or in a specific eye (e.g. personally tailored devices). For example, where one or more dimension of the device e.g. length and/or width of the device footprint and/or retention region height and/or slope (e.g. as described within this document) is scaled, for example, using one or more measurement parameter of the eye in question e.g. a radius and/or diameter and/or largest extent of the eye.

In some embodiments, for example, where the surface includes cavity/ies and/or protrusion/s and/or has a surface texture, radius of curvature of a surface (and/or edge and/or corner) is defined as a radius of a circle which matches curvature of at least 60%, or 80% or 90% of the surface (and/or edge and/or corner).

In some embodiments, the ophthalmic device includes multiple layers and/or portions with different material properties. In some embodiments, the device includes one or more of a lubricous layer, a mucoadhesive layer and an additional layer. In some embodiments, one or more layer includes therapeutic material. In some embodiments, a body of an ophthalmic device has a concave posterior surface. In some embodiments, concavity of a posterior surface in contact with the eye surface (e.g. sclera), increases adhesion of the device to the eye surface. The concavity, for example, providing suction force between the device and eye surface, adhering the device to the eye surface.

Optionally, in some embodiments, a radius of curvature of one or more portions of the posterior surface of the device is smaller than that of the portion of the eye to which the device is to be adhered to. For example, the sclera. In some embodiments, the body of the ophthalmic device is not hollow. In some embodiments, the body of the ophthalmic device is made of a continuous material, meaning it does not comprise macroscopic gaps and/or empty areas within it. In some embodiments, a gap or empty area is defined as macroscopic if it is larger than 0.2 mm. In some embodiments, the body of the ophthalmic device has an oval or round form. In some embodiments, the body of the ophthalmic device is symmetrical relative both to its longitudinal axis and relative to its transversal axis. For example, when looking at the ophthalmic device from the anterior surface, the device is symmetrical relative to its longitudinal axis and relative to its transversal axis.

In some embodiments, the device has an elliptical and/or oval cross-sectional shape. For example, the posterior surface may be elliptical and/or oval.

In some embodiments the posterior surface is smooth for example, potentially reducing irritation associated with contact between the eye surface and the device. In some embodiments, the posterior surface includes rough portion/s and or anchor/s potentially increasing adhesion by friction between the posterior surface and the eye surface. In some embodiments, edge regions (e.g. and optionally, in some embodiments, not a central region which is smooth) of the posterior surface are rough and/or include anchor/s and/or protrusions and/or cavities. In some embodiments, the posterior surface is smoother than the anterior surface. In some embodiments, both the posterior surface and the anterior surface are similarly smooth. In some embodiments, a device includes one or more feature to increase adherence between the device and the sclera. For example, roughness and/or anchors (e.g. cavity/ies, protrusion/s and/or hook/s) for example, located at a posterior surface and/or at edges of the device.

An aspect of some embodiments relates to an ophthalmic device, made of elastomeric matrices as disclosed herein, configured to remain underneath an eyelid (for example, having a retention portion) but that has mobility and/or allows fluid flow between the device and an eyeball surface. A potential advantage of mobility or fluid flow between the device and the eyeball being increased user comfort and/or reduced risk of bacterial growth underneath the device (e.g. associated with tear fluid exchange). In some embodiments, mobility of the device on the eye surface is associated with low suction between and/or an easily breakable suction seal between a posterior surface of the device and the eyeball surface. For example, in some embodiments, the posterior surface includes one or more portions which is has low concavity (e.g. with radius of curvature as described above), or is not concave, or is convex, potentially reducing the ability of the device to seal with the eyeball surface. In some embodiments, the device is able to form a seal with the eyeball surface, for example, having convex posterior surface and/or flexible edges where the seal is easily broken e.g. to allow the device to move on the eye surface (e.g. periodically). For example, in some embodiments, the device includes a sealing edge (e.g. flexible) only on a portion of the device. For example, in some embodiments, the device is elongate, potentially easing breaking of suction forces between the device and the eye surface. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

An aspect of some embodiments of the invention relates to an ophthalmic device, made of elastomeric matrices as disclosed herein, which maintains size and/or shape both in storage and in use where, in some embodiments, the device is provided in a dry form and optionally hydrates during residency on an eye surface. A potential benefit being reduced distortion of the device during hydration and/or use and/or maintained shape and/or maintained mechanical characteristics of the device during use. For example, while being able to store the device in a dry form where active pharmaceutical ingredient/s (API) are less likely to be degraded and/or degrade less rapidly e.g. potentially increasing shelf life of the device. In some embodiments, as the device hydrates (e.g. on an eye surface) the device increases in volume by less than 20%, or less than 15%, or less than 10%, or less than 5%. In some embodiments, one or more maximal cross sectional dimension as the device hydrates increases by less than 20%, or less than 15%, or less than 10%, or less than 5%.

An aspect of some embodiments of the invention relates to an ophthalmic device, made of elastomeric matrices as disclosed herein, which, when dry and when hydrated is soft. Where, in some embodiments, the device is provided in a dry form and optionally hydrates during residency on an eye surface. A potential advantage of a soft device in dry form, is enabling application of the device to the eye without prior hydration and/or softening. A potential benefit of a soft ophthalmic device is reduced irritation to the eye e.g. reducing tearing associated with presence of the device which, in some cases, flushes therapeutic material and/or nutrients.

In some embodiments, as further disclosed below in relation to the elastomeric matrices of the ophthalmic device, at least a portion of the ophthalmic device degrades and/or disintegrates within the eye. In some embodiments, degradation is by loss of mass. In some embodiments, the ophthalmic device is a biocompatible device which is designed to be partially or completely biodegradable and/or bioerodible. The term biodegradable is to be understood as going degradation in the living body, for example, by change of mechanical properties, mass loss, and/or change of shape. Chemical decomposition of the polymers in the matrix is not necessary in order to achieve degradation. Optionally, the PVOH itself remains in its original length, and bonds (e.g. hydrogen bonds) between PVOH, plasticizers, and water break. In some embodiments, the degradation allows clearing the ophthalmic device, or the elastomeric matrix it comprises, from the eye, for example, by tears, that may wash the matrix constituents, crumbs, and/or remains out of the eye. For example, in some embodiments, a portion of a device degrades e.g. once the device is adhered to the eye surface. The degrading one or more portions may include, for example one or more mucoadhesive portions and/or one or more lubricous portions. For example, in some embodiments, one or more portions of the device is selected to degrade within the eye, in about 1 minute to 30 minutes or 15 minutes to 1 hour or about 0.5 hour to 8 hours, or 4 to 24 hours, or 12 hours to 3 days, or 1 day to 7 days, or 3 days to 2 weeks, or 1 week to 1 month.

In some embodiments, one or more portions of the ophthalmic device degrade to enable eluting of therapeutic material. For example, in some embodiments, as a portion comprising therapeutic material degrades, it elutes the therapeutic material into eye tissue and/or eye fluid. For example, in some embodiments, a portion degrades to reveal a portion comprising therapeutic material. Where, in some embodiments, the degrading portion initially covers at least a portion of the portion comprising therapeutic material. In some embodiments, at least a portion of the device is degraded and/or its properties are deteriorated when exposed to a biological environment, for example, a biological system e.g. the eye, and/or to a similar in vitro environment simulating conditions of a biological system.

In some embodiments, degradation and/or deterioration of the device is manifested by change (e.g. reduction) in one or more of device's physical properties, for example one or more of device integrity, tensile strength, elastic module, elongation at breakage, etc.

In some embodiments, degradation occurs from the posterior surface towards the anterior surface, for example, the device maintaining a shape of the anterior surface, for a portion of time during which the device is degrading. In some embodiments, degradation from the posterior surface is more rapid than that of the anterior surface. In some embodiments, the device includes different portions which degrade at different rates. In some embodiments, lower speed degrading and/or non-degrading portions acting to maintain a shape (e.g. retention portion shape) for a time period. In some embodiments, the device is designed to break into portions, for example, upon application of manual pressure. Where, in some embodiments, a size and/or shape and/or brittleness of the device is selected to provide this feature. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

An aspect of some embodiments of the invention relates to an ophthalmic device, made of elastomeric matrices as disclosed herein, having one or more retention portions, and including a thin and/or conforming edge portion. In some embodiments, for example, where the device includes a thin edge portion and/or portion extending from a body of the device, the retention portion is defined as a region of the device fulfilling the requirements of height and/or slope e.g. as described above. In some embodiments, the device (e.g. curvature of one or more portions of the device e.g. to generate sufficient suction to hold the device in position) conforms to an eye. Where, for example, in some embodiments, an edge portion is sufficiently thin and/or flexible to conform to a surface of the eye. In some embodiments, the device has thin edges and/or edges of the device recessed into a surface of the eye. In some embodiments, moving from a leading edge of the device, the device includes a thin edge followed by a retention portion. In some embodiments, a step formed between an edge of the device and the eye surface, for example, by the thin edge next to the retention portion, is 1-100 microns, or 10-50 microns. In some embodiments, edge characteristics (e.g. shape and/or thickness) as described in this document are for all of or a portion of an edge region of the device. For example, a portion of a circumferential edge of the device. For example, for 20-99%, or 80-90%, of a circumference of the device. In some embodiments, different portions of a circumferential edge of a device have different properties e.g. shape and/or thickness.

In some embodiments, adherence to the sclera is associated with adherence of edges of the device to the sclera, for example influenced by an extent of conforming of the device to the sclera and/or of the sclera to the device. In some embodiments, flexibility and/or elasticity of the device increases sealing between the device edge and the eye surface. In some embodiments, the device includes one or more edge portion which is flexible and/or elastic e.g. sufficiently thin to be flexible. In some embodiments, the device has an edge designed for user comfort and/or to increase adherence to the eyeball.

In some embodiments, the ophthalmic device has a thin edge. In some embodiments, an edge (e.g. circumferential edge) of the device has average thickness of about 15 microns, or about 20 microns, or about 30 microns or about 50 microns. In some embodiments, the ophthalmic device has a thin edge of about 15 microns, or about 20 microns. In some embodiments, the ophthalmic device has an edge thickness of about 5-200 microns, or 5-200 microns. In some embodiments, for example, alternatively to having a thin edge. Or where, a device, in some embodiments, has an edge circumference where only a portion of the circumference is thin, in some embodiments, the edge of the device is blunt and/or curved and/or rounded for example, increasing comfort of wear for a user. In some embodiments, the edge of the device is sharp e.g. chisel shaped and/or knife edged. In some embodiments, the edge of the ophthalmic device is defined as a 0.1-1 mm edge region, or 0.1-0.5 mm edge region of the device. In some embodiments, an edge thickness is measured at 0.1 mm, or 0.5 mm, from a circumference of the device. In some embodiments, a thin edged device provides improved sealing between edges of the device and the eye surface. A potential advantage of sealing between the device edges and the eye surface is increased suction between the device and the eye surface.

A potential advantage of sealing between the device edges and the eye surface is reduced accumulation of debris (e.g. one or more protein, mucus, oil, and skin cells) of between the device and the eye surface potentially minimizing risk of infection associated with use of the ophthalmic device.

In some embodiments, a thin edged device conforms to a shape of the eye surface at the device edges. In some embodiments, an edges of the device provide anchoring of the device into the eye surface e.g. soft tissue of the conjunctiva e.g. the soft and thick scleral conjunctiva which is apparently, about 25-40 microns thick and is apparently even thicker in the fornix. In some embodiments, the device recesses into the eye surface at edges of the device and/or makes an indentation in the eye surface, for example, the recessing associated with one or more of pressure between edges of the device and/or softness of the eye surface. In some embodiments, portion/s of the eye are deformed in depth (e.g. by the device), for example at edges of the device (and/or at region/s adjacent to protrusions and/or cavities of the device) e.g. by 50 microns to 500 microns.

In some embodiments, tissue local to edges of the device deforms, for example, a region of tissue at most 0.5 mm, or 1 mm. In some embodiments, edges (and/or edge region/s) of a device conform to a shape of the eye surface. For example, in some embodiments, edge region/s are flexible and/or elastic and/or sufficiently thin to conform to the eye surface. In some embodiments, a device body is flexible and/or elastic e.g. sufficiently flexible and/or elastic to conform to the eye surface, at least partially. Potentially, device body and/or edges conforming to the eye surface improves user comfort and/or reduces risk of dislodgement of the device.

In some embodiments, the posterior surface includes one or more cavities or protrusion, for example, on the posterior surface of the device. Without being limited by theory, it is hypothesized that, in some embodiments, a cavity increases suction between the device and the eye surface e.g. localized suction at the cavity. In some embodiments, eye tissue enters the cavity (e.g. under suction forces of the cavity), potentially increasing adherence between the device and the eye. A potential advantage of cavity/ies on the posterior surface is extended residence time of the device on the eye.

In some embodiments, the device has a plurality of cavities, where in some embodiments, two or more of the cavities are about the same size and/or shape. Where, in some embodiments, two or more of the cavities are different from each other, for example, in size and/or shape. In some embodiments, an opening size of a cavity, is between 0.005 mm and 20 mm, or 0.005-2 mm, or 0.005-1 mm, or 0.005-0.5 mm, or 1-2 mm, 1-4 mm, or 2-6 mm 3-8 mm, or 4-10 mm, or 5-20 mm. In some embodiments, a cavity has a large opening, for example, extending over at least half of an area of the surface (e.g. posterior surface) and/or having an extent of 1-10 mm, or 1-8 mm, or 1-5 mm, or 5-20 mm. In some embodiments, an aperture of a cavity is macroscopic or microscopic in size. In some embodiments, a depth of a cavity is 5 microns-2 mm, or 5 microns-1 mm, or 5-400 microns, or 5-200 microns, 200-600 microns 400-800 microns 600-1500 microns 800-2000 microns. In an exemplary embodiment, a depth of a cavity is 400-1200 microns. In some embodiments, a cavity extends through a proportion of a thickness of the device, for example, through at 10-20% 20-30% 30-50% 50-90%, or 80-99%, or 90-99%, 50-70%, 60-90%, of a thickness of the device in a region of the cavity.

In some embodiments, the ophthalmic device includes one or more protrusions from one or both surfaces of the device. In some embodiments, protrusion/s have a rounded and/or blunt shape. In some embodiments, the device has a plurality of protrusions, where in some embodiments, two or more of the protrusions are about the same size and/or shape. Where, in some embodiments, two or more of the protrusions are different from each other, for example, in size and/or shape. In some embodiments, a size of a protrusion, above a surface (e.g. posterior and/or anterior) of the device is 5-400 microns, or 20-200 microns, or 200-800 microns or 400-1200 microns or 600-1500 microns lower or higher or intermediate ranges or sizes. In some embodiments, an extent of a footprint of a protrusion on the surface of the device is 5 microns to 3 mm, or 5 microns to 1 mm, or 5-700 microns, or 5-400 microns or 3-6 mm, or 4-8 mm, or 4-10 mm, or 6-12 mm.

A potential benefit of protrusion/s is increased adherence of the ophthalmic device to the eye surface. In some embodiments, protrusion/s increase friction between the eye surface and the eye surface. In some embodiments, protrusion/s increase (e.g. between the protrusions) increase the suction force between the device and the eye surface. In some embodiments, the ophthalmic device includes mucoadhesive material e.g. on the posterior surface of the device which potentially increases adhesion between the device and the eye surface on which the device is positioned.

Optionally, in some embodiments, the posterior surface includes one or more mucoadhesive part. Where mucoadhesive material/s in some embodiments, increase adhesion of the device to the eye surface. In some embodiments, the posterior surface includes a mucoadhesive layer. Where, in some embodiments, the layer covers the posterior surface. Alternatively, in some embodiments, the mucoadhesive layer is a non-continuous surface, where hole/s, for example, allow water penetration and/or drug transportation e.g. to a layer underneath the mucoadhesive layer. In some embodiments, the mucoadhesive layer has a ring-shape including an aperture in the center e.g. enabling water penetration and/or drug transportation. The mucoadhesive layer in the device, in some embodiments, is provided dry and/or semi-hydrated. Where, in some embodiments, upon application of the device to the eyeball, the mucoadhesive layer hydrates and becomes sticky, for example, in less than 10 seconds, or in less than 30 seconds, or in less than 1 minute. In some embodiments, the material/compound/polymer in a mucoadhesive layer has an adherence force of 80-200%, or of 100-200%, or of over 100%. Where, for example, pectin is defined as having an adherence force of 100%. In some embodiments, mucoadhesive portions include at least one mucoadhesive compound, which in some embodiments, is selected from the group consisting of gelatin, alginates, chitosan, amylose, collagen, sodium poly-acrylate, modified starch, elastin, polyacrylic acid and combinations thereof. In some embodiments, the device presents a low friction surface to the eyelid (e.g. is smooth). A potential advantage of which is reduced likelihood of dislodgement of the device. In some embodiments, the device presents a low friction surface to the eye surface (e.g. is smooth). A potential advantage of which is reduced likelihood of discomfort to the user. In some embodiments, the device presents a same low level of friction to the eyelid and to the eye surface.

Optionally, in some embodiments, one or more of portion of an anterior surface of the device is smooth and/or includes lubricious material. Where the anterior surface of the device is a surface which is in contact with the inner surface of the eyelid, at least part of the time. Potentially, in some embodiments, smooth and/or lubricous material reduces friction between the eyelid and the surface. Potentially, in some embodiments, the smooth and/or lubricous material minimizes irritation of the eye, e.g. the eyelid, e.g. associated with the ophthalmic device.

In some embodiments, the posterior surface is smoother than the anterior surface. In some embodiments, both the posterior surface and the anterior surface are smooth. In some embodiments, a lubricious material layer covers the anterior surface where, in some embodiments, the layer is continuous. In some embodiments, the lubricious layer is a non-continuous surface, (e.g. including one or more hole) for example to allowing water penetration and/or drug transportation. For example, in some embodiments, a lubricious material layer includes holes. In some embodiments, the anterior surface hosts one or more lubricious material portion where, in some embodiments, one or more of the portions is connected to other portion/s and/or one or more of the portions is not connected to other portions. Optionally, in some embodiments, the lubricous surface is temporary, where, in some embodiments, one or more portions of lubricous material degrades and/or disappears (e.g. is absorbed and/or expelled by the eye), for example, before other portion/s of the device degrade. In some embodiments, a material layer has variable thickness and/or variable material properties e.g. a lubricious layer and/or a mucoadhesive layer. Where, for example, upon attachment to the eye, a material has a larger extent on the anterior surface than after a time period of residence on the eye, where, in some embodiments, more rapidly degraded portion/s (e.g. thinner portions and/or portions having more a rapidly degraded composition) of the layer dissolve prior to other portion/s e.g. to reveal lower layers which in some embodiments comprise active pharmaceutical ingredient/s (API). While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

An aspect of some embodiments of the invention relates to a kit comprising an ophthalmic device (e.g. as described in this document and made with elastomeric matrices as disclosed herein) and instructions for use thereof. In some embodiments, the kit comprises means for delivering the device into the eye of a patient. For example, an applicator. In some embodiments, the ophthalmic device is packaged together with the applicator. Optionally, in some embodiments, the kit includes a degradation formulation which, when contacted with the device when the device is on an eye surface of a patient, structurally degrades the device and/or accelerates degradation of the device. In some embodiments, devices are provided in multi-device packs. Where, in some embodiments, two or more devices are interconnected to one another, or separated.

In some embodiments, the ophthalmic device is one or more of flexible, elastic, and supple e.g. to conform to a shape of the eyeball. In some embodiments, the ophthalmic device elasticity and/or softness is similar to the elasticity and/or softness of soft contact lenses. In some embodiments, the ophthalmic device has elasticity or softness, in one or more directions, with a Young's modulus of 0.01-1.5 MPa or 0.05-0.15-1.5 MPa, or 0.25-0.35 MPa. In some embodiments, as mentioned before, the required softness may be achieved with an elastomeric matrix formulated with water, PVOH, and a large amount plasticizers. The amount of the plasticizers may be considered large if it is larger than, or, preferably, at least double (in mass) than the mass amount of the PVOH. In some such embodiments, the mass of the PVOH and the plasticizers together is 70% or more of the total mass of the matrix excluding the water. In some embodiments, the ophthalmic device has elasticity, in one or more directions, with a Young's modulus of 0.01-2.00 MPa. In some embodiments, the ophthalmic device has elasticity, in one or more directions, (e.g. Young's modulus 0.01-200 MPa, or 0.05-0.15 MPa or 0.15-0.35 MPa) in a dry form. In some embodiments, the ophthalmic device has elasticity, in one or more directions, (e.g. Young's modulus 0.01-200 MPa, or 0.05-0.15 MPa, or 0.01-0.35 MPa) in a wet form while in the eye. In some embodiments, the ophthalmic device has elasticity allowing elongation of 10% to 800% or more (in dry form and/or in wet form). In some embodiments, the ophthalmic device is one or more of rigid, and shaped e.g. shaped to conform to a shape of the eyeball. In some embodiments, the ophthalmic device elasticity and/or hardness, in one or more directions, similar to the elasticity and/or hardness of hard or rigid contact lenses. In some embodiments, the ophthalmic device has elasticity, in one or more directions, with a Young's modulus of 0.1-200 MPa. In some embodiments, the ophthalmic device has this elasticity, in one or more directions, when in a dry form. In some embodiments, the ophthalmic device has this elasticity in the wet form e.g. while in the eye. In some embodiments, the ophthalmic device has layers with different material properties e.g. elasticity and/or flexibility (see below regarding exemplary elastomeric matrices of the ophthalmic device). In some embodiments, the ophthalmic device has regions having different material properties e.g. elasticity and/or flexibility. In some embodiments, the device is flexible, potentially enabling the device to shape to a surface of the eye e.g. potentially increasing user comfort (e.g. allowing the eyelid to move smoothly above the device with little or no interference and/or discomfort) and/or reducing dislodgement forces experienced by the device. In some embodiments, a flexible device has a concave posterior surface, where suction (e.g. associated with interaction between concavity of the posterior surface and the eye surface) between the device and the eye surface then flattens the device onto the eye surface. In some embodiments, elasticity of the device increases suction force between the device and the eye surface to adhere the device to the eye. Where, for example, in some embodiments, elasticity compensates for low concavity curvature of the posterior surface of the device. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

In some embodiments, flexibility and/or elasticity of the device increases sealing between the device edge and the eye surface. In some embodiments, one or more portions is configured to fit an eye surface and/or a portion of an eye surface and/or an orifice. In some embodiments, the device is selected to be compatible with size of the inferior or superior conjunctival sac where the device deforms the sac only slightly, for example, by at most 2 mm for example, by only an amount which is comfortable for a user and/or does not cause harm to the eye surface and/or eyelid and/or other tissue/s. For example, in some embodiments, only the retention portion deforms the sac. It has been found that devices with a height of 2 mm can be comfortable and, in some embodiments, up to a height of 4-6 mm. It has been found that, in some embodiments, a device width of 5-10 mm is comfortable. In some embodiments, the ophthalmic device has a thickness of less than 3 mm, or less than 2.5 mm, or less than 2 mm, or less than 1.5 mm, or less than 1 mm, or less than 0.8 mm, or less than 0.6 mm, or less than 0.5 mm, or less than 0.4 mm, or less than 0.3 mm. In some embodiments, the ophthalmic device has a thickness of greater than 3 mm, or greater than 2.5 mm, or greater than 2 mm, or greater than 1.5 mm, or greater than 1 mm, or greater than 0.8 mm, or greater than 0.6 mm, or greater than 0.5 mm, or greater than 0.4 mm, or greater than 0.3 mm.

In some embodiments, pressure between the eye and the device is increased by increasing the thickness of the device and/or the thickness of an edge region of the device. Where, increased pressure (and/or sealing between the eye and the device e.g. associated with pressure and/or contouring of the eye to the device or the device to the eye) in some embodiments, reduces tear fluid penetrations between the device and the eye surface.

In some embodiments, the device is small e.g. sufficiently small that it fits within a space underneath an eyelid (e.g. entirely). In some embodiments, a maximum and/or average extent of a device is about 4-12 mm, or 4-10 mm, or 4-8 mm, or 3-8 mm, 1-8 mm, or 4-6 mm. In some embodiments, the ophthalmic device has a size (e.g. maximum and/or average extent) of about 4 mm. In other embodiments, the device has dimensions of between about 0.5 to about 20 mm in maximum size. A potential advantage a small dimensioned device is increased user comfort and/or lower likelihood of dislodgement of the device during movement of an eyelid over the device.

In some embodiments, an ophthalmic device is elongated. Where, for example, in some embodiments, a dimension of the device measured along an axis of the elongation is about 0.5-20 mm. In some embodiments, the ophthalmic device is designed to be put in and out of the eye by the patient wearing it. In some such embodiments, the device is at least 3 mm long along its shorter side, to allow the user to see it and manipulate it conveniently when putting the device under the eyelid. In some such embodiments, the dimension of the device measured along an axis of the elongation is larger than 3 mm, for example, at least 4 mm or at least 5 mm. On the other hand, the device is preferably not too large, so as not to bother the wearer, so a maximum extent may be in some embodiments, about 8 mm. Where, in some embodiments, an extent of the device is measured as a maximum dimension of a smallest cuboid shape into which the device fits. In some embodiments, for example, associated with low curvatures of the device surfaces, the cuboid bounding shape is thinner in one dimension, (e.g. associated with a direction into the eye when the device is on the eye) than in the other two dimensions.

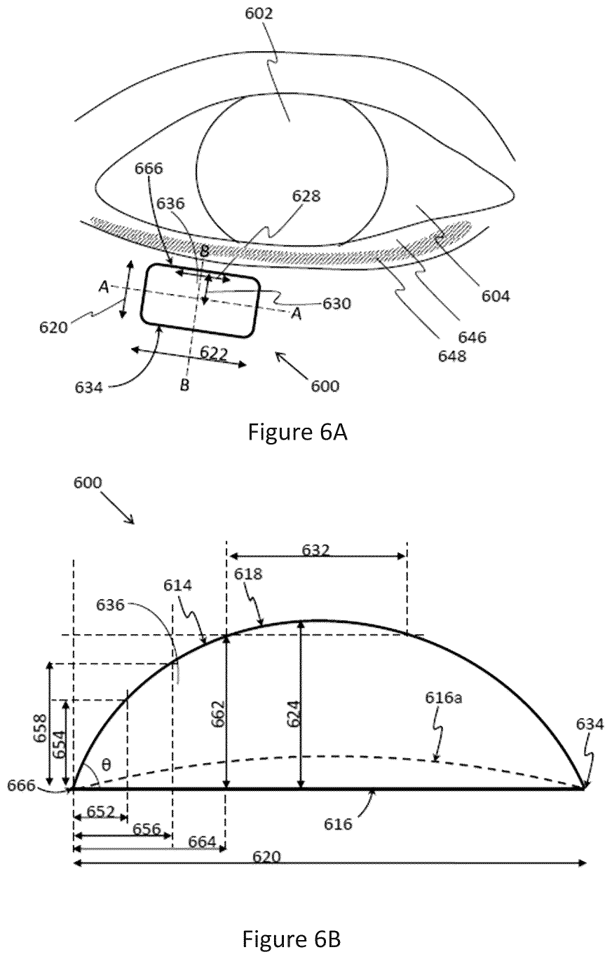

In some embodiments, the body of the ophthalmic device is not hollow. In some embodiments, the body of the ophthalmic device is made of a continuous material, meaning it does not comprise macroscopic gaps and/or empty areas within it. In some embodiments, a gap or empty area is defined as macroscopic if it is larger than 0.2 mm. In some embodiments, as shown for example in FIG. 6A, the body of the ophthalmic device is symmetrical relative to its longitudinal axis (schematically shown by line AA) and relative to its transversal axis (schematically shown by line BB). For example, when looking at the ophthalmic device 600 from the anterior surface, the device is symmetrical relative to its longitudinal axis (AA) and relative to its transversal axis (BB). In some embodiments, for example, in the embodiment schematically illustrated in FIG. 6A, the symmetries in relation to both axes are mirror reflection symmetry. In some embodiments, the device comprises an elliptical and/or oval cross-sectional shape. A potential advantage of the symmetry of the ophthalmic device is convenience of use where, in some embodiments, the device is applied by the user himself, and having no “wrong” orientation may increase convenience of use.

In some embodiments, an ophthalmic device as described herein may be used once to deliver active pharmaceutical ingredients (API) and/or to provide active pharmaceutical ingredient (API) availability for a time period longer than that of topically applied preparations such as eyedrops, which are typically available for less than a minute. Some embodiments provide drug delivery for a time period of about 5 minutes or longer, or longer than 10 minutes, or longer than an hour, or longer than a day. Where, in some embodiments, the device serves as controlled release vehicle for one or more active pharmaceutical ingredients (API).

A potential benefit of an ophthalmic device configured to reside on an eye surface is potentially increased residence time of a treatment drug in the eye. For example, as compared to periodically applied topical compositions (e.g. eye drops, ointment). A potential benefit of an ophthalmic device which elutes medication while residing on an eye surface is the ability to provide and/or maintain adequate concentration of drugs in the pre-corneal/pre-scleral tear film e.g. for extended periods of time, for example, of from 5 minutes to 24 hours or more. A potential benefit of an ophthalmic device which elutes medication while residing on an eye surface e.g. maintaining a position on the eye surface is the potential ability for medication to diffuse through eye tissue. For example, penetrating into the sclera and/or into the cornea and/or into the eyeball, and/or reaching more internal portion/s of the eye. Potentially enabling, in some embodiments, topical medication of those areas which are usually treated by injection e.g. into the eyeball. A potential benefit of an ophthalmic device (e.g. with extended residence and/or elution times) is the ability to provide and/or maintaining adequate concentration of drugs to intranasal region/s (e.g. as delivered by tear fluid) and/or to the brain and/or to the head and/or to another target organ and/or as systemic drug delivery.

In some embodiments, the ophthalmic device being in residence enables steady elution of treatment materials into the eye potentially reducing effects of periodic dosing of eyedrops or ointments such as, for example, a transient period of systemic and/or local overdose (potentially associated with high risk of side effects) followed by an extended period of sub-therapeutic levels before the administration of next dose. Where the rate of tissue drug uptake is high at early times after dosing, but declines rapidly. A long residence time, for example, as compared to, eye drops applied to the eye e.g. the surface of the eyeball, where blinking and natural tear flow combine to limit time that liquid medicament will remain effective e.g. to a few minutes. Potentially, less active pharmaceutical ingredient (API) per treatment is used in treatment of a subject using an ophthalmic device e.g. as opposed to topical preparations where active pharmaceutical ingredients (API) may be washed out before they are effective requiring higher quantities of active pharmaceutical ingredients (API) and/or where high patient compliance is more likely to be lower as application is frequent potentially resulting in more discarded un-used active pharmaceutical ingredients (API). In some embodiments, the ophthalmic device protects a portion of the eye. For example, from external impact/s and/or chemical changes. For example, to ameliorate and/or reduce ocular damage upon orbisculation (citrus fruit squirting juice into the eye). In some embodiments, the ophthalmic device protects a portion of the eye, enabling the portion to heal. For example, in some embodiments, an incision and/or wound in the eye is closed and/or covered by placing the ophthalmic device over the incision. Where, suction forces in between the eye surface and the device, potentially assisting in closing and/or maintaining closure of the incision and/or wound potentially hastening healing.

In some embodiments, a device is tailored to an individual subject, where, for example, in some embodiments, one or more of size, shape, curvature of surface/s, type of therapeutics, release time, residence time are selected for the individual and/or according to an individual treatment plan. Although throughout this document, ophthalmic use of the device is described, it should be understood that one or more embodiments and/or combinations of embodiments of the device as described within this document, in some embodiments, are used in treatment of other portions of a human or animal body. For example, for portions of the eye other than the sclera, e.g. the cornea, the cul-de-sac of the eye. For example, other mucosal surface/s e.g. the inner mouth e.g. the intranasal surface. For example, other tissue types and/or organs. Although throughout this document, ophthalmic use of the device is described, it should be understood that one or more embodiments and/or combinations of embodiments of the device as described within this document, in some embodiments, are used for other applications such as: to host e.g. contain, have attached to and/or provide a support for one or more type of electronics e.g. on the eye. For example, one or more active electronic element and/or passive modules and/or components e.g. an antenna, RFID. For example, one or more capacitor and/or resistor and/or, shape memory element, or may act as a support for other element or elements in the ocular region etc.) Where, for example, in some embodiments, electronics include on or more of sensor/s (e.g. temperature sensitive element, pressure sensitive element, humidity sensitive element), imager/s (e.g. camera), a user interface, a processor, transmitter, receiver, illumination element, led screen, power source. In some embodiments, electronics (e.g. using sensor/s) are used for therapeutics (e.g. Electroconvulsive therapy (ECT) treatment) and/or diagnostics. In some embodiments, one or more electronic part is used for entertainment (e.g. VR) and/or aesthetic enhancement. In some embodiments, the device is used for entertainment and/or for aesthetic enhancement. In some embodiments, more than one device as described in this document is used to treat a subject, for example, where multiple devices are in situ on an eye surface at the same time, for example, each device providing a particular dose. Where, in some embodiments, to provide a higher dose more than one device is applied. In some embodiments, multiple devices as described in this document are provided packaged together and/or where the multiple devices are provided connected. In some embodiments, the device includes and/or is constructed from non-toxic and/or biocompatible materials. In some embodiments, where therapeutic material is toxic in high concentrations, the entire device, in some embodiments, contains less than a toxic dose and/or the device elutes the therapeutic material at sub-toxic levels. In some embodiments one or more portions of the device include dye and/or pigment e.g. for ease of identification while in the eye, and/or during insertion and/or extraction, and/or during production. In some embodiments, dye and/or color is printed into one or more identifying shape and/or letter and/or numeral and/or barcode. In some embodiments, one or more identifier is embossed or stamped on the device. In some embodiments, the device includes one or more electronic identifier e.g. RFID on or within a body of the device. In some embodiments, device dye and/or pigment includes one or more of blue, red, fluorescent yellow, and/or any other color. In some embodiments, the ophthalmic device is manufactured from a film, for example, a multi-layer film. Where, in some embodiments, the film is cut into shape to form individual devices. In some embodiments, the film and/or individual devices are shaped for example, to form desired curvatures of surfaces. Where, in some embodiments, shaping is by casting of a solution onto a shaped support surface e.g. a mold. Where, in some embodiments, shaping is by application of mechanical and/or chemical treatment e.g. pressure and/or heat. Thus, in some embodiments, the ophthalmic device is obtainable by mixing the API with water and ophthalmic demulcents to obtain a mother solution; and drying the mother solution at room temperature, optionally in a mold shaped according to the desired shape of the device. In some such embodiments, the ophthalmic device is obtainable without heating the mother solution. In some embodiments, the ophthalmic device is obtainable without heating the API. In some embodiments, active pharmaceutical ingredient/s (API) are incorporated into one or more solution each of which is successively cast onto a support surface to provide a multi-layer film. In some embodiments, separately cast layers have one or more different properties (e.g. thickness, mucoadhesive, lubricious, containing active pharmaceutical ingredient/s (API)). In some embodiments, one or more surface is treated to provide it with different properties e.g. a surface is treated to become more adhesive and/or more lubricious.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Table of Contents

1. Introduction to the disclosure
2. Exemplary forms/geometries
   2.1 Exemplary methods of use
   2.2 Exemplary detailed method
   2.3 Exemplary ophthalmic devices
   2.4 Exemplary degradation
   2.5 Exemplary methods of treatment
   2.6 Exemplary materials
      2.6.1 Exemplary lubricant material
      2.6.2 Exemplary mucoadhesive material
      2.6.3 Exemplary therapeutic materials
      2.6.4 Exemplary nanoparticle and/or microparticle active pharmaceutical ingredient/s (API)
   2.7 Summary of Exemplary forms/geometries
3. Exemplary elastomeric matrix comprising PVOH
   3.1 Exemplary elastomeric matrix
   3.2 Exemplary mechanical properties
   3.3 Exemplary plasticizers
   3.4 Exemplary compositions of elastomeric matrices
   3.5 Exemplary composite elastomeric matrix
   3.6 Exemplary Medical/Ophthalmic device
   3.7 Exemplary method of treatment
   3.8 Exemplary method of preparation
   3.9 Summary of exemplary elastomeric matrix comprising PVOH
4. Exemplary elastomeric matrix comprising two or more types of PVOH
   4.1 Exemplary basic composition of an exemplary elastomeric matrix
   4.2 Exemplary degrees of hydrolysis (D.H.), degrees of polymerization (D.P.), degradation time and combinations thereof
   4.3 Exemplary amounts of components within an exemplary elastomeric matrix composition
   4.4 Exemplary mechanical properties
   4.5 Exemplary plasticizers
   4.6 Exemplary composite elastomeric matrix
   4.7 Exemplary Medical/Ophthalmic device
   4.8 Exemplary method of treatment
   4.9 Exemplary method of preparation
5. Additional exemplary information about exemplary ophthalmic devices
   5.1 Exemplary compositions of an exemplary ophthalmic device
   5.2 Exemplary process for manufacturing an exemplary ophthalmic device
   5.3 Exemplary shape of exemplary ophthalmic device
6. Exemplary use of the exemplary ophthalmic device in the treatment for Presbyopia
   6.1 Introduction
   6.2 Exemplary pharmaceutical active ingredients (API) for treating Presbyopia
   6.3 Exemplary method of preparation of the ophthalmic device with the pharmaceutical active ingredient (API) for the treatment for Presbyopia
   6.4 Exemplary potential advantages of using the exemplary ophthalmic device for the delivery of pharmaceutical active ingredients (API)
   6.5 Exemplary general mechanism of action of the ophthalmic device in the treatment for Presbyopia
   6.6 Exemplary preliminary clinical results
7. Exemplary use of the exemplary ophthalmic device in the treatment for Dry Eye
   7.1 Introduction
   7.2 Exemplary general mechanism of action of the ophthalmic device in the treatment for dry eye
   7.3 Exemplary potential advantages of using the exemplary ophthalmic device for the treatment for dry eyes
   7.4 Exemplary demulcent content comparison between the exemplary ophthalmic device and a typical OTC eye-drop

### 1. Introduction to the Disclosure

In some embodiments, the invention relates to an ophthalmic device made of an elastomeric matrix having specified compositions and forms (or geometries).

In the following chapters, exemplary embodiments of forms (geometries) will be described in chapter 2, followed by exemplary embodiments of elastomeric matrices and their compositions in chapters 3 and 4. Some exemplary ophthalmic devices shaped as described in chapter 2 and comprising elastomeric matrix composed as described in chapters 3 or 4 are described in chapter 5, but every form or geometry described in chapter 2 may be made of an elastomeric matrix composed as described in chapters 3 and 4, even if not explicitly recited in chapter 5.

Chapters 6 and 7 describe exemplary uses of ophthalmic devices described in chapters 2 through 5. Chapter 6 describes the use of such device for treating presbyopia, and chapter 7 describes the use of such device for treating dry eye disease (aka dry eye syndrome).

### 2. Exemplary Geometries/Shapes of Exemplary Ophthalmic Devices

In the following paragraphs exemplary geometrical/shape characteristics of an exemplary ophthalmic device will be disclosed.

### 2.1. Exemplary Methods of Use

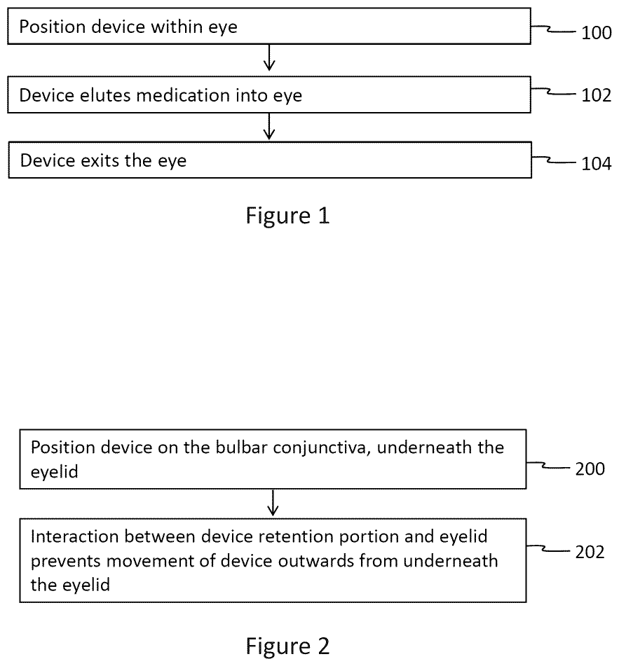

Referring now to FIG. 1 showing a method of use of an ophthalmic device, according to some embodiments of the invention. The ophthalmic device described in the chapter 2 may be composed as described in chapter 3 or 4, for example, as described in chapter 5.

At 100, in some embodiments, the ophthalmic device is positioned within an eye. For example, placed on an eye surface, for example, on the bulbar conjunctiva and/or onto a portion of the sclera.

At 102, in some embodiments, the ophthalmic device elutes medication into the eye.

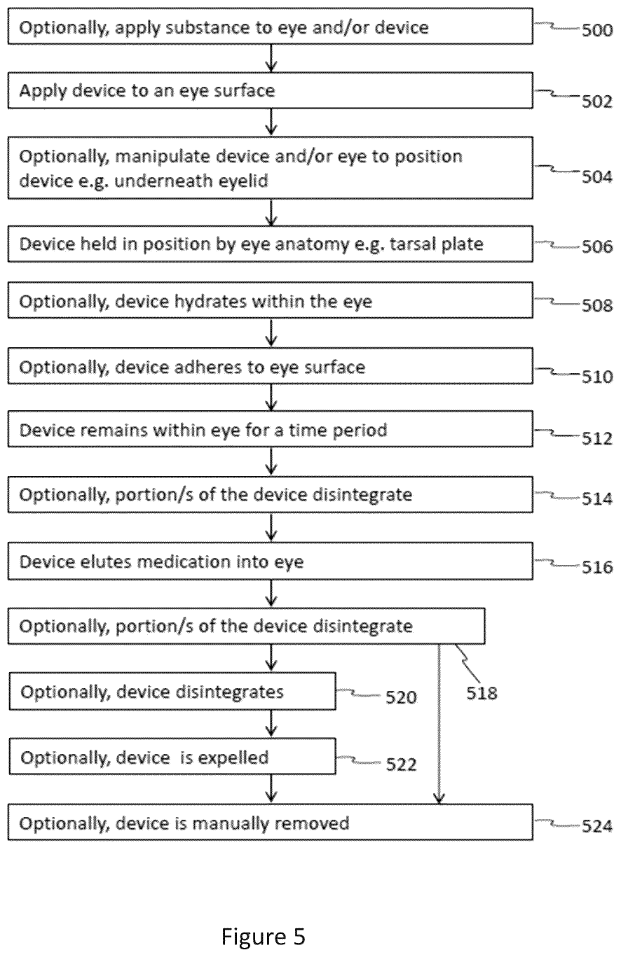

At 104, in some embodiments, the ophthalmic device exits the eye. Where, in some embodiments, the device exit is manual (e.g., by the user or a caregiver) and/or the device is expelled by the eye. Where, in some embodiments, prior to exit of the ophthalmic device, the device disintegrates/degrades, at least partially. In some embodiments, exiting the eye includes one or more feature of one or more of steps 514-524 in FIG. 5.

Referring now to FIG. 2 showing a method of treatment using an ophthalmic device, according to some embodiment of the invention.

At 200, in some embodiments, the ophthalmic device is positioned on the bulbar conjunctiva and at least partially underneath an eyelid of the eye e.g. fully underneath the eyelid. Where, in some embodiments, a retention portion of the ophthalmic device is positioned adjacent to an opening of the eyelid and deeper into the conjunctival sac than a tarsal plate of the eyelid e.g. when the eyelid is open and closed.

At 202, in some embodiments, interaction between the retention portion, the eyelid and/or eyeball, prevents escape of the ophthalmic device from underneath the eyelid. In some embodiments, interaction between the tarsal plate of the eyelid with the slope and/or height of the device acts to retain the device within the eyelid, behind the tarsal plate. In some embodiments, interaction between the tarsal plate and the device is associated with deformation of tissue of the eyelid around the device. In some embodiments, alternatively or additionally to force of the tarsal plate, reactive force of the tissue from deformation of the eyelid acts on the device to prevent movement of the device e.g. out of the eyelid and/or deeper into the cul-de-sac of the eye. Alternatively or additionally, in some embodiments, force between the eyeball and a posterior surface of the device acts to prevent movement of the device, where, in some embodiments, the force includes reactive force of pressure of the eyelid onto the device and then to the eyeball and/or suction forces between the device and eyeball.

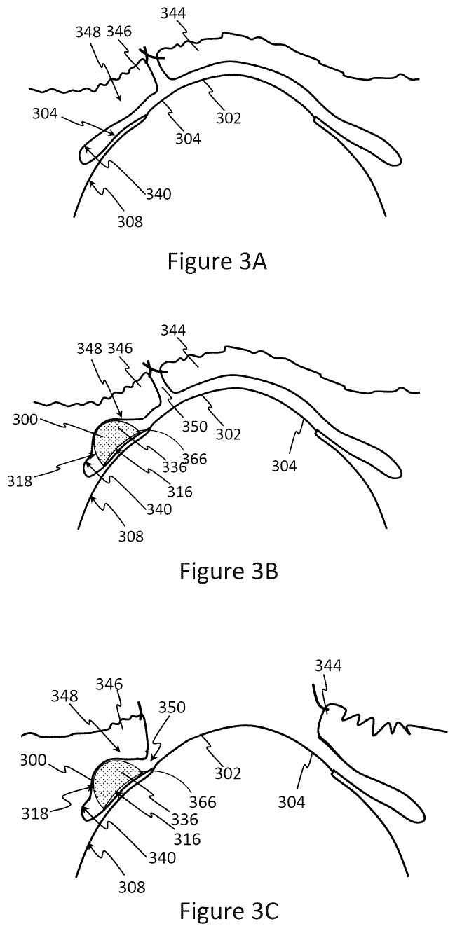

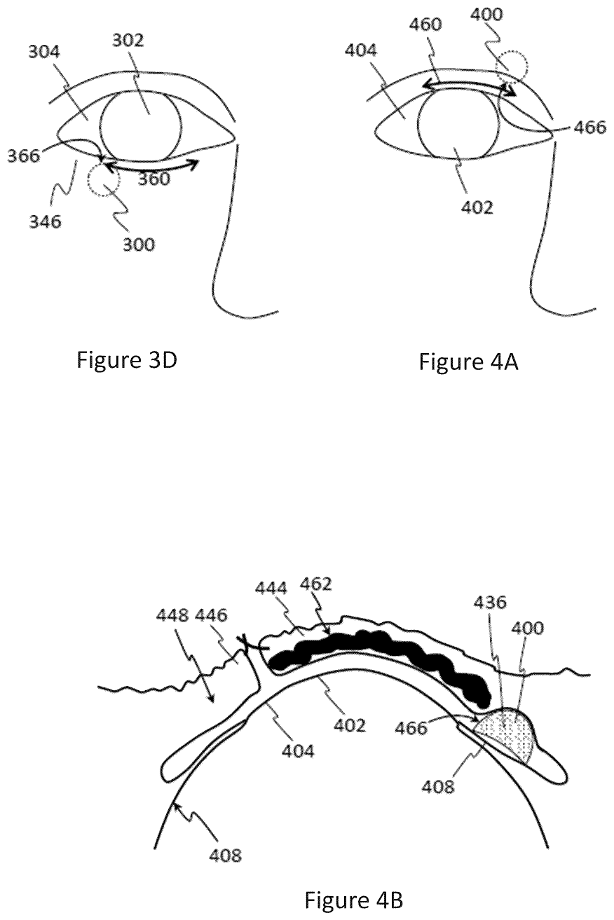

Referring now to FIG. 3A showing a simplified schematic cross sectional view of an eye.

Referring also to FIG. 3B showing a simplified schematic cross sectional view of an ophthalmic device 300 in position on an eye surface, when the eye is closed, according to some embodiments of the invention.

Referring also to FIG. 3C showing a simplified schematic cross sectional view of an ophthalmic device 300 in position on an eye surface, when the eye is open, according to some embodiments of the invention.

In some embodiments, cross sections of FIGS. 3A-C are cross sections taken in a superior-inferior direction e.g. are sagittal cross sections of the eye.

In some embodiments, device 300 is covered by a lower eyelid 346 when the eye is closed (FIG. 3B) and when the eye is naturally open (FIG. 3C). In some embodiments, device 300 is positioned close enough to the eyelid opening e.g. on the bulbar conjunctiva and not deep into the cul-del-sac direction that manual movement, of the eyelid (e.g. without causing the user discomfort) is sufficient to reveal the device, at least partially.

In some embodiments, device 300 is positioned on an eye surface which is a portion of a surface of conjunctiva around an eyeball 308. In some embodiments, ophthalmic device 300 resides on a bulbar conjunctiva portion of a sclera, a region of conjunctiva adjacent to the cornea and in between the fornix conjunctiva 340 and cornea 302.

In some embodiments, device 300 has a body including a posterior surface 316, and an anterior surface 318. In some embodiments, device 300 has a retention portion 336 which is, in some embodiments, a portion of device 300 adjacent to an opening 350 of eyelid 346. In some embodiments, retention portion 336 is sufficiently thick at a short distance from a leading edge 366 of device 300 and/or presents a sufficiently steep slope to eyelid 346 that device 300 is retained underneath eyelid 346, for example, by a tarsal 348 plate. In some embodiments, the body of the ophthalmic device is not hollow. In some embodiments, the body of the ophthalmic device is made of a continuous material, meaning it does not comprise macroscopic gaps and/or empty areas within it. In some embodiments, a gap or empty area is defined as macroscopic if it is larger than 0.2 mm. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

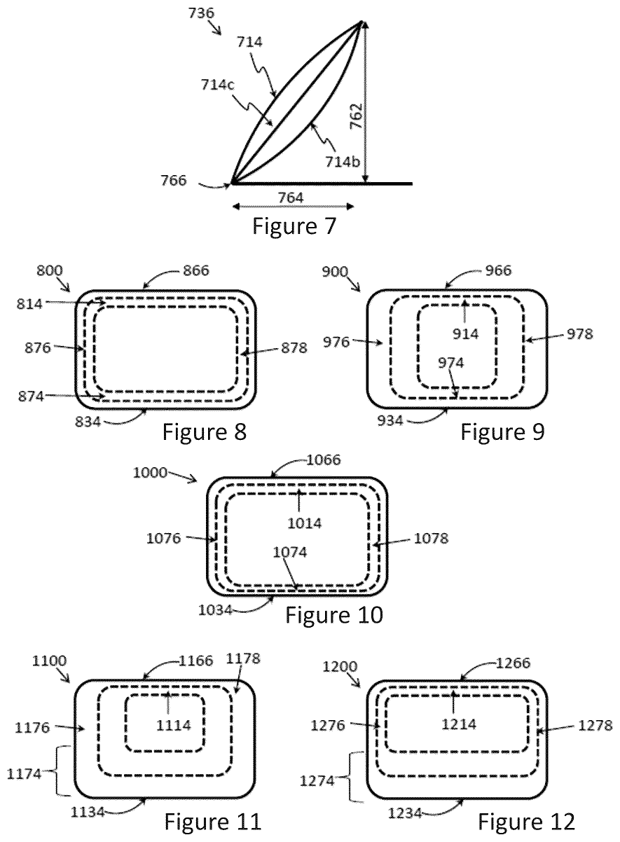

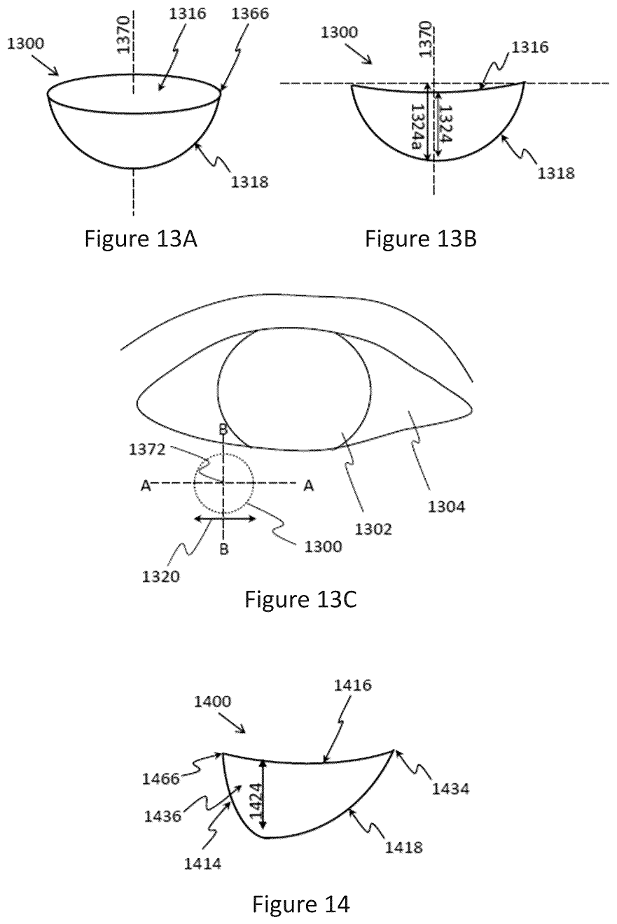

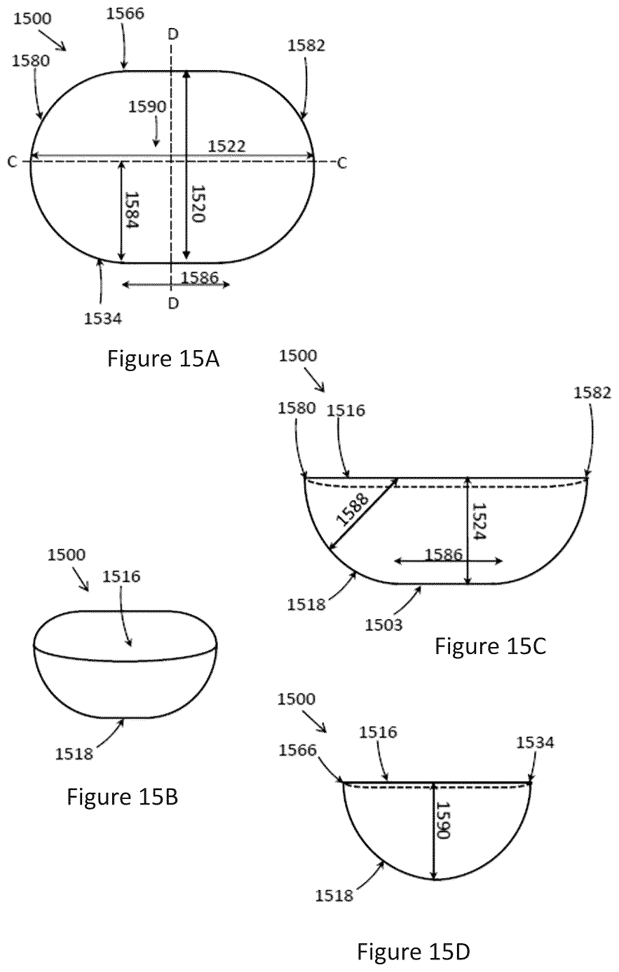

In some embodiments, retention portion 336 includes one or more feature of one or more of retention portions 636 FIGS. 6A-B, 736 FIG. 7, 1336 FIG. 13A-C, 1436 FIG. 14, 1536 FIG. 15A-D and/or other retention portion/s as described elsewhere in this document.

In some embodiments, reactive force associated with deformation of eyelid 346 and/or conjunctiva 308 around device 300 and/or interaction between tarsal plate 348 and retention portion 336 act to hold device 300 in position on the eye surface and/or underneath eyelid 346.

Referring now to FIG. 3D showing a simplified schematic top view of an ophthalmic device 300 in position on an eye surface, according to some embodiments of the invention.

In some embodiments, as shown for example in FIG. 3D, the ophthalmic device 300 is positioned on an eye surface (e.g. a sclera), for example, on an inferior portion of the bulbar conjunctiva underneath the lower eyelid 346 e.g. entirely underneath the lower eyelid 346.

Referring now to FIG. 4A showing a simplified schematic top view of an ophthalmic device 400 in position on an eye surface 404, according to some embodiments of the invention.

In some embodiments, as shown for example in FIG. 4A, the ophthalmic device 400 is positioned an eye surface 404 (e.g. a sclera), for example, on a superior portion of the bulbar conjunctiva underneath the upper eyelid 444 e.g. entirely underneath the upper eyelid 444. In some embodiments, when device 400 is positioned on a superior portion of the eye, positioning is deeper within the fornix (e.g. in comparison to position where the device is positioned on an inferior portion of the eye e.g. underneath the lower eyelid).

Referring now to FIG. 4B showing a simplified schematic cross sectional view of an ophthalmic device 400 in position on an eye surface 404, when the eye is closed, according to some embodiments of the invention.

Where, in some embodiments, eye surface 404 is a superior portion of the sclera. Where, in some embodiments, device is held underneath upper eyelid 344, for example, when the eye is open and closed.

In some embodiments, a depth to which device 400 is inserted and/or a length of the device extending underneath the upper eyelid is deeper than that of a device for use under the inferior eyelid. In some embodiments, increasing likelihood of the device remaining underneath the superior eyelid where, generally, the eyelid and tarsal plate have larger extent.

In some embodiments, reactive force associated with deformation of eyelid 444 and/or conjunctiva 408 around device 400 and/or interaction between superior tarsal plate 462 and retention portion 436 act to hold device 400 in position on the eye surface and/or underneath upper eyelid 444.

Referring now to both FIG. 3D and FIG. 4A, arrows 360, 460 illustrate ranges of exemplary positions for device/s 300, 400 e.g. with respect to visible portions of an eye.

Where, in some embodiments, ophthalmic device 300 is positioned underneath a central portion of lower eyelid 346. Where, in some embodiments, ophthalmic device 400 is positioned underneath a central portion of upper eyelid 444.

For example, a central 2-10 mm, or 2-7 mm, e.g. as measured along a contour of a junction between the eyelid and an eye surface (e.g. sclera 304, 404).

For example, at least 1 mm, or 2 mm, or 5 mm, from the inner and/or outer eye corners e.g. as measured along a contour of a junction between the eyelid and the eye surface (sclera 304, 404 and/or cornea 302, 402).

For example, a central 30-80%, or 40-70%. Where, in some embodiments, the portion is measured as a length along a contour of the eyelid (e.g. at a junction between the eyelid and the eye surface) with respect to a total length measured along the contour of the eyelid between corners of the eye, when the eye is closed and/or open.

In some embodiments, device/s 300, 400 are symmetrical in shape, e.g. in one or more dimension. In some embodiments, device/s 300, 400 have a circular footprint e.g. when residing on an eye surface (e.g. sclera 304, 404).

### 2.2. Exemplary Detailed Method

Referring now to FIG. 5 showing a method of use of an ophthalmic device, according to some embodiments of the invention.

At 500, in some embodiments, optionally, a substance is applied to an eye and/or to the ophthalmic device.

For example, in some embodiments, the device is stored in a dry or partially hydrated form and is hydrated before use (e.g. insertion into the eye). For example, by applying water and/or a hydrating solution to the device prior to positioning the device on an eye surface. In some embodiments, the device is soaked in a hydrating solution before applying to the eye e.g. for 5 seconds-20 mins. In some embodiments, the device is humidified by exposing to humid air and/or gas before applying to the eye e.g. for 1 minute-2 hours.

In some embodiments, one or more therapeutic material is applied to the device. In some embodiments, mucoadhesive material and/or lubricious material is applied to the device e.g. one or more portions of the device.

At 502, in some embodiments, the ophthalmic device is applied to an eye surface. In some embodiments, applying includes bringing the device into contact with the eye surface e.g. the sclera e.g. bulbar conjunctiva.

In some embodiments, applying includes retracting the eyelid under which the device is to be positioned and placing the device on an eye surface usually underneath the eyelid.

In some embodiments, the device is directly and/or manually applied to the eye surface. For example, by the patient themselves. In some embodiments, a caregiver applies the device. Optionally, in some embodiments, the device is applied using an applicator.

In some embodiments, for example, when the device is not rotationally symmetrical, the device is orientated prior to and/or during positioning on the eye surface. In some embodiments, an applicator is sized and/or shaped and/or has one or more indicator to aid positioning the device at a desired orientation within the eye. Orientations, for example, as described elsewhere in this document e.g. orientation with respect to a leading edge and/or shape (e.g. elongation) of the device.

At 504, optionally, in some embodiments, after application to the eye surface, the ophthalmic device is repositioned. For example, by manipulation of the eye and/or ophthalmic device and/or eyelid. For example, in some embodiments, the ophthalmic device is positioned protruding at least partially from underneath an eyelid and, in some embodiments, the ophthalmic device is repositioned (e.g. to be fully underneath the eyelid) by directly moving the ophthalmic device and/or by manipulating the eye (e.g. eyelid) to move the ophthalmic device. For example, in some embodiments, the ophthalmic device is placed on the sclera above the eyelid and then is manipulated in an inferior direction e.g. until it is covered by the eyelid and/or held in position by the tarsal plate. In some embodiments, suction forces between the device and the eye surface (e.g. the posterior surface of the device) are sufficiently low, at least initially, to enable repositioning of the device. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

At 506, in some embodiments, the ophthalmic device is held in place by eye anatomy when the device is worn, and the eye is rolled. For example, held underneath the eyelid e.g. by interaction between the tarsal plate and a retention portion of the device e.g. including one or more feature as illustrated in and/or described regarding device 300, tarsal plate 348 FIGS. 3B-C and/or device 400, tarsal plate 462 FIG. 4B.

At 508, in some embodiments, optionally, the device hydrates within the eye, for example, absorbing tear fluid.

At 510, optionally, in some embodiments, the ophthalmic device adheres to the eye surface.

Where, in some embodiments, adherence is associated with pressure between the ophthalmic device and the eye surface (e.g. posterior surface of the device). In some embodiments, curvature of the ophthalmic device posterior surface aids adherence of the device to the eye surface. In some embodiments, this adherence is amplified by pressure of the eyelid onto the device. In some embodiments, one or more sharp edge of the device aids adherence of the device to the eye surface. In some embodiments, one or more concavity and/or protrusion e.g. on a posterior surface of the device aids adhesion.

In some embodiments, when the device is applied to the eye surface (i.e. is in situ), material properties of a mucoadhesive layer of the device upon application and/or after the mucoadhesive layer properties change in situ (e.g. hydrate) adhere the device to the eye surface.

In an exemplary embodiment, adhesion of the ophthalmic device to the eye surface is sufficient to maintain the device on the eye surface (e.g. sclera) in event of escape of the device from underneath the eyelid. For example, for a short period of time for example, less than 10 minutes, for example, 1 minute-1 hour, for example, during blinking e.g. during a few blinks e.g. 1-10 blinks, or 1-100 blinks. In which time, in some embodiments, a user returns the device to underneath the eyelid e.g. according to one or more feature as described regarding step 504.

At 512, in some embodiments, the device remains on the eyeball for a time period. For example, remaining underneath the eyelid.

In some embodiments, the device is maintained in situ (in the eye) for a period of at least 1 minute, or for 1-10 minutes, or for at least 0.5 hours, or for 0.1-2 hours, or for 2-8 hours, or for over 8 hours, or for 8-24 hours, or 1-7 days, or 7 days-1 month, or 1-3 months, or 3 months-1 year, or 1-30 days, or 1 month-1 year.

In some embodiments, the device remains and/or is maintained in situ (residence time) and is expelled and/or removed from the eye after less than about 24 hours from administration. Where expelling and/or removal, in some embodiments, leaves the eye clean from the device (e.g. without leaving behind debris), for example, the eye being ready for an additional administration e.g. of an additional device.

At 514, optionally, in some embodiments, one or more portions of the device degrades and/or disintegrates.

In some embodiments, degradation of the device occurs by one or more of mechanical degradation (such as eyeball or eyelid movement), chemical degradation (such as tear fluid) or biological degradation (such as enzymatic activity).

In some embodiments, speed of degradation is increased by a user, for example, where, in some embodiments, pressure is applied to the device (e.g. manually) to break the device into more than one part and/or hasten such breakage. In some embodiments, upon breaking into more than one part, one or more of the parts are sized and/or shaped (e.g. having retention portion/s) to remain underneath the eyelid.

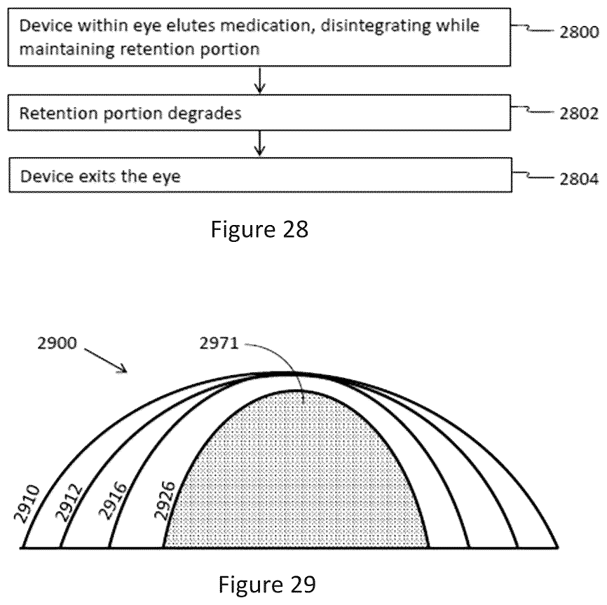

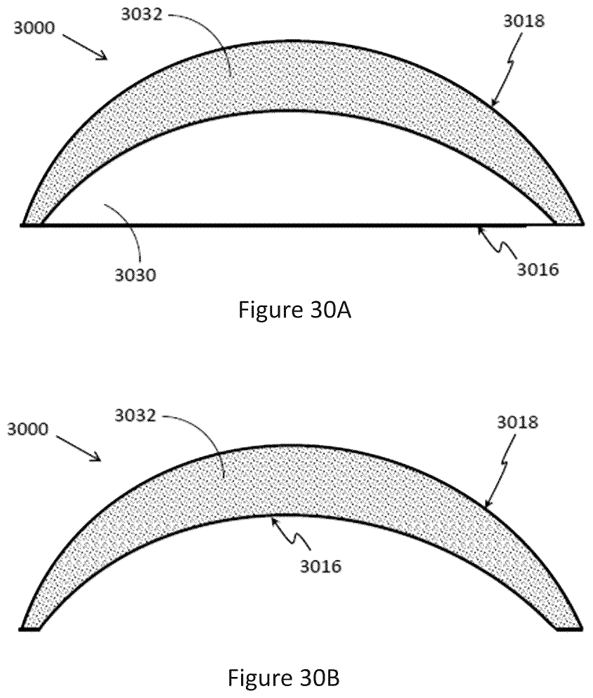

In some embodiments, the device includes portions which degrade at different rates. For example, including one or more features as illustrated in and/or described regarding FIG. 28 and/or FIG. 29 and/or FIGS. 30A-B.

At 516, in some embodiments, the device performs treatment, for example, eluting medication into the eye. In some embodiments, elution is associated with degradation of eluting portion/s of the device.

At 518, optionally, in some embodiments, one or more portions of the device disintegrates. For example, a portion of the device disintegrating to reveal therapeutic material and/or to reveal additional therapeutic material. In some embodiments, portion/s of the device associated with device adhesion disintegrate. For example, a mucoadhesive layer. For example, portions which contribute to mechanical adhesion e.g. portion/s of a device edge and/or portion/s to change curvature of one or more surface of the device.

At 520, optionally, in some embodiments, the device disintegrates. For example, where disintegration of the device mechanically reduces adhesion of the device to the eye and/or reduces size of the device and/or breaks the device into pieces. In some embodiments, the entire device degrades after residence on the eye surface for a time period of between about 0.1 hours to 1 year, or less than 0.1 hours (e.g. immediately e.g. within a minute after placement on the eye).

At 522, in some embodiments, the device ophthalmic and/or portion/s of the ophthalmic device expelled from the eye naturally e.g. expelled by movement of the eyelid and/or eyeball and/or by blinking and/or tearing. In some embodiments, the device is expelled using manipulation of the eyelids. In some embodiments, portions of the device are expelled (e.g. when step 520 has occurred). In some embodiments, the device e.g. where the device remains in one portion, is expelled naturally. For example, upon degradation of adhesive related portion/s of the device.

At 524, optionally, in some embodiments, the ophthalmic device and/or portion/s of the device are removed e.g. manually. For example, by application of mechanical force e.g. pinching and/or applying suction and/or during use of a removal device (e.g. suction cup, tweezers) and/or by flushing of the eye.

In some embodiments, the ophthalmic device is removed by a combination of manual and natural processes. For example, in some embodiments, pressure is applied to the device to break it into more than one piece or to hasten such breakage. Where, in some embodiments, one or more of the pieces are then expelled naturally and/or dissolved.

In some embodiments, concurrent to one or more step and/or in between steps of the method described steps 500-524, one or more additional treatment method is administered concomitantly, sequentially or simultaneously. For example, in some embodiments, topical medication is applied e.g. during residence time of the device on the eye surface.

### 2.3. Exemplary Ophthalmic Devices

Referring now to FIG. 6A showing a simplified schematic top view of an ophthalmic device 600 in position on an eye surface 604 underneath an eyelid 646, according to some embodiments of the invention.

In some embodiments, device 600 includes a retention portion 636 which is formed, when the device is in position on an eye surface, by a portion of device 600 adjacent to an opening of eyelid 646 (where, in some embodiments, eyelid 646 is a lower eyelid).

In some embodiments, retention portion 636 is a portion of device 600 extending from a leading edge 666 of the device inwards (e.g. towards a center of device 600).

In some embodiments, retention portion 636 extends inwards from leading edge 666 by at least a portion 630 of a depth 620 of device 600. In some embodiments, retention portion 636 extends laterally e.g. along leading edge 666 by at least a portion of a lateral length 622 of device 600.

Referring now to FIG. 6B showing a simplified schematic cross section of an ophthalmic device 600, according to some embodiments of the invention.

FIG. 6B illustrates a cross section of device 600 of FIG. 6A where the cross section is taken along line BB of FIG. 6A. FIG. 6B illustrates a range of cross sections of device 600 parallel to line BB of FIG. 6A e.g. for all of lateral extent 628 of retention portion 636. FIG. 6B illustrates each of a range of cross sections of device 600 of FIG. 6A where the cross sections are along line BB of FIG. 6A or parallel to line BB of FIG. 6A.

In some embodiments, the body of the ophthalmic device 600 is symmetrical relative to its longitudinal axis (schematically shown by line AA) and relative to its transversal axis (schematically shown by line BB). For example, when looking at the ophthalmic device 600 (or other ophthalmic devices for example 300, 400, 800, 900, 1300, 1400, 1500, 1600, 1700, 1900, 2000, 2500, 2600, 2700, 2900, 3000, 3100) from the anterior surface, the device is symmetrical relative to its longitudinal axis (AA) and relative to its transversal axis (BB). In some embodiments device 600 has an anterior surface 618 and a posterior surface 616. In some embodiments, device 600 is a volume between the surfaces 616, 618, e.g. one or both of the surfaces curving to meet the other surface. For example, as illustrated in FIG. 6B where anterior surface 618 curves to meet posterior surface 616. In some embodiments, when resident on an eye surface, posterior surface 616 is adjacent to the eye surface (e.g. a sclera). In some embodiments, when resident on an eye surface, anterior surface 618 is adjacent conjunctiva of an eyelid (e.g. of a lower eyelid). In some embodiments, anterior surface 618 is convex and/or has a convex portion. In some embodiments, posterior surface 616 is convex and/or has a convex portion, as schematically shown by dotted line 616a in FIG. 6B. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

In some embodiments, retention portion 636 has a holding height 662 at a holding distance 664 from leading edge 666. Where, in some embodiments, the holding height 662 is maintained (or exceeded) for a length 632 extending from leading edge towards trailing edge 634.

In some embodiments, retention portion 636 has concave and/or sufficiently steep slope 614 (e.g. as defined by slope of anterior surface 618) from leading edge 666 towards holding height 662. Where, in some embodiments, at a first distance 652 from leading edge 666, device 600 has a first height 654 and/or at a second distance 656 from leading edge 666, device 600 has a second height 658. In some embodiments, second distance 656 is at most 1 mm and first distance 652 is at least 1 mm.

Where distances from leading edge 666 are measured along anterior surface and/or from the leading edge along a surface as described regarding measurements of heights (e.g. elsewhere in this document).

In some embodiments, an angle θ of a slope of anterior surface 618 at leading edge 666 (e.g. of a tangent to the surface at the leading edge) or at an edge of the retention portion, is at least 35°, or at least 46°, or is 46-90°, or is 46-86°, or is 46-70°, or is lower or higher or intermediate angles or ranges. Where, in some embodiments, the angle is measured with respect to a planar surface on which the device is resting in a relaxed configuration. Where, in some embodiments, the angle is measured with respect to an eye surface, for example, an angle of the eye surface at the leading edge 666 and/or at an edge of the retention portion.

In some embodiments, anterior surface 618 has a radius of curvature 624 extending from leading edge 666 to holding height 662. Where, in some embodiments, radius of curvature 624 is equal to a peak height of the device e.g. as measured between posterior 616 and anterior 618 surfaces of device 600.

In some embodiments, e.g. as illustrated in FIG. 6B the device cross section (for one or more cross section) is symmetrical, e.g., has mirror symmetry. In some embodiments, the device is symmetrical in at least one direction, for example, about line BB and/or line AA as illustrated in FIG. 6A.

In some embodiments, ophthalmic device 600 is elongate. Where, in some embodiments, a length 622 of device 600 is larger than a width 620 of device 600. In some embodiments length 622 is 1.5-10 times.

A potential advantage of an elongate device is that long sides 666, 634 of the device provide an increased surface for interaction with the eyelid/s e.g. for retention of the device within the eyelid and/or when removing the device using pinch removal.

In some embodiments, device 600 is positioned underneath eyelid 646 where a direction of elongation of device 600 is along a direction of an eyelid contour e.g. a contour of the opening to eyelid 646 and/or along a contour of a tarsal plate 648 of eyelid 646. A potential advantage of elongation of device 600, is an increased length of interaction of the device and the eyelid e.g. the tarsal plate (e.g. in comparison to the length of interaction of a device of the same volume and/or footprint size but less elongated). A potential advantage of elongation of the device, in some embodiments, is reduced likelihood of rotation of the device when in situ. In some embodiments, length 622 is larger than a depth of a cul-de-sac of the eye e.g. potentially preventing device 600 from rotating so that a direction of elongation is aligned in a direction extending from the eyelid opening to the bottom of the cul-de-sac.

In some embodiments, width 620 and/or length 622 of device 600 is 2-10 mm. For example, in some embodiments, length 622 is 5-10 mm and/or width 620 is 3-5 mm and/or height 662 and/or height 624 is 2-2.5 mm. In some embodiments, holding height 662 is at least 2 mm at a distance 664 from leading edge 666 of 2 mm. Alternatively or additionally, in so some embodiments, at distance 652 of 0.5 mm height 654 is at least 1.3 mm. Alternatively or additionally, in some embodiments, at distance 656 of 1 mm height 658 is at least 1.7 mm.

In some embodiments, where distance 664 is 2 mm, height 662 is larger than 0.5-5 mm, or 0.5-2 mm, or 1-2 mm, or lower or intermediate or higher heights or ranges.

In some embodiments, holding height 662 is maintained for a region of the cross section e.g. length 632 being at least 1 mm.

In some embodiments, peak height 624 is at most 5 mm, or 4 mm, or about 3 mm or about 2 mm.

In some embodiments, for example, as described elsewhere in this document, anterior surface 616 is curved, e.g. concave. For example, including one or more feature of one or more of device 1300 FIGS. 13A-C, device 1400 FIG. 14 and/or other device/s elsewhere in this document.

In some embodiments, device 600 is sufficiently rigid and/or resistance to forces (e.g. suction forces) when on an eye that the device does not deform significantly when in an eye so that, in some embodiments, heights as described above are heights above a one or more of:
a plane on which the relaxed device rests,
a plane connecting circumferential edges of the device or a majority (e.g. at least 80%, or at least 90%, or at least 95%, or at least 99%) of a circumference of the device,
a curved surface on which the relaxed device rests e.g. a surface with curvature in one or more directions of an eyeball, e.g. a sphere with radius of curvature of the eyeball.

In some embodiments, distances measured from leading edge 666 are measured along the plane used for measurement of height and/or along the posterior surface and/or along the anterior surface and/or a plane equidistant between the surfaces.

In some embodiments, heights e.g. as described regarding one or more of 654, 658, 662, 624 are heights of the device in situ above an eye surface. Where, in some embodiments, anterior surface 616 is curved (e.g. concave or convex). In some embodiments, forces (e.g. suction between the device and eyeball and/or pressure of the eyelid onto the device via the anterior surface) on the device act to flatten the device when the device is in situ on the eye surface.

Alternatively, the heights described are for a relaxed device, where, in some embodiments, flattening of the device reduces one or more height of the device by at most 30%, or at most 5-50%, or at most 5-30%, or at most 5-20%, from their original height/s. For example, height reducing by at most 30% corresponding to, in some embodiments, upon flattening the maximum height being at least 70% of an original (prior to flattening) maximum height.

Referring now to FIG. 7 showing a simplified schematic illustrating cross sections of portions of devices, according to some embodiments of the invention.

FIG. 7 illustrates exemplary anterior surfaces 714, 714b, 714c of exemplary devices. Where, each of the device anterior surface contours, has a holding thickness 762 at a holding distance 764 from a leading edge 766 of the device. Convex anterior surface 714 (e.g. as described regarding anterior surface 618 of device 600 FIG. 6B) is illustrated for comparison purposes to, for example, an embodiment, where contour 714c extending from leading edge 766 to holding thickness 762 has a constant slope (e.g. within 10% of constant) and an embodiment where contour 714b is concave.

Referring now to FIGS. 8-12 showing simplified schematic top views of devices 800, 900, 1000, 1100, 1200, according to some embodiments of the invention.

Dashed lines are contour lines of an anterior surface of the device indicating, for the anterior surface, same distances above a height defined by a plane of footprints of the device as indicated in FIGS. 8-12 by solid lines.

In some embodiments, as shown for example in FIG. 8, device 800 has the same slope and/or edge contour extending around a perimeter (or extending around at least 50%, or 70%, or 90%) of the device (e.g. as indicated by contour lines).

Where, for example, a slope 814 of the anterior surface adjacent to a leading edge 866 and a slope 874 adjacent to a trailing edge 834 are the same. In some embodiments, slopes 876, 878 on lateral sides of the device are the same e.g. the same as each other and/or the same as slope/s 814, 874. A potential benefit being that holding forces on the device (e.g. of the eyelid on the device) of the device are maintained (or changed less) upon rotation of the device (e.g. while the device is in position e.g. underneath an eyelid). In some embodiments, slopes 876, 878, of lateral sides of device 800 are different, for example, to configure device 800 to changing eye anatomy e.g. in a direction along a contour of an eyelid.

In some embodiments, as shown for example in FIG. 9 and FIG. 11, different edges of the device have different slopes. For example, in some embodiments, a slope 914, 1114 of a retention portion is located in a central region of device 900, 1100 leading edge 966, 1166 where, in some embodiments, one or both of lateral edges of the device have lower gradient slopes 976, 978, 1176, 1178.

In some embodiments, as shown for example in FIG. 10, a slope 1074 of the anterior surface of the device extending to a trailing edge 1034 is higher than that of a retention portion slope 1014 adjacent to a leading edge 1066 of device 1000.

In some embodiments, as shown for example in FIG. 11 and FIG. 12, a region of the device adjacent to a leading edge 1166, 1266 hosts the retention portion and a slope of the device 1174, 1274 extending towards a trailing edge has lower gradient slope. A potential benefit being increased comfort as the device tapers in thickness as it extends further underneath the eyelid towards the apex of the conjunctival fornix. In some embodiments, one or both lateral sides of the device also have lower gradient slope 1176, 1178 than retention portion slope 1114.

Referring now to FIG. 13A showing a simplified schematic view of an ophthalmic device 1300, according to some embodiments of the invention. FIG. 13A shows an isometric view of ophthalmic device 1300. In some embodiments, the form of the ophthalmic device 1300 is a dome-like form or half a sphere. Also referring to FIG. 13B showing a simplified schematic cross sectional view of an ophthalmic device 1300, according to some embodiments of the invention. And also referring to FIG. 13C showing a simplified schematic top view of an ophthalmic device 1300, according to some embodiments of the invention.

In some embodiments, ophthalmic device 1300 has an anterior surface 1316 and a posterior surface 1318. Where, in some embodiments, anterior surface 1318 curves to meet posterior surface 1316 at an edge 1366 of device 1300.

In some embodiments, ophthalmic device 1300 is symmetric, for example about one or more axis of symmetry. In some embodiments, as shown for example in FIG. 13C, ophthalmic device 1300 is symmetric about line AA and/or line BB.

In some embodiments, device 1300 has rotational symmetry, for example about a central axis 1370 which extends through a center 1372 of device top view. Where axis 1370 is perpendicular to one or both of anterior 1318 and posterior 1316 surfaces.

In some embodiments, device 1300 footprint (e.g. as illustrated in FIG. 13C) has a diameter of 2-10 mm, or 3-8 mm, or 4-7 mm, or 5-6 mm. Where, in some embodiments, device 1300 has a height of 2-2.5 mm.

In some embodiments, posterior surface 1316 is curved, for example, in one or more directions. For example, in some embodiments, FIG. 13B illustrates a cross section taken of device along line AA and/or line BB of FIG. 13C.

A potential benefit of a symmetrical (e.g. rotationally symmetrical device) is that device characteristics e.g. regarding eye anatomy, are maintained if the device rotates in situ.

In some embodiments, not shown in FIG. 13A, 13B or 13C, anterior surface 1318, has higher radius of curvature (RA—Radius Anterior) than a radius of curvature of posterior surface 1316 (RP—Radius Posterior). For example, where, in some embodiments, RA is 1.2-10 or 1.2-5 times RP.

In an exemplary embodiment, a radius of curvature of anterior surface 1318 is equal (or about equal, for example, within 20%, or within 10%, or within 5%) of a maximum thickness 1324 of device 1300. For example device 1300 anterior surface 1318 having a hemisphere shape.

In an exemplary embodiment, a radius (e.g. half of width 1320) of the device footprint (FIG. 13C) and/or edges 1366 is 0.5-10 mm, or 1-10 mm, or 1-5 mm, or 2-4 mm, or 2.5-3 mm.

Hemisphere-shaped devices e.g. as described regarding FIGS. 13A-C, and having diameter 1320 of 5.5 mm, having a concave posterior surface 1316 and having height 1324a (as measured when the device is in a relaxed configuration on a planar surface) 2 mm were placed on the sclera under the lower eyelid of human volunteers and stayed there comfortably for several hours.

Elongated-Hemisphere-shaped devices or also defined as having an elongated elliptical half-sphere form e.g. as described regarding FIGS. 15A-D, having a concave posterior surface and having length 1522 of 6 mm, width 1520 of 4 mm, and height 1524 of 2 mm were placed on the sclera under the lower eyelid of human volunteer's eyes and stayed there comfortably for several hours.

FIG. 14 is a simplified schematic cross sectional view of an ophthalmic device 1400, according to some embodiments of the invention.

In some embodiments, curvature and/or slope of a surface 1418 of device 1400 changes along a cross section of the device. For example, in some embodiments, curvature of anterior surface 1418 changes moving from a leading edge 1466 towards a trailing edge 1434. Where leading edge 1466 and/or trailing edge 1434 include one or more feature as described regarding leading edge 666 and/or trailing edge 634 FIG. 6A, FIG. 6B respectively. For example, device 1400 having a retention portion 1436 with a slope 1414 and/or having a holding thickness 1424 where a curvature of anterior surface 1418 then reduces (e.g. from a maximal thickness portion of the device) towards trailing edge. Where, in some embodiments, slope 1414 and/or holding thickness 1424 have one or more feature of slope 614 and/or thickness 624 FIG. 6A and/or FIG. 6B. In some embodiments, such a cross section positions the retention portion non-centrally with respect to the top view and/or footprint of the device e.g. off center and towards a side of the device proximal to an opening of the eyelid when the device is in situ.

In some embodiments, (e.g. where anterior surface 1418 has changing curvature) posterior surface 1416 has constant curvature 1416. Alternatively, in some embodiments, posterior surface curvature 1416 also changes moving across one or more cross section of the device. For example, thinning of the device in a direction moving away from the leading edge, in some embodiments, being due to decrease in curvature of anterior surface combined with increase in curvature of the posterior surface. In some embodiments, (e.g. where anterior surface 1418 has constant curvature) thinning of the device in a direction moving away from the leading edge is due to increase in curvature of the posterior surface.

Referring now to FIG. 15A showing a simplified schematic top view of an ophthalmic device 1500, according to some embodiments of the invention. Also referring to FIG. 15B showing a simplified schematic view of an ophthalmic device 1500, according to some embodiments of the invention. FIG. 15B shows an isometric view of ophthalmic device 1500. And also referring to FIG. 15C showing a simplified schematic cross sectional view of an ophthalmic device, according to some embodiments of the invention. Lastly, also referring to FIG. 15D showing a simplified schematic cross sectional view of an ophthalmic device 1500, according to some embodiments of the invention.

In some embodiments, device 1500 is elongate. In some embodiments, device 1500 is defined as having an elongated-Hemisphere-shaped form or also defined as having an elongated elliptical half-sphere form. Where, in some embodiments, elongation of device 1500 includes one or more feature as described regarding device 600 FIG. 6A. In an exemplary embodiment, a device width 1520 is 1.1-10 times, or 1.1-5 times, or 1.2-3 times, or 1.2-2.5 times, or about 1.5-2 times a device length 1522.

In some embodiments, a leading edge 1566 of device 1500 is straight and/or a trailing edge 1534 is straight. In some embodiments, leading edge 1566 and trailing edge 1534 are parallel (or within 1, or 3, or 5, or 10 degrees of parallel). In some embodiments, leading edge 1566 and/or trailing edge 1534 are straight where compared to, for example, lateral edges 1580 and 1582. In other words, leading edge 1566 and/or trailing edge 1534 may comprise straight portions, whereas lateral edges 1580 and 1582 are curved, and therefore do not comprise straight portions.

In some embodiments, one or both of lateral edges 1580 and 1582 are curved. A potential benefit being increased comfort for a user wearing the device. In some embodiments, one or both of edges 1580 has a radius of curvature 1584. In an exemplary embodiment, radius of curvature 1584 is half (or about half) of a device footprint width 1520.

FIG. 15C illustrates a cross section of device 1500 taken laterally across the device e.g. along a direction of elongation e.g. along line CC (in FIG. 15A). In some embodiments, device cross section has a radius of curvature 1588 of an anterior surface 1518 extending from a first lateral edge 1580. In some embodiments, the cross section has the same radius of curvature 1588 for anterior surface 1518 extending from second lateral edge 1582 to a central region and/or a maximal thickness 1524 of the cross section. In some embodiments, a radius of curvature 1518 of the posterior surface, in one or more directions, is 1-15 mm, or 1-10 mm, and/or has a ratio to that of the sclera being 0.1-2, or 0.5-1.

FIG. 15D illustrates a cross section of device 1500 taken in a direction of leading edge 1566 towards trailing edge 1534 e.g. along line DD (in FIG. 15A). In some embodiments, FIG. 15D illustrates cross sections of device 1500 extending along a central region 1590 of device 1500 e.g. as illustrated by arrow 1586. In some embodiments, the cross section of FIG. 15D includes one or more feature of the cross section of FIG. 6B. In some embodiments, a radius of curvature 1590 of anterior surface 1518 is the same as a maximal thickness 1590 of the cross section. In some embodiments, the form of the ophthalmic device 1500 is a dome-like form or a half sphere.

In some embodiments, one or more of radii of curvature 1584, 1588, 1590 are related. In some embodiments, one or more of radii of curvature 1584, 1588, 1590 with respect to one or more of each other, are the same, or are about the same, or are within 5%, or 10%, or 20%, or 30%, of each other.

In some embodiments, lateral sides of device 1500 have the same curvature towards lateral edges 1580, 1582, in directions extending from a central region 1590 of device 1500, in some embodiments, lateral edge portions of device anterior surface having a quarter hemisphere shape.

FIG. 15C also illustrates (e.g. alternatively to FIG. 15D) a cross section of device 1500 taken along line DD (in FIG. 15A), the device, in some embodiments, having a planar central region of the anterior surface as defined by a base region 1502 of anterior surface 1518 in FIG. 15C.

In some embodiments, device 1500 has a maximal thickness, extending along a lateral central line CC of device 1500 footprint (FIG. 15A). In some embodiments, the maximal thickness extends along a central contour e.g. as illustrated by arrow 1586.

In an exemplary embodiment, width 1520 is 4.5 mm and length 1522 is 10 mm. Where, in some embodiments, thickness 1524 and/or one or more of radii of curvatures 1584, 1588, 1590 are about 2.25 mm.

In an exemplary embodiment, width 1520 is about 5 mm and length 1522 is about 6.5 mm. Where, in some embodiments, thickness 1524 and/or one or more of radii of curvatures 1584, 1588, 1590 are about 2.25 mm.

In an exemplary embodiment, width 1520 is about 4.5 mm and length 1522 is about 8.5 mm. Where, in some embodiments, thickness 1524 and/or one or more of radii of curvatures 1584, 1588, 1590 are about 2.25 mm.

In an exemplary embodiment, width 1520 is about 5.5 mm and length 1522 is about 10 mm. Where, in some embodiments, thickness 1524 and/or one or more of radii of curvatures 1584, 1588, 1590 are about 2.75 mm.

In an exemplary embodiment, width 1520 is about 4 mm and length 1522 is about 6 mm. Where, in some embodiments, thickness 1524 and/or one or more of radii of curvatures 1584, 1588, 1590 are about 2 mm.

In an exemplary embodiment, width 1520 is about 5.5 mm and length 1522 is about 8.5 mm. Where, in some embodiments, thickness 1524 and/or one or more of radii of curvatures 1584, 1588, 1590 are about 2.25 mm.

In an exemplary embodiment, width 1520 is about 4.5 mm and length 1522 is about 6.5 mm. Where, in some embodiments, thickness 1524 and/or one or more of radii of curvatures 1584, 1588, 1590 are about 2 mm. Where, in some embodiments, a weight of the device is 18 mg. In some embodiments, the minimum weight of the device is 8 mg.

Referring now to both FIG. 15C and FIG. 15D, posterior surface 1516 is curved, at least for a portion of the posterior surface 1516, in at least one direction. For example, as illustrated by dashed lines in FIG. 15C and FIG. 15D which illustrate alternative posterior surface/s. In some embodiments, the posterior surface of device 1500 is concave, in at least one direction.

A potential advantage of a concave posterior surface is increased adhesion of the device to an eye surface on which the device rests. In some embodiments, not shown in FIGS. 15A-D, portion/s of an anterior surface 1518, have higher radius of curvature RA than the posterior surface RP. For example, where, in some embodiments, RA is 1.2-10 times RP, or 1.2-5 times RP. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

Referring now to a cross section taken along line DD. In an exemplary embodiment, at 0.5 mm from leading edge 1566, height (and/or thickness) of the device is 1.32 mm, and at a distance of 1 mm from leading edge 1566, height (and/or thickness) of the device is 1.73 mm, and at a distance of 2 mm from leading edge 1566 height (and/or thickness) of the device is 2 mm. For example, in some embodiments, a radius of curvature 1588 and/or 1590 is 2 mm.

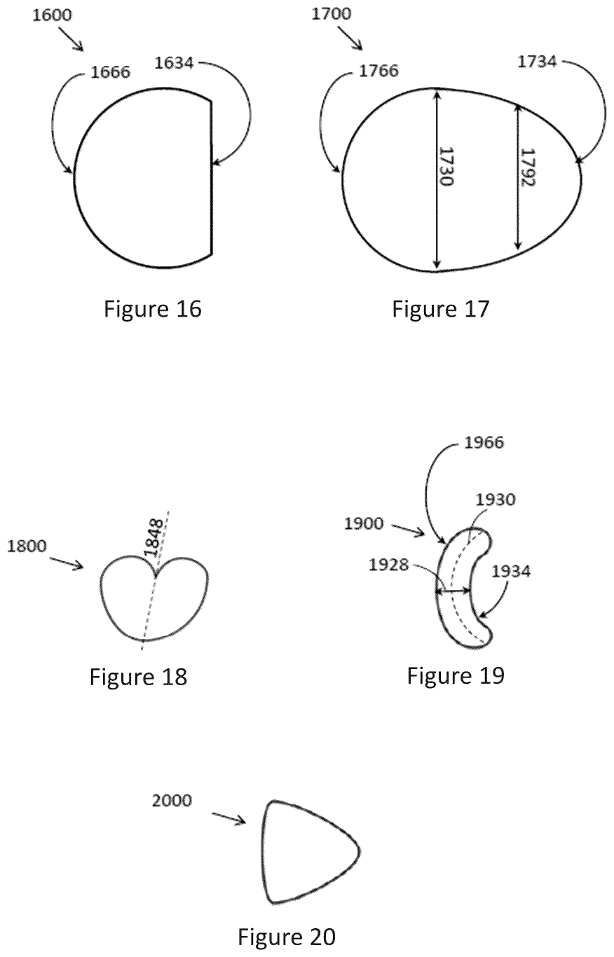

Referring now to FIGS. 16-20 showing simplified schematic top views of devices 1600, 1700, 1800, 1900, 2000, according to some embodiments of the invention.

In some embodiments, as shown for example in FIG. 16, device 1600 is truncated at one or more side. In some embodiments, device 1600 has a curved edge 1666 and a low curvature edge 1634 (e.g. where, in some embodiments, low curvature edge 1634 is straight). In some embodiments, a region of a low curvature edge 1634 has a high curvature in a direction perpendicular to a plane of the footprint (and/or of FIG. 16). For example, in some embodiments, edge 1634 hosting a retention portion e.g. having feature/s as described regarding retention portions elsewhere in this document (e.g. retention portion 636 FIG. 6B).

In some embodiments, as shown for example in FIG. 17, device 1700 has a top view shape which is tapered in one or more directions. For example, where, a maximum width 1730 of the device tapers towards one or more edge 1766, 1734 of device, for example, tapering more towards a first edge 1734 than towards a second edge 1766. In some embodiments, maximum width 1730 is located closer to one edge than the other e.g. towards the less tapered side of the device between maximum width 1730 and edge 1766. In some embodiments, second edge 1766 is a device leading edge and first edge 1734 is a trailing edge, leading and trailing edges including feature/s as described elsewhere in this document.

In some embodiments, as shown for example in FIG. 18, an ophthalmic device 1800 has a shape having a narrow portion 1848 and/or indentation in the external form.

In some embodiments, device 1800 shape facilitates removal e.g. by pinching (e.g. manually and/or using a removal implement). Where, for example, in some embodiments, a portion configured to fold which is optionally in a central region (in one or more dimension) of the device (e.g. fold region; 1848 FIG. 18) is thinner and/or has a smaller footprint extent, e.g. facilitating folding after applying pinching force at one or both of larger extent regions on either side of the smaller footprint portion. In some embodiments, device 1800 includes two retention portions, for example, one at each side of fold region 1848.

In some embodiments, as shown for example in FIG. 19, a device shape 1900 e.g. device footprint is selected for anatomical fit. For example, in some embodiments, device 1900 is shaped to position the device in contact with a portion of patient anatomy. For example, in some embodiments, an elongated device is curved and/or describes a curved shape. In some embodiments, ophthalmic device 1900 includes a curved shape top view and/or footprint. In some embodiments, a central longitudinal axis 1930 of the curved shape is 1-40 mm long, or 5-20 mm, long. In some embodiments, a radius of curvature of device 1900 is selected for the device to follow a curvature of an eyelid and/or portion of a cul-de-sac of an eyelid. Where, in some embodiments, a radius of curvature of 1928 is 20-100 mm, or 20-50 mm.

In some embodiments, the curve of device 1900 is sized and/or shaped to fit the eye e.g. a curve of an eyeball underneath an eyelid. In some embodiments, a retention portion is hosted adjacent to edge 1966. Alternatively or additionally, in some embodiments, a retention portion is hosted adjacent to edge 1934.

In some embodiments, as shown for example in FIG. 20, device 2000 has one or more corners e.g. three corners for device 2000, for example, connected by sides. In some embodiments, corners of device 2000 facilitate removal of the device by pinching, for example providing the same potential distance (e.g. associated with extent) over which to apply pinch force (e.g. as a circular footprint device) but with reduced device footprint area. In some embodiments, a retention portion is hosted in a central region of device 2000 footprint. In some embodiments, retention portion/s are hosted adjacent to one or more edge of the multi-edged device.

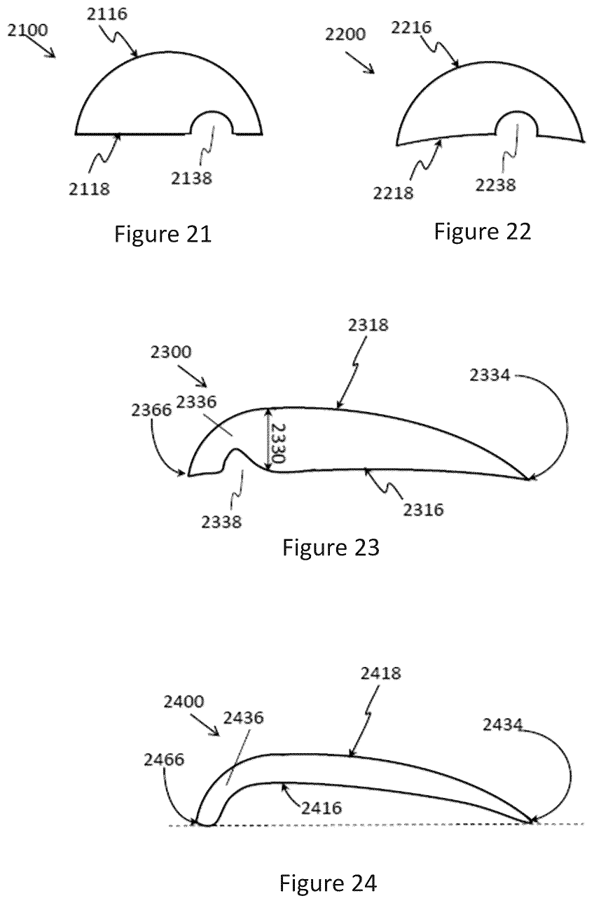

Referring now to FIGS. 21-24 showing simplified schematic cross sections of devices 2100, 2200, 2300, 2400 according to some embodiments of the invention.

In some embodiments, as shown for example in FIG. 21, a posterior surface 2118 of device 1300 includes one or more concavity 2138.

In some embodiments, as shown for example in FIG. 22, device 2200 includes both a curved posterior surface 2216 surface and one or more concavity 2238 on posterior surface 2218.

In some embodiments, as shown for example in FIG. 23, device 2300 includes a concavity 2338 located on a posterior surface 2316. In some embodiments, concavity 2338 is located at a region of a retention portion 2336. For example, the concavity being adjacent to a leading edge 2366 of device, for example, within 1-5 mm of leading edge 2366. For example, located on a same portion (e.g. half) of the device as the retention portion 2336. Optionally, in some embodiments, curvature of an anterior surface 2318 reduces in a direction moving from a maximum thickness 2330 of device 2300 towards a trailing edge 2334 of the device.

In some embodiments, as shown for example in FIG. 24, device 2400 has a highly concave posterior surface 2416, when compared for example, with the device 2300 shown in FIG. 23. In some embodiments, curvature of posterior surface 2416 changes along a length of the surface e.g. from first edge 2466 (which, in some embodiments is a leading edge) to second edge 2434 (which, in some embodiments, is a trailing edge). Posterior surface 2416, in some embodiments, having a higher curvature at a region adjacent to first edge 2466. In some embodiments, height (or thickness of the retention portion e.g. as defined within the overview section and/or with respect to FIGS. 6A-B) is measured between the dashed line illustrated in FIG. 24 and anterior surface 2418. Where, in some embodiments, the dashed line illustrates a planar surface.

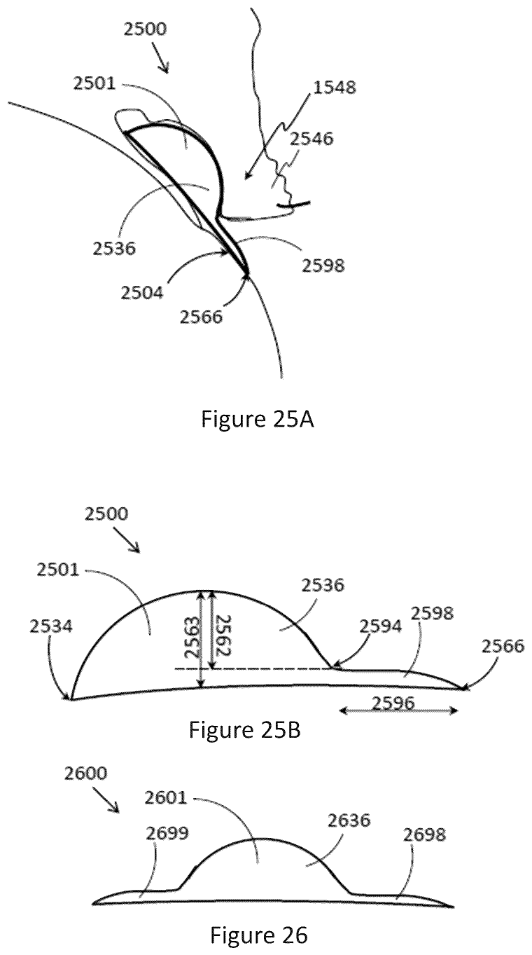

Referring now to FIG. 25A showing a simplified schematic cross sectional view of a device 2500 positioned on an eye surface, according to some embodiments of the invention. Referring also to FIG. 25B is a simplified schematic cross sectional view of a device 2500, according to some embodiments of the invention. In some embodiments, FIG. 25A and FIG. 25B illustrate the same device and a same cross sectional view of the same device 2500.

In some embodiments, device 2500 includes an extending portion 2598 which is not covered by eyelid 2546 e.g. when the eye is open e.g. as illustrated in FIG. 25A. Where, in some embodiments, a body 2501 of device 2500 is located underneath eyelid 2546 (e.g. when the eye is open e.g. as illustrated in FIG. 25A).

In some embodiments, extending portion 2598 extends from body 2501 of device 2500 which includes a retention portion 2536.

In some embodiments, extending portion 2598 is thin and/or has a thin edge. In some embodiments, a step formed between a leading edge 2566 of device 2500 and the eye surface (or a planar surface contacting the device edges) is 1-500 microns, or 1-100 microns, or 10-50 microns. Thinness and/or the small step potentially presenting less surface area to interact with the eyelid as is moves over the area of the extending portion, e.g. during blinking and/or eyeball movement.

In some embodiments, extending portion 2598 has a length 2596 of 0.5-6 mm. In some embodiments, body 2501 has one or more feature of devices e.g. as described elsewhere in this document. For example, one or more of the devices as described elsewhere in this document having an extending portion 2598 extending from the device leading edge.

In some embodiments, body 2501 of the device is held in position underneath the eyelid. For example, in some embodiments, body 2501 having a retention portion 2536 which includes one or more feature as described regarding and/or illustrated for retention portion 636 FIGS. 6A-B and/or elsewhere in this document.

In some embodiments, slope and/or thickness of retention portion 2536 is measured with respect to an edge 2594 of the retention portion with respect to extending portion 2598 and, for example, not by a leading edge 2566 of the device.

In some embodiments, extending portion 2598 is used to load a meniscus (the valley between the eyelid edge and the eyeball where the lacrimal/tear fluid accumulates and creates a reservoir of fluid) with one or more material and/or active pharmaceutical ingredient (API).

Referring now to FIG. 26 showing a simplified schematic cross sectional view of a device 2600, according to some embodiments of the invention.

In some embodiments, a device 2600 has extending portions 2698, 2699 extending from a device body 2601 in more than one direction. In some embodiments, a single extending portion surrounds a device body 2601 footprint. Where device body 2601 includes one or more feature as illustrated in and/or described regarding device body 2501 FIGS. 25A-B. Where one or both of extending portions 2698, 2699 have one or more feature as illustrated in and/or described regarding extending portion 2598 FIGS. 25A-B.

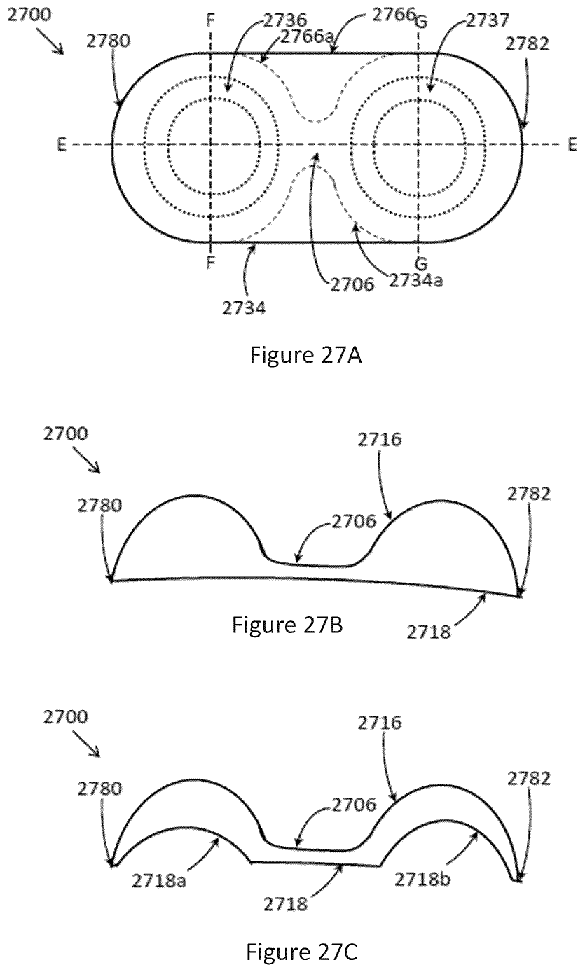

Referring now to FIG. 27A showing a simplified schematic top view of an ophthalmic device 2700, according to some embodiments of the invention. Referring also to FIG. 27B is a simplified schematic cross sectional view of an ophthalmic device 2700, according to some embodiments of the invention. FIG. 27B illustrates a cross sectional view of device 2700 of FIG. 27A, for example, taken along line EE of FIG. 27A. In FIG. 27A, dotted lines are contour lines of an anterior surface 2716 of device indicating, for the anterior surface, same distances above a height defined by a plane a device footprint as indicated in FIG. 27A by a solid line.

In some embodiments, device 2700 includes more than one retention portion 2736, 2737. Where, in some embodiments, retention portions 2736, 2737 are connected by a connecting portion 2706 which, in some embodiments, has a thickness which is below that required by a retention portion.

A potential advantage of multiple retention portions is potential retention of the device underneath an eyelid if a retention portion escapes from underneath the eyelid. A potential advantage of having more than one raised portion of the device (e.g. as provided by retention portions) is protruding surfaces easing pinch-removal e.g. as described elsewhere in this document. A potential advantage of having a connecting portion 2706, for example, instead of a single large retention portion is the ability to have a long device with reduced device volume.

Referring now back to FIG. 27A, which, in some embodiments, illustrates another embodiment having (instead of edge 2766), edge 2766a illustrated as a dashed line, which edge 2766a, in some embodiments, has an inlet, for example, extending towards central region 2706 where the central region 2706 has a smaller extent and/or footprint than central region 2706. In some embodiments, a device has a corresponding shape on an opposing edge 2734a illustrated as a dashed line (instead of edge 2734a).

In some embodiments, cross sections of device 2700 of FIG. 27A including edge inlet/s has the same cross sections as described for device 2700 of FIG. 27A without inlets.

Referring now to FIG. 27C showing a simplified schematic cross sectional view of a device 2700, according to some embodiments of the invention.

FIG. 27C illustrates an embodiment of one or both devices as illustrated by FIG. 27A, where, in some embodiments, each retention portion has a corresponding concave curve 2718a, 2718b, in posterior surface 2718a. Where concave surfaces include one or more feature as described regarding devices with a single retention portion e.g. elsewhere in this document. For example, including one or more feature of posterior surface 616 FIG. 6B and/or posterior surface 1516 FIGS. 15C-D and/or posterior surface 1316 FIG. 13B and/or FIG. 14. In some embodiments, the surfaces are concave in more than one direction, for example cross section taken along line FF and/or line GG having concave posterior surface, for example the cross section/s having one or more feature as described and/or illustrated regarding the cross section of FIG. 13B and/or FIG. 14.

### 2.4. Exemplary Degradation

Referring now to FIG. 28 showing a process of ophthalmic device degradation, according to some embodiments of the invention. Additionally see Chapter 4 below, regarding degradation according to specific elastomeric matrices, according to some embodiments of the invention.

At 2800, in some embodiments, a device positioned within an eye (e.g. underneath an eyelid of the eye) degrades while maintaining structural feature/s of a retention portion. The retention portion acting to maintain the device underneath the eyelid. Where, during degrading, in some embodiments, the device elutes one or more active pharmaceutical ingredients (API). In some embodiments, degradation comprises the loss of mass.

At 2802, in some embodiments, for example, after degradation and/or disintegration of other portion/s of the device, the retention portion degrades.

At 2804, in some embodiments, upon degradation of the retention portion, the device exits the eye, for example, upon exiting from underneath the eyelid.

FIG. 29 is a simplified schematic cross sectional view of a device 2900, according to some embodiments of the invention.

In some embodiments, contours 2910, 2912, 2916, 2926, illustrate an anterior surface of device 2900 over time, e.g. as device 2900 degrades during residence on an eye. In some embodiments, a slope and/or thickness of a retention portion is maintained e.g. through transition of device anterior surface from 2910 to 2912 and 2916 and only afterwards, in some embodiments do thickness (e.g. as illustrated) and/or slope of the retention portion reduce.

In some embodiments, device 2900 includes different portions with different degradation speeds. Where, in some embodiments, portion 2971 includes material which is less rapidly degraded (or is not degraded within the eye) than other portions of the device. In some embodiments, a less rapidly degraded portion of the device (e.g. portion 2971) is sized and/or shaped and/or positioned with respect to other portions of the device to maintain a retention portion e.g. during degradation of other portion/s of the device.

Referring now to FIGS. 30A-B showing simplified schematic cross sectional views of a device 3000, according to some embodiments of the invention.

In some embodiments, device 3000 includes different portions 3032, 3030 with different degradation times. In some embodiments, a posterior portion 3030 adjacent to a posterior surface 3016 of device 3000 is more rapidly degraded, than an anterior portion 3032 adjacent to an anterior surface 3018 of device 3000. For example as illustrated in transition in the device cross section illustrated in transition between FIG. 30A to FIG. 30B. In some embodiments, degradation of an anterior surface of device 3000, for example, combined, in some embodiments, with resistance of the device to collapse on the eye surface, maintains a slope and/or height of the device above the eye surface e.g. maintains a retention portion property/ies.

### 2.5. Exemplary Ophthalmic Devices

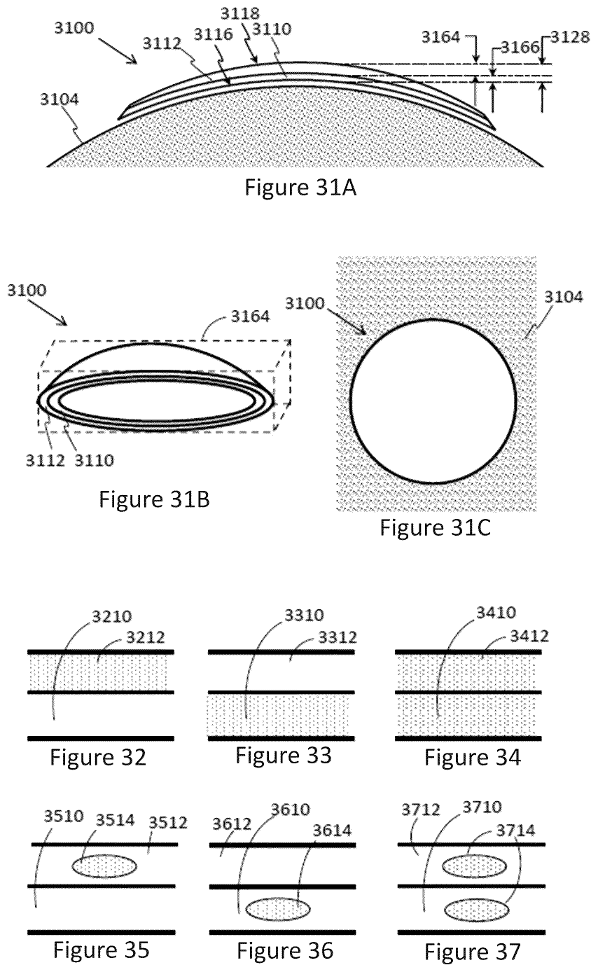

Referring now to FIG. 31A showing a simplified schematic cross section view of an ophthalmic device 3100 on an eye surface 3104, according to some embodiments of the invention. Also referring to FIG. 31B showing a simplified schematic view of an ophthalmic device 3100, according to some embodiments of the invention. Also referring to FIG. 31C showing a simplified schematic view of an ophthalmic device 3100, according to some embodiments of the invention.

In some embodiments, device 3100 includes a posterior surface 3116 and an anterior surface 3118.

In some embodiments, device 3100 includes a plurality of layers 3110, 3112. Where, in some embodiments, layers generally follow surfaces of the device.

In some embodiments, a first layer 3110 is adjacent to and/or in contact with eye surface 3104 and/or forms at least a part of posterior surface 3116. In some embodiments, first layer 3110 includes mucoadhesive material. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

In some embodiments, a second layer 3112 contacts, at least periodically, an inner surface of an eyelid, and/or forms at least part of anterior surface 3114. In some embodiments, second layer has a smooth outer surface and/or includes lubricious material. In some embodiments, one or both of first and second layers 3110, 3112, include and/or elute therapeutic material/s.

FIG. 31B, illustrates determining of an extent of device 3100 where, extent is, one or more dimension and/or average of two or more dimensions of a bounding box 3164 which is a smallest cuboid shape into which device 3100 fits.

In some embodiments, device 3100 includes one or more additional layer (e.g. as described elsewhere in this document).

In some embodiments, the ophthalmic device has a thickness 3128 (e.g. a maximum thickness and/or average thickness of a central 50% of the device) of less than 100 microns. In some embodiments, the ophthalmic device has a thickness of between about 1 to about 200 microns. In some embodiments, the device has a thickness of about 1, or about 2, or about 3, or about 4, or about 5, or about 6, or about 7, or about 8, or about 9, or about 10, or about 15, or about 20, or about 25, or about 30, or about 35, or about 40, or about 45, or about 50, or about 55, or about 60, or about 70, or about 80, or about 90, or about 100, or about 110, or about 120, or about 130, or about 140, or about 150, or about 160, or about 170, or about 180, or about 190, or about 200 microns. In some embodiments, the device has a thickness of between about 10 to about 100 microns.

In some embodiments, a first (e.g. mucoadhesive) layer 3166 and/or a second (e.g. lubricous) layer 3164 and/or an optional additional intermediate layer or layers (e.g. as described elsewhere in this document) has a thickness (e.g. a maximum thickness and/or average thickness at a central 50% of the device) of less than 100 microns. In further embodiments, one or more layer has a thickness of between about 1 to about 200 microns. In some embodiments, lubricious layer and/or a mucoadhesive layer and/or an intermediate layer has a thickness of about 1 microns, 2 microns, 3 microns, 4 microns, 5 microns, 6 microns, 7 microns, 8 microns, 9 microns, 10 microns, 15 microns, 20 microns, 25 microns, 30 microns, 35 microns, 40 microns, 45 microns, 50 microns, 55 microns, 60 microns, 70 microns, 80 microns, 90 microns, 100 microns, 110 microns, 120 microns, 130 microns, 140 microns, 150 microns, 160 microns, 170 microns, 180 microns, 190 microns, 200 microns. In some embodiments, one or more layer has a thickness of between about 1 to about 10 microns.

In some embodiments, one or more layer has about the same thickness as one or more other layer. In some embodiments, one or more layer has a different thickness than one or more other layer. In some embodiments, one or more layer has non-uniform thickness. For example, thinning from a central region of the device outward towards edge region/s of the device. For example, a layer having cavities and/or holes and/or protrusion/s.

Referring now to FIGS. 32-37 showing simplified schematic cross section views of portions of ophthalmic devices, according to some embodiments of the invention. In FIGS. 32-37, shaded areas indicate presence of active pharmaceutical ingredient/s (API).

Referring now to FIG. 32 and FIG. 35, in some embodiments, a second layer 3212, 3512 (e.g. including one or more feature as described regarding second layer 3112 FIGS. 31A-C) comprises active pharmaceutical ingredient/s (API). For example, dispersed within second layer 3112 and/or located within one or more region 3514 of second layer 3512.

Referring now to FIG. 33 and FIG. 36, in some embodiments, a first layer 3310, 3610 (e.g. including one or more feature as described regarding first layer 3110 FIGS. 31A-C) comprises active pharmaceutical ingredient/s (API). For example, dispersed within first layer 3110 and/or located within one or more region 3614 of second layer 3610.

Referring now to FIG. 34 and FIG. 37, in some embodiments, both a first layer 3410, 3710 (e.g. including one or more feature as described regarding first layer 3110 FIGS. 31A-C) and a second layer 3412, 3712 (e.g. including one or more feature as described regarding second layer 3112 FIGS. 31A-C) comprises active pharmaceutical ingredient/s (API). For example, dispersed within first layer 3410 and/or second layer 3412 and/or located within one or more region 3714 of second layer 3712 and/or first layer 3710. Where, in some embodiments, (not illustrated) one of the layers hosts a portion which has therapeutic material and the other layer has therapeutic material dispersed within it. In some embodiments, discrete region/s including therapeutic material include different therapeutic material than those layers which have therapeutic material dispersed within them. In some embodiments, discrete region/s are encapsulated by other material, the material for example, in some embodiments, eroding before the therapeutic material starts to elute into the eye.

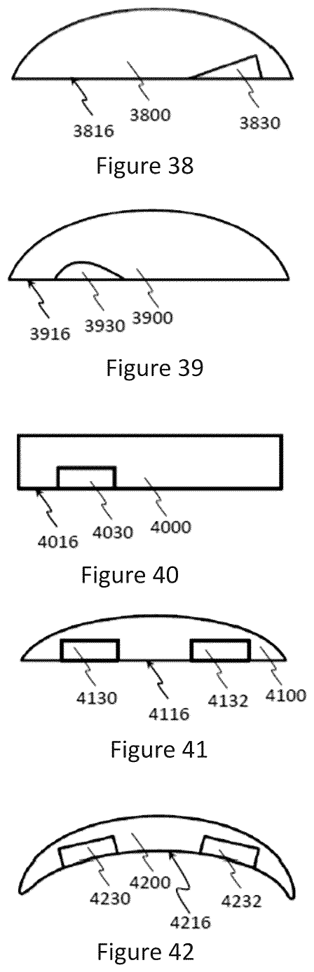

Referring now to FIGS. 38-44 showing simplified schematic cross sectional views of devices 3800, 3900, 4000, 4100, 4200, 4300, 4400, according to some embodiments of the invention.

In some embodiments, a device has a posterior surface which is flat or high radius of curvature e.g. posterior surface 3816 of device 3800 FIG. 38, e.g. posterior surface 3916 of device 3900 FIG. 39, e.g. posterior surface 3406 of device 4000 FIG. 40, e.g. posterior surface 4116 of device 4100 FIG. 41. Where, in some embodiments, a high radius of curvature is more than 5 mm, or more than 10 mm, or more than 9.6 mm or 8-12 mm, or 9-11 mm.

In some embodiments, a device has a posterior surface which is concave, e.g. posterior surface 4216 of device 4200, e.g. posterior surface 4316 of device 4300, e.g. posterior surface 4416 of device 4400. Where, in some embodiments, concave posterior surface 4216, 4316, 4416, includes one or more feature as illustrated in and/or described regarding concave and/or curved posterior/s elsewhere in this document.

In some embodiments, a device and/or a cross section of a device includes a single cavity e.g. where, in some embodiments, element 3830, 3930, 4030 of FIGS. 38-40 respectively is a cavity. Where, in some embodiments, the cavity 3830, 3930, 4030, is disposed on a posterior surface 3816, 3916, 4016, of the device.

In some embodiments, a device and/or a cross section of a device includes a single portion with different material, for example, mucoadhesive material e.g. where, in some embodiments, element 3830, 3930, 4030 of FIGS. 38-40 respectively is a cavity. Where, in some embodiments, the mucoadhesive portion 3830, 3930, 4030, is disposed on a posterior surface 3816, 3916, 4016, respectively of the device.

Referring now to FIG. 41 and FIG. 42. In some embodiments, a device and/or a cross section of a device has a single portion with different material 4130, 4230 e.g. mucoadhesive material and has a single cavity 4132, 4232. Where, in some embodiments, cavity 4130, 4230 and different material portion 4132, 4232 are disposed on a posterior surface 4116, 4216 of device 4100, 4200 respective.

In some embodiments, a device and/or a cross section of a device has two portions of different material/s where portions 4130, 4132, of device 4100 and/or portions 4230, 4232 of device 4200 have the same material or different materials.

In some embodiments, a device 4100, 4200 and/or a cross section of the device has two cavities 4130, 4132, and 4230, 4232 respectively.

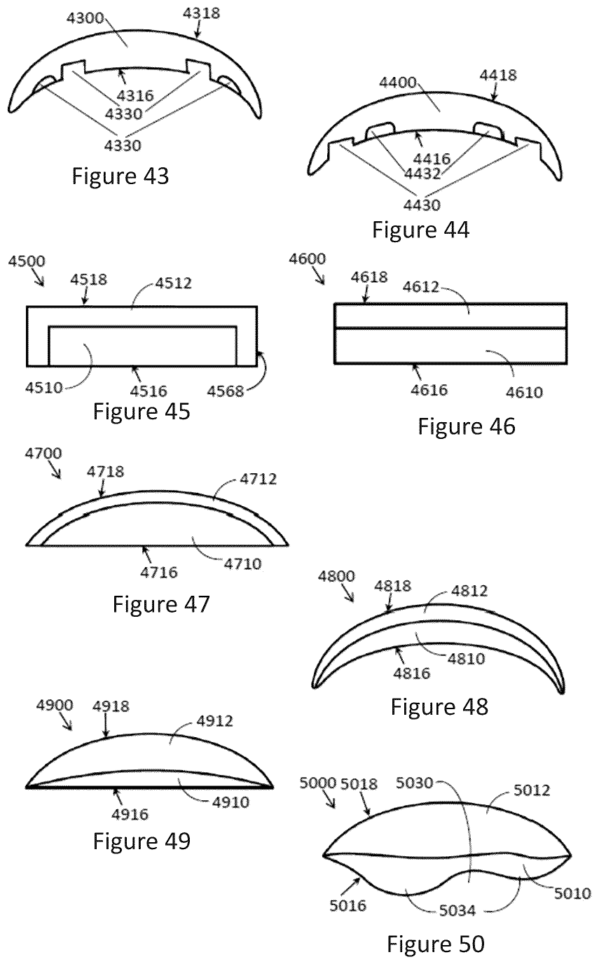

Referring now to FIG. 43 and FIG. 44, in some embodiments, a device 4300, 4400, has both more than one concavity 4330, 4432 and more than one different material portion 4332, 4432 all of which, in some embodiments, are disposed on posterior surfaces 4316, 4416 of device 4300, 4400 respectively. In some embodiments, one or more of the cavities and/or one or more of the different material portions are located within a body of the device and/or on a different device surface, for example, an anterior surface 4318, 4418 of the device.

Referring now to FIG. 43, in some embodiments, cavity/ies 4330 are disposed on posterior surface 4316 more centrally with respect to surface 4316 and/or device body 4300 than different material (e.g. mucoadhesive) portion/s 4332.

Referring now to FIG. 44, in some embodiments, different material (e.g. mucoadhesive) portion/s 4432 are disposed on posterior surface 4416 more centrally with respect to surface 4416 and/or device body 4400 than cavity/ies 4430.

Referring now to FIGS. 45-50 showing simplified schematic cross sectional views of double layer devices 4500, 4600, 4700, 4800, 4900, 5000, according to some embodiments of the invention.

In some embodiments, device 4500, 4600, 4700, 4800, 4900, 5000, has a posterior surface 4516, 4616, 4716, 4816, 4916, 5016 respectively and an anterior surface 4518, 4618, 4718, 4818, 4918, 5018 respectively.

In some embodiments, device 4500, 4600, 4700, 4800, 4900, 5000 includes a first layer 4510, 4610, 4710, 4810, 4910, 5010, and a second layer 4512, 4612, 4712, 4812, 4912, 5012. Where, in some embodiments, first layer has one or more feature as described regarding and/or illustrated regarding first layer 3110 FIGS. 31A-C and/or one or more of first layer 3210, 3210, 3310, 3410, 3510, 3610, 3710, FIGS. 31A-37 respectively. Where, in some embodiments, second layer has one or more feature as described regarding and/or illustrated regarding second layer 3112 FIGS. 31A-C and/or one or more of second layer 3212, 3212, 3312, 3412, 3512, 3612, 3712, FIGS. 31A-37 respectively.

In some embodiments, a device has a posterior surface which is flat or has low curvature (e.g. as defined elsewhere in this document) in dry and/or hydrated form. For example, posterior surface 4516 FIG. 45, posterior surface 4616 FIG. 46, posterior surface 4716 FIG. 47, posterior surface 4916 FIG. 49.

Referring now to FIG. 45 and FIG. 47, in some embodiments, a second layer 4512, 4712 (e.g. containing lubricous material) extends around a body of the device, for example, covering edge/s 4568 of the device and/or forming a part/s (e.g. at edge/s) of a proximal surface of the device 4516, 4716.

Referring now to FIGS. 45-47, in some embodiments, a first and/or second layer has about uniform thickness across an area of a device and/or along one or more cross section of the device. For example, layers 4510, 4512 FIG. 45, layers 4610, 4612, layer 4712 FIG. 47.

Referring now to FIGS. 47-50. In some embodiments, one or more layer has one or more of a maximal thickness at a center and/or central region of the device and a reducing thickness moving towards edge/s of the device from the central region. For example, layer 4710 FIG. 47, layers 4810, 4812 FIG. 48, layers 4910, 4912 FIG. 49, layer 5012 FIG. 50.

FIG. 45, illustrates a device 4500 with flat and/or low curvature posterior 4516 and anterior surfaces 4518, where second layer 4512 extends around edges of device 4500 to form a portion of anterior surface 4518. Where the layers are individually of about uniform thickness.

FIG. 46, illustrates a device with flat and/or low curvature posterior 4616 and anterior surfaces 4618 and where the layers are individually of about uniform thickness.

FIG. 47, illustrates a device 4700 with flat and/or lower curvature posterior surface 4716 and a convex anterior surface 4718 and where second layer 4712 extends around edges of device 4700 to form a portion of anterior surface 4718. In some embodiments, second layer 4712 is about uniform thickness. In some embodiments, first layer 4710 has a maximal thickness at a center and/or central region of the device and reducing thickness moving towards edge/s of device 4700 from the central region.

FIG. 48, illustrates a device 4800 with both a concave posterior surface 4816 and a convex anterior surface 4818. Where, in some embodiments, both first layer 4810 and second layer 4812, have a maximal thickness at a center and/or central region of the device and reducing thickness moving towards edge/s of device 4800 from the central region.

FIG. 49, illustrates a device 4900 where posterior surface 4916 is flat or has low curvature and where anterior surface 4918 is convex. Where, in some embodiments, both first layer 4910 and second layer 4912, have a maximal thickness at a center and/or central region of the device and reducing thickness moving towards edge/s of device 4900 from the central region.

FIG. 50, illustrates a device 5000 where, in some embodiments, posterior surface has a bounding plane which is convex. In some embodiments, posterior has one or more protrusions 5034 and/or one or more cavities 5030. For example, a cross section with two protrusions 5034 defining between them a cavity 5030. In some embodiments, thickness of one or both layers is irregular. In some embodiments, posterior surface 5016 has an irregular shape, a convex shape, or a shape having multiple concavities.

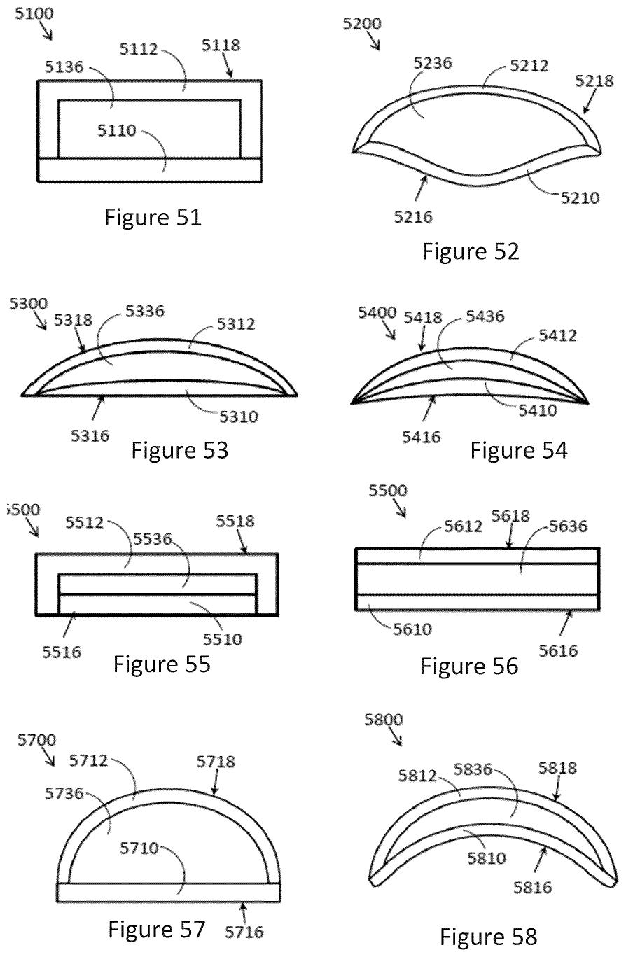

Referring now to FIGS. 51-58 showing simplified schematic cross sectional views of multi-layer devices 5100, 5200, 5300, 5400, 5500, 5600, 5700, 5800 according to some embodiments of the invention.

In some embodiments, device 5100, 5200, 5300, 5400, 5500, 5600, 5700, 5800, has a posterior surface 5116, 5216, 5316, 5416, 5516, 5616, 5716, 5816 respectively and an anterior surface 5118, 5218, 5318, 5418, 5518, 5618, 5718, 5818 respectively.

In some embodiments, device 5100, 5200, 5300, 5400, 5500, 5600, 5700, 5800 includes a first (e.g. mucoadhesive) layer 5110, 5210, 5310, 5410, 5510, 5610, 5710, 5810, and a second (e.g. lubricous) layer 5112, 5212, 5312, 5412, 5512, 5612, 5712, 5812. Where, in some embodiments, the first layer has one or more feature as described regarding and/or illustrated regarding first layer 3110 FIGS. 31A-C and/or one or more of first layer 3210, 3210, 3310, 3410, 3510, 3610, 3710, FIGS. 31A-37 respectively. Where, in some embodiments, the second layer has one or more feature as described regarding and/or illustrated regarding second layer 3112 FIGS. 31A-C and/or one or more of second layer 3212, 3212, 3312, 3412, 3512, 3612, 3712, FIGS. 31A-37 respectively.

In some embodiments, device 5100, 5200, 5300, 5400, 5500, 5600, 5700, 5800 includes an intermediate layer 5136, 5236, 5336, 5436, 5536, 5636, 5736, 5836, respectively. In some embodiments, the intermediate layer is disposed between the first and second layers. Where, in some embodiments, the intermediate layer is covered by other layers e.g. as illustrated in FIGS. 51-55 and FIGS. 57-58. In some embodiments, the intermediate layer includes therapeutic material (e.g. dispersed within the layer and/or in portion/s of the layer).

FIG. 51 illustrates a device 5100 having flat and/or low curvature posterior and anterior surfaces (5116 and 5118, respectively). Where, in some embodiments, second surface extends to cover edges of device 5100 e.g. to enclose sides of intermediate surface 5136. In some embodiments, one or more (e.g. all) of first layer 5110, second layer 5112 and third layer 5136 have about uniform thickness. In some embodiments, a thickness of intermediate layer 5136 is thicker than one or more of the other layers, for example, 1.5-10 times thicker.

FIG. 52 illustrates a device 5200 where a posterior surface 5216 is convex.

In some embodiments, thicknesses of a first 5210 and second layer 5212 are about the same and, in some embodiments about uniform. In some embodiments, an intermediate layer 5236 has a thicker central region, reducing in thickness towards edges of device 5200. In some embodiments, an average and/or maximal thickness of intermediate layer 5236 is at 1.5-10 times thicker than thickness of one or both of the first 5210 and second 5212 layers.

In some embodiments, a convex posterior surface, reduces suction between the eyeball and the device, potentially increasing comfort of the device for a user and/or easing removal of the device. In some embodiments, a device includes shape of anterior 5216 and posterior surfaces 5218 of device 5200 of FIG. 52 but has a different layer structure and/or interior composition as described in the previous paragraph.

FIG. 53 illustrates a device 5300 where each of a first layer 5310, an intermediate layer 5336, and a second layer 5312 have a thicker central region, the layers reducing in thickness towards edges of device 5300. In some embodiments, second layer 5312 is disposed around device 5300 to form part of posterior surface 5316. In some embodiments, posterior surface 5316 has higher radius of curvature than anterior surface 5318 (e.g. radius of curvatures including one or more feature as described elsewhere in this document e.g. in the overview section)

FIG. 54 illustrates a device 5400 where each of a first layer 5410, an intermediate layer 5436, and a second layer 5412 have a thicker central region, the layers reducing in thickness towards edges of device 5400. In some embodiments, thicknesses of the layers are about the same at a central region of the device and/or throughout one or more cross section of the device.

In some embodiments, device 5500 of FIG. 55 has the same features as device 5100 of FIG. 51 except intermediate layer 5536 is, in some embodiments, about the same thickness as one or both of a first layer 5510 and a second layer 5512.

FIG. 56, illustrates a device 5600 where each layer 5610, 5612, 5636, have constant thickness along the illustrated cross section. In some embodiments, intermediate layer 5636 includes therapeutic material. In some embodiments, intermediate layer 5636 is thicker than one or both of the other layers 5612, 5610. For example, a thickness of 1.5-10 times that of the mucoadhesive 5610 and/or lubricious 5612 layers. In some embodiments, intermediate layer 5636 includes a plurality of layers.

FIG. 57 illustrates a device 5700 which has a convex anterior surface 5718, and a flat or low curvature posterior surface 5716. In some embodiments, mucoadhesive layer 5716 has uniform thickness across one or more cross section of the device. In some embodiments, lubricous layer 5712 has uniform thickness on its path around a circumference of intermediate layer 5736. In some embodiments, intermediate layer 5736 is thicker in a central region of one or more cross section of device 5700 and, in some embodiments, has reducing thickness towards edge/s of the device. In some embodiments, lubricous layer 5712 does not form part of posterior surface 5716.

FIG. 58 illustrates a device 5800 which has a convex anterior surface 5818, and a concave posterior surface 5816. In some embodiments, mucoadhesive layer 5816 has uniform thickness across one or more cross section of the device. In some embodiments, lubricous layer 5812 has uniform thickness on its path around a circumference of intermediate layer 5836. In some embodiments, intermediate layer 5836 is thicker in a central region of one or more cross section of device 5800 and, in some embodiments, has reducing thickness towards edge/s of the device. In some embodiments, lubricous layer 5812 does not form part of posterior surface 5816.

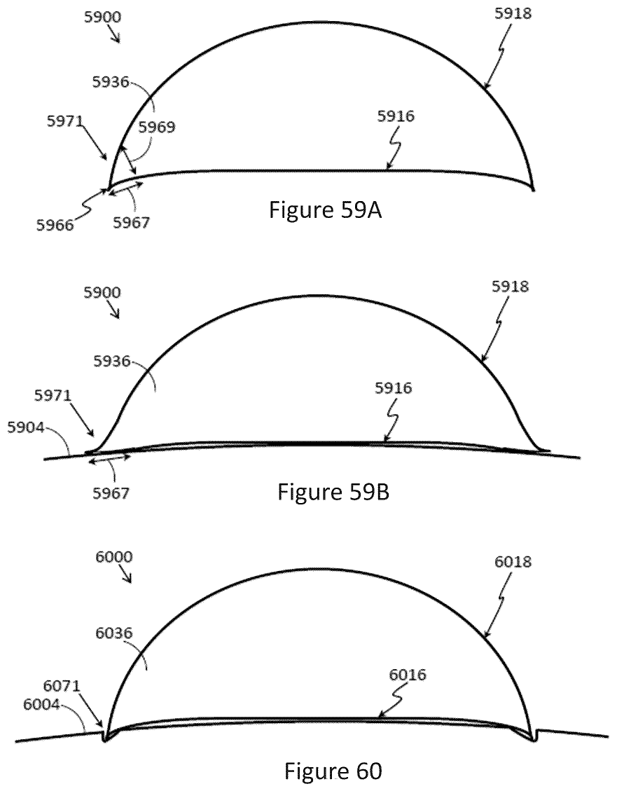

Referring now to FIG. 59A showing a simplified schematic cross section of an ophthalmic device 5900, according to some embodiments of the invention.

In some embodiments, device 5900 includes both a retention portion 5936 (e.g. including one or more feature as described regarding retention portions elsewhere in this document) and a thin edge 5971.

In some embodiments, an edge (e.g. circumferential edge) of the device has average thickness 5969, at distance 5967, as defined as a shortest distance between a posterior surface 5916 and an anterior surface 5918 of the device, of about 15 microns, or about 20 microns, or about 30 microns or about 50 microns. In some embodiments, the ophthalmic device has a thin edge of about 15 microns, or about 20 microns. In some embodiments, the ophthalmic device has an edge thickness of about 5-200 microns, or 5-200 microns.

In some embodiments, distance 5967 is 0.1-1 mm, or 0.1-0.5 mm of the device. In some embodiments, distance 5967 is about 0.1 mm, or about 0.5 mm.

In some embodiments, thin edge 5971 extends for a region of device 5900. For example, a portion of a circumferential edge of the device. For example, for 20-99%, or 80-90%, of a circumference of the device. For example, for a side of a device e.g. for the trailing edge only or the leading edge only. For example, for more than one side of the device e.g. leading edge and trailing edge, e.g. two opposing sides.

FIG. 59A illustrates device 5900 in a relaxed state.

FIG. 59B is a simplified schematic cross section of an ophthalmic device 5900, according to some embodiments of the invention.

FIG. 59B illustrates the device of FIG. 59A when in position of an eye surface (e.g. underneath an eyelid). In some embodiments, thin edge 5971 is flexible and conforms to an eye surface 5904, when device 5900 is on the eye surface.

Referring now to FIG. 60 showing a simplified schematic cross section of an ophthalmic device, according to some embodiments of the invention.

FIG. 60 illustrates the device of FIG. 59A when in position on an eye surface, where the thin edge recesses into the eye surface, for example, as described in the overview section of this document.

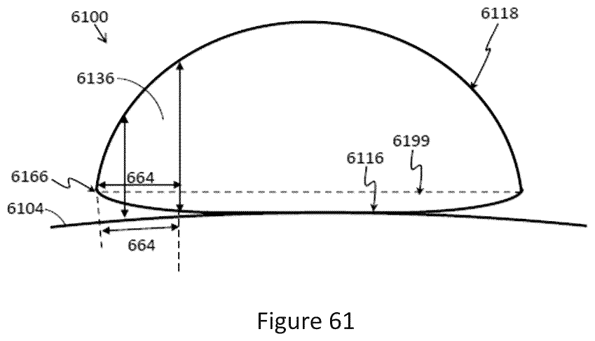

Referring now to FIG. 61 showing a simplified schematic cross section of an ophthalmic device 6100, according to some embodiments of the invention.

In some embodiments, device 6100 has a convex posterior surface 6116. In some embodiments, device 6100 has a retention portion 6136 which includes one or more feature of retention portions as described elsewhere in this document (e.g. retention portion 636 FIG. 6B) where, in some embodiments, heights are measured from an eye surface 6104 e.g. as illustrated by vertical arrows in in FIG. 61.

Alternatively, in some embodiments, heights are measured from a planar surface on which the device rests. Alternatively, in some embodiments, heights are measured from a plane 6199 connecting edges (or a majority of a circumferential edge) of the device or from a plane which delineates a largest area cross section of the device, the plane being located between anterior 6118 and posterior surfaces 6116.

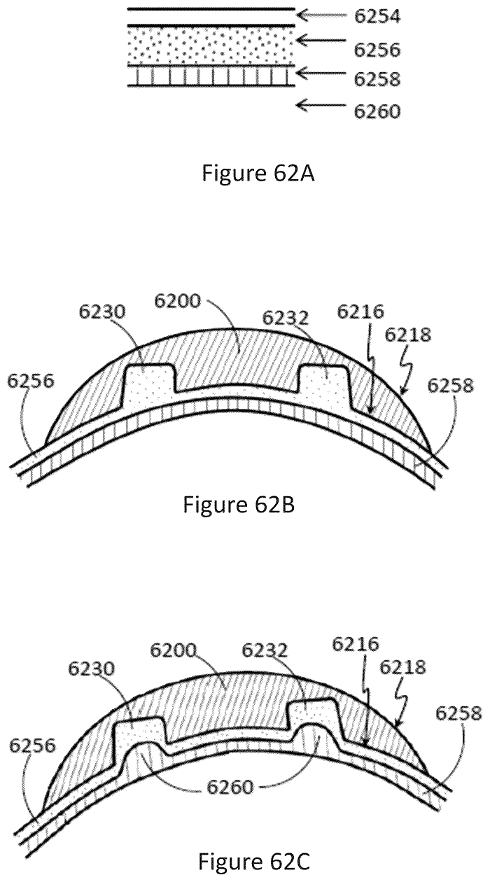

Referring now to FIG. 62A showing a simplified schematic cross sectional view of a portion of an eye surface, according to some embodiments of the invention.

Referring now to FIG. 62B showing a simplified schematic cross section view of an ophthalmic device 6200 on an eye surface, according to some embodiments of the invention.

Referring now to FIG. 62C showing a simplified schematic cross section view of an ophthalmic device 6200 on an eye surface, according to some embodiments of the invention.

In some embodiments, above a stroma 6260 is an eye epithelial layer 6258 over which resides an aqueous tear film layer 6256, above which resides a lipid layer 6254.

FIG. 62A illustrates a cross section of eye anatomy, for example prior to application of device 6200 on the eye.

FIG. 62B illustrates device 6200 and eye anatomy immediately and/or soon after application of device 6200.

FIG. 62C illustrates device 6200 and eye anatomy a time period after application of device where, in some embodiments, the time period is about 1 minute to 1 hour after application.

In some embodiments, as shown for example in FIG. 62B, device body 6200 includes a posterior surface 6216 and an anterior surface 6218. In some embodiments, body 6200 tapers in thickness towards edges of the body.

In some embodiments, device 6200 includes cavities 6230, 6232 which, in some embodiments, are disposed on posterior surface 6216.

In some embodiments, after application of the device tear fluid fills cavities 6232, 6234 epithelial 6258 as before application of the device. In some embodiments, tear fluid e.g. an aqueous and/or a lipid covers the device (not illustrated).

In some embodiments, as shown for example in FIG. 62C, after a time period, in eye tissue conforms to a shape of device 6200. For example, epithelial tissue 6258 locally protrudes 6260 towards cavities 6230/6232 e.g. under suction at the cavity entrances. Potentially epithelial protrusion/s 6260 anchor device 6200 in position e.g. reducing likelihood of movement and/or expelling of the device.

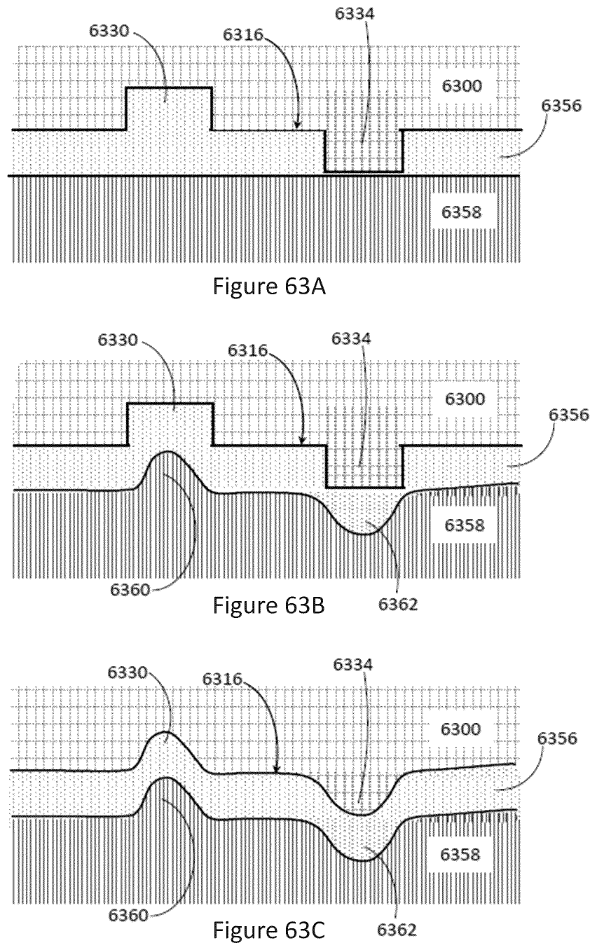

Referring now to FIGS. 63A-C are simplified schematic cross sectional views of a portion of a device 6300 on an eye surface 6358, according to some embodiments of the invention.

FIG. 63A shows device 6300 and eye anatomy 6358 immediately and/or soon after application of device 6300.

FIG. 63B illustrates device 6300 and eye anatomy 6358 a first time period after application of device. Where, in some embodiments, a first time period is 1 minute to 1 hour.

FIG. 63C illustrates device 6300 and eye anatomy 6358 a second time period after the cross section illustrated in FIG. 63B, for example, a further 1 minute to 1 hour after illustration of device 6300 and eye anatomy 6358 in FIG. 63B.

In some embodiments, as shown for example in FIG. 63A, device body 6300 includes a posterior surface 6316 which includes a cavity 6330 and a protrusion 6334.

In some embodiments, after application of the device, tear fluid fills cavity 6330, and epithelial 6358 is as before application of the device.

In some embodiments, as shown for example in 63B, after a first time period, eye tissue conforms to a shape of device 6300. For example, epithelial tissue 6358 locally protrudes to form protrusion 6360 towards cavity 6330 and/or recesses to form a recession 6362 in pliable epithelia tissue at a region adjacent to protrusion 6334. Where recessed material, in come embodiments, potentially anchors device 6300, potentially reducing likelihood of movement and/or expelling of device 6300.

In some embodiments, as shown for example in FIG. 63C, biodegrading and/or bioerosion and/or mechanically erosion of device 6300 has occurred. Where, in some embodiments, protrusion 6334 has reduced in size (e.g. in one or more dimension) and/or corner/s of protrusion 6334 have rounded. Where, in some embodiments, cavity 6330 corners have been rounded. In some embodiments, (not illustrated) the cavity becomes broader in one or more dimension and/or changes depth e.g. becomes shallower or deeper.

In some embodiments, proximal surface 6316 of FIG. 63C illustrates exemplary proximal surface shape, for example, including protrusion/s 6334 and/or cavity/ies 6330.

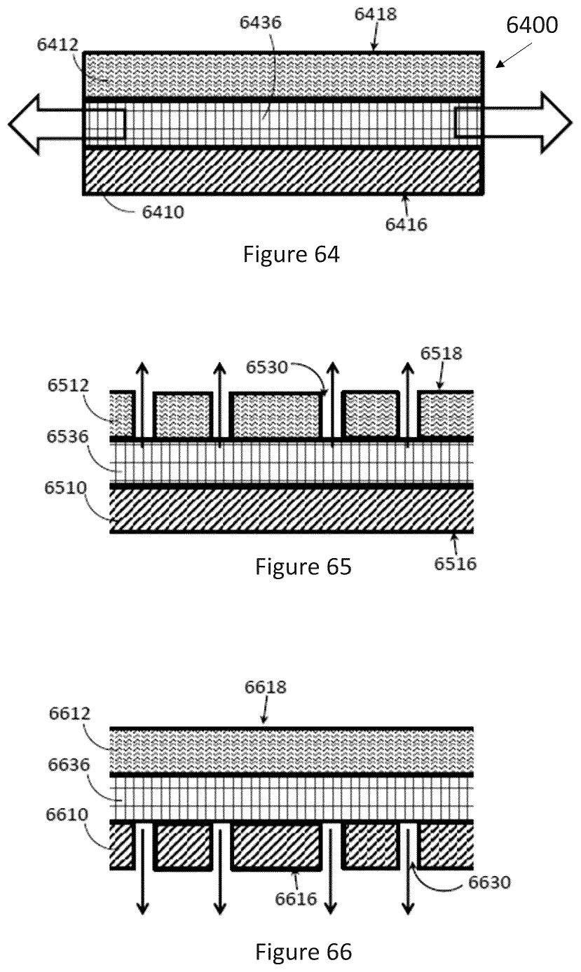

Referring now to FIGS. 64-66 showing simplified schematic cross sectional views of multi-layer devices 6400, 6500, 6600, according to some embodiments of the invention.

FIGS. 64-66, illustrate active pharmaceutical ingredient (API) elution as illustrated in the figures by arrows.

In some embodiments, as shown for example in FIG. 64, an intermediate layer 6436 includes therapeutic material and is not covered by other layer/s (6412, 6410) at edges of device 6400. The layer, in some embodiments, eluting active pharmaceutical ingredient/s (API) through edges e.g. as illustrated by arrows (and optionally not via surfaces 6418, 6419).

In some embodiments, as shown for example in FIG. 65, (e.g. alternatively or additionally to eluting through edges of the device) an intermediate layer 6536 includes therapeutic material and is not fully covered by a second lubricous layer 6512, active pharmaceutical ingredient/s (API) eluting through the second layer. For example, through channel/s and/or holes 6530 in the layer 6512 e.g. as illustrated by arrows on FIG. 65. In some embodiments, alternatively or additionally to having channel/s and/or holes, second layer 6512 includes material which allows diffusion through the material of active pharmaceutical ingredient/s (API). In some embodiments, active pharmaceutical ingredient/s (API) eluting is not performed via surfaces 6518 of layer 6512 or 6516 of 6510.

In some embodiments, as shown for example in FIG. 66, (e.g. alternatively or additionally to eluting through edges and/or through a second lubricious surface 6612 of the device) an intermediate layer 6636 includes therapeutic material and is not fully covered by a first mucoadhesive layer 6610, active pharmaceutical ingredient/s (API) eluting through the first layer. For example, through channel/s and/or holes 6630 in the layer 6610 e.g. as illustrated by arrows on FIG. 66. In some embodiments, alternatively or additionally to having channel/s and/or holes, first layer 6610 includes material which allows diffusion through the material of active pharmaceutical ingredient/s (API).

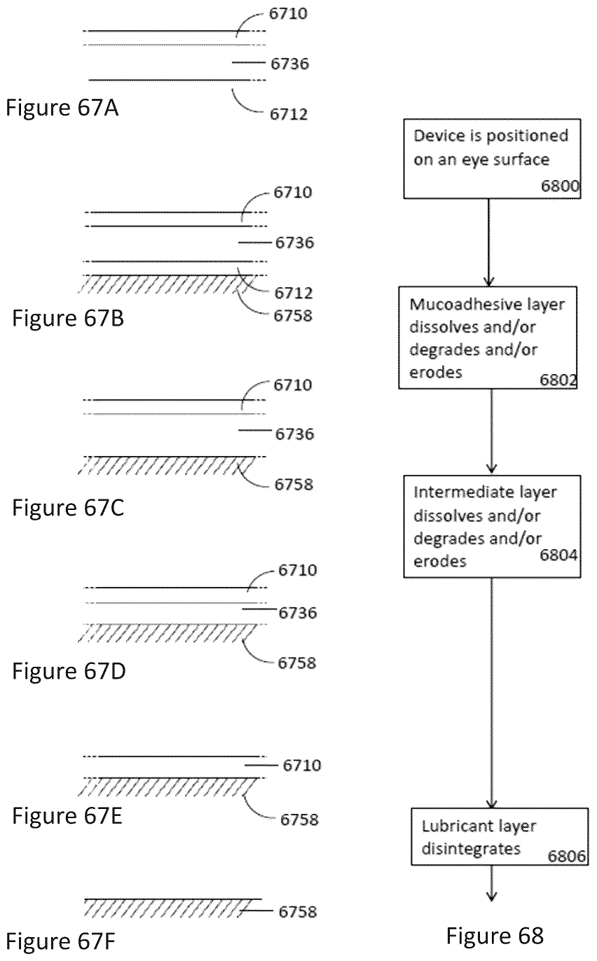

Referring now to FIGS. 67A-F showing simplified schematic cross sections of a portion of a device as residence time of the device progresses, according to some embodiments of the invention.

Referring now to FIG. 68 showing a flowchart of a disintegration progression of a device, according to some embodiments of the invention.

FIG. 67A, illustrates the device layer structure before the device is placed on an eye surface. The device including a first mucoadhesive layer 6710, an intermediate layer 6736 and a second lubricous layer 6712.

At 6800, in some embodiments, device 6700 is positioned on an eye surface 6758.

At 6802, in some embodiments, lubricous layer 6712 degrades and/or dissolves and/or erodes, for example, transitioning device 6700 from the configuration illustrated in FIG. 67B to that illustrated in FIG. 67C.

At 6804, in some embodiments, intermediate layer 6736 partially degrades and/or dissolves and/or erodes, for example, transitioning device 6700 from the configuration illustrated in FIG. 67C to that illustrated in FIG. 67D and then to that illustrated in FIG. 67E.

At 6806, in some embodiments, mucoadhesive layer 6710 dissolves, and/or degrades and/or erodes, for example, transitioning the eye from the configuration illustrated in FIG. 67E to that illustrated in FIG. 67F.

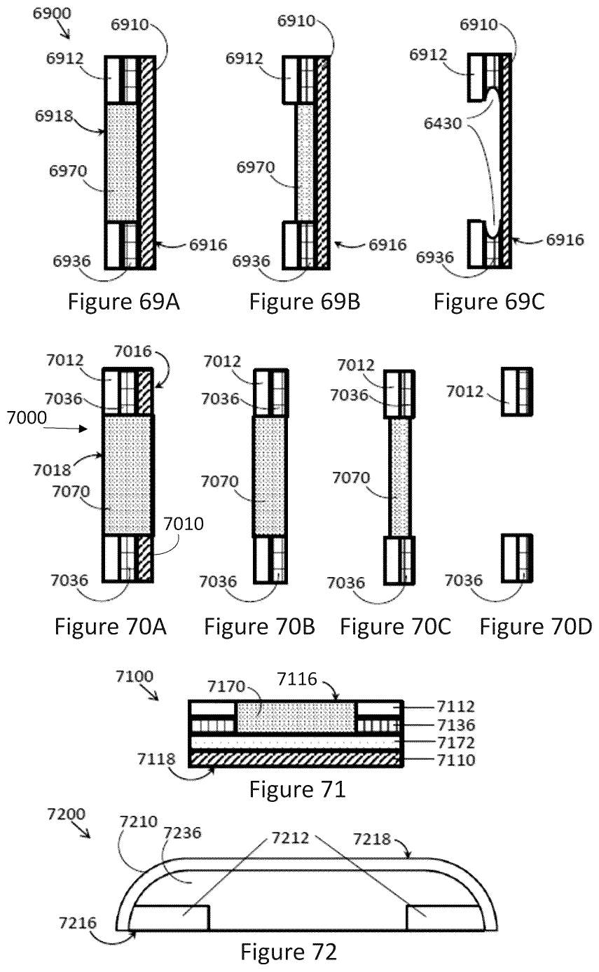

Referring now to FIGS. 69A-C showing simplified schematic cross sections of a device 6900 as residence time of the device progresses, according to some embodiments of the invention.

In some embodiments, FIGS. 69A-C illustrate a portion of a device (e.g. a central portion, where edges are not illustrated).

In FIGS. 69A-C, in some embodiments, like shading indicates portions with the same material characteristics (e.g. lubricious, mucoadhesive, containing therapeutic material).

In some embodiments, device 6900 includes a posterior surface 6916 and an anterior surface 6918, where one or both of surfaces 6916, 6918 have one or more feature (e.g. curvature, thickness) as illustrated and/or described elsewhere in this document.

In some embodiments, device 6900 includes a first mucoadhesive layer 6910, and a second lubricious layer 6912.

Where, in some embodiments, mucoadhesive layer 6910 covers most (e.g. over 55-99%, or 60-90%) or all of posterior surface 6916. A potential benefit of a mucoadhesive layer with a large surface area is increased stability, for example, associated with increased adhesion to the eye surface.

Where, in some embodiments, lubricious layer 6912 covers a portion of anterior surface 6918 e.g. edge region/s.

In some embodiments, device 6900 includes a third layer 6970. Where, in some embodiments, third layer 6970 includes rapidly dissolving and/or degrading and/or abrading material. Where, in some embodiments, the third layer disintegrates more rapidly than other layer/s of the device 6900. For example, as illustrated in transition between FIG. 69A and FIG. 69B where third layer 6970 has reduced in thickness.

In some embodiments, mucoadhesive layer 6910 is also more readily eroded (e.g. associated with proximity to the eye surface which is not illustrated and/or associated with material characteristics of the layer) and/or dissolved and/or degraded (e.g. associated with material characteristics) than one or more other layer of the device. For example, as illustrated in transition between FIG. 69A and FIG. 69B where mucoadhesive layer 6910 has reduced in thickness.

In some embodiments, intermediate layer 6936 includes therapeutic material, where upon being revealed by disappearance of third layer 6970, intermediate layer 6936 begins to dissolve and/or degrade and/or erode, e.g. eluting medication. In some embodiments, intermediate layer is more readily dissolved and/or degraded and/or eroded than one other layer e.g. lubricious layer 6912 and/or mucoadhesive layer 6916 e.g. as illustrated by cavities 6930 having formed in intermediate layer 6936.

Referring now to FIGS. 70A-D showing simplified schematic cross sections of a device as residence time of the device progresses, according to some embodiments of the invention.

FIGS. 70A-C illustrate a portion of a device (e.g. a central portion, where edges are not illustrated).

In FIGS. 70A-C, in some embodiments, like shading indicates portions with the same material characteristics (e.g. lubricious, mucoadhesive, containing therapeutic material).

In some embodiments, device 7000 has a posterior surface 7016 and an anterior surface 7018, where one or both of surfaces 7016, 7018 have one or more feature (e.g. curvature, thickness) as illustrated and/or described elsewhere in this document.

In some embodiments, device 7000 includes a first mucoadhesive layer 7010, and a second lubricious layer 7012. In some embodiments, device 7000 includes an intermediate therapeutic material 7036 layer.

Where, in some embodiments, one or more than one of (e.g. all of) lubricious layer 7012, mucoadhesive layer 7016, are disposed at edge region/s of device 7000, for example, not being present in a central region of device 7000.

In some embodiments, device 7000 includes a third layer 7070. Where, in some embodiments, third layer 7070 includes rapidly dissolving and/or degrading and/or abrading material. Where, in some embodiments, the third layer disintegrates more rapidly than other layer/s of the device 7000. For example, as illustrated in progression from FIG. 70A, to FIG. 70B, to FIG. 70C where third layer 7070 reduces in thickness and then in transition between FIG. 70C to FIG. 70D where third layer 7070 disappears. In some embodiments, e.g. illustrated in transition between FIG. 70A to FIG. 70B, mucoadhesive layer 7010 disappears first, e.g. exposing intermediate therapeutic material layer 7036. In some embodiments, third layer 7070 disappears leaving one or more remaining layer/s at edges of device e.g. as illustrated in FIG. 70D. In some embodiments, device 7000 is placed over a region of the eye e.g. covering a cornea, where disappearance of third layer 7070 creates a hole in the device e.g. to position device 7000 on sclera surrounding cornea.

Referring now to FIG. 71 is a simplified schematic cross section of a device 7100, according to some embodiments of the invention.

FIG. 71 illustrates a portion of a device (e.g. a central portion, where edges are not illustrated).

In FIG. 71, like shading indicates portions with the same material characteristics (e.g. lubricious, mucoadhesive, containing therapeutic material).

In some embodiments, device 7100 has a posterior surface 7116 and an anterior surface 7118, where one or both of surfaces 7116, 7118 have one or more feature (e.g. curvature, thickness) as illustrated and/or described elsewhere in this document.

In some embodiments, device 7100 includes a first mucoadhesive layer 7110, and a second lubricious layer 7112. In some embodiments, device 7100 includes an intermediate therapeutic material 7136 layer. In some embodiment, mucoadhesive layer 7110 covers most or all of device posterior surface 7116.

Where, in some embodiments, one or more than one of lubricious layer 7112, and intermediate therapeutic material layer 7136, are disposed at edge region/s of device 7100, for example, not being present in a central region of device 7100.

In some embodiments, device 7100 includes a third layer 7170. Where, in some embodiments, third layer 7170 includes rapidly dissolving and/or degrading and/or abrading material. Where, in some embodiments, the third layer disintegrates more rapidly than other layer/s of the device 7100.

In some embodiments, device 7100 includes a degradable layer 7172, which, in some embodiments, degrades more rapidly than one or more of mucoadhesive layer 7110, lubricious layer 7112 and therapeutic layer 7136. Where, in some embodiments, degradable layer 7172 degrades and/or dissolves and/or erodes less rapidly than third layer 7170.

Where, in some embodiments, third layer 7170 is first to disappear, for example, revealing portion/s of therapeutic layer 7136 to initiate and/or increase therapeutic material elution. Where, in some embodiments, degrading layer 7172 then erodes e.g. to further reveal the therapeutic material layer and/or to disintegrate the device.

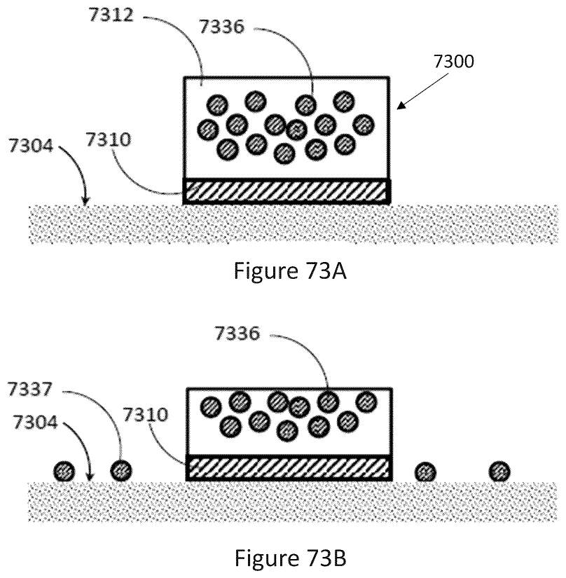

Referring now to FIG. 72 showing a simplified schematic cross section of a device 7200, according to some embodiments of the invention.

In some embodiments, device 7200 has a posterior surface 7216 and an anterior surface 7218, where one or both of surfaces 7216, 7218 have one or more feature (e.g. curvature, thickness) as illustrated and/or described elsewhere in this document.

In some embodiments, anterior surface 7218 includes a low curvature (or flat) region disposed at a central region of device 7200. In some embodiments, curves towards posterior surface 7216.

In some embodiments, device 7200 includes a first mucoadhesive layer 7210, and a second lubricious layer 7212. In some embodiments, device 7200 includes an intermediate therapeutic material 7236 layer. In some embodiments, second surface 7212 curves around edge/s of device 7200, for example, in some embodiments, forming an edge portion of posterior surface 7216.

In some embodiment, mucoadhesive layer 7210 occupies edge regions of device 7200 and/or posterior surface 7216.

A potential advantage mucoadhesive being disposed locally at edges of device 7200 e.g. as recessions in intermediate therapeutic material 7236 layer is adhesive advantages of mucoadhesive material while maintaining more of a volume of the device for therapeutic material. In some embodiments, intermediate therapeutic material layer 7236 forms at least a part of posterior surface 7216. A potential benefit being ingredient elution rapidly after attachment of device 7200 to an eye surface.

Referring now to FIGS. 73A-B showing simplified schematic cross sectional views of a device 7300 on an eye surface 7304, according to some embodiments of the invention.

For simplicity, device 7300 is illustrated as having a rectangular cross section, however device 7300, in some embodiments, has a shape (shape e.g. including curvature of a posterior and/or anterior surface of the device) having one or more feature of device/s described elsewhere within this document.

In some embodiments, device 7300 includes a layer 7312 which includes therapeutic material particles 7336. Where material particles 7336, in some embodiments, include one or more feature of microparticles and/or nanoparticles e.g. as described elsewhere in this document.

In some embodiments, material particles 7336 are devices having retention portions, for example, as described elsewhere in this document. Where, in some embodiments, device 7300 includes 1-10 particles 7336 e.g. each having a retention portion.

Where, in some embodiments, other material of layer 7312 is rapidly dissolvable and/or degradable and/or erodible. Erosion, in some embodiments, revealing and allowing dispersion of particles 7336, for example, as shown in transition between FIG. 73A to FIG. 73B. In some embodiments, e.g. alternatively to what is illustrated in FIG. 73B, particles are dispersed within the eyelid and remain underneath the eyelid, for example, retention portions meaning that eye tissue retains the particles 7336 underneath the eyelid.

In some embodiments, particles 7336 include therapeutic material. Optionally, in some embodiments particles 7336 include mucoadhesive material. Potentially, once a particle is released from layer 7312 if it contacts eye surface 7304 (e.g. under movement/s of one or more of the eyelid, eyeball, and tear fluid) it adheres to the eye surface. In some embodiments, particles adhere to one or more mucosal surface e.g. including the eyeball and/or eyelid. In some embodiments, the particles then degrade (e.g. over about 10 minutes or about 1 hour, or about 12 hours, or about 1 day, or about 3 days) to release therapeutic material to eye tissue.

Optionally, in some embodiments, device includes one or more additional layer (e.g. than layer 7312). For example, in some embodiments, device 7300 includes a mucoadhesive layer 7310.

In some embodiments, therapeutic material layer 7312 including particles is incorporated into one or more of the device embodiments as described within this document. Including, for example, covering and/or access features to therapeutic material layer 7312.

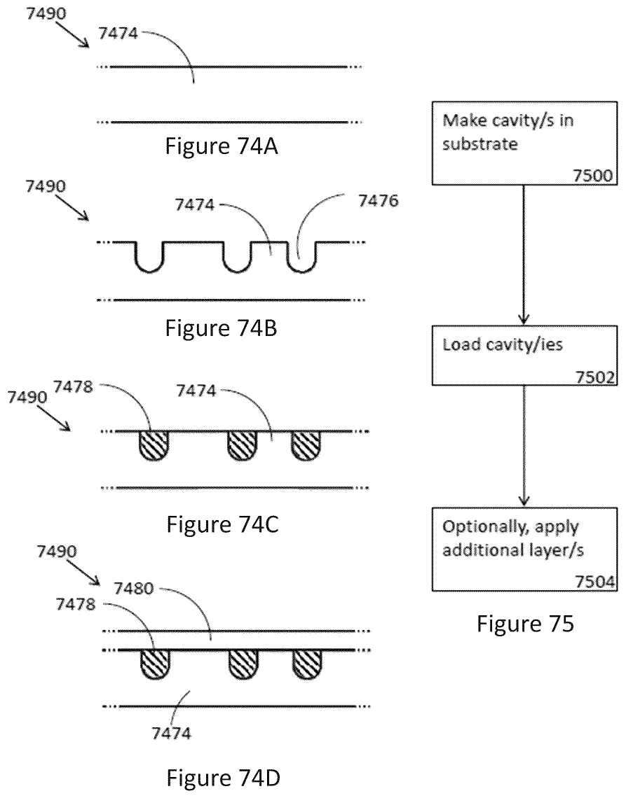

Referring now to FIGS. 74A-D showing simplified schematic cross sectional views of a film, according to some embodiments of the invention.

FIGS. 74A-D illustrate cross sections views of a portion of a device. FIGS. 74A-D show different, portions, for example, sequential portions, of a manufacture process, according to some embodiments of the invention.

Referring now to FIG. 75 showing a method of manufacture, according to some embodiments of the invention.

In some embodiments, as shown for example in FIG. 74A, a substrate 7474 is a film e.g. including one or more feature of films as described elsewhere in this document. For example, in some embodiments, the film includes a single layer. For example, in some embodiments, the film includes a plurality of layers e.g. each layer with different composition and/or material characteristics.

At step 7500, and, for example, referring to FIG. 74B: In some embodiments, cavity/ies are made in substrate 7474. For example, by and/or using one or more of; Jigs, Press, Punch, Laser engraving, drilling, Molecularly imprinted polymer (MIP), 3D printing.

At step 7502, and, for example, referring to FIG. 74C: In some embodiments, cavity/ies are filled e.g. with a different material 7478 to that of substrate 7474.

At step 7504, and, for example, referring to FIG. 74D: In some embodiments, one or more additional layer 7480 is applied on top of the substrate layer 7474. In some embodiments, additional layer 7480 has different characteristics (e.g. different material characteristics) than other portion/s 7474, 7478.

In some embodiments, substrate 7474 includes mucoadhesive material, filling material 7478 includes therapeutic material, and additional layer 7480 includes lubricous material.

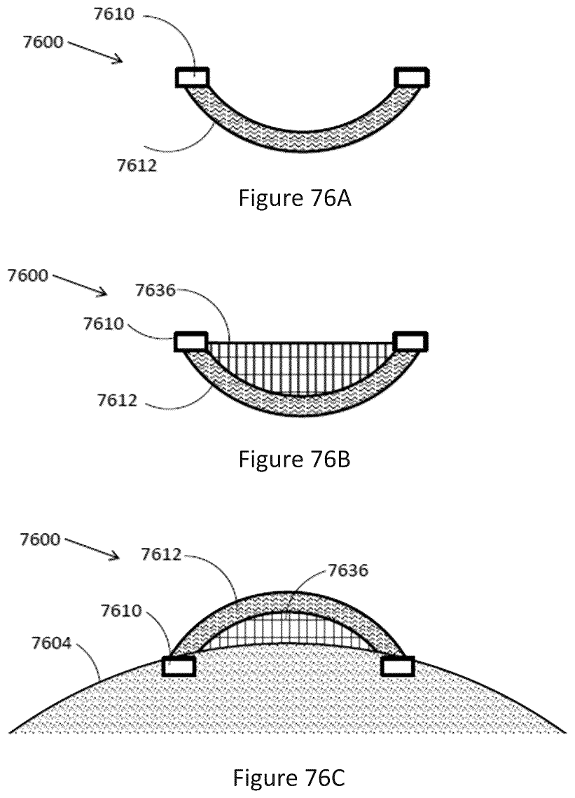

Referring now to FIGS. 76A-C showing simplified schematic cross sectional views of a device 7600, according to some embodiments of the invention.

In some embodiments, device 7600 includes a first layer 7610 and a second layer 7612. In some embodiments, a device posterior surface 7616 is concave. In some embodiments, the concavity is filled with an additional material 7636. For example, during manufacture and/or by a user, for example just prior to use. Where, in some embodiments, additional material 7636 includes active pharmaceutical ingredient/s (API). Where, in some embodiments, additional material 7636 includes mucoadhesive. Where, in some embodiments, device 7600 is used to extend residence time of a topical application in contact with the eye. For example, where device 7600 seals to an eye surface 7604 holding additional material 7637 in contact with the eye surface e.g. for a time period which is, in some embodiments, longer than residence time of eye drops and/or ointment for example. Where, in some embodiments, the time period is 10 minutes to 12 hours, or 10 minutes to 2 hours.

In some embodiments, device 7600 includes lubricous material 7612 and/or mucoadhesive material 7610.

### 2.5. Exemplary Methods of Treatment

In some embodiments, an exemplary method of treating an eye, comprises inserting an ophthalmic device according to any one of the embodiments as disclosed herein, between an eyelid and a surface of said eye; and allowing the release or releasing at least one medication into said eye. In some embodiments, inserting comprises inserting under an inferior eyelid. In some embodiments, inserting comprises inserting under a superior eyelid. In some embodiments, inserting comprises inserting behind a tarsal plate in said eye. In some embodiments, inserting comprises positioning said device on a bulbar conjunctiva and at least partially underneath said eyelid.

### 2.6. Exemplary Materials

In some embodiments, one or more portions of an ophthalmic device includes one or more of; film forming agent/s, plasticizer/s, binder/s, and excipient/s.

In some embodiments the device contains one or more of anti-infective and anti-inflammatory pharmacotherapies (including: Glucocorticoids, NTHEs, and biological agents or combination of them) and other technologies and means, such as: silver, gold, zinc oxide, titanium dioxide, selenium, copper, polyammonium salts, signaling inhibiting and antimicrobial peptides, cytokines, enzymes.

In some embodiments, the device comprises Thermosensitive and/or PH-sensitive and/or Ion sensitive compounds.

Without being bound by theory, the molecular weight of the compounds and/or a degree of cross-linking within materials of the device contribute to device consistency (e.g. hardness and/or rigidity) and/or contribute to the device rheological properties (e.g. viscosity). Where, in some embodiments, molecular weight and/or cross-linking and/or consistency and/or rheological properties define the device residence time and/or active pharmaceutical ingredient (API) release rate.

In some embodiments the device composition comprises at least one charged compound. In some embodiments the device composition comprises at least one charged compound and at least one compound having an opposite charge, constructing a PEC (Poly Electrolyte Complex) formation upon liquid adsorption.

Non-limiting exemplary material/s for:
A non-degradable portion
one or more degradable portion e.g. including
one or more fast degrading portion
one or more slower degrading portion
a lubricious portion (e.g. a lubricious layer and/or film) and/or
a mucoadhesive portion (e.g. a mucoadhesive layer and/or film) and/or
other portion/s of the ophthalmic device include
one or more of (and/or similar compound/s and/or material/s and/or polymer/s and/or combinations thereof) are herein listed:
Polyethylene-Phthalate polymer film; e.g. purchased as film (MYLAR-A®; DuPont, USA).
Polyvinyl Alcohol 4-88; e.g. purchased as powder (Merck, Germany).
Methocel LV-50; e.g. purchased as powder (DOW, USA).
Poly-Vinyl-Alcohol; e.g. purchased as powder (Merck, Germany).
Poly-Vinyl-Alcohol[PVA]:Poly-Ethylene-Glycol[PEG] graft-copolymer, e.g. e.g. purchased as powder (Kollicoat-IR®; BASF, Germany).
Hydroxy-Propyl-Cellulose polymer; e.g. purchased as powder (Klucel-LF®; Ashland, USA) Povidone (Kollidone 25; BASF, Germany).
Carbomer, Carbopol, Carbopol 971, Carbopol 974, Carbopol 934, Carbopol 2020.
Cross-linked Poly-Acrylic-Acid; e.g. purchased as powder (Carbopol 974; Lubrizol, USA).
Cross-linked Sodium Polyacrylic-Acid; e.g. purchased as powder (FavorPac®; Evonik, Germany).
Cross-linked Sodium Polyacrylic-Acid; e.g. purchased as powder (Luquasorb®; BASF, USA).
CMC Sodium, Polycarbophil, Tragacanth, Poly acrylic acid polymers, Sodium alginate, (Poly) Hydroxy Ethyl Cellulose, HPMC, Gum Karaya, Gelatin, Guar Gum, Starch, Modified starch, Pectin, Psyllium, Amberlite-Resin, Hydroxy Propyl cellulose.
Chitosan; e.g. purchased as powder (KiOnutrime-CsG; kitozyme, Belgium).
Chitosan; e.g. purchased as powder (Chitoclear, Premix, Iceland or 90/200/A1; Kraeber, Germany or Protasan UP CL 114, 113, 213, 214; Novamatrix, Norway).
Alginate, Gellan gum, Eudragit.
Polyethylene oxide, Carboxymethyl cellulose (CMC), Poly(methyl vinyl ether), Poly(methyl vinyl ether) co maleic anhydride, Hydroxy Propyl methyl cellulose, Methyl Ethyl cellulose, poly hydroxyethyl methacrylate.
Methylcellulose or hypromellose or Povidone (Kollidone 25; BASF, Germany), or polyvinyl pyrrolidone (PVP).
Cross-linked Poly Glutamic-Acid; e.g. purchased as powder (PGA; hayashibira, Japan).
Cross-linked Dextrane gel; e.g. purchased as powder (Sephadex G-100; GE Medical).
Lactose monohydrate; e.g. purchased as powder (Pharmatose 200, Fonterra, New Zealand).
Cellulose acetate, Ethyl cellulose, Methyl cellulose, Modified cellulose.
Silicone, Acrylates, Polyethylenes (including ultra high molecular weight, Polyethylene terephthalate), Polyester.
Polypropylene, Polytetrafluoroethylene (PTFE, ePTFE), Polyether ether ketone (PEEK), Nylon.
Biocompatible metal alloy.
Polymer foam.
Polymethyl methacrylate (PMMA), polyhydroxyethylmethacrylate (pHEMA).
Cellulose acetate butyrate, Siloxane acrylates, t-Butyl Styrene, Fluoro siloxane acrylates, perfluroethers.
Silicone Hydrogel, Lotrafilcon A, Lotrafilcon B, Galyfilcon A, Senofilcon A, Senofilcon C, Sifilcon A, Comfilcon A, Enfilcon A, Balafilcon A, Delefilcon A, Narafilcon B, Narafilcon A, Stenfilcon A, Somofilcon A, Fanfilcon A, Samfilcon A, Elastofilcon.
Tefilcon, Tetrafilcon A, Crofilcon, Helfilcon A/B, Mafilcon, Polymacon, Hioxifilcon B.
Surfilcon A, Lidofilcon A, Lidofilcon B, Netrafilcon A, Hefilcon B, Alphafilcon A, mafilcon A, Omafilcon B, Vasurfilcon A, Hioxifilcon A, Hioxifilcon D, Nelfilcon A, Hilafilcon A, Hilafilcon B, Acofilcon A, Nesofilcon A.
Bufilcon A, Deltafilcon A, Phemfilcon.
Bufilcon A, Perfilcon A, Etafilcon A, Focofilcon A, Ocufilcon B, Ocufilcon C, Ocufilcon D, Ocufilcon E, Ocufilcon F, Phemfilcon A, Methafilcon A, Methafilcon B, Vilfilcon A.
Lotrafilcon A, Balafilcon A, Senofilcon A, Galyfilcon A, Samfilcon A, Comfilcon A, Enfilcon A and similar.
PHEMA (polyhydroxyethilmethacrylate), MA (methacrylic acid), MMA (methyl methacrylate), GMA (glyceryl methacrylate), DAA (diacetone acrylamide, PVOH (polyvinyl alcohol), PVA (Poly-Vinyl-Acetate).
Silicone rubber, Acrylic resins, Polyurethane, Polypropylene, and Polymethylmethacrylate, Polycarbonate urethane, Polyimides.
Polyglycolide, polylactide, polyhydroxobutyrate, hyaluronic acid, and hydrogels, poly(2-hydroxyethyl-methacrylate).
Polylactic acid (PLA), Polyglycolic acid (PGA), poly(lactic-co-glycolic) acid (PLGA), and poly(caprolactone).
Polyglycolide, poly-L-lactide, poly-D-lactide, poly(amino acids), polydioxanone, polycaprolactone, polygluconate, polylactic acid-polyethylene oxide copolymers, polyorthoesters, polyhydroxybutyrate, polyanhydride, polyphosphoester, poly(alpha-hydroxy acid).
Polycaprolactone (PCL), polyesteramide (PEA).
Protein, Fatty acids, Amino acids, Carbohydrates, collagen.
Poloxamer, Synperonics, Pluronics, Kolliphor, Pluronic F127 (BASF), Synperonic PE/F 127 (Croda), poloxamer 188 (Pluronic® F-68), poloxamer 407 (Pluronic® F-127), polypropylene glycol, polyoxyethylene, poly(propylene oxide), carboxylated polystyrene, PEGylated polystyrene, dendrimers, Poly (amidoamine) (PAMAM).
Poly-Ethylene-Glycol (Sigma, USA).
Triethyl citrate [TEC] and Acetyl tributyl citrate [ATBC]; e.g. purchased as liquids (Merck, Germany).
Tris(hydroxymethyl)aminomethane.
DEP [Diethyl-Phthalate]; e.g. purchased as liquid (Spectrum, USA).
Glycerol Monostearate [GMS]; e.g. purchased as liquid (Cognis, Germany).
Ethanol, Methanol, Isopropyl Alcohol [IPA], Ethyl Acetate, Acetic-acid, and Acetone (BioLab, Israel) are organic solvents used for the polymers dissolving.
Hypromellose Phthalate [HP-55] and Hypromellose Acetate Succinate [AQOAT AS-LF]; e.g. purchased as powders (Shin-Etsu, Korea) CAP [Cellulose-Acetate-Phthalate] and CA [Cellulose-Acetate]; e.g. purchased as powders (Eastman, USA).
Polysaccharides (β-cyclodextrin, dextrans, inulin, etc.), sugars (glucose, lactose, leucrose, maltose, raffinose, sucrose, trehalose, etc.), Polyol (maltitol, mannitol, sorbitol, xylitol, etc.), oligosaccharides, carbohydrates.
Thermosensitive or PH-sensitive or Ion sensitive pressure sensitive deflection sensitive compounds.
Excipients, including: Antiadherents, Binders and Adhesives (including mucoadhesives), Coatings, Colors, Disintegrants, Flavors, Glidants, Lubricants, Preservatives, Sorbents, Sweeteners, Vehicles.
One or more solvent, for example, one or more of ethanol, methanol, isopropanol, methylene chloride, ethyl acetate, acetone, water, ethanol, polyols (such as glycerol, propylene glycol, polyethylene glycol).

### 2.6.1. Exemplary Lubricant Material

In some embodiments, a lubricant portion (e.g. a lubricant layer) includes one or more of; Cellulose Acetate, Ethyl cellulose, PVOH (Poly-Vinyl-Alcohol) Poly-Vinyl-Alcohol[PVOH]:Poly-Ethylene-Glycol[PEG] graft-copolymer or PVA (Poly-Vinyl-Acetate) or PVA (Poly-Vinyl-Acetate) or Methyl cellulose. Exemplary lubricant materials include cellulose derivatives, liquid polyols, polyvinyl alcohol (PVOH), dextran, povidone, gelatin, and hyaluronic acid. Examples of the cellulose derivatives include carboxymethyl cellulose, hydroxyethyl cellulose, hydroxypropyl methyl cellulose (HPMC), and methyl cellulose. Examples of the liquid polyols include glycerol, polyethylene glycol (e.g., PEG 300 and PEG 400), polysorbate, and propylene glycol.

### 2.6.2. Exemplary Mucoadhesive Material

In some embodiments, the term “mucoadhesive” encompasses any compound that is capable of adhering to biological tissue. In some embodiments the mucoadhesive is a polymer. In some embodiments the mucoadhesive, in some embodiments, is charged or neutral.

In some embodiments, a mucoadhesive portion (e.g. a mucoadhesive layer) includes one or more ingredient configured to provide mucoadhesive properties.

In some embodiments, a mucoadhesive portion (e.g. a mucoadhesive layer) includes one or more, of: Poly-Vinyl-Alcohol (also called PVA or PVOH), hydroxypropylcellulose (Klucel), Polyvinyl alcohol-polyethylene glycol graft-copolymer (Kollicoat-IR), methylcellulose, hypromellose methylcellulose, hydroxypropyl methylcellulose (Methocel), Povidone (polyvinylpyrrolidone).

### 2.6.3. Exemplary Active Pharmaceutical Ingredients (API)

In some embodiments, a therapeutic material portion or layer of a device according to embodiments described in this document, contains one or more active pharmaceutical ingredients (API), for example, one or more drug and/or natural compound or synthetic compound.

In some embodiments, one or more therapeutics agents is in one or more of solid form, powder, ointment, liquid, bids (e.g. polymer bids), dispersion, solution, pellets, microparticles, microspheres, and microcapsules.

In some embodiments, therapeutic material contains one or more active pharmaceutical ingredients (API) as microparticles in the 1-1000 μm size range.

In some embodiments, therapeutic material contains active pharmaceutical ingredients (API) and/or excipients and/or combinations thereof.

In some embodiments, an ophthalmic device (e.g. according to one or more embodiment as described in this document) contains a dose of one or more active pharmaceutical ingredient (API) within a range from about 1 μg to about 10,000 μg, or above 10,000 μg, or between 0 and 1 μg, or between 1 μg and 5 μg, or between 5 μg and 10 μg, or between 10 μg and 50 μg, or between 50 μg and 100 μg, or between 100 μg and 500 μg, or between 500 μg and 1000 μg, or between 1000 μg and 5000 μg, or between 5000 μg and 10000 μg, or between 10000 μg and 30000 μg.

In some other embodiments, an ophthalmic device (e.g. according to one or more embodiment as described in this document) contains a mass of active pharmaceutical ingredient (API) of between about 1 microgram to about 20 milligrams.

In some embodiments, the device is about 0.1% to about 99.5%, or about 0.5% to about 90%, drug release material e.g. in combination with pharmaceutically acceptable carrier material/s.

In some embodiments, the device has a concentration of active pharmaceutical ingredient (API) less than 0.01%, or between 0.01% and 0.1%, or between 0.1% and 1%, or between 1% and 5%, or between 5% and 10%, or between 10% and 30%, or between 30% an 60%, or between 60% and 90%, or between 90% and 100%.

In some embodiments, the ophthalmic device (e.g. a therapeutic material layer and/or region) includes one or more of the following active pharmaceutical ingredients (API):

Aflibercept, Atropine, Bevacizumab, Brimonidine/timolol, Ciclosporin, Ciprofloxacin, Cyclopentolate, Diisopropyl fluorophosphate, Diquafosol, Dorzolamide/timolol, chothiophate, Latanoprost/timolol, Latanoprostene bunod, Lifitegrast, Loteprednol, fluocinolone, fluorometholone, difluprednate, prednisolone, triamcinolone, rimexolone, Mydriatics and cycloplegics, Nandrolone sulfate, Naphazoline/pheniramine, Nedocromil, Netarsudil, Ofloxacin, Pegaptanib, Pilocarpine, Pirenoxine, Ranibizumab, Ripasudil, Tauroursodeoxycholic acid, Tavilermide, Tropicamide/hydroxyamfetamine, Verteporfin, Acthar, Bevacizumab, Cefazolin, Ceftazidime, Cocaine, EDTA, Gatifloxacin, Gentamicin, Idoxuridine, Iluvien®, Indocyanine Green, Infliximab, Interferon, Intravitreal Injections, Iohexol, Iopamidol, Iopamidol-M, Lissamine Green, Membrane Blue, Mitomycin, Moxifloxacin, Rituximab, Serum eye drops, Single-Use Povidone, Subconjunctival Injections, Tetracaine PF, Tobramycin, TPA, Vancomycin, Cyclopentolate, Phenylephrine, Proparacaine, Steroids, Tropicamide, Amikacin, Amphotericin-B, Dexamethasone, Mitomycin, Small molecules, Biological drugs, small polypeptides, large polypeptides, proteins, autoimmune drugs, anti-cancer drugs and/or agents, anti-inflammatory agents, anti-infection agents, antibiotics, cannabinoids, Synthetic compounds, Natural compounds, Drug, in some embodiments is hydrophilic or hydrophobic, Drug formulation, in some embodiments is hydrophilic or hydrophobic.

While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

Excipients, including: Antiadherents, Binders, Coatings, Colors, Disintegrants, Flavors, Glidants, Lubricants, Preservatives, Sorbents, Sweeteners, Vehicles

In some embodiments, a therapeutic material includes enhancer materials for example: for penetration enhancement and/or for membrane pore openers.

In some embodiments, a therapeutic material includes materials for improved solubility in human fluids (including serum and/or blood).

In some embodiments, a therapeutic material includes prodrug form of another drug.

In some embodiments, a therapeutic material includes a biosimilar form of another drug.

The therapeutics agent, in some embodiments is at any dosage form including modified release dosage form such as: immediate-release dosage, delayed-release dosage, extended-release dosage, sustained-release dosage, stimuli inducing release and/or targeted-release dosage.

Techniques for modified release dosage, in some embodiments is using any of the following release approaches: Diffusion, Dissolution, Osmotic, Ion-exchange resin, Floating, Bio-adhesive, Matrix, etc.

Processes for modified release form include: microencapsulation, molecularly imprinted polymer (MIP), 3D drug printing

The therapeutics agent, in some embodiments is at any formulation contains any excipient and other compound available.

2.6.4. Exemplary Nanoparticle and/or Microparticle Active Pharmaceutical Ingredient/s (API)

In some embodiments, a therapeutic material portion or layer of a device (e.g. according to one or more embodiment described in this document) contains one or more active pharmaceutical ingredient (API) in the form of nanoparticles and/or microparticles. In some embodiments, nanoparticle and/or microparticle active pharmaceutical ingredient/s (API) are covered and/or contain mucoadhesive compounds and/or polymer/s. Where, in some embodiments, covering of the particles/s increases residence time and/or duration of drug elution of the particle/s. The terms “therapeutic material”, “drug”, “pharmaceutically active agent” and “active pharmaceutical ingredient” are used herein interchangeably.

For example, Atropine microparticles.

For example, atropine sulfate in albumin-chitosan microparticles e.g. including one or more feature as described in “Formulation and characterization of atropine sulfate in albumin-chitosan microparticles for in vivo ocular drug delivery” by Richard T Addo et al, J Pharm Sci. 2015 May; 104(5):1677-90.

Where, in some embodiments, microparticles have a mean size of about 2 microns, atropine concentration of 10% and a release time of about 50 hours. Such particles, in some embodiments are over coated with additional polymer/s with one or more different properties to achieve, in some embodiments, an accumulated extended release pattern over 7 days or longer.

For example, microparticles including one or more feature as described in “Development of Water-Compatible Molecularly Imprinted Polymers Based on Functionalized s-Cyclodextrin for Controlled Release of Atropine” by Yahui He et al, Polymers 2020, 12(1), 130.

For example, Timolol nanoparticles.

For example, particles including one or more feature as described in “Chitosan nanoparticles for prolonged delivery of timolol maleate” by Sunil A Agnihotri 1, Tejraj M Aminabhavi, Drug Dev Ind Pharm. 2007 November; 33(11):1254-62.

In some embodiments, the particles have a mean particle size ranged between 118 and 203 nm, while, in some embodiments, zeta potential ranges between +17 and +22 mV. In some embodiments, entrapment efficiency of the nanoparticles ranges between 47.6 and 63.0%. In some embodiments, e.g. based on results in-vitro release studies performed in phosphate buffer saline of pH 7.4, release of timolol maleate is slow, e.g. by up to 24 hours. The particles, in some embodiments are over coated with additional polymer/s e.g. with different properties to arrive at an accumulated extended release pattern over 7 days or longer.

For example, timolol microparticles including one or more feature as described in “Sustained delivery of timolol maleate from poly(lactic-co-glycolic acid)/poly(lactic acid) microspheres for over 3 months” by James P Bertram 1, Sandeep S Saluja, Jodi McKain, Erin B Lavik, J Microencapsul. 2009 February; 26(1):26.

In some embodiments, microspheres are fabricated using a 50:50 blend of PLGA 502H and PLA. In some embodiments, the microspheres deliver timolol maleate over 50-200 days, or 50-150 days, or 90-120 days, or about 107 days.

### 2.7 Summary of Exemplary Forms/Geometries

An aspect of the disclosed invention includes an ophthalmic device comprising a body having a posterior surface which is adjacent to an eye surface when said ophthalmic device is worn; an anterior surface defining a height of said body above said eye surface; a retention portion, configured to reside between an eyelid and said eye surface, said retention portion bordered by a portion of said anterior surface, and having a height difference of at least 1 mm over at most a 1 mm distance. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

In some embodiments, the body is not hollow. In some embodiments, the retention portion is made of a continuous material. In some embodiments, the body has an oval or round form. In some embodiments, the body is symmetrical in its longitudinal axis and in its transversal axis. In some embodiments, the retention portion has a height of at least 1 mm for an area of at least 1 mm2. In some embodiments, the height difference is in a direction moving away from an edge of said body. In some embodiments, the distance is measured along a surface of said portion of said anterior surface. In some embodiments, the height difference is measured between said retention portion an edge of said body. In some embodiments, the height difference is measured between a first portion of said retention portion and a second portion of said retention portion. In some embodiments, the retention portion allows said device to entirely reside between an eyelid and an eye surface. In some embodiments, the edge of said body is a leading edge of said body disposed adjacent to an opening of an eyelid when said ophthalmic device is worn underneath said eyelid. In some embodiments, the body has a maximal height above said eye surface of 3 mm. In some embodiments, the retention portion has a slope of at least 45° for at least a length of 1 mm along said anterior surface. In some embodiments, the body comprises a first portion and a second portion, where said first portion degrades more rapidly than said second portion when said body is in residence on said eye surface. In some embodiments, the second portion forms part or all of said retention portion. In some embodiments, the body has an average thickness of at least 400 microns. In some embodiments, the body has a maximal thickness of at least 400 microns. In some embodiments, the height difference is of at least 1.5 mm. In some embodiments, when in residence on said eye surface, said anterior surface is adjacent to an inner surface of said eyelid, and said retention portion is held in position by a tarsal plate. In some embodiments, the body comprises therapeutic material and is configured to elute the therapeutic material into said eye when residing on said eye surface. In some embodiments, an extent of said body is 2-8 mm. In some embodiments, the body is elongate. In some embodiments, the body is rotationally symmetrical around an axis connecting said anterior surface and said posterior surface. In some embodiments, the retention portion includes a maximal height portion of said body. In some embodiments, the height of said body reduces moving from said retention portion towards edges of said device. In some embodiments, the posterior surface is concave. In some embodiments, a softness of said device is 0.3-1.5 Mpa. In some embodiments, as mentioned before, the required softness may be achieved with an elastomeric matrix formulated with water, PVOH, and a large amount plasticizers. The amount of the plasticizers may be considered large if it is larger than, or, preferably, at least double (in mass) than the mass amount of the PVOH. In some such embodiments, the mass of the PVOH and the plasticizers together is 70% or more of the total mass of the matrix excluding the water. In some embodiments, the anterior surface is convex. In some embodiments, the device is configured to move with respect to said eye surface, during wear. In some embodiments, the posterior surface has sufficiently low suction forces onto said eye surface to enable said movement. In some embodiments, the posterior surface has low curvature. In some embodiments, the posterior surface is convex. In some embodiments, a radius of curvature of said anterior surface is smaller than a radius of curvature of said posterior surface. In some embodiments, the device is configured to reside on said eye surface for at least 15 minutes. In some embodiments, an edge region of said body, at less than 0.5 mm from an edge of said body, has a thickness of less than 50 microns. In some embodiments, the posterior surface comprises mucoadhesive material. In some embodiments, the anterior surface is smooth and/or comprises lubricious material. In some embodiments, the body comprises material which disintegrates within an eye. In some embodiments, the body comprises one or both of biodegradable and bioerodible material. In some embodiments, the posterior surface comprises one or more cavities. In some embodiments, the posterior surface comprises one or more protrusions. In some embodiments, the device comprises one or more electronic element. In some embodiments, the device is supplied in dry or semi-hydrated form. In some embodiments, the body comprises more than one layer with different material characteristics. In some embodiments, said body comprises one or more of: Hydroxy Propyl Cellulose (HPC); Hydroxy Propyl Methyl Cellulose (HPMC); Carboxymethyl cellulose (CMC); PolyVinyl Alcohol (PVOH); PolyEthylene Glycol (PEG); Cellulose Acetate (CA); Polyvinyl alcohol-polyethylene glycol graft-copolymer (Kollicoat); Poly Acrylic Acid (Carbopol or Carbomer for example); Hyaluronic Acid; Ethyl cellulose; Propylene Glycol; Tri Ethyl Citrate (TEC); Glycerol; Dextran; and combinations thereof. In some embodiments, the device comprises one or more anchor configured to hold said device on said eye surface. In some embodiments, the anchor comprises sharp edges of said body. In some embodiments, a radius of curvature of one or more edge of said body is less than 100 microns. In some embodiments, the retention portion has an anterior surface having a slope of at least 45° for at least a length of 1 mm. In some embodiments, the device comprise a section comprising a length of at least 1 mm where a height between said anterior surface and said posterior surface is constant. In some embodiments, the retention portion allows said device to remain between an eyelid and an eye surface. In some embodiments, the retention portion prevents said device from sliding under an upper eyelid. The invention also discloses an ophthalmic device, comprising a body comprising a posterior surface which is adjacent to an eye surface when said ophthalmic device is worn; an anterior surface which is adjacent to an eyelid when said ophthalmic device is worn; a retention portion defined by heights between said posterior surface and said anterior surface; where a first height of said heights measured from a first point on said posterior surface to a corresponding point on said anterior surface differs by at least 1 mm, from a second height of said heights measured from a second point on said posterior surface to a corresponding point on said anterior surface, said second point being distanced at most 1 mm from said first point.

The invention also discloses an ophthalmic device, comprising a body comprising a posterior surface which is adjacent to an eye surface when said ophthalmic device is worn; an anterior surface which is adjacent to an eyelid when said ophthalmic device is worn; a retention portion defined by heights between said posterior surface and said anterior surface; where at least some of said heights between said posterior surface and said anterior surface differ from each other by at least 1 mm when measured from two points on said posterior surface distanced from each other by at most 1 mm. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.).

The invention also discloses an ophthalmic device comprising a body having a posterior surface; an anterior surface defining a height of said body above a planar surface on which said ophthalmic device is disposed in a relaxed configuration, said posterior surface adjacent to said planar surface; and a retention portion, configured to reside between an eyelid and an eye surface, said retention portion bordered by a portion of said anterior surface, and having a height difference of at least 1 mm over at most a 1 mm distance.

The invention also discloses an ophthalmic device comprising a body comprising a posterior surface which is adjacent to an eye surface when said ophthalmic device is worn; an anterior surface defining a height of said body above said eye surface; a retention portion, configured to reside between an eyelid and said eye surface, said retention portion bordered by a portion of said anterior surface, said portion of said anterior surface having a slope of at least 450 with respect to said eye surface for at least a length of 1 mm along said anterior surface.

The invention also discloses an ophthalmic device comprising a body comprising a posterior surface which is adjacent to an eye surface when said ophthalmic device is worn; an anterior surface defining a height of said body above a planar surface on which said ophthalmic device is disposed in a relaxed configuration, said posterior surface adjacent to said planar surface; and a retention portion, configured to reside between an eyelid and said eye surface, said retention portion bordered by a portion of said anterior surface, said portion of said anterior surface and having a slope of at least 45° with respect to said planar surface for at least a length of 1 mm along said anterior surface.

The invention also discloses an ophthalmic device comprising a body having a first portion and a second portion, where said first portion degrades more rapidly than said second portion when said body is in residence on an eye surface.

The invention also discloses a method of treating an eye, comprising: inserting an ophthalmic device according to the ophthalmic devices as disclosed herein, between an eyelid and a surface of said eye; releasing at least one medication into said eye.

In some embodiments, the inserting comprises inserting under an inferior eyelid. In some embodiments, the inserting comprises inserting under a superior eyelid. In some embodiments, the inserting comprises inserting behind a tarsal plate in said eye. In some embodiments, the inserting comprises positioning said device on a bulbar conjunctiva and at least partially underneath said eyelid.

### 3. Exemplary Elastomeric Matrix Comprising PVOH

While conceiving the present invention, the present inventors envisioned a elastomeric matrix that is free of chemical crosslinker residues and stable during soaking in aqueous medium in terms of shape and consistency. The envisioned elastomeric matrix was both soft and elastic under dry and wet conditions, and substantially did not change shape, after being immersed in or exposed to liquid. While reducing the present invention to practice, the present inventors have surprisingly found that such a matrix can be formed by adding to polyvinyl alcohol a large amount of plasticizers. Particularly, the amount of plasticizer (or plasticizers), optionally organic plasticizer, should be at least twice that of the polyvinyl alcohol. In some embodiments, the devices are stable for a specified period (e.g., three months) in the sense that several parameters remain the same (on average of a sample of devices) before and after the specified period, during which they are kept at predetermined conditions, e.g., 25° C.±2° C. and relative humidity of 60%±5%. For example, the parameters may include: appearance (e.g., lack of tears and bubbles, general shape), pH, and weight. In some embodiments, in case there is an API, the parameters also include that the API remains in the device at the end of the period, its quantity is the same, and impurities are within predetermined limits. The term “the same” means within predetermined allowed minor differences.

### 3.1. Exemplary Elastomeric Matrix:

While optimizing the finding discussed hereinabove, it was surprisingly found by the present inventors that an elastomeric matrix comprising less than 35% by weight (% wt) of polyvinyl alcohol in the composition, maintains its elastomeric matrix form under dry conditions; swells by less than 50% by volume (or less than 15% along each of its three dimensions) under wet conditions and substantially maintains its size, shape and form when moving from dry to wet conditions and back. Manipulating these properties (e.g., decreasing the tendency to swell without compromising pliability), were found to require the use of at least two plasticizers.

Thus, according to an aspect of some embodiments of the present invention, there is provided an elastomeric matrix, which includes poly(vinyl alcohol) (PVOH), at least two plasticizers none of which is water, and water. The matrix exhibits particular desired properties when the combined mass ratio of the plasticizers with respect to PVOH is at least 2:1, and, optionally, the combined mass content of PVOH and the plasticizers is at least 70% wt of the total weight of the matrix, excluding the mass of water, namely the total mass of the PVOH and the plasticizer(s) is at least 70% wt, at least 80% wt or at least 90% wt of the mass of all non-water ingredients of the matrix. In some embodiments, said PVOH comprises PVOH of two or more types that differ from one another in one or both of hydrolysis degree (HD) and chain length. In some embodiments, each of said two or more types of PVOH constitutes at least 10% of the total amount of PVOH in the matrix. In some embodiments, the two or more types of PVOH include one type that has long chains and is fully hydrolyzed, and one type that has at least one of: short chains and is partially hydrolyzed; long chains and is partially hydrolyzed; short chains and is fully hydrolyzed. In some embodiments, short chains are of 200 to 2000 monomeric units; long chains are of 2200 to 5000 monomeric units; fully hydrolyzed PVOH is of hydrolysis degree of 97% or more; and partially hydrolyzed is of hydrolysis degree of 95% or less.

The term “combined mass ratio”, as used herein, refers to the combined amount of a plurality ingredients (e.g., the plasticizers) relative to the amount of another ingredient of the matrix (e.g., the PVOH), wherein all the amounts are provided in terms of mass (e.g., gram). The combined mass ratio is calculated by summing the mass of the plurality of ingredients to obtain a combined mass; and dividing the combined mass by the mass of the other ingredient.

The term “combined mass”, as used herein, refers to the combined amount of a plurality ingredients, provided in terms of mass. The combined mass is calculated by summing the masses of the ingredients making up the plurality.

According to another aspect of some embodiments of the present invention, there is provided an elastomeric matrix for use as an ophthalmic device, which includes poly(vinyl alcohol) (PVOH), a plasticizer which is not water, and water, wherein the mass ratio of the plasticizer to PVOH is at least 2:1, and, optionally, the mass content of all non-water ingredients of the matrix is at least 70% wt of the total weight of the matrix excluding the mass of water. In some embodiments, the elastomeric matrix for use as an ophthalmic device includes at least two plasticizers, none of which is water, and the combined mass ratio of the plasticizers with respect to PVOH is at least 2:1, and the combined mass content of PVOH and the plasticizers is at least 70% wt of the total weight of the matrix, excluding the mass of water, namely the total mass of the PVOH and the plasticizer(s) is at least 70% wt, at least 60% wt or at least 50% wt of the mass of all non-water ingredients of the elastomeric matrix for use as an ophthalmic device.

In some embodiments, the mass ratio of the plasticizer(s) to PVOH is 5.67:1. For example, the mass ration is less than 5:1, less than 10:1, less than 15:1, less than 20:1, or less than 30:1. In some embodiments the mass ratio of the plasticizer(s) to PVOH ranges from 5-2:1 (from 5:1 to 2:1), or 30:1 to 5:1. Optionally, the mass ration is 2.5:1, 3:1, 4:1, 5:1. In some embodiments, said PVOH comprises PVOH of two or more types that differ from one another in one or both of hydrolysis degree (HD) and chain length. In some embodiments, each of said two or more types of PVOH constitutes at least 10% of the total amount of PVOH in the matrix. In some embodiments, the two or more types of PVOH include one type that has long chains and is fully hydrolyzed, and one type that has at least one of: short chains and is partially hydrolyzed; long chains and is partially hydrolyzed; short chains and is fully hydrolyzed. In some embodiments, short chains are of 200 to 2000 monomeric units; long chains are of 2200 to 5000 monomeric units; fully hydrolyzed PVOH is of hydrolysis degree of 97% or more; and partially hydrolyzed is of hydrolysis degree of 95% or less.

According to some embodiments of the present invention, the mass content of PVOH constitutes less than 5% wt of the total weight of non-water ingredients of the matrix. Alternatively, the mass content of PVOH constitutes less than 10% wt, less than 15% wt, less than 20% wt, less than 25% wt, or less than 30% wt of the total weight of non-water ingredients of the matrix.

The term “elastomeric matrix”, as used herein, refers to a crosslinked polymeric structure form that displays rubber-like elasticity and can undergo deformation under the influence of a force and regain its original shape once the force has been removed. In the context of some embodiments of the present invention, the elastomeric matrix is a form of physically crosslinked polymeric structure having an open-cell porous microstructure that can incorporate sequestered and releasable substances within its interconnected voids. According to some embodiments of the present invention, the elastomeric matrix is a network of hydrogen-bonded polymers and plasticizers. According to some embodiments of the present invention, the elastomeric matrix is a network of hydrogen-bonded polymers, plasticizers and water. In some embodiments, the elastomeric matrix provided herein is essentially devoid of covalent crosslinking. Being essentially devoid of covalent crosslinking may be expressed as being water soluble in hot water. The amount and temperature of the water required to dissolve the matrix depends on the size of the matrix and its formulation, including the specific PVOH types that the matrix includes. The solubility of the matrix is about the solubility of the less soluble of those PVOH types.

In some embodiments, the elastomeric matrix provided herein has a porosity of about 50-90%. In some embodiments, the matrix is characterized by a porosity of at least about 50%, 60%, 70%, 80% or at least about 90%.

The consistency of the elastomeric matrix provided herein may resemble that of a hydrogel, however, in distinction from hydrogels, the elastomeric matrix provided herein is stable under dry conditions, and does not have to be immersed in water in order to retain its pliability. The mechanical properties of the elastomeric matrix provided herein resemble that of rubber (elastomeric) under dry and/or wet conditions (e.g., soaked in an aqueous medium).

Unless explicitly stated otherwise, any reference to the elastomeric matrix provided herein is made to the elastomeric matrix under dry conditions. The term “dry conditions” refers to the elastomeric matrix provided herein itself, without referring to a solution that the matrix may or may not be soaked in/with. According to embodiments of the present invention, the elastomeric matrix provided herein is definable and shape-stable regardless of it being or not being soaked-in or wet with a liquid medium. In some embodiments, the devices (matrices) are stable for a specified period (e.g., three months) in the sense that several parameters remain the same (on average of a sample of devices) before and after the specified period, during which they are kept at predetermined conditions, e.g., 25° C.±2° C. and relative humidity of 60%±5%. For example, the parameters may include: appearance (e.g., lack of tears and bubbles, general shape), pH, and weight. In some embodiments, in case there is an API, the parameters also include that the API remains in the device at the end of the period, its quantity is the same, and impurities are within predetermined limits. The term “the same” means within predetermined allowed minor differences.

Water may be structurally fixed in the elastomeric matrix, may be free to evaporate/flow out of the matrix, or may not be present in the matrix. The exact amount of water (fixed and/or free) varies depending on the nature of the ingredients in the matrix and the conditions it is kept in. When referring to the “non-water” elements/ingredients of the elastomeric matrix provided herein, it is meant to refer to all its ingredients excluding water. The mass content of the “non-water” elements is not to be confused with the mass content of the matrix under dry conditions, which may include a mass of water.

When referring to “dry conditions” it should be taken as relating to conditions wherein the elastomeric matrix as provided herein is exposed to air at room temperature in an open or closed container and is not immersed in liquid, such as water or any other aqueous medium or liquid. In the context of ophthalmic devices, “dry conditions” describes a state that is the opposite of how contact lenses are stored; contact lenes are typically made of a hydrogel, which should always be kept under aqueous medium in order to retain their shape and malleability. In sharp contrast, the elastomeric matrix provided herein does not have to be kept wet and does not change its shape (although it may change in size) when going from wet to dry conditions and back.

Hence, when the elastomeric matrix is said to have less than 35% wt polyvinyl alcohol of the total weight of the non-water ingredients of the matrix, it is meant that the mass content of PVOH constitutes less than 35% wt of the total mass content of the matrix excluding any water, structurally fixed in the matrix (if any) or free to flow out of the matrix.

A relatively small amount of water in the matrix allows it to have longer shelf-life, and to be storable under dry conditions (i.e., not soaked in a liquid), whereas excessive amount of water is not required and undesired. Assuming only water evaporates from the wet elastomeric matrix, the amount of water remaining after conventional drying is seen as forming a part of the matrix. In the context of the present invention, conventional drying is drying by exposing the outer surface of the matrix to ambient air under room temperature and ambient humidity. The room temperature may range 15-30° C., and the relative humidity may range 30-75%. Exact conventional drying time may vary with the specific content of the matrix, but is typically between 24 hours and four days.

The amount of water can be determined, among other methods, by the Karl Fischer method, and the direct measurement of loss of mass upon drying, or LOD method. The Karl Fischer method was conducted volumetrically using Karl Fischer instrument model Titrando 852 (Metrohm), with Hydranal composite 5 (Honeywell) as titrant. Methanol was used as the solvent. The LOD test was conducted under a temperature of 85° C., using MX-50 moisture analyzer by A&D Company Ltd, Japan, on samples of 700 mg or larger.

According to these methods, which give similar results for a given sample, in some embodiments of the present invention, the elastomeric matrix includes less than about 50% wt of water therein. In some embodiments, the amount of water is less than about 40% wt, less than about 30% wt, less than about 25% wt, less than about 20% wt, less than about 15% wt, less than about 10% wt, or less than about 5% wt of the total mass of the matrix.

Due to its unique composition, the elastomeric matrix provided herein swells under wet conditions, while preserving its shape, by less than 50% in volume. In some embodiments, the matrix swells by no more than 40% by volume, or less than 35%, or less than 20%, or less than 15%, or less than 10%, or less than 5% by volume under wet conditions.

One way to measure swelling under wet conditions is weighing the device before and after wetting, and compare the results. For example, the device may be weighed, then entered to water, than taken out of the water, wiped to soak water droplets from its surface, and weighed again. Any weight gain (if found) may be attributed to volume gain, under the reasonable assumption that the density of the matrix is not very different from that of water. For example, weight gain of 10% may be interpreted as volume gain of 10%. In some embodiments, the elastomeric matrix comprised in the device is characterized by substantially isotropic swelling when being immersed in simulated tear fluid and at room temperature for 5, 10, or 15 minutes. In some embodiments, the elastomeric matrix swells by less than 50% by volume when immersed in simulated tear fluid for 5, 10, or 15 minutes at room temperature.

It is noted herein that the matrix may be cast, molded, cut or otherwise made with a distinct 3D shape, which essentially does not change when conditions change from dry to wet and vice versa. This property is referred to herein as shape retention. In other words, while the overall volume of a piece of the matrix provided herein may change (swell or shrink) under changing wetting conditions, the overall shape of the piece remains essentially the same, without distortions, compression or deformations. Hence, another characteristic of the elastomeric matrix provided herein is substantially uniform size variability along all directions and orientations under changing wetting conditions. Substantially uniform meaning that the size varies along each direction by the same amount±20%. This characteristic may also be referred to herein as isotropic swelling and shrinking.

In some embodiments, swelling may be measured with a caliber, measuring length, width, and thickness. This may allow estimating if the swelling is isotropic or not. In isotropic swelling, the change in dimension along each direction is the same, within ±20%. For example, swelling may be considered isotropic if it is between 12% and 18% along each of the three dimensions. In some embodiments, the elastomeric matrix is characterized by substantially isotropic swelling when being immersed in simulated tear fluid and at room temperature for 5, 10, or 15 minutes. In some embodiments, the elastomeric matrix swells by less than 50% by volume when immersed in simulated tear fluid for 5, 10, or 15 minutes at room temperature.

The composition and method of preparing the elastomeric matrix provided herein also determine the degree of crystallinity of the matrix. Degree of crystallinity (DoC) is the fraction of the ordered molecules in a polymeric substance, which typically ranges between 10% and 80% for many known polymeric substances. Higher values can be achieved in materials having small molecules, or in polymeric substances prepared and/or stored at temperatures just under the melting point. The degree of crystallinity may be evaluated using any method known in the field, as such. Most methods of evaluating the degree of crystallinity assume a mixture of perfect crystalline and totally disordered areas; the transition areas are expected to amount to several percent. These methods include density measurement, differential scanning calorimetry (DSC), X-ray diffraction (XRD), infrared spectroscopy and nuclear magnetic resonance (NMR).

The elastomeric matrix provided herein does not require cooling to below room temperature and once set and dried, does not require heating to above room temperature.

In some embodiments, the elastomeric matrix is obtainable by mixing the API with water and ophthalmic demulcents to obtain a mother solution; and drying the mother solution at room temperature. In some such embodiments, the elastomeric matrix is obtainable without heating the mother solution. In some such embodiments, the elastomeric matrix is obtainable without heating the API. In some embodiments, the elastomeric matrix is obtainable without heating the mother solution. In some embodiments, the elastomeric matrix is obtainable without heating the API. According to some embodiments, the degree of crystallinity of the matrix is lower than 70%, lower than 60%, lower than 50%, lower than 40%, lower than 30%, or lower than 20%.

The elastomeric matrix provided herein is highly suitable for use as an ophthalmic device in ophthalmic uses, including uses that require the matrix to be transparent and uses that do not pose such a requirement. Thus, the elastomeric matrix provided herein according to some embodiments, is characterized by relatively low transparency. The transparency of a substance refers to the optical distinctness with which an object can be seen when viewed through a film/sheet made from the substance. The transparency of an object made from the elastomeric matrix provided herein can be measured by its total transmittance, which is the ratio of transmitted light to the incident light, while accounting for influencing factors: reflection, absorption and dispersion. For example, subtracting the absorption/dispersion and reflection, the total transmittance of a sample is the incident light (100%) minus absorption/dispersion (X %) and minus reflection (Y %); in other terms, the total transmittance=incident light−(absorption/dispersion+reflection). In some embodiments, non-transparent elastomeric matrices may be preferred, e.g., due to being easier to produce without compromising functionality. For example, non-transparent elastomeric matrices may be obtained with PEG as a plasticizer and/or by producing the matrices at room temperature.

According to some embodiments of the present invention, the elastomeric matrix is characterized by total transmittance (transparency) through optical length of 0.5 a millimeter of less than 80%, less than 75%, less than 70%, less than 65%, less than 60%, less than 55%, less than 50%, less than 40%, or less than 30% total transmittance of light therethrough.

According to some embodiments of the present invention, PVOH used for preparing the elastomeric matrix provided herein is characterized by a degree of hydrolysis (D.H.) of more than 80%, more than 85%, more than 90%, or more than 95%. In some embodiments, the degree of hydrolysis of the PVOH is less than 100%.

The degree of polymerization (D.P.) of a polymer is estimated by dividing the molecular weight of the polymer by the molecular weight of the monomer unit. In some embodiments of the present invention, the PVOH that is used for preparing the elastomeric matrix provided herein is further characterized by a degree of polymerization of that ranges 500-5000. In some embodiments, the PVOH is a mix of different degrees of polymerization, for example, it may contain one PVOH with high molecular weight (e.g., chain length of 3000 monomers or more) and one PVOH of low molecular weight (e.g., of chain length of 1000 monomer or less).

In some embodiments, the elastomeric matrix provided herein is a non-biodegradable matrix. In some embodiments, the elastomeric matrix provided herein is a biodegradable matrix. In some embodiments, the elastomeric matrix is stable in liquid at room temperature more than 1 month. In some embodiments said elastomeric matrix undergoes, at least partially and gradually, dissolution and/or erosion when in contact with liquid (water, bodily fluids). In some embodiments, the elastomeric matrix provided herein is bioerodible, namely the amount of polymer bulk in the matrix decreases, due to either physical, e.g., dissolution, and/or chemical processes occurring when the matrix is in contact with bodily tissues and/or bodily liquids. In some embodiments, said bodily liquid is tear fluid. In some embodiments, the devices (matrices) are stable for a specified period (e.g., three months) in the sense that several parameters remain the same (on average of a sample of devices) before and after the specified period, during which they are kept at predetermined conditions, e.g., 25° C.±2° C. and relative humidity of 60%±5%. For example, the parameters may include: appearance (e.g., lack of tears and bubbles, general shape), pH, and weight. In some embodiments, in case there is an API, the parameters also include that the API remains in the device at the end of the period, its quantity is the same, and impurities are within predetermined limits. The term “the same” means within predetermined allowed minor differences.

In some embodiments, the elastomeric matrix provided herein further comprises at least one inorganic ion and/or salts thereof, and/or at least one organic ion and/or salts thereof.

In some embodiments, the elastomeric matrix provided herein further comprises at least one buffering agent. In some embodiments, the buffering agent is tromethamine, potassium phosphate, citric acid, and any combinations thereof.

In some embodiments, the elastomeric matrix provided herein further comprises at least one surfactant. In some embodiments, the surfactant is selected from sorbitan esters (Spans), sorbitan tristearate (Tweens), poloxamers, Tritons, Betains and any combinations thereof.

In some embodiments, a matrix of the invention is substantially devoid of chemical (covalent) crosslinks. Being essentially devoid of covalent crosslinking may be expressed as being water soluble in hot water. The amount and temperature of the water required to dissolve the matrix depends on the specific PVOH types that the matrix includes. In some embodiments, the solubility of the matrix is about the solubility of the less soluble those PVOH types.

### 3.2. Exemplary Mechanical Properties:

The elastomeric matrix provided herein can substantially maintain its mechanical properties, such as rheological properties, elongation at break, tensile strength, yield strength, elongation at yield, elastic modulus and so forth, when exposed to wet conditions.

When referring herein to “dry conditions”, it should be understood to relate to conditions wherein a matrix of the invention is exposed to air at room temperature in an open or closed container and is not immersed in liquid, and particularly not in aqueous liquid. When referring to “wet conditions”, it should be understood to relate to conditions wherein a matrix of the invention is in direct contact with liquid (immersed in the liquid or after immersion in the liquid and before the liquid evaporated or wiped away).

In some embodiments, the liquid is a naturally occurring bodily fluid, including but not limited to blood, saliva, tears, excreta, body tissue, tissue fluids), an aqueous solution (e.g., of the at least one plasticizer), a buffer solution, and any combinations thereof. In some embodiments, the liquid is naturally occurring or simulating a naturally occurring fluid, such as artificial tear fluid. In some embodiments, a simulated tear fluid is based on an aqueous solution containing about 0.67% sodium chloride, about 0.2% sodium bicarbonate and about 0.008% calcium chloride.

In some embodiments, the elastomeric matrix provided herein is characterized by an elongation at break of at least 50%, 100%, 200%, 300%, 400%, 500%, 700%, at least 800%, or at least 900% or 1000% under dry conditions. In some embodiments, the elongation at break of the matrix is at least 100% under wet conditions (immersed in liquid or after immersion in liquid).

It was surprisingly found that in some embodiments of the present invention wherein the water content was relatively low, the elongation at break was surprisingly high. Thus, according to some embodiments of the present invention, the elastomeric matrix having a water content of 30% wt or less, 25% wt or less, or 20% wt or less, exhibited relatively high elongation at break of 100% and higher, and even 300% and higher or 500% and higher.

In some embodiments, the elastomeric matrix provided herein is characterized by a tensile strength in the range 0.1 to 1.5 MPa or 0.05-2 MPa under dry conditions. In some embodiments, the elastomeric matrix provided herein is characterized by a tensile strength substantially in the range 0.1-0.4 MPa under wet conditions. In some embodiments, the tensile strength of the matrix is at least 0.1 MPa under wet conditions (immersed in liquid or after immersion in liquid).

In some embodiments, yield strength of the elastomeric matrix provided herein is at least 0.05-0.5 MPa under dry conditions. In some embodiments, yield strength of the elastomeric matrix provided herein is at least 0.01-0.5 MPa under wet conditions.

In some embodiments, the mechanical properties of the elastomeric matrix provided herein are maintained under dry conditions. In some embodiments, said mechanical properties are maintained under dry conditions for at least 1 year.

In some embodiments, the elastic modulus of the elastomeric matrix provided herein is at least 0.01-0.5 MPa, optionally at least 0.05-0.5 MPa, under dry conditions. In some embodiments, the elastic modulus of the elastomeric matrix provided herein is at least 0.01-0.5 MPa, optionally at least 0.05-0.5 MPa under wet conditions. In some embodiments, the elastic modulus of the elastomeric matrix provided herein is at least 0.01 MPa, at least 0.05 MPa, or at least 0.1 MPa or at least 0.2 MPa under dry conditions. In some embodiments, the elastic modulus of the elastomeric matrix provided herein is at least 0.01 MPa, at least 0.05 MPa, or at least 0.1 MPa under wet conditions.

An exemplary elastomeric matrix, according to the fourth column in Table 5 below, was measured to have Young Modulus of 0.41 MPa, tensile strength of 0.51 MPa, and elongation at break of 456%.

### 3.3. Exemplary Plasticizers:

The term “plasticizer”, as used herein, refers to a wide range of substances that together with PVOH bestow the mechanical properties of the elastomeric matrix provided herein. Without being bound by any particular theory, it is assumed that the PVOH and the plasticizer(s) interact and form a hydrogen-bonded network that allows the matrix to exhibit shape stability and elasticity under wet and dry conditions. The hydrogen bonded network may include plasticizer molecules linked to two (or more) PVOH residues.

In some embodiments of the present invention, the plasticizer is an organic materials, i.e., matter in its various forms that contain carbon atoms.

According to embodiments of the present invention, at least one of the plasticizers is characterized by exhibiting at least two hydrogen bond forming functional groups, namely at least two H-bond acceptors, at least two H-bond donors, or at least one H-bond acceptor and at least one H-bond donor.

According to some embodiments of the present invention, a plasticizer exhibits more than two H-bond forming functional groups, or more than 3, more than 4, more than 5, more than 6, more than 7, or more than 8H-bond forming functional groups. In some embodiments, a plasticizer comprises a plurality of H-bond forming functional groups.

According to some embodiments, a plasticizer is characterized by a molar mass of less than 1,000 g/mol, or less than 500 g/mol. In some embodiments wherein the elastomeric matrix includes at least two plasticizers, the matrix may include at least one plasticizer which is an oligomer characterized by a molar mass of more than 1,000 g/mol. In some embodiments, the oligomer is characterized by a molar mass that ranges 1,000-2000 g/mol.

According to some embodiments of the present invention, a plasticizer is characterized by forming non-rigid crosslinked bridges with other elements of the matrix, such as PVOH. In this context, a plasticizer is characterized by exhibiting at least one, or at least two rotatable bonds in its structure, not including the bonds connecting the hydroxyl group to the molecule. In some embodiments, a plasticizer is characterized by exhibiting at least one bond having a variable dihedral angle. A dihedral angle is the angle between half-planes through two sets of three atoms, having two atoms in common. In some embodiments, the dihedral angle is defined between half-planes through two sets of three non-hydrogen atoms, having two non-hydrogen atoms in common. For example, ethylene glycol exhibits one variable dihedral angle which is defined by two sets of three atoms, each having an oxygen and two carbon atoms common to the two sets of non-hydrogen atoms.

In some embodiments, the plasticizers are biocompatible, namely the plasticizers do not elicit a non-tolerable local or systemic effect in a recipient/user. In some embodiments, a plasticizer is an ophthalmic compatible (acceptable) substance, namely it is biocompatible, and particularly does not elicit a non-tolerable ophthalmic effect in the recipient/user.

In the context of the present invention, according to some embodiments the elastomeric matrix comprises one or more plasticizer, and in other embodiments, the elastomeric matrix comprises at least two plasticizers. It is noted that when referring herein to a particular characteristic of a plasticizer, it is meant to be taken as referring to a single plasticizer or to each of the plurality of plasticizers individually.

According to some embodiments of the present invention, when the matrix includes more than one plasticizer, one is characterized by a viscosity that is at least 5-times higher than the viscosity of the other plasticizer. In some embodiments, at least one of the plasticizers is characterized by a viscosity of at least 1000 cp, and the other plasticizer is characterized by a viscosity of less than 200 cp. Alternatively, one plasticizer has a viscosity of 500 cp or above and the other 50 cp or below. For example, in one exemplary matrix, one of the plasticizers is glycerol, exhibiting a viscosity of about 1400 cp, and the other plasticizer is polyethylene glycol, exhibiting a viscosity of about 100 cp. In another example, one of the plasticizers is glycerol, and the other propylene glycol, exhibiting viscosity of about 40 cp. All viscosities are of the liquid plasticizer at 20° C.

In some embodiments, a plasticizer constitutes at least 30% wt, 40% wt, 50% wt, 60% wt, 70% wt, 80% wt, or at least 90% wt of the total weight of the matrix. In some embodiments, the plasticizer constitutes 30-90% wt of the weight of the matrix and any sub-range therebetween.

In some embodiments, the plasticizer is selected from polyols (e.g. ethylene glycol, diethylene glycol (DEG), triethylene glycol (TEG), and tetraethylene glycol), propylene glycols, glycerol, esters of glycerol (e.g. triacetin), polyprotic organic acids, e.g., oxalic acid, maleic acid, citric acid, etc.), polyamines (e.g., spermine, spermidine, diethylenetriamine, triethylenetetramine, tris(2-aminoethyl)amine, a polyethylenimine (PEI; polyaziridine, etc.), Trypan blue, alkyl gluceths, aliphatic polyether glycos (e.g., polyethylene glycol, prolypropylene glycol, polysorbate 80), polyoxyethylenes, ethanolamines, erythritols, tromethamine, urea, saccharides, amino acids (e.g., glycine, aspartate/aspartic acid etc.), polyphenols (e.g. tannic acid) and any combinations thereof.

In some embodiments, the plasticizer is an ophthalmic emollient or demulcent, as described by the FDA in 21 CFR 349.12. In the present document, the terms lubricant and demulcent are used interchangeably. Thus, according to some embodiments of the present invention, the plasticizers are ophthalmic demulcents selected from the group consisting of cellulose derivatives, carboxymethylcellulose sodium, hydroxyethyl cellulose, hypromellose, hydroxypropyl cellulose, methylcellulose, hemicellulose, dextran, gelatin, liquid polyols, glycerin, polyethylene glycol 300, polyethylene glycol 400, polysorbate 80, propylene glycol, povidone, and any combination thereof. While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

In some embodiments, the matrix comprises glycerol as a single plasticizer. In some embodiments, the matrix comprises propylene glycol as a single plasticizer. In some embodiments, the matrix comprises glycerol and propylene glycol as plasticizers. In some embodiments, the matrix comprises glycerol and propylene glycol, each alone or a mixture thereof mixed with PEG, as plasticizers. In some of embodiments, PEG is used as a single plasticizer, and in some of these embodiments, the amount of PEG and the amount of PVOH are substantially the same.

### 3.4. Exemplary Compositions of Elastomeric Matrices:

The invention further provides a composition of an elastomeric matrix immersed in liquid and/or combined with additional one or more elastomeric matrix(es), each of the matrixes being as disclosed herein.

In some embodiments, a composition of the invention comprises at least two elastomeric matrices as disclosed herein, which may be similar or different in composition, constitution and properties. In some embodiments, one formulation may be cast into a mold and let dry to provide an elastomer, and another solution may be cast above said elastomer to provide a bilayer elastomeric composition of matrices. In some embodiments, a plurality of minute elastomers of one kind (or of several kinds) may be mixed with a formulation that when settles provides an elastomer of another kind, so when the formulation settles it provides a “raisin cake” composition of the one or more elastomeric minute samples in a “dough” of another elastomer.

In some embodiments, the composition of the invention comprises a solution comprising at least one plasticizer, with at least one elastomeric matrix being immersed in said solution. In some embodiments, said composition of the invention further comprises a fluid selected from water or water-based solution or solvent, wherein said at least one elastomeric matrix is immersed in said fluid.

In some embodiments, said composition is stored, prior to use thereof, under dry conditions. In some other embodiments, said composition is stored, prior to use thereof, under wet conditions.

### 3.5. Exemplary Composite Elastomeric Matrix:

While the elastomeric matrix provided herein can be prepared to suit the desired properties, some applications require the matrix to exhibit unique properties, which require to add another type of polymer(s) which is not PVOH, yet typically produced from hydrogen-forming monomers. This type of elastomeric matrices is referred to herein as a composite matrix.

In general, composite materials are the products of a connection between two different chemical entities. The connection between the different entities can be through covalent, non-covalent, ionic or other type or bond. The resulting properties sometimes represent a simple or complex weighted average of the individual properties of each ingredient, and sometimes products with very different properties from those of the two original components are obtained.

In the context of the present invention, it was found that by adding additional polymers that are not PVOH, new materials with a myriad of diverse characteristics can be obtained while keeping the basic characteristics of the elastomeric matric provided herein, namely low amount of polymers, softness, and shape retention under varying dry and wet conditions.

The addition of the non-PVOH polymer can be used for example in order to further modify:
stability in dry and wet conditions;
bio-degradability;
rheological properties;
bio-adhesiveness;
compatibility with active materials;
active material release profile; and
imprinting active materials.

In some embodiments, the composite elastomeric matrix is made by blending PVOH and a non-PVOH polymer capable of hydrogen bonding with PVOH. In some embodiments, said non-PVOH polymer is neutral (not charged). In some embodiments, said non-PVOH polymer has anionic or cationic functional groups (charged). In some embodiments, said non-PVOH polymer is a synthetic polymer. In some embodiments, said non-PVOH polymer is an oligosaccharide or polysaccharide. In some embodiments said non-PVOH polymer is an acrylic polymer.

It is clear to those skilled in the art that additional polymers and various types of molecular links (e.g. ionic, complexes etc.) can be used to create composite materials with PVOH, using either covalent or non-covalent interactions in order to connect the two polymers. The additional polymer that is not PVOH can be selected from various polymers, including naturally occurring polymers and macromolecules, synthetic polymers and the likes. For example, without limitation, the polymer of hydrogen bond forming monomers that is not PVOH is a polyacrylic acid, a polyvinyl pyrrolidone, cellulose, chitin, glycogen, starch, gellan, dextran, inulin, pectin, arabinoxylan, and any mixture thereof.

According to some embodiments of the present invention, the mass content of the non-PVOH polymer in the elastomeric matrix provided herein is smaller than the mass content of PVOH. In some embodiments, the mass ratio between PVOH and the non-PVOH polymer ranges from about 100:1 to 2:1. Some exemplary embodiments of composite matrices are provided in the Examples section that follows below.

### 3.6. Exemplary Medical/Ophthalmic Device:

The present invention further includes the aspect of an ophthalmic/ocular device that can be used in treating ophthalmic/ocular pathologies and diseases, wherein the device comprises or consists of the elastomeric matrix provided herein.

In the context of the ophthalmic device, according to some embodiments of the present invention, the matrix comprises or consists of ophthalmically acceptable ingredients, as these are discussed hereinabove.

The elastomeric matrix provided herein is particularly useful for sequestering and eluding active pharmaceutical ingredients (API), since it is unreactive, stable and benign. The ophthalmic device that comprises the elastomeric matrix provided herein, is configured for drug delivery, both for bolus as well as for sustained release regimens. Hence, according to some embodiments, the matrix includes therein or thereon at least one active pharmaceutical ingredient (API). Preferably, the active pharmaceutical ingredient (API) is selected for treating an ophthalmic symptom, disease or disorder.

The ophthalmic device described herein is generally sized and shaped to be positioned on an outer surface of the eye with at least a portion of the ocular device positioned under one or both eyelids in a manner that does not contact or interfere with the cornea. In one aspect, the ophthalmic device provided herein is configured to be positioned on a surface of the eye, at least partially underneath at least one of the upper and/or lower eyelids and outside a cornea of the eye for delivering at least one active pharmaceutical ingredient (API) to an eye for an extended period of time, for example of between 5 minutes and 24 hours. Optionally more than 24 hours.

The elastomeric matrix provided herein is characterized by a porous microstructure, which allows entrapping a liquid therein. The liquid may be, for example, a solution or a colloid comprising an active pharmaceutical ingredient (API), and this liquid can be dispersed in the matrix and/or entrapped in the pores of the matrix. Hence, in some embodiments, the device includes at least one active pharmaceutical ingredient (API) dispersed, sequestered, impregnated within the elastomeric matrix provided herein.

The active pharmaceutical ingredient (API) can include, for example, a small molecule, a macromolecule, a cell or a tissue.

In some embodiments, the active pharmaceutical ingredient (API) is in a liquid form. In some embodiments, the active pharmaceutical ingredient (API) is in solid form. In some embodiments, the active pharmaceutical ingredient (API) is soluble in water, or soluble in an organic solvent, or amphiphilic.

In some embodiments, the active pharmaceutical ingredient (API) is encapsulated, or microencapsulated, or in a microparticle form, or in a nanoparticle form.

In the context of the present invention, the active pharmaceutical ingredient (API) is, without limitation, an analgesic agent, an antacid, an antianxiety agent, an antiarrhythmic, an antibacterial agent, an antibiotic agent, an anticoagulant, a thrombolytic agent, an anticonvulsant, an antidepressant, an antiemetic agent, an antifungal agent, an antihistamine, an antihypertensive, an anti-inflammatory agent, an antineoplastic agent, an antipsychotic agent, an antipyretic agent, an antiviral agent, a barbiturate, a bronchodilator, a beta-blocker, a corticosteroid, a cold cure, a cytotoxic agent, a decongestant, a diuretic agent, a expectorant, a hormone, a hypoglycemic agent, an immunosuppressive agent, a laxative, a muscle relaxant, a sedative, a sex hormone, a sleeping drug, a tranquilizer, a vitamin, and any combinations thereof.

In some embodiments, the active pharmaceutical ingredient (API) is an ophthalmological active pharmaceutical ingredient (API). In some embodiments, the ophthalmological active pharmaceutical ingredient (API) is a lubricant, an anti-angiogenic agent, a mydriatic agent, an anesthetic, an anti-infective agent, an anti-inflammatory agent, an antihistamine, a glaucoma treatment agent, a surgical agent, a diagnostic agent or any combination thereof.

While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

The active pharmaceutical ingredient (API) can include, without limitation, bimatoprost, travoprost, latanoprost, tafluprost, NSAID, steroid, antihistamine, carbonic anhydrase inhibitor (CAI), dorzolamide, cyclosporine, antibiotic, doxycycline, tetracycline, azithromycin, fatty acid, long chain fatty acid, fatty alcohol, cetyl alcohol, stearyl alcohol, non-penetrating steroid, free acid of steroid, lipid, ketorolac, silicone oil, olopatadine, prostaglandin, prostaglandin analog, prostamide, small-molecule integrin antagonist, lifitegrast, loteprednol, and fluoromethalone or a combination thereof.

The active pharmaceutical ingredient (API) can include a prostaglandin analogue. The prostaglandin analogue can include at least one of bimatoprost, latanoprost, travoprost, and tafluprost. The active pharmaceutical ingredient (API) can be for lowering the intraocular pressure of the eye. The active pharmaceutical ingredient (API) can be for treating dry eye. The active pharmaceutical ingredient (API) can include at least one of cyclosporine, steroid, loteprednol, fluoromethalone, non-penetrating steroid, free acid of steroid, nonsteroidal anti-inflammatory, ketorolac, small-molecule integrin antagonist, lifitegrast, doxycycline, azithromycin, lipid, fatty alcohol, cetyl alcohol, stearyl alcohol, fatty acid, long chain fatty acid, oil, or silicone oil. The active pharmaceutical ingredient (API) can include a steroid. The steroid can include at least one of loteprednol or fluoromethalone.

When preparing an ophthalmic device that comprises the elastomeric matrix according to some embodiments of the present invention, a formula is prepared according to the preparation procedure provided below. Briefly, a liquid formula is obtained which can be used to prepare the final device. The active pharmaceutical ingredient (API) can be mixed into said liquid formula which leads to a matrix and a device that includes the active pharmaceutical ingredient (API). Hydrophilic active pharmaceutical ingredients (API) easily dissolve in the formula, whereas hydrophobic active pharmaceutical ingredients (API) may be suspended (e.g., as a colloid) in the formula.

The amount of the active pharmaceutical ingredient (API) in the matrix depends on the active pharmaceutical ingredient (API) and the treatment regimen. The nature of the matrix and the requirement therefrom, allow the matrix include 30% of its non-water constituents to be active pharmaceutical ingredients (API).

In some embodiments, the matrix of the device further comprises at least one ophthalmically acceptable carrier/supplemental/adjuvant/additive/non-pharmaceutical agent.

The drug release profile depends on the active pharmaceutical ingredient (API), and the regimen. A skilled artisan would appreciate the means and technology present available for controlling sustained release of drugs from various devices and under various conditions. For example, encapsulation of active pharmaceutical ingredients (API), addition of materials that modifies the interaction between the agent and the matrix, and the likes.

In some embodiments, the active pharmaceutical ingredient (API) is released from the matrix in the presence of a liquid, as this liquid is defined hereinabove. When referring to “presence of liquid” it should be understood to relate to the condition wherein the matrix of the invention is exposed to liquid, partially or entirely immersed therein.

In some embodiments, the active pharmaceutical ingredient (API) is maintained within the elastomeric matrix under dry conditions. In some embodiments, the active pharmaceutical ingredient (API) is maintained within the matrix during storage. In some embodiments, the active pharmaceutical ingredient (API) is maintained within the matrix under dry conditions for at least 1 year.

### 3.7. Exemplary Method of Treatment:

The elastomeric matrix of the present invention can be used for administration of active pharmaceutical ingredients (API) to a cell or a tissue/membrane. The invention further provides a method of administering at least one active pharmaceutical ingredient (API) to a cell or a tissue/membrane, which is realized by contacting the cell or tissue/membrane with a therapeutically effective amount of the active pharmaceutical ingredient (API) present in the elastomeric matrix as disclosed herein. In some embodiments, the tissue is selected from ocular, orthodontic, muscle, mucosal, dermatological, connective, cardiac and any combinations thereof.

As used herein, the phrase “therapeutically effective amount” describes an amount of an active pharmaceutical ingredient (API) being administered, which will relieve to some extent one or more of the symptoms of the medical condition being treated. In the context of the present embodiments, the phrase “therapeutically effective amount” describes an amount of the active pharmaceutical ingredient (API) being administered and/or re-administered, which will relieve to some extent one or more of the symptoms of the condition being treated by being at a level that is harmful to the target cell(s) or microorganism(s), and cause a disruption to the life-cycle of the target cell(s) or microorganism(s).

In the context of embodiments of the present invention, the therapeutically effective amount may refer to the active pharmaceutical ingredient (API) as a whole or to the amount of one or more active pharmaceutical ingredient (API) releasably sequestered in the matrix. The efficacy of any active pharmaceutical ingredient (API), can be determined by several methodologies known in the art.

According to another aspect of embodiments of the present invention, any one of the matrices described herein is identified for use in treating a subject diagnosed with a medical condition treatable by at least one active pharmaceutical ingredient (API) sequestered and controllably releasable from the matrix.

According to another aspect of embodiments of the present invention, there is provided a use of any of the elastomeric matrix described herein as delivery vehicle for use as a medicament. In some embodiments, the medicament is for treating a subject diagnosed with a medical condition treatable by at least one of the drugs sequestered and controllably releasable from the matrix.

In any of the methods and uses described herein, the matrix can be used for medicinal purposes as a part of a medical device.

### 3.8 Exemplary Method of Preparation:

The present invention further provides a method of preparing the elastomeric matrix as disclosed herein, wherein the method comprises the steps of:
(a) dissolving polyvinyl alcohol in water to form a solution;
(b) optionally heating the solution;
(c) mixing the plasticizer(s) into the solution to form a mixture;
(d) optionally heating the mixture;
(e) adding the mixture into a mold;
(f) allowing the mixture set in the mold,
to thereby form the elastomeric matrix of the invention.

In some embodiments, the solution obtained in (a) has less than 20% wt PVOH.

In some embodiments, the plasticizers make 30% wt or more of the mixture formed in (c).

In some embodiments of the method of preparing the matrix of the invention, the optional heating is achieved at a temperature ranging 60-100° C.

In further embodiments of the method, step (e) is conducted at room temperature, for example, after the mixture is let to cool while mixing. In some embodiments of the method, step (e) is conducted in an open form, e.g., in an open flask or mold. In some embodiments, the elastomeric matrix is poured into the mold. In some embodiments, the elastomeric matrix is pumped into the mold.

In some embodiments of the method, at least one active pharmaceutical ingredient (API) is added to the solution or to the mixture, preferably, when the solution or mixture is at about room temperature, as not to encourage reactions between the active pharmaceutical ingredient (API) and other ingredients in the solution. This also allows using active pharmaceutical ingredients (API) that are heat sensitive. Thus, in some embodiments, the method of preparing the elastomeric matrix does not include heating the API-containing mixture. In some embodiments, the method does not include heating the API.

In some embodiments of the method, the elastomeric matrix that is formed in step (f) is immersed, e.g., for 5 minutes, in a solution comprising at least one plasticizer or water or an aqueous solution or a solvent.

It is expected that during the life of a patent maturing from this application many relevant elastomeric matrices will be developed and the scope of the phrase “elastomeric matrix” is intended to include all such new technologies a priori.

### 3.9 Summary of Exemplary Elastomeric Matrix Comprising PVOH

The present disclosure provides an elastomeric matrix formed from polyvinyl alcohol and plasticizers. In some embodiments, the elastomeric matrix is soft and elastic in its dry form. In some embodiments, the elastomeric matrix may be stored for long periods under dry conditions without losing its softness and plasticity, and thus may enjoy particularly long shelf life. In some embodiments, the elastomeric matrix substantially does not change properties, such as shape, volume, and elasticity, after being immersed or exposed to liquid, and thus may be suitable for applications that require such stability. In some embodiments, the elastomeric matrix can be loaded with additional compounds, such as active pharmaceutical ingredients (API), and can be used as or in an ophthalmic device. In some embodiments, the elastomeric matrix disclosed herein may be used as implants, for topical application, or for insertion into body cavities. In some embodiments, the elastomeric matrix used as an implant may be self-applied or applied by a physician, for example, during chirurgical procedure.

Thus, according to an aspect of some embodiments of the present invention, there is provided an elastomeric matrix that includes: poly(vinyl alcohol) (PVOH), one or more plasticizers none of which is water, and water, where a combined mass ratio of the plasticizers to the PVOH is at least 2:1; and a combined mass content of the PVOH and the plasticizers is at least 70% wt of the total weight of the matrix excluding the water.

According to another aspect of some embodiments of the present invention, there is provided an elastomeric matrix for use as an ophthalmic device, which includes: poly(vinyl alcohol) (PVOH), one or more plasticizers which are not water, and water, wherein: a mass ratio of the plasticizer to the PVOH is at least 2:1; and a combined mass content of the PVOH and the plasticizer is at least 70% wt of the total weight of the matrix excluding the water. In some embodiments, the elastomeric matrix provided herein includes at least two plasticizers, none of which is water. In some embodiments, the mass ratio of the plasticizer to the PVOH is less than 20:1. In some embodiments, the elastomeric matrix provided herein is characterized by substantially isotropic swelling and shrinking. In some embodiments, the elastomeric matrix provided herein is characterized by having substantially the same shape under wet or dry conditions (shape stability; shape invariability; shape retention). In some embodiments, the elastomeric matrix provided herein is characterized by swelling by less than 50% by volume under wet conditions. In some embodiments, the elastomeric matrix is characterized by substantially isotropic swelling when being immersed in simulated tear fluid and at room temperature for 5, 10, or 15 minutes. In some embodiments, the elastomeric matrix swells by less than 50% by volume when immersed in simulated tear fluid for 5, 10, or 15 minutes at room temperature. In some embodiments, the elastomeric matrix provided herein is characterized by a degree of crystallinity lower than 50%. In some embodiments, the elastomeric matrix provided herein is characterized by total light transmittance of less than 80%. In some embodiments, the PVOH is characterized by a degree of hydrolysis of more than 90%. In some embodiments, the PVOH is characterized by a degree of polymerization between 500 and 5000. In some embodiments, the plasticizer or plasticizers include at least two hydrogen bond forming functional groups. In some embodiments, the plasticizer or plasticizers is characterized by a molar mass of less than 1,000 g/mol. In some embodiments in which the matrix includes more than one plasticizers, at least one of the plasticizers is an oligomer characterized by a molar mass of more than 1,000 g/mol. In some embodiments in which the matrix includes more than one plasticizers, at least one of the plasticizers is characterized by a viscosity of at least 1000 cp, and at least one other of the plasticizers is characterized by a viscosity of less than 200 cp. In some embodiments, the plasticizer is independently selected from the group consisting of a polyol, a polyprotic organic acid, a polyamine, an alkyl gluceth, an aliphatic polyalkylene glycol, an ethanolamine, a saccharide, an oligosaccharide, an amino acid, a polyphenol, tromethamine, urea, tannic acid, and any salt thereof and/or combinations thereof. In some embodiments, the polyol is selected from the group consisting of ethylene glycol, diethylene glycol, triethylene glycol, tetraethylene glycol, propylene glycol, triacetin erythritol, a poly-glycol, a poloxamer, and a co-polymer thereof, and glycerol and esters thereof. In some embodiments, the polyprotic organic acid is selected from the group consisting of oxalic acid, maleic acid, citric acid, and any salt thereof. In some embodiments, the polyamine is selected from the group consisting of spermine, spermidine, diethylenetriamine, triethylenetetramine, tris(2-aminoethyl)amine, a polyethylenimine, and any salt thereof. In some embodiments, the aliphatic polyalkylene glycol is selected from the group consisting of a polyethylene glycol, a polypropylene glycol, a poly-glycol, a poloxamer, and a polysorbate. In some embodiments, the aliphatic polyalkylene glycol is polyethylene glycol. In some embodiments, the mass content of the PVOH is substantially equal to a mass content of the aliphatic polyalkylene glycol. In some embodiments, the aliphatic polyalkylene glycol is selected from the group consisting of polyethylene glycol, polypropylene glycol, and any mixture thereof. In some embodiments, the plasticizer is biocompatible. In some embodiments, the plasticizer is ophthalmically acceptable. According to some embodiments, the plasticizer in the elastomeric matric provided herein are any one of glycerol, propylene glycol, sugar alcohols such as mannitol and sorbitol, citrate salt, and EDTA salt. In some embodiments, PEG and polypropylene glycol represent preferred second plasticizers. Other preferred plasticizers include di-carboxylic acids, such as maleic acid, fumaric acid, tannic acid, etc., sugars, such as fructose, maltose, etc., DMSO, amino acids, tri-ethyl citrate, and di-propylene glycol. In some embodiments, the mechanical properties of the elastomeric matric provided herein include Young modulus of 0.05-2 MPa, tensile strength of 0.05-2 MPa, and elongation at break of 50-1000% or 100% to 900%. In some embodiments, the mass content of water constitutes less than 50% wt of the total mass content of the elastomeric matrix of provided herein. In some embodiments, the mass content of PVOH constitutes less than 5% wt of the total mass content of non-water ingredients of the elastomeric matrix of provided herein. In some embodiments, the elastomeric matrix of provided herein is essentially devoid of covalent crosslinking. Being essentially devoid of covalent crosslinking may be expressed as being water soluble in hot water. The amount and temperature of the water required to dissolve the matrix depends on the specific PVOH types that the matrix includes. In some embodiments, the solubility of the matrix is about the solubility of the less soluble those PVOH types.

In some embodiments, the elastomeric matrix of provided herein includes a non-PVOH polymer that is characterized by a plurality of hydrogen bond forming residues. In some embodiments, the mass ratio of the PVOH to the non-PVOH polymer is at least 2:1. In some embodiments, the non-PVOH polymer is selected from the group consisting of a polyacrylic acid, a polyvinyl pyrrolidone, a cellulose, a chitin, a glycogen, a starch, a gellan, a dextran, an inulin, a pectin, an arabinoxylan, and any mixture thereof. In some embodiments, the matrix of provided herein further includes an active pharmaceutical ingredient (API).

According to another aspect of some embodiments of the present invention, there is provided an ophthalmic device that includes the elastomeric matrix of provided herein. In some embodiments, the elastomeric matrix consists of ophthalmically acceptable ingredients. In some embodiments, the elastomeric matrix includes an active pharmaceutical ingredient (API). In some embodiments, the device is configured to be positioned on a surface of an eye. In some embodiments, the surface is at least partially underneath at least one of the upper and lower eyelids and outside a cornea of the eye. In some embodiments, the device is configured for delivering at least one active pharmaceutical ingredient (API) to an eye for an extended period of time, for example, of between 5 minutes and 24 hours. Optionally more than 24 hours.

### 4. Exemplary Elastomeric Matrix Comprising Two or More Types of PVOH

### 4.1. Exemplary Basic Composition of an Exemplary Elastomeric Matrix

In some embodiments, an exemplary elastomeric matrix for use, for example but not limited to, as an ophthalmic device, comprises poly(vinyl alcohol) (PVOH), one or more, two or more, or three or more plasticizers, none of which is water, and water. In some embodiments, each of the one or more, two or more, or three or more plasticizers is an organic plasticizer. In some embodiments, the combined mass ratio (i.e., the ratio between the combined mass of the one or more plasticizers to the combined mass of PVOH of the various types included in the matrix is at least 2:1, for example 2.5:1, 3:1, 4:1 or higher or intermediate ratios. In some embodiments, the mass content of all non-water ingredients of the matrix is at least 70% wt of the total weight of the matrix excluding the mass of water, namely the total mass of the PVOH and the plasticizer(s) is at least 70% wt, at least 80% wt or at least 90% wt of the mass of all non-water ingredients of the matrix.

In some embodiments, the mass ratio of the plasticizer(s) to PVOH is less than 5:1, less than 10:1, less than 15:1, less than 20:1, or less than 30:1. For example 5.6:1. In some embodiments the mass ratio of the plasticizer(s) to PVOH ranges from 10-2:1 (from 10:1 to 2:1), or 30:1 to 3:1.

In some embodiments, the mass content of PVOH (of all types combined) constitutes less than 33% wt of the total weight of non-water ingredients of the matrix. For example, the mass content of PVOH constitutes, in some embodiments, between 3% and 10% wt or less, between 5% and 15% wt, less than 20% wt, less than 25% wt, or less than 30% wt of the total weight of the PVOH and plasticizers together (non-water ingredients of the matrix).

The term “combined mass ratio”, as used herein, refers to the combined amount of one or more ingredients of a first kind (e.g., the one or more plasticizers) relative to the amount of one or more other ingredients of the matrix (e.g., the types of PVOH), wherein all the amounts are provided in terms of mass (e.g., gram). The combined mass ratio is calculated by summing the mass of the one or more ingredients of the first kind to obtain a first combined mass; summing the mass of the one or more ingredients of the second kind to obtain a second combined mass and dividing the first combined mass by the second combined mass.

The term “combined mass”, as used herein, refers to the combined amount of a plurality ingredients, provided in terms of mass. The combined mass is calculated by summing the masses of the ingredients making up the plurality.

The term “elastomeric matrix”, as used herein, refers to a crosslinked polymeric structure form that displays rubber-like elasticity and can undergo deformation under the influence of a force and regain its original shape once the force has been removed.

In some embodiments, an elastomeric matrix comprising less than 33% by weight (% wt) of polyvinyl alcohol has the advantage of maintaining its elastomeric matrix form under dry conditions; swells by less than 50% by volume (or less than 15% along each of its three dimensions) under wet conditions; and substantially maintains its geometry when moving from dry to wet conditions. Manipulating these properties were found to require the use of at least two plasticizers.

In the context of some embodiments of the present invention, the elastomeric matrix is a form of physically crosslinked polymeric structure that can release one or more of its ingredients (for example plasticizers and/or active pharmaceutical ingredients (API)) to water (or any wet environment) with or without change in its mechanical properties, for example, having an open-cell porous microstructure that can incorporate sequestered and/or added releasable substances within its interconnected voids. According to some embodiments of the present invention, the elastomeric matrix is a network of hydrogen-bonded polymers and plasticizers. According to some embodiments of the present invention, the elastomeric matrix is a network of hydrogen-bonded polymers, plasticizers and water. In some embodiments, the elastomeric matrix provided herein is essentially devoid of covalent crosslinking. Being essentially devoid of covalent crosslinking may be expressed as being water soluble in hot water. The amount and temperature of the water required to dissolve the matrix depends on the specific PVOH types that the matrix includes. In some embodiments, the solubility of the matrix is about the solubility of the less soluble those PVOH types.

In some embodiments, the elastomeric matrix provided herein has a porosity of about 50-90%. In some embodiments, the matrix is characterized by a porosity of at least about 50%, 60%, 70%, 80% or at least about 90%.

In some embodiments, the elastomeric matrix is free of chemical crosslinker residues and stable during soaking in aqueous medium in terms of geometry and consistency, at least at the first stages in the aqueous medium, for example during the first 1 to 2 minutes after insertion in the aqueous medium, or for longer times, up to, for example, several hours, depending on the formulation. In some embodiments, the elastomeric matrix is both soft and elastic under dry and wet conditions.

In some embodiments, the consistency of the elastomeric matrix provided herein may resemble that of a hydrogel, however, in distinction from hydrogels, the elastomeric matrix provided herein is stable under dry conditions, and does not have to be immersed in water in order to retain its pliability. In some embodiments, the mechanical properties of the elastomeric matrix provided herein resemble that of rubber (elastomeric) under dry and/or wet conditions (e.g., soaked in an aqueous medium).

Unless explicitly stated otherwise, any reference to the elastomeric matrix provided herein is made to the elastomeric matrix under dry conditions. The term “dry conditions” refers to the elastomeric matrix provided herein itself, without referring to a solution that the matrix may or may not be soaked in/with.

In some embodiments, water is structurally fixed in the elastomeric matrix, is free to evaporate/flow out of the matrix, or is not present in the matrix. In some embodiments, the exact amount of water (fixed and/or free) varies depending on the nature of the ingredients in the matrix and the conditions it is kept in. In some embodiments, when referring to the “non-water” elements/ingredients of the elastomeric matrix provided herein, it is meant to refer to all its ingredients excluding water. In some embodiments, the mass content of the “non-water” elements is not to be confused with the mass content of the matrix under dry conditions, which may include a mass of water.

When referring to “dry conditions” it should be taken as relating to conditions wherein the elastomeric matrix as provided herein is exposed to air at room temperature in an open or closed container and is not immersed in liquid, such as water or any other aqueous medium or liquid.

In some embodiments, in the context of ophthalmic devices, “dry conditions” describes a state that is the opposite of how contact lenses are stored; contact lenses are typically made of a hydrogel, which should always be kept under aqueous medium in order to retain their shape and malleability. In sharp contrast, the elastomeric matrix provided herein does not have to be kept wet to retain shape and malleability; and does not change its shape (although it may change in size) when going from wet to dry conditions and back.

Hence, in some embodiments, when the elastomeric matrix is said to have less than a certain % wt polyvinyl alcohol of the total weight of the non-water ingredients of the matrix, it is meant that the mass content of PVOH constitutes less than the certain % wt of the total mass content of the matrix excluding any water, structurally fixed in the matrix (if any) or free to flow out of the matrix.

In some embodiments, a relatively small amount of water in the matrix allows it to have longer shelf-life, and to be storable under dry conditions (i.e., not soaked in a liquid), whereas excessive amount of water is not required and undesired. In some embodiments, assuming only water evaporates from the wet elastomeric matrix, the amount of water remaining after conventional drying is seen as forming a part of the matrix. In the context of the present invention, conventional drying is drying by exposing the outer surface of the matrix to ambient air under room temperature and ambient humidity. In some embodiments, the room temperature may range 15-30° C., and the relative humidity may range 30-75%. In some embodiments, the exact conventional drying time may vary with the specific content of the matrix, but is typically between 24 hours and four days. In some embodiments, the drying is accelerated, e.g., by heating to about 60° C., and the drying time shortens accordingly. In some embodiments, the drying is slowed down, e.g., by drying the solution in a closed mold and/or with cooling, e.g., to about 10° C.

In some embodiments, the amount of water can be determined, among other methods, by the Karl Fischer method, and the direct measurement of loss of mass upon drying, or LOD method. The Karl Fischer method was conducted volumetrically using Karl Fischer instrument model Titrando 852 (Metrohm), with Hydranal composite 5 (Honeywell) as titrant. Methanol was used as the solvent. The LOD test was conducted under a temperature of 85° C., using MX-50 moisture analyzer by A&D Company Ltd, Japan, on samples of 700 mg or larger.

According to these methods, which give similar results for a given sample, in some embodiments of the present invention, the elastomeric matrix includes less than about 50% wt of water therein. In some embodiments, the amount of water is less than about 40% wt, less than about 30% wt, less than about 25% wt, less than about 20% wt, less than about 15% wt, less than about 10% wt, or less than about 5% wt of the total mass of the matrix. In some embodiments, the elastomeric matrix includes between about 50% wt of water and about 5% wt of water.

In some embodiments, due to its unique composition, the elastomeric matrix provided herein swells under wet conditions, while preserving its shape, by less than 50% in volume. In some embodiments, the matrix swells by no more than 40% by volume, or less than 35%, or less than 20%, or less than 15%, or less than 10%, or less than 5% by volume under wet conditions.

It is noted herein that, in some embodiments, the matrix may be cast, molded, cut or otherwise made with a distinct 3D shape, which essentially does not change when conditions change from dry to wet and vice versa. This property is referred to herein as shape retention. In other words, while the overall volume of a piece of the matrix provided herein may change (swell or shrink) under changing wetting conditions, the overall shape of the piece remains essentially the same, without distortions, compression or deformations. Hence, in some embodiments, another characteristic of the elastomeric matrix provided herein is substantially uniform size variability along all directions and orientations under changing wetting conditions. Substantially uniform meaning that the size varies along each direction by the same amount±20%. This characteristic may also be referred to herein as isotropic swelling and shrinking. In some embodiments, the elastomeric matrix is characterized by substantially isotropic swelling when being immersed in simulated tear fluid and at room temperature for 5, 10, or 15 minutes. In some embodiments, the elastomeric matrix swells by less than 50% by volume when immersed in simulated tear fluid for 5, 10, or 15 minutes at room temperature.

In some embodiments, the composition and method of preparing the elastomeric matrix provided herein also determine the degree of crystallinity of the matrix. Degree of crystallinity (DoC) is the fraction of the ordered molecules in a polymeric substance, which typically ranges between 10% and 80% for many known polymeric substances. Higher values can be achieved in materials having small molecules, or in polymeric substances prepared and/or stored at temperatures just under the melting point. Most methods of evaluating the degree of crystallinity assume a mixture of perfect crystalline and totally disordered areas; the transition areas are expected to amount to several percent. These methods include density measurement, differential scanning calorimetry (DSC), X-ray diffraction (XRD), infrared spectroscopy and nuclear magnetic resonance (NMR).

In some embodiments, the elastomeric matrix provided herein does not require cooling to below room temperature and once set and dried, does not require heating to above room temperature. In some embodiments, the elastomeric matrix contains a pharmaceutically active ingredient (API), and is obtainable without heating the API or any mixture or solution containing the API. In some embodiments, the degree of crystallinity of the matrix is lower than 70%, lower than 60%, lower than 50%, lower than 40%, lower than 30%, or lower than 20%. In some embodiments, the matrix does not change its shape after freezing to temperature of −18° C. or lower and thawing to room temperature. In some embodiments, a potential advantage is that is potentially allows using the matrix with active pharmaceutical ingredients (API) that require deep freezing.

In some embodiments, the elastomeric matrix provided herein is highly suitable for use as an ophthalmic device in ophthalmic uses, including uses that require the matrix to be transparent and uses that do not pose such a requirement. Thus, the elastomeric matrix provided herein according to some embodiments, is characterized by relatively low transparency. The transparency of a substance refers to the optical distinctness with which an object can be seen when viewed through a film/sheet made from the substance. The transparency of an object made from the elastomeric matrix provided herein can be measured by its total transmittance, which is the ratio of transmitted light to the incident light, while accounting for influencing factors: reflection, absorption and dispersion. For example, subtracting the absorption/dispersion and reflection, the total transmittance of a sample is the incident light (100%) minus absorption/dispersion (X %) and minus reflection (Y %); in other terms, the total transmittance=incident light−(absorption/dispersion+reflection). In some embodiments, non-transparent elastomeric matrices may be preferred, e.g., due to being easier to produce without compromising functionality and, additionally, it facilitates the manipulation by a user since it potentially allows the user to visualize better the matrix. For example, non-transparent elastomeric matrices may be obtained with PEG as a plasticizer and/or by producing the matrices at room temperature.

According to some embodiments of the present invention, the elastomeric matrix is characterized by total transmittance (transparency) through optical length of 0.5 millimeter of less than 80%, less than 75%, less than 70%, less than 65%, less than 60%, less than 55%, less than 50%, less than 40%, or less than 30% total transmittance of light therethrough.

In some embodiments, the elastomeric matrix provided herein further comprises at least one inorganic ion and/or salts thereof, and/or at least one organic ion and/or salts thereof.

In some embodiments, the elastomeric matrix provided herein further comprises at least one buffering agent. In some embodiments, the buffering agent is tromethamine, phosphate, citrate, and any combinations thereof.

In some embodiments, the elastomeric matrix provided herein further comprises at least one surfactant. In some embodiments, the surfactant is selected from sorbitan esters (Spans), sorbitan tristearate (Tweens), poloxamers, Tritons, Betains and any combinations thereof.

In some embodiments, a matrix of the invention is substantially devoid of chemical (covalent) crosslinks. Chemical or covalent crosslinks are covalent bonds between monomers of the same polymeric chain, two (or more) polymeric chains, or between a monomer of a polymeric chain and another molecule (e.g., plasticizer). Being essentially devoid of covalent crosslinking may be expressed as being water soluble in hot water. The amount and temperature of the water required to dissolve the matrix depends on the specific PVOH types that the matrix includes. In some embodiments, the solubility of the matrix is about the solubility of the less soluble those PVOH types.

### 4.2 Exemplary Degrees of Hydrolysis (D.H.), Degrees of Polymerization (D.P.), Degradation Time and Combinations Thereof.

The inventors have found a surprising effect when combining two types of PVOH having different degrees of hydrolysis for use in an elastomeric matrix, e.g. for ophthalmic devices.

According to some embodiments of the invention, at least two types of PVOH are used for the preparation of the elastomeric matrix. In some embodiments, the difference between the types of PVOH are one or more of a degree of hydrolysis and a degree of polymerization. In some embodiments, a potential advantage of combining at least two types of PVOH is that it allows to manipulate changes over time in mechanical properties of the matrix (for example, tensile strength and/or elastic module) under wet conditions. Additionally or alternatively, in some embodiments, a potential advantage of combining at least two types of PVOH is that it allows manipulating the time that takes for the elastomeric matrix to degrade in wet conditions.

In some embodiments, a first type of PVOH used for preparing the elastomeric matrix is characterized by a full degree of hydrolysis (D.H.) of from 97% to 100%.

In some embodiments, a second type of PVOH used for preparing the elastomeric matrix is characterized by a partially degree of hydrolysis (D.H.) of below 95%, for example, from 80% to 93%. In some embodiments, the partially degree of hydrolysis of the PVOH is less than 90%.

It should be understood to a person having skills in the art that the abovementioned percentages are average percentages, and that in each type of PVOH a degree of hydrolysis is distributed around and within the abovementioned numbers.

The degree of polymerization (D.P.) of a polymer is estimated by dividing the molecular weight of the polymer by the molecular weight of the monomer unit.

In some embodiments, a first type of PVOH used for preparing the elastomeric matrix is characterized by a degree of polymerization comprising long chains that include chains with more than 2500 units and/or molecular weight of more than 100 kg.

In some embodiments, a second type of PVOH used for preparing the elastomeric matrix is characterized by a degree of polymerization comprising shorter chains, that include, e.g., chains with less than 1500 units or less or 1000 units or less.

It should be understood to a person having skills in the art that the abovementioned percentages are average percentages, and that in each type of PVOH a degree of polymerization is distributed around and within the abovementioned numbers.

In some embodiments, molecular weight scales with chain length, but the scaling depends partially on the degree of hydrolysis, because unless the degree of hydrolysis is 100%, not all the repeating units are the same, and the molecular weight of a hydrolyzed unit is different from that of a non-hydrolyzed one. For example, for fully hydrolyzed PVOH: chain length≅3.8+22.3 MW[kg], while for partially hydrolyzed PVOH: chain length≅3.6+20.6 MW[kg].

In some embodiments of the present invention, the PVOH that is used for preparing the elastomeric matrix provided herein is further characterized by a degree of polymerization of that ranges 500-5000. In some embodiments, the PVOH is a mix of different degrees of polymerization, for example, it may contain one PVOH with high molecular weight (e.g., chain length of 3000 monomers or more) and one PVOH of low molecular weight (e.g., of chain length of 1000 monomer or less).

In some embodiments, the elastomeric matrix provided herein is a biodegradable matrix. In some embodiments, the elastomeric matrix is stable in liquid at room temperature more than 1 month. In some embodiments said elastomeric matrix undergoes, at least partially and gradually, dissolution and/or erosion when in contact with liquid (water, bodily fluids). In some embodiments, the elastomeric matrix provided herein is bioerodible, namely the amount of polymer bulk in the matrix decreases, due to either physical, e.g., dissolution, and/or chemical processes occurring when the matrix is in contact with bodily tissues and/or bodily liquids. In some embodiments, said bodily liquid is tear fluid.

In some embodiments, the quantities of the different types of PVOH in an elastomeric matrix correlate with the time that takes the elastomeric matrix to degrade. For example, when a ratio between fully hydrolyzed PVOH and partially hydrolyzed PVOH increases, so is the time under wet conditions to full dissolution of the matrix.

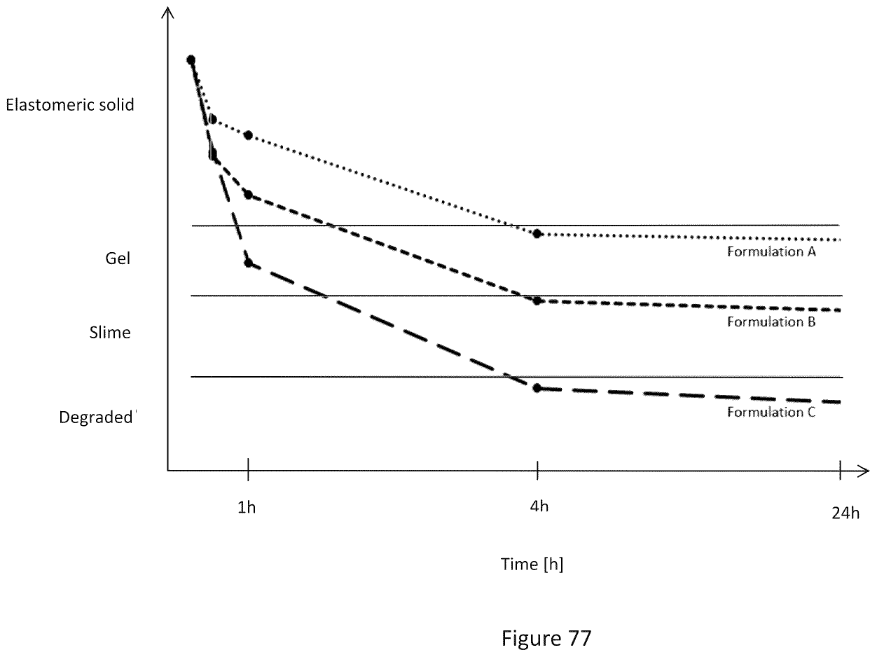

Referring now to FIG. 77, showing a graph illustrating exemplary degradation characteristics of three different elastomeric matrix compositions, as they evolve in time under wet conditions, according to some embodiments of the invention. The graph shows degradation characteristics of three matrices that differ from one another only in the amounts and types of PVOH included in their compositions.

The X axis is a time axis. Matrices with various compositions may behave qualitatively similar to the behavior illustrated in the graph. One difference that may exist between behaviors of different matrices is the time duration, in which they move from one phase to another.

The Y axis is a phase axis. The values listed (elastomeric solid, gel, slime, and degraded) are qualitative descriptions of the matrix. The points are measured values of compressive modulus, presented on a logarithmic scale.

The upper (dotted) line illustrates characteristics of a first elastomeric matrix, in which all the PVOH is fully hydrolyzed and has long chains, the middle line (short dashes) illustrates characteristics of a second elastomeric matrix, in which about half the PVOH is fully hydrolyzed and has long chains, and the rest is partially hydrolyzed, and the lower (long dash) line illustrates characteristics of a third elastomeric matrix, in which about two thirds of the PVOH are partially hydrolyzed and has short chains and the rest of the PVOH is fully hydrolyzed.

The upper line shows that the first matrix stays mainly elastomeric, and may gain some gel-like characteristics after 4 hours in liquid. In some embodiments, these characteristics of gelling continue further, as illustrated by the gentle downward slope of the line towards 24 hours. It should be mentioned that some elastomeric matrices comprising only fully hydrolyzed long chains (not shown) do not show any signs of gelling after 48 hours, a week, or even three months.

The middle (dashed) line shows that the second matrix transitions from an elastomeric state to a gel-like state after about an hour and a half under wet conditions. In the gel-like phase the matrix retains the shape it had in the elastomeric phase, but becomes much more pliable, which may be expressed in lower values of elastic modulus of tension and/or elastic modulus of compression. In some embodiments, the gel-like phase may be characterized in smaller elongation at break and/or smaller tensile force than the matrix in the elastomeric phase.

The second matrix transfers into a slime phase when kept under wet conditions for about four hours. In slime phase, the matrix may have a catarrhal consistency, which may be expressed in complete collapse under application of even a slight compression force and/or breakage under application of even a slight tension force.

The third matrix (lowest line) is shown in the figure to go through the same phases in different times: the transition from elastomeric phase to gel-like phase takes place sooner than in each of the other matrices, and the transition into the slime phase takes place before the similar transition happens with the second matrix. The third matrix, however, also enters a fully degraded phase, in which the matrix does not show any measurable rejection to compression. Thus, FIG. 1 illustrates that in some embodiments, an elastomeric matrix composition having long chain fully hydrolyzed PVOH and short chain and/or partially hydrolyzed PVOH—the higher the quantity of short chain partially hydrolyzed PVOH the faster is the degradation process under wet conditions.

It is noted that the transition between the various phases is not necessarily sharp, and the matrix may be in numerous intermediate phases, for example, from being elastomeric to being gel-like or from being gel-like to being slime. It is also noted that not all matrices go through all the phases. While it is preferable that each elastomeric matrix is in the elastomeric phase at least under dry conditions, not all matrices go through gel-like phase, and not all the matrices go through the slime phase. It is assumed that all the matrices go to complete degradation, although not every matrix was followed in the lab for long enough to confirm that. Such details of what exact phases the matrix go through and at what time periods may depend, for example, on the exact types of PVOH used, as well as on the total amount of PVOH (e.g., as part of the total dry material in the matrix) and other ingredients of the composition and their amounts (e.g., the plasticizers).

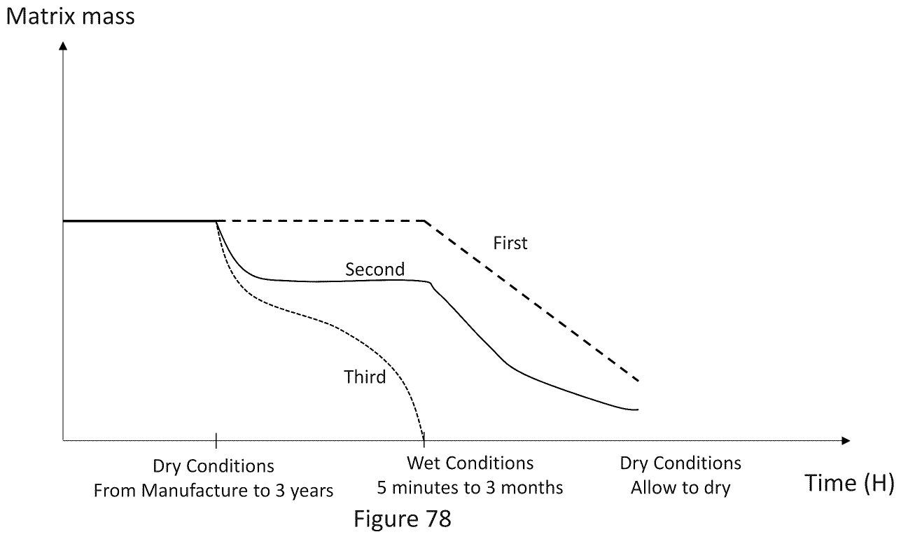

Referring now to FIG. 78 showing a graph illustrating degradation of some matrices during wetting and drying cycle, according to some embodiments of the invention. The degradation is represented in the graph as the change in the mass of the matrix. This change in mass may be accompanied with other changes (e.g., in mechanical properties and/or chemical composition).

The first phase of the cycle, which may be referred to as packaged stage, goes from production of the matrix for as long as it is kept under dry conditions, possibly to the end of its shelf life. In some embodiments, the package stage may last for 3 months, 6 months, a year, two years, or even 3 years or more.

In the packaged stage, each of the matrices, illustrated by the graph, retains its respective mass. The three matrices referred to in the graph all begin with the same initial mass, and remain at the same mass for the entire first phase. Hence, the lines representing them are indistinguishable from one another. In some matrices according to embodiments of the present invention, not illustrated in the graph, some mass loss may occur during the first phase, but in most embodiments such change is not larger than 25%.

In some embodiments, during the first phase, other characteristics of the matrices also do not change, or change only nominally. These characteristics may include, for example, shape, size, chemical composition, and mechanical properties.

The second phase of the cycle, which may be referred to as a wetting stage, goes on for as long as the matrix is under wet conditions. This may last, for example, from about 5 minutes to an hour, a day, a week, a month, or even three months. In case the matrix is used for medical application, the behavior at the second phase may represent the behavior of the matrix when in contact with body fluid.

In the wetting stage, the three matrices behave differently.

In a matrix of the first kind, the mass changes of which are marked by a dashed line, the mass does not change much during the wetting stage. However, the chemical composition of the matrix may change substantially. For example, the organic elastomers making part of the matrix may be replaced by the liquid by which the matrix is wet (e.g., water or body fluids).

A matrix of the first kind tends to keep its shape and size after wetting, but their mechanical properties may change, for example, they may become softer and more pliable than before wetting.

In some embodiments, the PVOH of a matrix of the first kind is mainly long chained and fully hydrolyzed (although it may include two or more types of fully hydrolyzed long chain PVOH). In some embodiments, some of the PVOH in a matrix of the first kind may have shorter length and/or lower degree of hydrolysis, but these should preferably be in small amounts, e.g., 10% or less of the total PVOH content.

Still in the wetting stage, the degradation behavior of a matrix of a second kind is shown in the figure by a solid line. A matrix of the second kind loses a substantial portion of its weight during the wetting stage, but maintains most of its weight, no matter how long is the wetting stage. The chemical composition of such a matrix may change by the wetting both by replacement of plasticizers by water (similarly to the composition change going on a matrix of the first kind), and may also lose some of its PVOH to dissolve or disperse in the liquid. The mechanical properties of a matrix of the second kind change during the wetting stage, and the matrix becomes softer and more pliable. In some embodiments, a matrix of the second type loses some of its size during wetting stage, but remains intact.

The chemical composition of a matrix of the second kind includes a substantial part (e.g., at least 50%) of long-chain highly hydrolyzed PVOH, and the rest of the PVOH (e.g., no more than 50%) is of short chains and/or partial hydrolysis degree. It is noted that the transition between being a matrix composition of the first or second kind is not sharp, and the question if a certain composition of PVOH of different types will result in a matrix of the first or second kind may depend on the specific types of PVOH used, as well as on the kinds and amounts of the plasticizers.

Still in the wetting stage, the degradation behavior of a matrix of a third kind is shown in the figure by a dotted line. A matrix of the third kind loses nearly all its mass when under wet conditions for long enough, and loses size and shape to the extent that if not taken out of the water soon enough, it becomes shapeless and slimy. The chemical composition of such a matrix may change by losing plasticizers and PVOH. The mechanical properties of a matrix of the third kind change dramatically during the wetting stage, and the matrix may completely dissolve during the wetting stage.

The chemical composition of a matrix of the third kind includes mainly short-chain and/or partially hydrolyzed PVOH, and only small amounts of the PVOH (e.g., less than 40%) is of long chains and/or full hydrolysis degree. It is noted that the composition change required to go from a matrix of the second to a matrix of the third kind is not the same for all the compositions of matrices of the second kind, and the question if a certain composition of PVOH of different types will result in a matrix of the second or third kind may depend on the specific types of PVOH used as well as on the plasticizers used and their amounts.

The third phase of the cycle, which may be referred to as a drying stage, starts when the matrix is taken out of the liquid, goes on for as long as the dry conditions are maintained. In case the matrix is used for medical application, the behavior at the third phase may represent the behavior of the matrix after body fluids drained or dried (e.g., after bleeding, sweating, or tearing stops or significantly decreases).

In the drying stage, a matrix of the first kind may lose a substantial part of its mass (e.g., up to 70%), mainly because water evaporation. Shape may be kept, and size may be reduced. Re-wetting may cause the dried matrix to regain shape and size, at least partially. Flexibility and pliability are lost during drying, but may be partially regained by re-wetting.

A matrix of the second kind, goes through similar processes during the drying stage, but may lose larger part of its mass (e.g., up to 85%). Changes in size and shape will be similar to those going on matrices of the first kind, but larger. In some embodiments, a matrix of the second kind does not regain flexibility and pliability upon re-wetting.

A matrix of the third kind does not live long enough to reach the drying stage, as it may dissolve during the wetting stage. If the environment of a matrix of the third kind becomes dry before a substantial part of the mass of the matrix is dissolved, some regaining of shape and size may be achieved by re-wetting.

In some embodiments, e.g. for medical use, the required combination of types of PVOH within the elastomeric matrix may be designed to achieve a product that keeps geometrical form in dry conditions (meaning before positioning in the body). Keeping form under dry conditions potentially increases the shelf-life of the matrix, whether used as part of an ophthalmic device or as part of any other medical device. In some embodiments, keeping form under dry conditions is important since the user needs to manipulate the ophthalmic device in order to position it properly in the body of the patient, for example, in the case of ophthalmic device, on the surface of the eye. Therefore, in some embodiments, e.g., for ophthalmic devices, the combination of types of PVOH in the elastomeric matrix provides a matrix that keeps its geometry and mechanical properties in dry conditions.

In some embodiments, critical values of the combination between types of PVOH are according to the following:

$$Where : \frac{total PVOH}{total PVOH + Plasticizers} = from about 5 \% wt to about 33 \% wt$$

Matrices according to some preferred embodiments have the following PVOH compositions:

| Total PVOH | $Minimum \frac{LCFH PVOH}{Total PVOH}$ | $Maximum \frac{Short chain PVOH}{Total PVOH}$ |
|---|---|---|
| From about | 10% wt | 90% wt |
| 20% wt to | | |
| about 30% wt | | |
| From about | 20% wt | 80% wt |
| 10% wt to | | |
| about 20% wt | | |
| From about | 30% wt | 70% wt |
| 5% wt to | | |
| about 10% wt | | |

It is noted that the right column covers embodiments wherein the PVOH is fully hydrolyzed as well as embodiments wherein the PVOH is partially hydrolyzed, or a mix of fully and partially hydrolyzed PVOH.

Without being bound to theory, the degradation behavior of the various matrices according to embodiment of the present invention may be affected by the physical crosslinking, and the latter may be affected, for example, by the different types of PVOH, their relative amounts, the total amount of PVOH relative to the plasticizers or other ingredients of the matrix, the plasticizers included in the composition, and the amount of each plasticizer. Additionally, or alternatively, the degradation behavior of a matrix may be affected by chemical crosslinking (e.g., by boric acid), for example, when it is gentle enough not to interfere with the desired characteristics of the matrix.

4.3. Exemplary Amounts of Components within an Exemplary Elastomeric Matrix Composition

In some embodiments, an exemplary elastomeric matrix comprises one or more of the following components:
1. Water from about 5% wt to about 50% wt of the matrix;
2. PVOH+plasticizers from about 35% wt to about 95% wt of the matrix; where:
   i. The PVOH is from about 5% wt to about 33% wt of the PVOH+plasticizers;
   ii. The weight of the plasticizers is at least twice the weight of the PVOH
3. Miscellaneous ingredients from about 0% wt to about 30% wt of the weight of PVOH+plasticizers+miscellaneous ingredients.

In some embodiments, the miscellaneous ingredients may include, for example, active pharmaceutical ingredients (API), buffering agents, etc.

### 4.4. Exemplary Mechanical Properties

In some embodiments, the elastomeric matrix provided herein can substantially change its mechanical properties, such as rheological properties, elongation at break, tensile strength, yield strength, elongation at yield, elastic modulus and so forth, when exposed to wet conditions.

When referring herein to “dry conditions”, it should be understood to relate to conditions wherein a matrix of the invention is exposed to air at room temperature in an open or closed container and is not immersed in liquid, and particularly not in aqueous liquid. When referring to “wet conditions”, it should be understood to relate to conditions wherein a matrix of the invention is in direct contact with liquid (immersed in the liquid or after immersion in the liquid and before the liquid evaporated or wiped away).

In some embodiments, the liquid is a naturally occurring bodily fluid, including but not limited to blood, saliva, tears, excreta, body tissue, tissue fluids), an aqueous solution (e.g., of the at least one plasticizer), a buffer solution, and any combinations thereof. In some embodiments, the liquid is naturally occurring or simulating a naturally occurring fluid, such as artificial tear fluid. In some embodiments, a simulated tear fluid is based on an aqueous solution containing about 0.67% sodium chloride, about 0.2% sodium bicarbonate and about 0.008% calcium chloride.

In some embodiments, the elastomeric matrix provided herein is characterized by an elongation at break of at least 50%, 100%, 200%, 300%, 400%, 500%, 700%, at least 800%, or at least 900% or at least 1000% under dry conditions. In some embodiments, the elongation at break of the matrix is at least 100% under wet conditions (immersed in liquid or after immersion in liquid).

It was surprisingly found that in some embodiments of the present invention wherein the water content was relatively low, the elongation at break was surprisingly high. Thus, according to some embodiments of the present invention, the elastomeric matrix having a water content of 30% wt or less, 25% wt or less, or 20% wt or less, exhibited relatively high elongation at break under dry conditions of 50% or higher, 100% or higher, and even 300% or higher or 500% or higher.

In some embodiments, the elastomeric matrix provided herein is characterized under dry conditions by a tensile strength in the range 0.01 MPa to 1, 2, 3, 4, or 10 MPa. The module of elasticity (Young module) varies within similar ranges.

In the table below (Table 2), summarized are some mechanical properties measured under dry conditions with matrices that all had the same composition, except for differing in the types of PVOH.

TABLE 2

| PVOH | #1 | #2 | #3 | #4 | #5 |
|---|---|---|---|---|---|
| LCFH/total | 1 | 0.5 | 0.4 | 0.5 | 0.4 |
| SCPH1/total | 0 | 0.5 | 0.6 | 0 | 0 |
| SCPH2/total | 0 | 0 | 0 | 0.5 | 0.6 |
| Module Young | 0.5 ± 0.05 | 0.17 ± 0.02 | 0.07 ± 0.001 | 0.17 ± 0.06 | 0.030 ± 0.005 |
| (tension)[MPa] | | | | | |
| Tensile strength | 1.04 ± 0.01 | 0.19 ± 0.04 | 0.07 ± 0.02 | 0.22 ± 0.04 | 0.050 ± 0.002 |
| [MPa] | | | | | |
| Elongation at | 467 ± 1 | 165 ± 61 | 123 ± 57 | 212 ± 25 | 188 ± 28 |
| break [%] | | | | | |
| Compressive | 0.63 | 0.48 | 0.29 | 0.51 | 0.22 |
| module [MPa] | | | | | |

These examples exemplify that increasing the amount of short chain partially hydrolyzed PVOH may lower elastic module, tensile strength, and elongation at break. In these examples, the difference between PVOH of the same hydrolysis degree (88%) and slightly different chain length (750 and 630) was reflected in modest changes in mechanical properties.

Mechanical measurements presented herein were performed using computer-controlled Mark10 model F105 tension/compression test frame, equipped with 10N or 100N force sensor equipped with standard grips and head. Software: “IntelliMESUR”.

Wet measurements were performed in Simulated tear fluid (STF) with pH adjusted to ~7.2.

Tensile measurements done using “Dog bone” model. Dog bone dimensions: total length 6 cm. Grip width 2 cm, neck width 0.8 cm, thickness 0.5-3.5 mm. Measurement speed 40 mm/min.

Tensile measurements of wet samples were performed after soaking a “dog bone” sample in 5 ml STF. After designated time the dog bone was taken out of the liquid, mounted on the grips and measured immediately.

Press measurements performed using 12.7 mm flat head and cylinder shaped samples. Sample dimensions: 4 mm diameter, height 2.5-3.0 mm. Measurement speed 20 mm/min.

A set of individual samples were soaked separately in small containers and each was measured after designated time. The samples were pressed inside the liquid without being touched or moved until the measurement.

An exemplary elastomeric matrix, the dry material of which was composed of 20% PVOH, all of which 28-99, 20% polyethylene glycol, and 60% glycerol, was measured to have Young Modulus of 0.41 MPa, tensile strength of 0.51 MPa, and elongation at break of 456%.

### 4.5. Exemplary Plasticizers

The term “plasticizer”, as used herein, refers to a wide range of substances that together with PVOH bestow the mechanical properties of the elastomeric matrix provided herein. Without being bound by any particular theory, it is assumed that the PVOH and at least one of the plasticizer(s) interact and form a hydrogen-bonded network that allows the matrix to exhibit shape stability and elasticity under wet and dry conditions. The hydrogen bonded network may include plasticizer molecules linked to two (or more) PVOH residues.

In some embodiments of the present invention, the plasticizer is an organic material, i.e., matter in its various forms that contain carbon atoms.

According to embodiments of the present invention, at least one of the plasticizers is characterized by exhibiting at least two hydrogen bond forming functional groups, namely at least two H-bond acceptors, at least two H-bond donors, or at least one H-bond acceptor and at least one H-bond donor.

According to some embodiments of the present invention, a plasticizer exhibits more than two H-bond forming functional groups, or more than 3, more than 4, more than 5, more than 6, more than 7, or more than 8H-bond forming functional groups. In some embodiments, a plasticizer comprises a plurality of H-bond forming functional groups.

According to some embodiments, a plasticizer is characterized by a molar mass of less than 1,000 g/mol, or less than 500 g/mol. In some embodiments wherein the elastomeric matrix includes at least two plasticizers, the matrix may include at least one plasticizer which is an oligomer characterized by a molar mass of more than 1,000 g/mol. In some embodiments, the oligomer is characterized by a molar mass that ranges 1,000-2000 g/mol.

According to some embodiments of the present invention, a plasticizer is characterized by forming non-rigid crosslinked bridges with other elements of the matrix, such as PVOH. In this context, a plasticizer is characterized by exhibiting at least one, or at least two rotatable bonds in its structure, not including the bonds connecting the hydroxyl group to the molecule. In some embodiments, a plasticizer is characterized by exhibiting at least one bond having a variable dihedral angle. A dihedral angle is the angle between half-planes through two sets of three atoms, having two atoms in common. In some embodiments, the dihedral angle is defined between half-planes through two sets of three non-hydrogen atoms, having two non-hydrogen atoms in common. For example, ethylene glycol exhibits one variable dihedral angle which is defined by two sets of three atoms, each having an oxygen and two carbon atoms common to the two sets of non-hydrogen atoms.

In some embodiments, the plasticizers are biocompatible, namely the plasticizers do not elicit a non-tolerable local or systemic effect in a recipient/user. In some embodiments, a plasticizer is an ophthalmic compatible (acceptable) substance, namely it is biocompatible, and particularly does not elicit a non-tolerable ophthalmic effect in the recipient/user.

In the context of the present invention, according to some embodiments the elastomeric matrix comprises one or more plasticizers, and in other embodiments, the elastomeric matrix comprises at least two plasticizers. It is noted that when referring herein to a particular characteristic of a plasticizer, it is meant to be taken as referring to a single plasticizer, some of the plasticizers, or to each of the plurality of plasticizers individually.

According to some embodiments of the present invention, when the matrix includes more than one plasticizer, one is characterized by a viscosity that is at least 5-times higher than the viscosity of the other plasticizer. In some embodiments, at least one of the plasticizers is characterized by a viscosity of at least 1000 cp, and the other plasticizer is characterized by a viscosity of less than 200 cp. Alternatively, one plasticizer has a viscosity of 500 cp or above and the other 50 cp or below. For example, in one exemplary matrix, one of the plasticizers is glycerol, exhibiting a viscosity of about 1400 cp, and the other plasticizer is polyethylene glycol, exhibiting a viscosity of about 100 cp. In another example, one of the plasticizers is glycerol, and the other propylene glycol, exhibiting viscosity of about 40 cp. All viscosities are of the liquid plasticizers, at 20° C.

According to some embodiments, all plasticizers are liquid at room temperature.

In some embodiments, the one or more plasticizers constitutes at least 30% wt, 40% wt, 50% wt, 60% wt, 70% wt, 80% wt of the total weight of the matrix. In some embodiments, the plasticizer constitutes 30-80% wt of the weight of the matrix and any sub-range therebetween.

In some embodiments, the plasticizer is selected from polyols (e.g. ethylene glycol, diethylene glycol (DEG), triethylene glycol (TEG), and tetraethylene glycol), propylene glycols, glycerol, esters of glycerol (e.g. triacetin), polyprotic organic acids, e.g., oxalic acid, maleic acid, citric acid, etc.), polyamines (e.g., spermine, spermidine, diethylenetriamine, triethylenetetramine, tris(2-aminoethyl)amine, a polyethylenimine (PEI; polyaziridine, etc.), Trypan blue, alkyl gluceths, aliphatic polyether glycos (e.g., polyethylene glycol, prolypropylene glycol, polysorbate 80), polyoxyethylenes, ethanolamines, erythritols, tromethamine, urea, saccharides, amino acids (e.g., glycine, aspartate/aspartic acid etc.), polyphenols (e.g. tannic acid) and any combinations thereof.

In some embodiments, the plasticizer is an ophthalmic lubricant, emollient or demulcent, as described by the FDA in 21 CFR 349.12. In the present document, the terms lubricant and demulcent are used interchangeably.

Examples of lubricants that may be used as plasticizers include dextran, gelatin, povidone, hyaluronic acid or pharmaceutically acceptable salt thereof, polyols, cellulose and cellulose derivatives.

Examples of polyols include glycerol, polyethylene glycol (e.g., PEG 300 or PEG 400), propylene glycol, and polysorbate (e.g., polysorbate 70).

Examples of cellulose derivatives include carboxymethyl cellulose sodium, hydroxyethyl cellulose, methylcellulose, and hydroxypropyl methylcellulose (HPMC).

Thus, according to some embodiments of the present invention, the plasticizers are ophthalmic demulcents selected from the group consisting of cellulose derivatives, carboxymethylcellulose sodium, hydroxyethyl cellulose, hypromellose, hydroxypropyl cellulose, methylcellulose, hemicellulose, dextran, gelatin, liquid polyols, glycerin, polyethylene glycol 300, polyethylene glycol 400, polysorbate 80, propylene glycol, povidone, and any combination thereof. While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

In some embodiments, the matrix comprises glycerol as a single plasticizer. In some embodiments, the matrix comprises propylene glycol as a single plasticizer. In some embodiments, the matrix comprises glycerol and propylene glycol as plasticizers. In some embodiments, the matrix comprises glycerol and propylene glycol, each alone or a mixture thereof mixed with PEG, as plasticizers. In some of embodiments, PEG is the amount of PEG and the amount of PVOH are substantially the same.

### 4.6. Exemplary Compositions of Elastomeric Matrices

While the elastomeric matrix provided herein can be prepared to suit the desired properties, some applications require the matrix to exhibit unique properties, which require to add another type of polymer(s) which is not PVOH of any type, yet typically produced from hydrogen-forming monomers. This type of elastomeric matrices is referred to herein as a composite matrix.

In general, composite materials are the products of a connection between two different chemical entities. The connection between the different entities can be through covalent, non-covalent, ionic or other type or bond. The resulting properties sometimes represent a simple or complex weighted average of the individual properties of each ingredient, and sometimes products with very different properties from those of the two original components are obtained.

In the context of the present invention, it was found that by adding one or more polymers that are not PVOH of any type, new materials with a myriad of diverse characteristics can be obtained while keeping the basic characteristics of the elastomeric matrix provided herein, namely low amount of polymers, softness, and shape retention under varying dry and wet conditions.

The addition of the non-PVOH polymer can be used for example in order to further modify one or more of stability in dry and/or wet conditions, bio-degradability, rheological properties, bio-adhesiveness, compatibility with active materials, active material release profile and imprinting active materials.

In some embodiments, the composite elastomeric matrix is made by blending PVOH of one or more types with a non-PVOH polymer capable of hydrogen bonding with PVOH. In some embodiments, said non-PVOH polymer is neutral (not charged). In some embodiments, said non-PVOH polymer has anionic or cationic functional groups (charged). In some embodiments, said non-PVOH polymer is a synthetic polymer. In some embodiments, said non-PVOH polymer is an oligosaccharide or polysaccharide. In some embodiments said non-PVOH polymer is an acrylic polymer.

It is clear to those skilled in the art that additional polymers and various types of molecular links (e.g. ionic, complexes etc.) can be used to create composite materials with PVOH of one or more types, using either covalent or non-covalent interactions in order to connect the two polymers. The additional polymer that is not PVOH can be selected from various polymers, including naturally occurring polymers and macromolecules, synthetic polymers and the likes. For example, without limitation, the polymer of hydrogen bond forming monomers that is not PVOH is a polyacrylic acid, a polyvinyl pyrrolidone, cellulose, chitin, glycogen, starch, gellan, dextran, inulin, pectin, arabinoxylan, and any mixture thereof.

According to some embodiments of the present invention, the mass content of the non-PVOH polymer in the elastomeric matrix provided herein is smaller than the mass content of PVOH of all the types together. In some embodiments, the mass ratio between PVOH (of the one or more types) and the non-PVOH polymer ranges from about 100:1 to 2:1. Some exemplary embodiments of composite matrices are provided in the Examples section that follows below.

### 4.7 Exemplary Medical/Ophthalmic Device

The present invention further includes the aspect of a medical device (e.g., an ophthalmic/ocular device) that can be used in treating pathologies and diseases, wherein the device comprises or consists of the elastomeric matrix provided herein. In some embodiments, the device may be used topically, in a body cavity, or the like. In some embodiments, the device may be injected into a body part or tissue.

In the context of the ophthalmic device, according to some embodiments of the present invention, the matrix comprises or consists of ophthalmically acceptable ingredients, as these are discussed hereinabove. For example, any ingredient allowed for ophthalmic use by any regulatory authority may be referred to as ophthalmically acceptable ingredients. In some embodiments, all the plasticizers are ophthalmic demulcents. While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

In some embodiments, the elastomeric matrix provided herein is particularly useful for sequestering and/or releasing active pharmaceutical ingredients (API), since it is unreactive, stable and benign. In some embodiments, the ophthalmic device that comprises the elastomeric matrix provided herein, is configured for drug delivery, both for bolus as well as for sustained release regimens. Hence, according to some embodiments, the matrix includes therein or thereon at least one active pharmaceutical ingredient (API). Optionally, the active pharmaceutical ingredient (API) is selected for treating an ophthalmic symptom, disease or disorder. Optionally, the active pharmaceutical ingredient (API) is selected for treating a buccal symptom, disease or disorder (see below).

In some embodiments, the ophthalmic device described herein is generally sized and shaped to be positioned on an outer surface of the eye with at least a portion of the ocular device positioned under one eyelid in a manner that does not contact or interfere with the cornea. In some embodiments, the ophthalmic device provided herein is configured to be positioned on a surface of the eye, at least partially underneath the upper or lower eyelid and outside a cornea of the eye for delivering at least one active pharmaceutical ingredient (API) to an eye for an extended period of time, for example of between 5 minutes and 24 hours. Optionally more than 24 hours.

In some embodiments, the elastomeric matrix provided herein is characterized by a porous microstructure, which allows entrapping a liquid therein. In some embodiments, the liquid may be, for example, a solution or a colloid comprising an active pharmaceutical ingredient (API), and this liquid can be dispersed in the matrix and/or entrapped in the pores of the matrix. Hence, in some embodiments, the device includes at least one active pharmaceutical ingredient (API) dispersed, sequestered, impregnated within the elastomeric matrix provided herein.

In some embodiments, the active pharmaceutical ingredient (API) can include, for example, a small molecule, a macromolecule, a cell or a tissue.

In some embodiments, the active pharmaceutical ingredient (API) is in a liquid form. In some embodiments, the active pharmaceutical ingredient (API) is in solid form. In some embodiments, the active pharmaceutical ingredient (API) is soluble in water, or soluble in an organic solvent, or amphiphilic.

In some embodiments, the active pharmaceutical ingredient (API) Is encapsulated, or microencapsulated, or in a microparticle form, or in a nanoparticle form.

In the context of the present invention, the active pharmaceutical ingredient (API) is, without limitation, an analgesic agent, an antacid, an antianxiety agent, an antiarrhythmic, an antibacterial agent, an antibiotic agent, an anticoagulant, a thrombolytic agent, an anticonvulsant, an antidepressant, an antiemetic agent, an antifungal agent, an antihistamine, an antihypertensive, an anti-inflammatory agent, an antineoplastic agent, an antipsychotic agent, an antipyretic agent, an antiviral agent, a barbiturate, a bronchodilator, a beta-blocker, a corticosteroid, a cold cure, a cytotoxic agent, a decongestant, a diuretic agent, a expectorant, a hormone, a hypoglycemic agent, an immunosuppressive agent, a laxative, a muscle relaxant, a sedative, a sex hormone, a sleeping drug, a tranquilizer, a vitamin, and any combinations thereof.

In some embodiments, the active pharmaceutical ingredient (API) is an ophthalmological active pharmaceutical ingredient (API). In some embodiments, the ophthalmological active pharmaceutical ingredient (API) is a lubricant, an anti-angiogenic agent, a mydriatic agent, an anesthetic, an anti-infective agent, an anti-inflammatory agent, an antihistamine, a glaucoma treatment agent, a surgical agent, a diagnostic agent or any combination thereof.

While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

In some embodiments, the active pharmaceutical ingredient (API) can include, without limitation, bimatoprost, travoprost, latanoprost, tafluprost, NSAID, steroid, antihistamine, carbonic anhydrase inhibitor (CAI), dorzolamide, cyclosporine, antibiotic, doxycycline, tetracycline, azithromycin, fatty acid, long chain fatty acid, fatty alcohol, cetyl alcohol, stearyl alcohol, non-penetrating steroid, free acid of steroid, lipid, ketorolac, silicone oil, olopatadine, prostaglandin, prostaglandin analog, prostamide, small-molecule integrin antagonist, lifitegrast, loteprednol, and fluoromethalone or a combination thereof.

In some embodiments, the active pharmaceutical ingredient (API) can include a prostaglandin analogue. In some embodiments, the prostaglandin analogue can include at least one of bimatoprost, latanoprost, travoprost, and tafluprost. In some embodiments, the active pharmaceutical ingredient (API) can be for lowering the intraocular pressure of the eye. In some embodiments, the active pharmaceutical ingredient (API) can be for treating dry eye. In some embodiments, the active pharmaceutical ingredient (API) can include at least one of cyclosporine, steroid, loteprednol, fluoromethalone, non-penetrating steroid, free acid of steroid, nonsteroidal anti-inflammatory, ketorolac, small-molecule integrin antagonist, lifitegrast, doxycycline, azithromycin, lipid, fatty alcohol, cetyl alcohol, stearyl alcohol, fatty acid, long chain fatty acid, oil, or silicone oil. In some embodiments, the active pharmaceutical ingredient (API) can include a steroid. The steroid can include at least one of loteprednol or fluoromethalone.

In some embodiments, when preparing an ophthalmic device that comprises the elastomeric matrix according to some embodiments of the present invention, a formula is prepared according to the preparation procedure provided below. Briefly, a liquid formula is obtained which can be used to prepare the final device. The active pharmaceutical ingredient (API) can be mixed into said liquid formula which leads to a matrix and a device that includes the active pharmaceutical ingredient (API). Hydrophilic active pharmaceutical ingredients (API) easily dissolve in the formula, whereas hydrophobic active pharmaceutical ingredients (API) may be suspended (e.g., as a colloid) in the formula.

The amount of the active pharmaceutical ingredient (API) in the matrix depends on the active pharmaceutical ingredient (API) and the treatment regimen. The nature of the matrix and the requirement therefrom, allow the matrix to include up to 30% of its non-water constituents to be active pharmaceutical ingredients (API). For example from about 5% to about 25%, optionally form about 3% to about 27%, or any percentage between 0% and 30%.

In some embodiments, the matrix of the device further comprises at least one ophthalmically acceptable carrier/supplement/adjuvant/additive/non-pharmaceutical agent.

In some embodiments, the drug release profile depends on the active pharmaceutical ingredient (API), and the regimen. A skilled artisan would appreciate the means and technology present available for controlling sustained release of drugs from various devices and under various conditions. For example, encapsulation of active pharmaceutical ingredients (API), addition of materials that modifies the interaction between the agent and the matrix, and the likes.

In some embodiments, the active pharmaceutical ingredient (API) is released from the matrix when the matrix is in the presence of a liquid, such as water, saline, tear simulating fluid, saliva, blood, or any body fluid. When referring to “presence of liquid” it should be understood to relate to the condition wherein the matrix of the invention is exposed to liquid, partially or entirely immersed therein.

In some embodiments, the active pharmaceutical ingredient (API) is maintained within the elastomeric matrix under dry conditions. In some embodiments, the active pharmaceutical ingredient (API) is maintained within the matrix during storage. In some embodiments, the active pharmaceutical ingredient (API) is maintained within the matrix under dry conditions for at least six months.

### 4.8. Exemplary Method of Treatment

In some embodiments, the elastomeric matrix of the present invention can be used for administration of active pharmaceutical ingredients (API) to a cell, tissue, or membrane. The invention further provides a method of administering at least one active pharmaceutical ingredients (API) to a cell or a tissue/membrane, which is realized by contacting the cell or tissue/membrane with a therapeutically effective amount of the active pharmaceutical ingredient (API) present in the elastomeric matrix as disclosed herein. In some embodiments, the tissue is selected from ocular, buccal, dental, orthodontic, muscular, mucosal, dermatological, connective, cardiac and any combinations thereof.

As used herein, the phrase “therapeutically effective amount” describes an amount of an active pharmaceutical ingredient (API) being administered, which will relieve to some extent one or more of the symptoms of the medical condition being treated. Thus, any amount may be therapeutically effective, depending on the condition to be treated, the effectiveness of the active pharmaceutical ingredient (API), the body size of the patient, etc. In the context of the present embodiments, the phrase “therapeutically effective amount” describes an amount of the active pharmaceutical ingredient (API) being administered and/or re-administered, which will relieve to some extent one or more of the symptoms of the condition being treated. In some embodiments, relieve may be achieved by being at a level that is harmful to the target cell(s) or microorganism(s), and cause a disruption to the life-cycle of the target cell(s) or microorganism(s). In some embodiments, relieve may be achieved by being at a level that is pharmaceutically effective to induce a therapeutic effect on a patient, for example, an effective quantity of muscle relaxant, pain reliever and/or antibiotics.

In the context of embodiments of the present invention, the therapeutically effective amount may refer to the active pharmaceutical ingredient (API) as a whole or to the amount of one or more active pharmaceutical ingredient (API) releasably sequestered and/or included in the matrix. The efficacy of any active pharmaceutical ingredient (API), can be determined by several methodologies known in the art.

According to another aspect of embodiments of the present invention, any one of the matrices described herein is suitable for use in treating a subject diagnosed with a medical condition treatable by at least one active pharmaceutical ingredient (API) sequestered (or included in the matrix) and controllably releasable from the matrix.

According to another aspect of embodiments of the present invention, there is provided a use of any of the elastomeric matrixes described herein as delivery vehicle for use as a medicament. In some embodiments, the medicament is for treating a subject diagnosed with a medical condition treatable by at least one of the drugs sequestered (or included in the matrix) and controllably releasable from the matrix.

In any of the methods and uses described herein, the matrix can be used for medicinal purposes as a part of a medical device.

### 4.9. Exemplary Method of Preparation

The present invention further provides a method of preparing the elastomeric matrix as disclosed herein, wherein the method comprises the steps of:
Option 1: Preparing individual solutions, each comprising a different type of PVOH. Mixing parts of the individual solutions according to the required ratio.
Qption 2: Dissolving two or more types of PVOH in water to form a solution.
   Optionally heating the solution (either option 1 or option 2)
   Mixing the plasticizer(s) into the solution to form a mixture;
   Optionally heating the mixture;
   Adding the mixture into a mold;
   Allowing the mixture set in the mold, to thereby form the elastomeric matrix of the invention.

In some embodiments, the mixture has less than 20% wt PVOH.

In some embodiments, the plasticizers make 30% wt or more of the mixture.

In some embodiments of the method of preparing the matrix of the invention, the optional heating is to a temperature in the range of 60-100° C.

In further embodiments of the method, adding the mixture into a mold is conducted at room temperature, for example, after the mixture is let to cool while mixing. In some embodiments of the method, adding the mixture into a mold is conducted in an open form, e.g., in an open flask or mold. In some embodiments, the elastomeric matrix is poured into the mold. In some embodiments, the elastomeric matrix is pumped into the mold.

In some embodiments of the method, at least one active pharmaceutical ingredient (API) is added to the solution, preferably, when the solution is at about room temperature, as not to encourage reactions between the active pharmaceutical ingredient (API) and other ingredients in the solution. In some embodiments, this also allows using active pharmaceutical ingredients (API) that are heat sensitive. In some embodiments, the API is not heated at any stage of the method, and the elastomeric matrix or ophthalmic device containing it is obtained without heating the API.

In some embodiments of the method, the elastomeric matrix that is formed in step (f) is immersed, e.g., for 5 minutes, in a solution comprising at least one plasticizer or water or an aqueous solution or a solvent.

### 4.10 Summary of Exemplary Elastomeric Matrix Comprising Two or More Types of PVOH

An aspect of the disclosed invention includes an elastomeric matrix, comprising poly(vinyl alcohol) (PVOH); one or more organic plasticizers; where a combined mass ratio of the one or more organic plasticizers to the PVOH is at least 2:1; and water, where the PVOH comprises PVOH of two or more types that differ from one another in one or both of hydrolysis degree (HD) and chain length.

In some embodiments, the PVOH comprises PVOH of two or more types that differ from one another in hydrolysis degree (HD). In some embodiments, the two types of the PVOH differ in chain length by at least 1000, and have similar degrees of hydrolysis, both between 97% and 100%. In some embodiments, a first type of the two or more types has a hydrolysis degree from 97% to 100%. In some embodiments, a second type of the two or more types has a hydrolysis degree is lower than 95% or lower than 93%. In some embodiments, a second type of the two or more types has a hydrolysis degree from 80% to 93%. In some embodiments, a first type of the two or more types has chains with more than 2500 units. In some embodiments, a second type of the two or more types has chains with less than 1500 units. In some embodiments, a relation between a first type of PVOH and a second type of PVOH of the two or more types is from about 3:1 to about 1:3. In some embodiments, a first type of the two or more types has chains with more than 2500 units and hydrolysis degree of 97% to 100%, and a second type of the two or more types has chains with less than 1000 units and hydrolysis degree of 80% to 93%. In some embodiments, the second type of PVOH makes more than 50% of the PVOH. In some embodiments, the at least two types of PVOH determine a time of degradation of said elastomeric matrix. In some embodiments, the degradation comprises change in mechanical properties under wet conditions. In some embodiments, the degradation comprises change in shape under wet conditions. In general, the degradation may be due to breakage of bonds (e.g., hydrogen bonds) between the different constituents of the matrix (e.g., PVOH, plasticizers, and water) while each of the constituents stays intact. In some embodiments, the degradation allows clearing the ophthalmic device, or the elastomeric matrix it comprises, from the eye, for example, by tears, that may wash the matrix constituents, crumbs, and/or remains out of the eye.

In some embodiments, the at least two types of PVOH determine a time development of mechanical properties of the elastomeric matrix under wet conditions. In some embodiments, a combined mass content of the PVOH and the one or more organic plasticizers is at least 70% wt of the total weight of the matrix excluding water. In some embodiments, the elastomeric matrix is used as an ophthalmic device. In some embodiments, the combined mass ratio of the one or more organic plasticizers to the PVOH is less than 20:1. In some embodiments, the elastomeric matric is characterized by substantially isotropic swelling and shrinking. In some embodiments, the elastomeric matrix swells by less than 50% by volume under wet conditions. In some embodiments, the elastomeric matrix is characterized by substantially isotropic swelling when being immersed in simulated tear fluid and at room temperature for 5, 10, or 15 minutes. In some embodiments, the elastomeric matrix swells by less than 50% by volume when immersed in simulated tear fluid for 5, 10, or 15 minutes at room temperature. In some embodiments, the one or more organic plasticizers is independently selected from the group consisting of a polyol, a polyprotic organic acid, a polyamine, an alkyl gluceth, an aliphatic polyalkylene glycol, an ethanolamine, a saccharide, an oligosaccharide, an amino acid, a polyphenol, tromethamine, urea, tannic acid, and any salt thereof and/or combinations thereof. In some embodiments, the polyol is selected from the group consisting of ethylene glycol, diethylene glycol, triethylene glycol, tetraethylene glycol, propylene glycol, triacetin erythritol, a poly-glycol, a poloxamer, and a co-polymer thereof, and glycerol and esters thereof. In some embodiments, the polyprotic organic acid is selected from the group consisting of oxalic acid, maleic acid, citric acid, and any salt thereof. In some embodiments, the polyamine is selected from the group consisting of spermine, spermidine, diethylenetriamine, triethylenetetramine, tris(2-aminoethyl)amine, a polyethylenimine, and any salt thereof. In some embodiments, the aliphatic polyalkylene glycol is selected from the group consisting of a polyethylene glycol, a polypropylene glycol, a poly-glycol, a poloxamer, and a polysorbate. In some embodiments, the said aliphatic polyalkylene glycol is polyethylene glycol. In some embodiments, a mass content of said PVOH is substantially equal to a mass content of said aliphatic polyalkylene glycol. In some embodiments, the aliphatic polyalkylene glycol is selected from the group consisting of polyethylene glycol, polypropylene glycol, and any mixture thereof. In some embodiments, the water constitute less than 50% wt of the total mass content of the matrix. In some embodiments, the mass content of said PVOH is less than 25% wt of the total weight of non-water ingredients of the matrix. In some embodiments, the mass content of said PVOH is less than a third of the total weight of said one or more organic plasticizers. In some embodiments, the elastomeric matrix is essentially devoid of covalent crosslinking. Being essentially devoid of covalent crosslinking may be expressed as being water soluble in hot water. The amount and temperature of the water required to dissolve the matrix depends on the specific PVOH types that the matrix includes. The solubility of the matrix is about the solubility of the less soluble of those PVOH types. In some embodiments, the elastomeric matrix further comprises an active pharmaceutical ingredient (API).

An aspect of the disclosed invention includes an ophthalmic device comprising the elastomeric matrix according to what is disclosed herein.

In some embodiments, the elastomeric matrix consists of ophthalmically acceptable ingredients. In some embodiments, the device weights between 3, 5, or 8 mg and 50 mg. In some embodiments, the minimum weight of the device is 8 mg.

In some embodiments, the elastomeric matrix comprises a active pharmaceutical ingredient (API). In some embodiments, the ophthalmic device is configured to be positioned on a surface of an eye. In some embodiments, the surface is at least partially underneath at least one of the upper eyelid, the lower eyelid and outside a cornea of the eye. In some embodiments, the ophthalmic device is configured for delivering at least one active pharmaceutical ingredient (API) to an eye for an extended period of time of between 5 minutes and 24 hours.

An aspect of the disclosed invention includes a medical device comprising two elastomeric matrices, wherein the two matrices differ from one another in one or more of ratio between the mass content of PVOH included in the matrix and the mass content of the matrix; ratio between the mass content of PVOH included in the matrix and the combined mass contents of the one or more plasticizers; the types of PVOH; and mass ratios between the types of PVOH.

In some embodiments, the two matrices touch across a touching surface. In some embodiments, the touching surface is a closed surface. In some embodiments, the two matrices degrade under wet conditions at different rates. In some embodiments, the two matrices show distinct mechanical properties under dry conditions.

An aspect of the disclosed invention includes an elastomeric matrix, comprising poly(vinyl alcohol) (PVOH); one or more organic plasticizers; where a combined mass ratio of said one or more organic plasticizers to said PVOH is at least 2:1; and water; where the PVOH comprises only PVOH of chains shorter than 2000 units or shorter than 1000 units and degree of hydrolysis smaller than 90%, and the water makes less than 10% or less than 5% of the matrix.

5. Additional Exemplary Information about Exemplary Ophthalmic Devices

### 5.1. Exemplary Compositions of Drug Products

In some embodiments, the softness and suppleness of the drug product are achieved thanks to its unique composition of excipients. In some embodiments, the excipients may include relatively small amounts of water (e.g., between 10% wt and 45% wt of the drug product) and large amounts of ophthalmic demulcents (at least 50% wt of the drug product). In some embodiments, the required softness may be achieved with an elastomeric matrix formulated with water, PVOH, and a large amount plasticizers. The amount of the plasticizers may be considered large if it is larger than, or, preferably, at least double (in mass) than the mass amount of the PVOH. In some such embodiments, the mass of the PVOH and the plasticizers together is 70% or more of the total mass of the matrix excluding the water. In some embodiments, the drug product consists essentially of the water, demulcents, and drug. In some embodiments, the ophthalmic demulcents preferably include up to 25% wt polyvinyl alcohol (PVOH). As used herein, an ophthalmic demulcent is a water soluble agent, that when applied topically to the eye protects and lubricates mucous membrane surfaces, for example, to relieve dryness and irritation. While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC). Non limiting examples of demulcents include PVOH, dextran, gelatin, povidone, hyaluronic acid or pharmaceutically acceptable salt thereof, polyols, cellulose and cellulose derivatives. In some embodiments, examples of polyols include glycerol, polyethylene glycol (e.g., PEG 300 or PEG 400), propylene glycol, and polysorbate (e.g., polysorbate 70). In some embodiments, examples of cellulose derivatives include carboxymethyl cellulose sodium, hydroxyethyl cellulose, methylcellulose, and hydroxypropyl methylcellulose (HPMC). In some embodiments, the PVOH preferably includes long-chain fully hydrolyzed PVOH, and optionally further includes PVOH of other grades. As used herein, long chain PVOH is PVOH characterized by an average chain length of 2500 monomeric units or more. PVOH having average chain length of 1000 monomeric units or less is referred to herein as short chain PVOH. In some embodiments, the degree of hydrolysis (DH) is the percentage of vinyl alcohol monomeric units out of all the monomeric units in the PVOH. As PVOH is usually synthesized by hydrolyzing acetate groups of poly vinyl acetate, PVOH may contain non-hydrolyzed acetate groups. As used herein, PVOH is considered fully hydrolyzed if at least 97% of the monomer units are vinyl alcohol units (i.e., degree of hydrolysis 97% or more), and partially hydrolyzed if 90% or less of the monomer units are vinyl alcohol units (i.e., degree of hydrolysis 90% or less). In some embodiments, further diversity of the mechanical properties of the drug product may be obtained by including in the composition of the drug product PVOH of two or more grades, which differ from each other by chain length and/or degree of hydrolysis. For example, the drug product may include, in addition to the above mentioned long chain fully hydrolyzed PVOH, partially hydrolyzed PVOH and/or PVOH of short and/or medium length. In some embodiments, for example, when a substantial part of the PVOH is short chain partially hydrolyzed PVOH, the resulting drug product may be even softer than when all the PVOH is long chain fully hydrolyzed PVOH. The substantial part may be, for example, at least 10% wt, at least 20% wt, or at least 35% wt of the PVOH. In some embodiments, the behavior of the drug product on the eye may also depend on the exact composition of the PVOH comprised in the drug product. The behavior of the drug product on the eye may be characterized, for example, by its tendency to dissolve in the tear fluid, its rate of dissolution, the constituency changes occurring when residing on the eye, if any, etc. In some embodiments, for example, a drug product containing only long chain fully hydrolyzed PVOH may be soft and pliable, and snugly adhere to the eye surface for at least 8 hours. In some embodiments, an ophthalmic device of the same composition, except for half the PVOH being short chain partially hydrolyzed, may be softer, and may soften even more when residing on the sclera near the eyelid, to have a slimy constituency, and lose about 80% of its dry weight within several hours on the eye. In some embodiments, an ophthalmic device of the same composition, except for the part of the short chain partially hydrolyzed PVOH making about two thirds of the weight of the PVOH in the drug product may be similarly slimy, but stick to the eyelid near the sclera, rather than lie on the sclera near the eyelid. In some embodiments, in addition to PVOH, the drug product preferably comprises a substantial portion of demulcents comprising two or more hydrogen bond forming groups, such as polyols. In some embodiments, the demulcents consist essentially of PVOH and polyols. In some embodiments, some preferred polyols are polyethylene glycol, glycerol, and propylene glycol. For example, in some embodiments, glycerol and propylene glycol make together at least 35%, at least 45%, or at least 55% of the demulcents. In some embodiments, the ratio between the glycerol and the propylene glycol may vary from 100% glycerol to 80% propylene glycol. Some embodiments with less than 20% wt of the demulcents being glycerol may not be of satisfactory soft. Without being bound to theory, it is assumed that the uniquely high viscosity of glycerol may play a role in determining the kinetics of the drug product formation. However, too high levels of glycerol may make the drug product less comfortable to the eye, as it tends to cause a burning sensation in the first minutes after administration, so some mixture of glycerol and propylene glycol may be preferable. In some embodiments, PEG (e.g., PEG 300 and/or PEG 400) may also be included in the drug product. Optionally, the mass content of the PEG is about the same as that of the PVOH. Without being bound to theory, it may be suggested that PEG reduces the amount of free water in the drug product, and helps the PVOH molecules to physically cross link to each other directly, or via other polyols such as glycerol and propylene glycol. In some embodiments, an ophthalmic device may further include an active pharmaceutical ingredient (API), suitable for treating an ophthalmic condition. In some embodiments, one or more of the demulcents may function as an API. Alternatively (or in addition) an API which is not a demulcent may be included in the drug product. In some embodiments, optionally, the ophthalmic device may be formed as an elastomeric matrix (comprising the water and the demulcents recited above) carrying the API. For example, the API may be impregnated in the elastomeric matrix, e.g., dissolved in the water of the matrix. While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

### 5.2. Exemplary Process for Manufacturing an Exemplary Ophthalmic Device

In some embodiments, an ophthalmic device as described herein may be manufactured by mixing the water with the ophthalmic demulcents (and with the API, if it is not one of the demulcents) to obtain a mother solution; and drying the mother solution, e.g., in an open mold. In some embodiments, obtaining the mixture of the demulcents with the water may require heating the water, e.g., to 80° C., so as to allow the PVOH to dissolve. In some embodiments, other demulcents may be added during heating and/or after the PVOH solution is cooled to room temperature. In some embodiments, the API is preferably added at room temperature, and mixed with the water and demulcents until the solution is clear. In some embodiments, adding the API at room temperature may have an advantage in that some APIs may be heat sensitive, and their activity may be changed or lost if handled at high temperature, as by mixing with a hot solution. It is noted that unless the active pharmaceutical ingredient (API) needs constant refrigeration, there is no need to cool the mother solution, or any other ingredient during any stage of the manufacturing process, to below room temperature, and there is no need to heat the matrix or drug product when or after it is dried. In some embodiments, the ophthalmic device is obtainable without heating the mother solution. In some embodiments, the ophthalmic device is obtainable without heating the API. In some embodiments, the drying itself may be carried out in a mold. Optionally, in a mold open to the atmosphere, at room temperature and ambient humidity. An interesting characteristics of ophthalmic devices according to the present disclosure is that when dried in a convex mold, they form a thick layer of varying thickness (typically having a maximal thickness of between 0.2 mm and 1 mm). In some embodiments, the mold is shaped so that the drug product dried therein has a shape configured to long residence on the eye, for example, with a retention portion as described herein. While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

### 5.3. Exemplary Shape of Exemplary Ophthalmic Device

In some embodiments, an exemplary ophthalmic device as described above is configured to reside on the bulbar conjunctiva of an eye, covered by an eyelid. In some embodiments, the exemplary ophthalmic device is preferably small enough not to cover the cornea when residing on the bulbar conjunctiva. It is noted that the cornea may be irritated more easily than the bulbar conjunctiva, and therefore, it is preferable that the drug product is shaped and sized not to cover the cornea. Furthermore, it is preferable that the exemplary ophthalmic device is small, and covers only minimal area of the sclera near the bulbar conjunctiva. This is a considerable advantage, for example, over devices that circumvent the cornea, but has one portion at a lower conjunctiva and another portion at an upper conjunctiva of the same eye. In some embodiments, the compositions described herein, while being very soft and non-irritating, are not inert enough to be comfortable when they are so huge. In some embodiments, the drug product is small enough to be fully covered by the eyelid underneath which it is administered. In some embodiments, preferably, the exemplary ophthalmic device is configured to reside under a lower eyelid, where there is more space for the drug product, and it may cause less irritation to the eye, and may be held in the eye for longer periods without disturbing the patient. In some embodiments, the exemplary ophthalmic device is configured to reside on the bulbar conjunctiva of the eye and includes a shaped body having two distinct surfaces: a posterior surface, configured to conform to the sclera, and an anterior surface, configured to minimize disturbance to the eyelid when the posterior surface resides on the sclera at the bulbar conjunctiva. While explanations were provided in relation to an eye surface, it should be understood that, in some embodiments, as mentioned above, the posterior and anterior surfaces are distinct from each other irrespective of being inside the eye or outside of it. In some embodiments, the posterior and anterior surfaces are shaped differently (e.g., one is convex and the other concave, one curved and the other flat, etc.). In some embodiments, preferably, both surfaces are smooth, to minimize irritation to the eye and to the eyelid. In some embodiments, the retention portion may be defined between a portion of the posterior surface and a corresponding portion of the anterior surface. In some embodiments, the retention portion is shaped with a sharp ascend, for example, from the edge of the ophthalmic device towards its center. In some embodiments, the ascend may be referred to as sharp if an angle between a reference surface and the ophthalmic device is larger than 45°. In some embodiments, the reference surface may be the eye surface on which the drug product rests. Alternatively, the reference surface may be any surface, flat or convex, on which the ophthalmic device rests. In some embodiments, the reference surface is defined as a surface (either real or imaginary) facing the posterior surface of a device in a relaxed configuration. In some embodiments, the sharp ascend is not from an edge of the ophthalmic device but between two inner points of the ophthalmic device. In some embodiments, the sharp ascend is from a leading edge of the ophthalmic device inwards. An edge may be referred to as leading, if it is disposed adjacent to an opening of an eyelid when the ophthalmic device is underneath that eyelid. In some embodiments, the retention portion may be defined by heights between the posterior surface and the anterior surface. In some embodiments, each such height may be measured from a point on the posterior surface (hereinafter the posterior point) to a corresponding point on the anterior surface (hereinafter the anterior point). In some embodiments, at the region of sharp ascend, two heights distanced from each other by 1 mm or less differ in height by 1 mm or more. In some embodiments, the ophthalmic device is configured for self-administration, that is, the ophthalmic device is configured to be properly inserted to the eye by the patient himself or in homecare. In some embodiments, the ophthalmic device configured for self-administration may be, for example, large enough to be conveniently handled by the patient. For this, the ophthalmic device may be large enough, for example, it may be at least 3 mm long along each dimension parallel to the eye. In some embodiments, the thickness of the ophthalmic device is at least 0.2 mm, the minimal weight is 3 mg, it is visible (e.g., opaque and/or colored) and symmetric. In some embodiments, as for the symmetry, it is advantageous that the ophthalmic device will find an optimal position on the eye regardless of the specific way the patient inserted it. In some embodiments, the minimum weight of the device is 8 mg.

### 6. Exemplary Use of an Exemplary Ophthalmic Device in the Treatment for Presbyopia

### 6.1 Introduction

Presbyopia is a physiological condition associated with the aging of the eye that results in progressively worsening ability to focus clearly on close objects. Also known as age-related farsightedness or age-related long sight, it affects many adults over the age of 40. A common sign of presbyopia is difficulty reading small print which results in having to hold reading material farther away. Other symptoms associated can be headaches and eyestrain.

Presbyopia is usually treated by wearing eyeglasses, but many patients prefer coping with their presbyopia without wearing glasses or contact lenses, so in recent years, some active pharmaceutical ingredients (API) that may help in temporarily ameliorating the symptoms have been identified and suggested for use. One (and possibly only) such commercially available drug product is VUITY® eye drops by Allergan, Inc., which includes pilocarpine as an active pharmaceutical ingredient (API).

In some embodiments, the exemplary ophthalmic device (having a composition as disclosed in Chapter 3, 4 or 5 and optionally a shape/form as disclosed in Chapter 2 or 5, is used for the delivery of active pharmaceutical ingredients (API) for the treatment of Presbyopia.

### 6.2 Exemplary Active Pharmaceutical Ingredients (API) for Treating Presbyopia

In some embodiments, exemplary active pharmaceutical ingredients (API) for the treatment of Presbyopia include one or more of the following: Pilocarpine, Phentolamine Mesylate, Oxymetazoline, Carbachol, Phospholine Iodine, Brimonidine, Aceclidine, and Lipoic acid choline ester.

6.3 Exemplary Method of Preparing and Ophthalmic Device with the Active Pharmaceutical Ingredient (API) for the Treatment for Presbyopia

In some embodiments, an exemplary method of preparing an ophthalmic device (as disclosed elsewhere herein) with the addition of at least one active pharmaceutical ingredient (API) for the treatment of Presbyopia, comprises:
a. Preparing the formulation for the ophthalmic device as described for example in Chapter 3 or Chapter 4.
b. Adding buffer solution until reaching a pH suitable for the active pharmaceutical ingredient (API).
c. Mixing for 5 to 60 minutes.
d. Adding the at least one active pharmaceutical ingredient (API) (for example pilocarpine).
e. Mixing for 5 to 60 minutes.
f. Adding the mixture into the cast.

In some embodiments, the total quantity of water in the formulation of the ophthalmic device, e.g., for the treatment of Presbyopia is no more than 50% wt of the ophthalmic device. In some embodiments, a potential advantage of having no more than 50% wt of water in the ophthalmic device is that the other materials included in the ophthalmic device (not counting the active pharmaceutical ingredient (API)) contribute to potentially reducing the disturbances caused by the API itself (as described herein).

In some embodiments, the buffer preferably has minimal interaction with both the active material and the matrix, e.g. will not compromise the active material stability and will cause minimal additional cross-linking. In some embodiments, with active pharmaceutical ingredients (API) requiring acidic environment (e.g., pilocarpine), the buffer preferably exhibits an acidic pH (e.g., of from 1 to 6, or from 3 to 5). Suitable buffers exhibiting acidic pH include, for example, buffers based on citrates, phosphates, acetates, gluconic acids and/or lactic acid. In some embodiments, with active pharmaceutical ingredients (API) requiring neutral or basic environment, the buffer preferably exhibits a neutral or basic pH (e.g., of from 7 to 14, or from 7 to 12). Exemplary such buffers include, for example, buffers based on hydroxyalkyl amines and/or sulfonic acids, such as, for example, TRIS (tris(hydroxymethyl)aminomethane), HEPES (tris(hydroxymethyl)aminomethane), and the like.

### 6.4 Exemplary Potential Advantages of Using the Exemplary Ophthalmic Device for the Delivery of Active Pharmaceutical Ingredients (API)

To date, it is known to deliver to the eye active pharmaceutical ingredients (API) for the treatment of Presbyopia by using eye-drops. In some embodiments, potential advantages of using the exemplary ophthalmic device as disclosed herein are:

Potentially incurring less irritation to the eye: this is allowed due to the slow release of the active pharmaceutical ingredients (API) in contrast to the very concentrated and immediate exposure of the eye to the active pharmaceutical ingredients (API) when using eye-drops. Additionally, in some embodiments, the ophthalmic device comprises large amounts of demulcents, that at least some of them are released together with the release of the active pharmaceutical ingredients (API). In some embodiments, the demulcents potentially reduce the irritation to the eye. While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

Potentially allowing more of the active pharmaceutical ingredients (API) to be absorbed: this is allowed since the ophthalmic device allows for a longer residence time and/or release time in comparison, for example, to eye-drops, and such longer times may provide a longer absorption window.

Potentially creates similar constriction effect with lower dose, for example, the inventors successfully treated presbyopia using less than half of the pilocarpine amount in a single drop of Vuity™ eye-drops.

Potentially allowing delivering an exact amount of active pharmaceutical ingredients (API): In eye drops, the amount of active pharmaceutical ingredients (API) delivered by a single drop depends on the size of the drop (which may depend on the angle by which the dispenser is held in respect to the eye), and what part of the drop actually entered the eye. Other reasons for delivering inexact amount of active pharmaceutical ingredients (API) may include not following the instruction for applying eye drops (there are many versions of such instructions, that change among products, and many users are not aware of them), size of the drop varies from drop to drop, drop location on the eye may vary between application, head tilting may vary between applications, pocket size under eyelid may vary between users, etc. In contrast, an ophthalmic device as described herein carries a predetermined amount of active pharmaceutical ingredients (API), and all of it is slowly released to the surface of the eye.

Potentially providing exact repetitive administrations: as eye drops may vary from one another in size, as described above, the amount of active pharmaceutical ingredients (API) delivered to an eye each time an eye drop is applied to that eye may differ, for example, depending on the size of the drop. The ophthalmic device, on the other hand, is a unitary dosage form, and all the units in a series may be made identical, allowing repeated delivery of the same amount of active pharmaceutical ingredients (API) by each device of the series.

Potentially allowing to personalize the dose of the active pharmaceutical ingredients (API) for each patient: since the ophthalmic device allows the delivery of a known amount of active pharmaceutical ingredients (API), a specific user can try ophthalmic devices having different amounts of active pharmaceutical ingredients (API) and see which one is more suitable for his needs.

Potentially easier to apply: some patients find it difficult to apply eye-drops. The use of the ophthalmic device as disclosed herein might potentially reduce these difficulties, since the ophthalmic device is easy to apply, does not require head tilting, and does not disturb the user when positioned under the eyelid.

Potentially increases patient compliance, thanks to, for example, being comfortable, having reduced side effects, having a longer therapeutic effect, etc.

Potentially reduces the number of required administrations since the administrations are ensured and controlled.

Potentially reduces the side effect of retinal detachment: retinal detachment occurs when there are too strong forces to constrict the pupil. Such forces may be caused for example by a large quantity of the active pharmaceutical ingredients (API) being provided abruptly, for example as happens with eye-drops. Since the ophthalmic device releases the active pharmaceutical ingredients (API) slowly and over a longer period of time, the effect on the pupil may be reduced and/or achieved more slowly.

Potentially reduces headaches as side effect, which may be caused, for example, when a large quantity of the active pharmaceutical ingredients (API) is provided abruptly, for example as happens with eye-drops.

### 6.5 Exemplary General Mechanism of Action of the Ophthalmic Device in the Treatment for Presbyopia

In some embodiments, the active pharmaceutical ingredients (API) is eluted for a time period of between 5 minutes and 12 or 24 hours, including any intermediate values and subranges therebetween.

Referring now to the table below (Table 3) showing the release rate of an exemplary active pharmaceutical ingredients (API) from an exemplary ophthalmic device, according to some embodiments of the invention.

TABLE 3

| | % released from exemplary |
|---|---|
| Arbitrary time units | ophthalmic device |
| 0 | 0 |
| 0.2 | |
| 1 | 50 |
| 2 | 70 |
| 3 | 80 |
| 6 | 90 |
| 12 | 99 |
| 0 | 0 |

As can be seen in the table, the active pharmaceutical ingredients (API) is slowly released from the matrix of the ophthalmic device. In some embodiments, the rate of release depends on the formulation of the ophthalmic device, the nature of the active pharmaceutical ingredients (API), and/or the interaction between them.

In some embodiments, the user does not need to remove the ophthalmic device, as it is configured to degrade in the eye until nothing is left.

### 6.6 Exemplary Preliminary Clinical Results

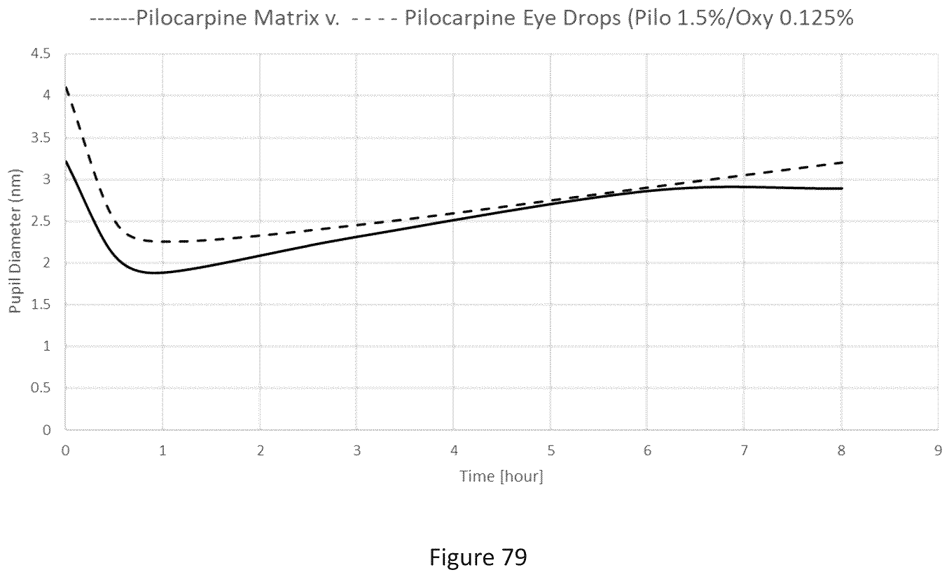

Referring now to FIG. 79, showing a graph plotting the results of a preliminary clinical trial in comparison with published data related to eye-drops.

In the graph, the solid line represent the preliminary clinical results obtained with an ophthalmic device according to an embodiment of the present disclosure, containing pilocarpine as a sole API, while the dotted line represents the published results related to eye-drops (Combinations of Pilocarpine and Oxymetazoline for the Pharmacological Treatment of Presbyopia, Francis W. Price Jr. et al, American Academy of Ophthalmology—www(dot)ncbi(dot)nlm(dot)nih(dot)gov/pmc/articles/PMC9562347/.

In the publication, eye-drops tested in the clinical trial contained 1.5% pilocarpine and 0.0125% oxymetazoline. Thus, a 40 μl drop contained 600 μg pilocarpine. The authors of the publication averaged the results over 31 patients, selected for initial large pupil. The data shows results after 14 days of daily usage, which they report to have cumulative effect.

The preliminary clinical trial consisted of data averaged from 11 patients, not selected for pupil size. The results of the preliminary clinical trial are for the first time the ophthalmic device was applied. The ophthalmic device contained only 200 μg of pilocarpine, equivalent to pilocarpine content of a third of a 40 μg drop of the eye-drops used in the publication.

As can be seen in the graph in FIG. 79, the two lines show similar effectiveness, despite of the device according to the present disclosure containing half to third of the amount of the active pharmaceutical ingredients (API) in one eye-drop of the publication, and a single use of the device in comparison to repeated use of the eye-drops. Additionally, the time at which the pupil returned to a diameter of 3 mm is much longer (more than 8 hours) using the ophthalmic device as disclosed herein than using the eye-drops (about 6 hours).

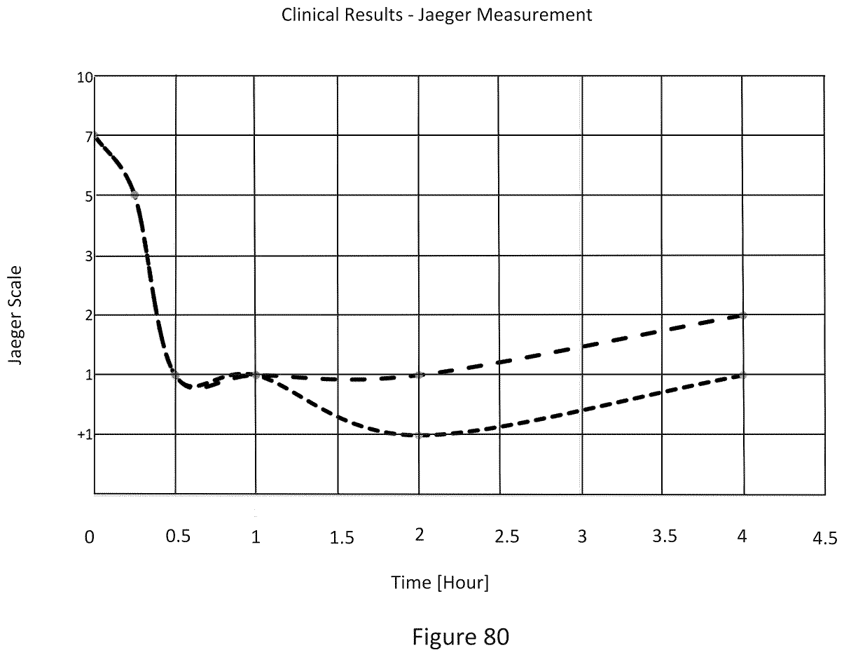

Referring now to FIG. 80, presenting a graph showing test results of near visual acuity (NVA) for two patients treated with an exemplary matrix described herein. The first point was measured before the matrix was applied to the eye. The second point was measured after application of an ophthalmic disclosed herein, containing 200 μg of pilocarpine. In both patients, improvement of 6 points in Jaeger scale can be seen. The effectiveness of NVA improvement treatment is usually reported as the percentage of patients that improved by 2 points or more on the same scale. Thus, FIG. 80 shows that the ophthalmic device achieves not only pupil size reduction (as illustrated by FIG. 79) but also effective treatment for presbyopia.

### 7. Exemplary Use of an Exemplary Soft Elastomeric Ophthalmic Device in the Treatment of Dry Eye Disease

### 7.1 Introduction

Dry eye disease is a common condition that occurs when tears are not able to provide adequate lubrication to the eyes. There are many potential causes of dry eye disease. For example, dry eye may occur if the eye does not produce enough tears or if the eye produces poor-quality tears. Tear instability may lead to inflammation and damage of the eye's surface. Treatments for dry eyes include lifestyle changes and eye drops.

In some embodiments, the exemplary ophthalmic device (having a shape/form as disclosed in Chapter 2, and a composition as disclosed in Chapter 3 or Chapter 4) is used for the delivery of agents for the treatment of dry eye.

### 7.2 Exemplary General Mechanism of Action of the Ophthalmic Device in the Treatment for Dry Eye

In some embodiments, one or more of the ingredients making the ophthalmic device have a double function of a plasticizer (as described, for example, in Chapter 3 or 4) and as an ophthalmic demulcent, which may act as an active pharmaceutical ingredient (API) for treating dry eye. In some embodiments, all the ingredients making the ophthalmic device (other than the water) are considered ophthalmic demulcents. In some embodiments, a substantial portion of the “non-water” ingredients (e.g., 70% or more, 90% or more, or 95% or more or intermediate portions) are agents active in treating dry eye. More specifically, the degradation of the matrix provides the necessary agents for the treatment of dry eye.

In some embodiments, a user can use the ophthalmic device as needed and it provides treatment for long periods of time, for example, from 5 minutes to 12 hours or 24 hours, as described herein.

21CFR349 provides a list of demulcents, and each of these demulcents may make part of an ophthalmic device as described herein for treating dry eye disease.

In some embodiments, the user does not need to remove the ophthalmic device, as it is configured to degrade in the eye until nothing is left to be removed, for example, because the degradation products (the chemical species formed upon biodegradation of the device) are naturally expelled from the eye as foreign body and/or cleared from the eye with the tears.

### 7.3 Exemplary Potential Advantages of Using the Exemplary Ophthalmic Device for the Treatment for Dry Eye Disease

To date, the most common treatment for dry eye is the application of eye-drops, usually several times a day. In some embodiments, potential advantages of using the exemplary ophthalmic device as disclosed herein are:

Potentially increases compliance since it is easy to use and does not disturb the eye.

Potentially interrupts the tear film less than eye-drops upon application, because the ophthalmic device delivers the ingredients slowly, rather than by a fall of a drop onto the eye. Chronic tear film disruption is one cause to dry eye symptoms, so not interrupting the tear film is important in patients with dry eye more than in other patients.

Potentially provides demulcents in greater amounts than in eye drops (see below table 4).

Potentially provides long availability of the demulcents on the eye (from 5 minutes to 12 or 24 hours).

Potentially provides two phases of demulcents delivery: the demulcents provided to the eye in the first phase, are those acting as plasticizers, and any demulcent dissolved or dispersed in them or in the water or aqueous solution making part of the ophthalmic device. The demulcent provided to the eye in the second phase comprises mainly slowly dissolving or dispersing demulcents, such as some types of PVOH.

### 7.4 Exemplary Demulcent Content Comparison Between the Exemplary Ophthalmic Device and a Typical OTC Eye-Drop

The following table (Table 4) shows a comparison between the content of demulcents in an exemplary ophthalmic device as disclosed herein and in a drop of an OTC eye-drop product.

TABLE 4

| | In one 10 mg | In one 50 mg |
|---|---|---|
| | exemplary | drop of OTC |
| | ophthalmic device | product |
| PVOH [mg] | 0.5-3 | 0.05-2.0 |
| PEG [mg] | 0-3 | 0.1-0.5 |
| Glycerol [mg] | 0-6 | 0.1-0.5 |
| Propylene glycol [mg] | 0-6 | 0.1-0.5 |

The content of the ophthalmic device is calculated for a 10 mg product (dry material), so the total equals 10 mg. Products of up to about 40 mg (dry material) may be used, and they may contain up to 4 times the demulcents detailed in the table.

The content of the OTC eye-drop product is calculated based on the range of allowed concentrations in OTC products according to 21CFR349 (FDA regulation), multiplied by 50 mg.

21CFR349 provides a list of demulcents (incorporated herein by reference), and each of these demulcents may make part of an ophthalmic device as described herein for treating dry eye disease. The demulcents may be selected for use in an ophthalmic device for treating dry eye by their effect on the various desired properties of the ophthalmic device as described, for example, in Chapters 3 or 4. Such properties may include, for example, pliability, limited or no swelling in the eye, for example, in the first 5 to 15 minutes, etc.

21CFR349 also provides a list of active pharmaceutical ingredients (API) for treating dry eye, which are not demulcents. Each of these active pharmaceutical ingredients (API) may also make part of an ophthalmic device for treating dry eye, for example, when it is dissolved or dispersed in another ingredient of the matrix, for example, in a demulcent or non-demulcent plasticizer. While in some embodiments, an ophthalmic demulcent may be considered a pharmaceutically active ingredient, whenever a drug product or ophthalmic device is described in the present disclosure and claims as comprising both an API and ophthalmic demulcents, an ophthalmic demulcent, for example, each of the following ophthalmic demulcents, is not considered an API: PVOH, dextran, gelatin, povidone, hyaluronic acid or pharmaceutically acceptable salt thereof, glycerol, polyethylene glycol (e.g., PEG 300 or PEG 400), propylene glycol, polysorbate (e.g., polysorbate 70), carboxymethyl cellulose sodium, hydroxyethyl cellulose, methylcellulose, and hydroxypropyl methylcellulose (HPMC).

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not change by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Exemplary Elastomeric Matrix

### Example 1

### Preparation of an Elastomeric Matrix

The preparation of an elastomeric matrix according embodiments of the present invention, may start with the preparation of a PVOH solution.

Briefly, 420 ml of purified water were added into 500 ml round bottom flask equipped with an overhead stirrer. The flask was placed into a heating mantle, and 80 gr of PVOH were added in portions while stirring and heating until all the PVOH dissolved.

The PVOH solution was left to stir and heat for additional 1-2 hours, and thereafter the solution was left to cool to room temperature while stirring.

Plasticizers and 80 gr of the PVOH solution were added in a 500 ml glass beaker to obtain a mixture according to table 5 (dry matter basis). Optionally purified water was added to dilute concentration of solutes. The mixture was heated with magnetic stirring until fully dissolved, and thereafter heating was turned off and the mixture was left to cool.

Table 1 presents some exemplary formulations for preparing exemplary elastomeric matrices as provided herein. The values represent mass content in percent of the total weight of non-water ingredients.

TABLE 5

| | | | | | | | | | | | | | | |
|---|---|---|---|---|---|---|---|---|---|---|---|---|---|---|
| PVOH | 33 | 25 | 20 | 20 | 20 | 15 | 15 | 15 | 15 | 10 | 10 | 10 | 10 | 5 |
| Polyethylene glycol | 35 | 25 | 25 | 20 | 15 | | 10 | 5 | 20 | 15 | 10 | 20 | 5 | 5 |
| Glycerol | 20 | 25 | 30 | 60 | 34 | 35 | 30 | 40 | 30 | 37 | 40 | 35 | 37.5 | 45 |
| Propylene glycol | 12 | 25 | 25 | | 30 | 35 | 30 | 40 | 31 | 34 | 40 | 35 | 37.5 | 45 |
| Poly propylene | | | | | | 15 | | | | | | | | |
| glycol | | | | | | | | | | | | | | |
| Kollicoat ® IR | | | | | | | 15 | | | | | | 10 | |
| Tromethamine | | | | | 1 | | | | | | | | | |
| Dextran | | | | | | | | | 4 | | | | | |

### Example 2

### Comparative Study

In order to study the criticality of the mass ratio between PVOH and the plasticizers, 20 gr of PVOH were mixed with 36 gr glycerol in a 500 ml glass beaker, and water was added thereto in order to achieve a solution of 20% wt PVOH. It is noted herein that this formulation for producing a matrix that is characterized by a mass content plasticizer to PVOH ratio of 36:20, namely 1:1.8, which is outside the scope of the present invention. The mixture was heated until complete dissolution. Heating was turned off and the solution was left to cool.

The resulting elastomeric matrix show inferior stability in both dry and wet conditions compared to a comparable matrix wherein the mass content ratio of plasticizer to PVOH is 2:1.

Another comparative example was prepared according to the procedure described hereinabove, using 35% wt of high molecular weight PVOH, 11% wt of polyethylene glycol, 27% wt of glycerol, and 27% wt of propylene glycol.

This formulation produced substances that exhibited swelling in water of more than 20%, and have not retained the shape it had under dry conditions.

### Example 3

### Preparation of Device

A solution of each of the various formulations specified in Table 5 was poured into a respective mold (open or closed) and left to dry. Optionally, a coating machine was used to create a film of desired thickness. The film was left to dry.

### Example 4

### Composite Elastomeric Matrices

To prepare a composite elastomeric matrix, according to some embodiments of the present invention, a formulation according to Table 5 was prepared and 10 gr of the formulation were mixed with a non-PVOH polymer in order to reach a ratio of polyvinyl alcohol to the non-PVOH polymer according to Table 6 below. The mixture was stirred until complete dissolution and further processed to create a device (e.g., a film).

TABLE 6

| | Ratio of PVOH |
|---|---|
| | to non-PVOH |
| Non-PVOH polymer | polymer |
| Sodium carboxymethyl cellulose (CMC Na) | 100:1 |
| Chitosan | 90:10 |
| Hydroxypropyl methylcellulose (HPMC) | 100:1 |
| Hydroxypropyl cellulose (HPC) | 100:1 |
| Polyvinyl pyrrolidone (PVP) | 75:25 |
| Polyacrylic acid | 75:25 |
| Dextran | 80:20 |

### Exemplary Elastomeric Matrix Comprising Two or More Types of PVOH

### Example 5

### Preparation of an Elastomeric Matrix

The preparation of an elastomeric matrix according embodiments of the present invention, may start with the preparation of a PVOH solution comprising two types of PVOH. Optionally, the two (or more) types of PVOH together are mixed with the water as described below. Alternatively, two separate mixtures of PVOH are prepared, and then mixed together according to the required ratio between the two types of PVOH and the PVOH concentrations in the mixtures. Alternatively, one PVOH is prepared as a solution, and the other is added AS IS, for example with the plasticizers.

Briefly, 420 ml of purified water were added into 500 ml round bottom flask equipped with an overhead stirrer. The flask was placed into a heating mantle, and 80 gr of PVOH (e.g., 40 gr of PVOH of a first type and 40 gr of PVOH of a second type) were added in portions while stirring and heating until all the PVOH dissolved.

The PVOH solution was left to stir and heat for additional 1-2 hours, and thereafter the solution was left to cool to room temperature while stirring.

Plasticizers and 80 gr of the PVOH solution were added in a 500 ml glass beaker to obtain a mixture according to table 7 (dry matter basis). Optionally purified water was added to dilute concentration of solutes. The mixture was heated with magnetic stirring until fully dissolved, and thereafter heating was turned off and the mixture was left to cool.

Table 7 presents some exemplary formulations for preparing exemplary elastomeric matrices as provided herein. The values in rows 1, 7, 8, and 9 represent mass content in percent of the total weight of non-water ingredients. In table 7, the total amount of PVOH is provided in row 1. This amount may be divided between two or more types of PVOH, for example, as depicted in rows 2 to 6, showing the percentage of each type of PVOH out of the total amount of the PVOH, shown in row 1. In some embodiments, one type may be long chain highly hydrolyzed PVOH, and the other type may be short chain partially hydrolyzed. The ratio between them may be, for example, between 25% and 75% long chain fully hydrolyzed, and the balance short chain partially hydrolyzed. The inventors were surprised to see that while short chain partially hydrolyzed PVOH alone results in matrices of very low quality (e.g., when immersed in water, such matrices might swell non-isotropically, and add more than 100% to their volume), replacing as little as a quarter of the short chain partially hydrolyzed PVOH with long chain fully hydrolyzed PVOH was enough to provide satisfying matrices.

There are many types of PVOH commercially available, and in the following examples, PVOH from Merck's Emprove® product line was used, except for Sigma Aldrich 8.21039, which is PVOH for synthesis. Average molecular weight, chain length, and hydrolysis degree of the used products are provided in the table above (Table 1).

LCFH₁ and LCFH₂ are two different types of long chain fully hydrolyzed PVOH taken from the table above. Similarly, SCPH₁ and SCPH₂ are two different types of short chain partially hydrolyzed PVOH taken from the table above.

TABLE 7

| | # | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|---|
| 1 | Total PVOH | 30 | 25 | 25 | 20 | 20 | 20 | 20 | 15 | 15 | 15 | 15 | 10 | 10 | 10 | 10 | 10 | 5 | 5 |
| 2 | % LCFH1 | 30 | 60 | 25 | 30 | 35 | 30 | 35 | 65 | 50 | 30 | 40 | 80 | 70 | 80 | 75 | 50 | 90 | 90 |
| 3 | % SCPH1 | | | | | | 70 | | | 50 | 70 | | 20 | | | | | 10 | |
| 4 | % SCPH2 | | | 75 | | | | 65 | | | | 60 | | 30 | 20 | 25 | | | |
| 5 | % LCFH2 | | 40 | | 70 | 65 | | | 35 | | | | | | | | 50 | | 10 |
| 6 | % LCPH | 70 | | | | | | | | | | | | | | | | | |
| 7 | Polyethylene | 10 | 10 | 10 | 25 | 20 | 25 | 20 | 5 | 25 | 5 | 15 | 15 | 10 | 15 | 10 | 20 | 5 | 5 |
| | glycol | | | | | | | | | | | | | | | | | | |
| 8 | Glycerol | 30 | 32.5 | 32.5 | 30 | 60 | 30 | 60 | 40 | 30 | 40 | 30 | 37 | 40 | 37 | 40 | 35 | 45 | 50 |
| 9 | Propylene | 30 | 32.5 | 32.5 | 25 | | 25 | | 40 | 30 | 40 | 40 | 38 | 40 | 34 | 40 | 35 | 45 | 40 |
| | glycol | | | | | | | | | | | | | | | | | | |

### Example 6

### Preparation of Device

A solution of each of the various formulations specified in Table 5 or in Table 7 was poured into a respective mold (open or closed) and left to dry. Optionally, a coating machine was used to create a film of desired thickness. The film was left to dry.

### Example 7

### Clinical Results

An ophthalmic device comprising equal amounts of long chain fully hydrolyzed PVOH and short chain partially hydrolyzed PVOH with no active pharmaceutical ingredients (API) other than the ophthalmic demulcents (PVOH, glycerol, propylene glycol, and PEG 400) was administered for 3 to 6 hours to each of the participants. The participants were 29 healthy people, (ages 18 to 60). The participants reported the device was almost imperceptible, probably thanks to the degradation properties of the matrix, which allowed the device to fit itself to the individual eye anatomy of the wearer. The devices retained in the eyes, distance visual acuity has not declined, and slip lamp bio-microscopy including fluorescein corneal staining conducted after removing the device from the eye, showed no significant difference from the same tests performed before the administration.

When taken out of the eye, the devices have been softer than before insertion to the eye. Some of the devices were taken out of the eye as crumbs. Some of the devices were dried and weighed after being taken out of the eye. Their weight was found to be about 70% lower than the weight of the device before it was inserted to the eye.

### Dry Eye Disease

### First in Human

Nearly 40 healthy subjects experienced using ophthalmic devices according to the invention for 6 hours. All reported that the device was easy to use and almost imperceptible, probably thanks to the degradation properties of the matrix, which allowed the device to fit itself to the individual eye anatomy of the wearer. No serious adverse events were observed during the study. Few participants experienced mild discomfort or tearing in the initial few minutes after the application of the device, which disappeared spontaneously later on (within 15 minutes).

According to Visual Analogue Scale (VAS) questionnaires, wear of the device over time was mostly imperceptible and no discomfort was reported at the end of the treatment before removal of the device. The device was found safe for use.

During the treatment, there were reports of enhanced moisture in the treated eye, improved feeling (eyelid easily sliding), relaxation of the eye, and improved or clear vision, probably related to tear film stabilization. In some subjects, who reported some level of dryness in their eyes at baseline, there was an improvement in their dryness symptoms. Some subjects applied the device independently, and most subjects easily removed the device by themselves.

When taken out of the eye, the devices have been softer than before insertion to the eye. Some of the devices were taken out of the eye as crumbs. Some of the devices were dried and weighed after being taken out of the eye. Their weight was found to be about 70% lower than the weight of the device before it was inserted to the eye.

### 5 Days Trial

10 patients suffering from mild to moderate dry eye disease took part in a clinical trial, in which each patient inserted under the lower eyelids of both eyes an ophthalmic device according to an embodiment of the present invention; consisting essentially of water, PVOH, and plasticizers Once daily for 5 consecutive days.

All the participants were trained to apply the device by themselves. All participants reported that the device was easy to use and almost imperceptible, probably thanks to the degradation properties of the matrix, which allowed the device to fit itself to the individual eye anatomy of the wearer. No serious adverse events were observed during the study. Few participants experienced mild discomfort or tearing in the initial few minutes after the application of the device, which disappeared spontaneously later on, within 15 minutes from application. No serious adverse events were observed throughout the 5-day study. According to Visual Analogue Scale (VAS) questionnaires, wear of the device over time was mostly imperceptible, at least 15 minutes from the beginning of the daily treatment until removal of the device. The device was found safe for use.

When taken out of the eye, the devices have been softer than before insertion to the eye. Some of the devices were taken out of the eye as crumbs. Some of the devices were dried and weighed after being taken out of the eye. Their weight was found to be about 70% lower than the weight of the device before it was inserted to the eye.

### Presbyopia

A total of 18 subjects diagnosed with presbyopia, aged 45-61 were enrolled in the study. All subjects completed the study as planned. Following insertion of a device according to an embodiment of the invention, comprising 200 mcg of pilocarpine, under an eyelid of one of the eyes, mean pupil diameter decreased compared to the untreated eye, the mean minimum pupil diameter in the treated eye observed 1 hour after insertion. The mean pupil diameter in the treated eye increased over time up to 8 hours post-insertion. Two hours after insertion, monocular near visual acuity measured using the Jaeger chart decreased to J1 in all subjects but one, who had J1+. BCDVA remained at 6/6 during the study.

All participants reported that the device was easy to use and almost imperceptible, probably thanks to the degradation properties of the matrix, which allowed the device to fit itself to the individual eye anatomy of the wearer. No serious adverse events were observed during the study. Some subjects reported minor discomfort for up to 5 minutes after insertion. All subjects reported that the device was easy to use and almost imperceptible. IOP remained within normal range during the study. No clinically significant changes in slit lamp bio-microscopy were observed between baseline and endpoint.

### Example 8

### Mechanical Properties

### Compression Module

Compression module of an elastomer (or any other specimen) is the slope of the elastic portion of a stress-strain graph measured when the elastomer is compressed. The table below shows compression module of some elastomeric matrices according to some embodiments of the invention, as function of composition and immersion time in STF (simulated tear fluid). The only difference between the first 6 matrices is the types of PVOH used in the formulations, and their relative amounts. The seventh formulation further differs from the first six in the total amount of PVOH and the various plasticizers.

| Time in STF | | | | | | | |
|---|---|---|---|---|---|---|---|
| before the | 100% | 50%:50% | 30%:70% | 70%:30% | 50%:50% | 30%:70% | 50%:50% |
| measurement | LCFH | LCFH:SCPH | LCFH:SCPH | LCFH:SCFH | LCFH:SCFH | LCFH:SCFH | LCFH:LCPH |
| 0 min [MPa] | 1.0 | 0.84 | 0.45 | 0.73 | 0.62 | 0.60 | 0.64 |
| 5 min [MPa] | 0.55 | 0.36 | 0.14 | 0.43 | 0.35 | 0.46 | 0.14 |
| 15 min [MPa] | 0.28 | 0.14 | 0.06 | 0.20 | 0.18 | 0.25 | 0.14 |
| 75 min [MPa] | 0.16 | 0.04 | 0.03 | 0.10 | 0.07 | 0.06 | Dissolved |

As can be seen in the table above, in all the matrices, the compression module decreases during immersion in STF. At any given time, when some of the LCFH PVOH is replaced with SCPH PVOH, matrices of different compositions differ from each other in the compression module, with matrices having more SCPH PVOH having lower compression module. When different portions of LCFH PVOH are replaced with SCFH PVOH, there is no clear trend in the differences between the compression modules of the matrices at any given time, and the differences between them are within the estimated error (i.e., the differences are not meaningful).

In some embodiments, the change in compression module is an indication of the degradation of the matrix. Thus, the results, summarized in the above table in regard of matrices in which some of the LCFH PVOH is replaced by SCPH PVOH, demonstrate that the different amounts of the two types of PVOH determine the rate of degradation of these matrices.

The above table also demonstrates that the compression module is not adequate as a measure of the degradation of the other matrices in the table. As will be seen in the next table, for these embodiments, the compression strength is a mechanical property appropriate to demonstrate the matrix degradation.

### Compression Strength

Compression strength is the pressure at which a specimen breaks. In a stress-strain graph, it appears as a sharp drop in the graph. The following table shows compression strength results extracted from the same graphs, from which the compression module values presented in the above table have been extracted. The measurements were done using a 10N probe. When the graph showed no sharp drop, the table says “Max Load”, and this implies that a 10N load is not sufficient to break the matrix by compression.

| Time in STF | | | | | | | |
|---|---|---|---|---|---|---|---|
| before the | 100% | 50%:50% | 30%:70% | 70%:30% | 50%:50% | 30%:70% | 50%:50% |
| measurement | LCFH | LCFH:SCPH | LCFH:SCPH | LCFH:SCFH | LCFH:SCFH | LCFH:SCFH | LCFH:LCPH |
| 0 min | Max | Max | Max | Max | Max | Max | Max |
| 5 min | Load | Load | Load | Load | Load | Load | Load |
| 15 min | | | | | | 0.22 | |
| 20 min | | | | | | 0.20 | |
| 30 min | | | | | | 0.12 | |
| 40 min | | | | | 0.22 | 0.09 | |
| 75 min | | | | 0.08 | 0.11 | 0.08 | |

As can be seen in the table, from all tested compositions, only those in which some of the LCFH PVOH is replaced by SCFH PVOH has broken under the 10N load. Among the matrices that broke, the more LCFH PVOH, the longer should the matrix stay in STF in order to break.

In some embodiments, the change in compression strength is an indication of the degradation of the matrix. Thus, the results, summarized in the above table in regard of matrices in which some of the LCFH PVOH is replaced by SCFH PVOH, demonstrate that the different amounts of the two types of PVOH determine the rate of degradation of these matrices.

The above table also demonstrates that the change in the compression strength (at least when measured with a 10N load) is not adequate as a measure of the degradation of the other matrices in the table. As was shown in the preceding table, for those embodiments, the compression module is a mechanical property appropriate to demonstrate the matrix degradation.

### Swelling

The swelling of four matrices according to embodiments of the present invention, having formulations like the first four shown in the preceding tables were measured by measuring thickness (i.e., height) and diameter of disk-like matrices before immersion in STF and after different periods of immersion. The matrices were imaged, and the images processed to estimate the thickness and the diameter. The results detailed in the following table show that after 15 minutes (or less) of immersion in STF, all the matrices swelled by no more than 50%±5%.

| | | Thickness | Diameter | Volume |
|---|---|---|---|---|
| Formulation | time [Min] | % Swelling | % Swelling | % Swelling |
| LCFH 100% | 2 | 3 | 8 | 19 |
| | 5 | 4 | 11 | 29 |
| | 10 | 9 | 10 | 31 |
| | 15 | 13 | 14 | 47 |
| LCFH 50% | 2 | 0 | 11 | 20 |
| SCPH 50% | 5 | 17 | 9 | 39 |
| | 10 | 24 | 11 | 52 |
| | 15 | 26 | 11 | 55 |
| LCFH 30% | 2 | 0 | 11 | 20 |
| SCPH 70% | 5 | 17 | 9 | 39 |
| | 10 | 8 | 8 | 26 |
| | 15 | 10 | 12 | 38 |
| LCFH 50% | 2 | 0 | 9 | 13 |
| SCFH 50% | 5 | 4 | 8 | 22 |
| | 10 | 13 | 12 | 40 |
| | 15 | 21 | 13 | 54 |

In some embodiments, the maximal swelling observed in the matrices is: At 2 minutes: 21%; At 5 minutes: 40%; At 10 minutes: 52%; At 15 minutes: 55%; At 30 minutes: 75%; All numbers are ±5%

### Simulated Tear Fluid (STF)

The simulated tear fluid (STF) of the following composition: 6.76 gr NaCl; 2.05 gr sodium bicarbonate; 0.08 gr calcium chloride dehydrate; 1.7 mL acetic acid 10%; in 1 Liter of purified water.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An ophthalmic device, configured to reside between an eye surface of an eyeball and an eyelid, comprising:
a body made of an elastomeric matrix, wherein said elastomeric matrix comprises: water, one or more types of PVOH, and one or more organic plasticizers,
said body characterized by:
i. a posterior surface which is adjacent to an eye surface when said ophthalmic device is worn;
ii. an anterior surface, which is adjacent to said eyelid covering said eye surface when said ophthalmic device is worn;
iii. a retention portion, defined between a portion of said anterior surface and a portion of said posterior surface;
wherein a combined mass ratio of said one or more organic plasticizers to said one or more types of PVOH is at least 2:1.

2. The ophthalmic device according to claim 1, wherein said retention portion is configured to cause the ophthalmic device to move with the eyelid when the eyelid moves in respect to the eyeball.

3. The ophthalmic device according to claim 1, wherein an angle of a slope of said anterior surface extending from said posterior surface is larger than 45 degrees.

4. The ophthalmic device according to claim 1, wherein a relation between a width and a length of said ophthalmic device is from about 1:1 to about 1:3.

5. The ophthalmic device according to claim 1, wherein said body swells isotropically when immersed in simulated tear fluid for 15 minutes or less.

6. The ophthalmic device according to claim 1, further comprising at least one ophthalmic acceptable active pharmaceutical ingredients (API).

7. The ophthalmic device according to claim 1, wherein said water makes between 5 wt % and 50 wt % of said elastomeric matrix.

8. The ophthalmic device according to claim 1, wherein said ophthalmic device is characterized by one or more of the following:
a. being configured to gain no more than 50% weight when immersed in simulated tear fluid for 15 minutes or less;
b. being obtainable from a liquid solution comprising water, the ophthalmically acceptable active pharmaceutical ingredient (API), the PVOH, and the water-soluble organic liquids, without freezing;
c. having at least one of the following mechanical properties:
i. tensile strength of between 0.05 MPa and 10 MPa;
ii. Young modulus of between 0.05 MPa and 10 MPa; and
ii. elongation at break of between 50% and 1000%;
d. being configured to lose at least 50% of its weight within 2 hours in an eye;
e. being sized and shaped to be fully covered by the lower eyelid when worn under a lower eyelid;
f. being configured for self-administration;
g. being configured for wearing away from a fornix' eye;
h. being essentially devoid of covalent crosslinking.

9. (canceled)

10. The ophthalmic device according to claim 1, comprising two or more types of PVOH.

11. The ophthalmic device according to claim 10, wherein a ratio between said two or more types of PVOH is such that said ophthalmic device disintegrates under simulated tear fluid (STF) within 24 hours and/or during the first 10 minutes.

12. (canceled)

13. The ophthalmic device according to claim 1, wherein said ophthalmic device is one or more of:
a. visible to naked eye on a background of bulbar conjunctiva;
b. elongated and symmetrical, so that all orientations of the device are equivalent as long as an elongated side of the device lies at a given direction.

14. (canceled)

15. The ophthalmic device according to claim 1, wherein said retention portion is one or more of:
a. defined by heights between said posterior surface and said anterior surface; and wherein a first height of said heights measured from a first point on said posterior surface to a corresponding point on said anterior surface differs by at least 1 mm, from a second height of said heights measured from a second point on said posterior surface to a corresponding point on said anterior surface, said second point being distanced at most 1 mm from said first point;
b. configured to keep said ophthalmic device in place when the device is worn, and the eye is rolled.

16. The ophthalmic device, according to claim 1, wherein said body includes a plurality of ophthalmic lubricants;
wherein at least one of the following is true:
a. each of said plurality of ophthalmic lubricants is selected from the group consisting of polyvinyl alcohol, a polyol, glycerol, polyethylene glycol, propylene glycol, polysorbate, hyaluronic acid or pharmaceutically acceptable salt thereof, cellulose, carboxymethyl cellulose sodium, hydroxyethyl cellulose, methylcellulose, dextran, gelatin, polysorbate, hydroxypropyl methylcellulose, and povidone;
b. said ophthalmic lubricants comprise polyethylene glycol (PEG), and the mass content of the PEG in the elastomeric matrix is about equal that of the PVOH; and
c. at least 35% of said ophthalmic lubricants consist of glycerol and propylene glycol.

17. (canceled)

18. The ophthalmic device according to claim 1, wherein said PVOH comprises PVOH of two or more types that differ from each other by chain length, degree of hydrolysis, or both; and wherein at least 35% wt of said two or more types of PVOH has a degree of hydrolysis lower than 90%.

19-21. (canceled)

22. The ophthalmic device according to claim 1, wherein said elastomeric matrix is impregnated with an ophthalmically acceptable active pharmaceutical ingredient.

23. The ophthalmic device according to claim 1, obtainable by mixing the API with water and ophthalmic demulcents to obtain a mother solution; and drying the mother solution at room temperature; and wherein said obtainable is obtainable without heating one or more of the mother solution and the API.

24-28. (canceled)

29. The ophthalmic device according to claim 1, wherein a first height perpendicular to a reference surface and measured from a first point on said posterior surface to a corresponding point on said anterior surface differs by at least 1 mm, from a second height perpendicular to the reference surface and measured from a second point on said posterior surface to a corresponding point on said anterior surface, said second point being distanced at most 1 mm from said first point.

30-32. (canceled)

33. The ophthalmic device according to claim 1, comprising two or more types of PVOH and wherein at least one of the following is true:
a. each of said two or more types of PVOH constitutes at least 10% of the total amount of PVOH in the matrix;
b. the two or more types of PVOH include one type that has long chains and is fully hydrolyzed, and one type that has at least one of:
i. short chains and is partially hydrolyzed;
ii. long chains and is partially hydrolyzed;
iii. short chains and is fully hydrolyzed;
wherein:
iv. short chains are of 200 to 2000 monomeric units;
v. long chains are of 2200 to 5000 monomeric units;
vi. fully hydrolyzed PVOH is of hydrolysis degree of 97% or more; and
vii. partially hydrolyzed is of hydrolysis degree of 95% or less,
c. two types of the PVOH differ in chain length by at least 1000, and have similar degrees of hydrolysis, both between 97% and 100%;
d. a first type of said two or more types has a hydrolysis degree from 97% to 100%;
e. a second type of said two or more types has a hydrolysis degree is lower than 93%;
f. a second type of said two or more types has a hydrolysis degree from 80% to 93%;
g. a first type of said two or more types has chains with more than 2500 units;
h. a second type of said two or more types has chains with less than 1500 units;
i. a relation between a first type of PVOH and a second type of PVOH of said two or more types is from about 3:1 to about 1:3;
j. a first type of said two or more types has chains with more than 2500 units and hydrolysis degree of 97% to 100%, and a second type of said two or more types has chains with less than 1000 units and hydrolysis degree of 80% to 93%;
k. said second type of PVOH makes more than 50% of said PVOH;
l. said at least two types of PVOH determine rate of degradation of said ophthalmic device; and
m. said rate of degradation comprises a rate by which a predetermined change in a predetermined mechanical property takes place in simulated tear fluid (STF).

34-46. (canceled)

47. The ophthalmic device according to claim 1, wherein a combined mass content of said PVOH and said one or more organic plasticizers is at least 70% wt of the total weight of the matrix excluding said water.

48. The ophthalmic device according to claim 1, wherein said elastomeric matrix is characterized by one or more of:
a. substantially isotropic swelling when being immersed in simulated tear fluid and at room temperature for 5 minutes;
b. swelling by less than 50% by volume when immersed in simulated tear fluid for 5 minutes at room temperature.

49-51. (canceled)
